SEQUENCE LISTING (159 sequences)

SEQ ID NO: 1 - DNA, 16668 bp, Staphylococcus aureus Bacteriophage 44 AHJD
tccatttctttactaaacttaaaaatgctgtgcaacaacttaaccaacttatctaacctattacatattcatcaaatacaaaatttatgtatctattgacttttattcaaaattatgatttcaacatataataaaattaatttacttatttaaatattctatgatataattagttataaaatatttggaggtgtataaatgacagaatttgatgaaatcgtaaaaccagacgacaaagaagaaacttcagaatcaactgaagaaaatttagaatcaactgaagaaacttcagaatcaactgaagaatcaactgaagaatcaactgaagaatcaactgaagataaaacagtagaaacaatcgaagaagaaaatgaaaacaaattagaacctactacaacagatgaagatagttcgaaatttgaccctgttgtattagaacaacgtattgcttcattagaacaacaagtgactacttttttatcttcacaaatgcaacaaccacaacaagtacaacaaacacaatcagatgtaacagaatcaaacaaagaagataacgactattcagatgaagaactagttgataagttagatttagattaggaggaatttaaacatgtatgagggaaacaacatgcgttctatgatgggtacatcatatgaagattcaagattaaataaacgaacagaattaaatgaaaacatgtcaattgatacaaataaaagtgaagatagttatggtgtacaaattcattcactttcaaaacaatcatttacaggtgacgttgaggaggaataataaattatggcacaacaatctacaaaaaatgaaactgcacttttagtagcaaagtcagctaaatcagcgttacaagattttaatcatgattattcaaaatcttggacatttggcgacaaatgggataattcaaatacaatgttcgaaacatttgtaaataaatatttattccctaagattaatgagactttattaatcgatattgcattaggtaatcgttttaattggttagctaaagagcaagattttattggacaatatagtgaagaatacgtgattatggacacagtaccaattaacatggacttatctaaaaatgaggaattaatgttgaaacgtaattatccacgtatggcaactaagttatatggtaacggaattgtgaagaaacaaaaattcacattaaacaacaatgatacacgtttcaatttccaaacattagcagacgcaactaattacgctttaggtgtatacaaaaagaaaatttctgatattaatgtattagaagaaaaagaaatgcgtgcaatgttagttgattactcattgaatcaattatccgaaacaaatgtacgtaaagcaacatcaaaagaagatttagcaagcaaagtttttgaagcaatcctaaacttacaaaacaacagtgctaaatataatgaagtacatcgtgcatcaggtggtgcaattggacaatatacaactgtatcaaaattaaaagatattgtgattttaacaacagattcattaaaatcttatcttttagatactaagattgcaaacacattccagattgcaggcattgatttcacagatcacgttattagttttgacgacttaggtggcgtgtttaaagtaacaaaagaatttaagttacaaaaccaagattcaattgactttttacgtgcgtatggagattatcaatcacaattaggagatacaattccagttggtgctgtatttacttatgatgtatctaaacttaaagagtttactggcaacgttgaagaaattaaaccaaaatcagatttatatgcgtttattttggatattaattcaattaaatataaacgttacacaaaaggtatgttaaaaccaccattccataaccctgaatttgatgaagttacacactggattcattactattcatttaaagccattagtccattctttaataaaattttaattactgaccaagatgtaaatccaaaaccagaggaagaattacaagaataaaaggagcgtaaaatatgaacaacgataaaagaggtttaaacgttgagttatcaaaggaaatcagcaaaagagttgttgaacatcgcaacagatttaaacgtcttatgtttaatcgttatttggaatttttaccgctactaatcaactataccaatcgtgatacggttggtatagattttattcagttagaatcagctttaagacaaaacattaatgtagttgttggtgaagctagaaataagcaaattatgattcttggttatgtaaataacacttactttaatcaagcaccaaatttttcatcaaactttaatttccaatttcaaaaacgattaactaaagaagatatatattttattgtacctgactatttaatacctgatgattgtctacaaattcataagctatatgataactgtatgagtggtaactttgttgtcatgcaaaataaaccaattcaatataatagtgatatagaaattatagaacattatactgatgaattagcagaagttgctttatctcgcttttctttaatcatgcaagcaaaatttagcaagatatttaaatcagaaattaatgacgagtcaatcaatcaacttgtgtccgaaatatataacggtgcaccatttgttaaaatgtcacctatgtttaatgcagatgacgatatcattgatttaacaagtaatagcgtaatcccagcattaactgaaatgaaacgggaatatcaaaacaaaattagtgaattaagtaactatttaggcattaattcattagccgttgataaagaaagcggtgtttcagacgaagaggcaaaaagtaatcgtggatttaccacatcaaacagtaatatctatttaaaaggtcgtgaaccaattacgtttttatcaaagcgttatggtttagatattaaaccgtattacgatgatgaaacaacgtctaaaatatcaatggtagacacactttttaaagatgaaagcagtgatataaatggctagatacacaatgactttatacgatttcattaaatcagaattgattaaaaaaggtttcaatgaatttgtaaatgataataaattaacgttttatgatgatgaatttcaattcatgcaaaaaatgctgaagttcgacaaagacgttttagctatcgttaatgaaaaagtatttaaaggtttttcattgaaagatgaattatcagatttactttttaaaaaatcatttacgattcattttttagatagagaaatcaacagacaaacagttgaagcatttggcatgcaagtgattactgtatgtattacacatgaggattatttaaatgtggtttattcatcaagtgaagttgaaaaatacttacaatcacaaggcttcacagaacacaatgaagatacaacaagtaacactgatgaaacatcgaatcaaaatgctacatctttagacaattcaactggcatgactgcaaacagaaacgcttatgtgtcattaccacaaagtgaggttaacattgatgttgataatacaacgttacgattcgctgataataatacgattgataacggtaaaactgtgaataaatcgagtaacgaaagtaatcaaaacgcaaaacgtaatcaaaatcaaaaaggtaatgcaaaaggtacacaattcactaagcagtatttaattgataatattgataaagcgtacgatttaagaaagaaaattttaaatgaatttgataaaaaatgttttttacaaatttggtagaggtggttaaataatggcatataatgaaaacgattttaaatattttgatgacattcgtccatttttagacgaaatttataaaacgagagaacgttatacaccgttttacgatgatagagcagattataatactaattcaaaatcatattatgattatatttcaagattatcaaaactaattgaagtattagcacgtcgtatttgggactatgacaatgaattaaaaaaacgtttcaaaaattgggacgacttaatgaaagcatttccagagcaagcgaaagacttatttagaggttggttaaacgacggtacgattgacagtattattcatgacgagtttaaaaaatatagcgcaggattaacatcggcatttgctttatttaaagttactgaaatgaaacaaatgaatgactttaaatcagaagttaaagacttaattaaagatattgaccgtttcgttaatgggtttgaattaaatgagcttgaaccaaagtttgtgatgggctttggtggtattcgcaacgcagttaaccaatctattaatattgataaagaaacaaatcacatgtactctacacaatccgattctcaaaaacctgaaggtttttggataaataaattaacacctagtggtgacttaatttcaagcatgcgtattgtacagggtggtcatggtacaacaatcggattagaacgtcaatccaatggtgaaatgaaaatctggttacatcacgatggtgttgcaaaactgttacaagtcgcatataaagataattatgtattagatttagaagaggctaaaggtttaacagattatacaccacagtcacttttaaacaaacacacatttacaccgttaattgatgaagcaaatgacaaactcattttaagattcggtgacggaacaatacaggttcgttcaagagcagacgtaaaaaatcacattgataatgtagaaaaagaaatgacaattgataattcagaaaacaatgataatcgttggatgcaaggcattgctgttgatggtgatgatttatactggttaagtggtaacagttcagttaattcacatgttcaaatcggtaaatattcattaacaacaggtcaaaagatttatgattatccatttaagttatcatatcaagacggtattaatttcccacgtgataactttaaagagcctgagggtatttgcatttatacaaatccaaaaacaaaacgtaaatcgttattacttgctatgacaaacggcggtggtggaaaacgtttccataatttatatggtttcttccaacttggtgagtatgaacactttgaagcattacgcgcaagaggttcacaaaactataaattaacaaaagacgacggtcgtgcattatctattccagaccatatcgacgatttaaatgacttaacgcaagctggtttttattatattgacgggggtactgcagaaaaacttaagaatatgccaatgaatggtagcaagcgtataattgacgctggttgtttcattaatgtataccctacaacacaaacattaggtacggttcaagaattaacacgtttctcaacaggtcgtaaaatggttaaaatggtgcgtggtatgactttagacgtatttacgttaaaatgggattatggattatggacaacaatcaaaactgacgcaccatatcaagaatatttggaagcaagtcaatacaataactggattgcttatgtaacaacagctggtgagtattacattacaggtaaccaaatggaattatttagagacgcgccagaagaaattaaaaaagtgggtgcatggttacgtgtgtcaagtggtaacgcagtcggtgaagtaagacaaacattagaggctaatatatcggaatataaagaattcttcagtaatgttaatgcggaaacaaaacatcgtgaatatggttgggtagcaaaacatcaaaaataggagtgatataaatgaaatcacaacaacaagcaaaagaatggatatataagcatgagggggcaggtgttgactttgatggtgcatatggatttcaatgtatggacttatcagttgcttatgtgtattacattactgacggtaaagttcgcatgtggggtaatgctaaagacgcgataaataatgactttaaaggtttagcgacggtgtataaaaatacaccgagctttaaacctcaattaggggacgttgctgtatatacaaatggacaatatggacatattcaatgtgtgttaagtggaaatcttgattattatacatgcttagaacaaaactggttaggcggcggttttgacggttgggaaaaagcaaccattagaacacattattatgacggtgtaactcactttattagacctaaattttcaggtagtaatagcaaagcattagaaacatcaaaagtaaatacatttggaaaatggaaacgaaaccaatacggcacatattatagaaatgaaaatggtacatttacatgtggttttttaccaatatttgcacgtgtcggtagtccaaaattatcagaacctaatggctattggttccaaccaaacggttatacaccatataacgaagtttgtttatcagatggttacgtatggattggttataactggcaaggcacacgttattatttaccagtgcgccaatggaatggaaaaacaggtaatagttacagtgttggtattccttggggggtgttctcataatgggtattttagcctttttctttgaatttagttggaaaagatacaaataagaggtgtaaacaatggctgatagaatcgtaagaagtttaagacaagttgaaacaattgaacgtttattggaggaaaaaaatgagaaagttaacgaattttaagtttttctataacacaccgtttacagactatcaaaacacgattcattttaatagtaataaagaacgtgatgattattttttaaatggtcgtcattttaaatcgttagactattcaaaacaaccgtataattttatacgtgatagaatggaaatcaatgttgatatgcagtggcatgacgcacaaggtattaactacatgacgtttttatcagattttgaggatagaagatattacgcttttgtaaaccaaatcgaatacgtgaatgacgttgtggttaaaatatattttgtcattgataccattatgacgtatacacaagggaatgtattagagcaactctcaaacgtcaatattgaacgtcaacatttatcaaaacgcacgtataactatatgttaccaatgttacgtaataatgatgatgtgttaaaagtatcaaataaaaactatgtttataaccaaatgcaacaatatttggaaaatttagtattattccagtcaagcgctgatttatcaaagaaatttggtactaaaaaagagccaaacttagatacgtcaaaaggtacgatttatgacaatatcacatcaccagtcaacttatacgttatggaatatggtgactttattaactttatggataaaatgagtgcctatccatggattacgcaaaactttcaaaaggttcaaatgttacctaaagactttattaatacaaaagacttagaggacgttaaaaccagtgaaaaaattacaggattaaaaacattaaaacagggtggtaaatcaaaagaatggagtctaaaagatttatcattaagtttctcaaatcttcaagagatgatgttatctaaaaaagatgaatttaaacatatgatacgtaatgagtatatgacaattgaattttatgactggaatggaaatacgatgttactcgacgctggtaagatttcacaaaaaactggtgttaagttacgtacaaaatcaattattggttatcataatgaagttcgagtatatccagtagattataacagtgctgaaaacgacagaccaatactcgctaaaaataaagaaatattgattgatacgggttcattcttaaatacaaatataacatttaatagttttgcacaagtaccaatattaatcaataatggtatcttaggacaatcacaacaagccaaccgacaaaaaaatgcagaaagtcaattaattacaaatcgtattgataatgtattaaatggtagcgacccgaaatcacgcttttatgacgctgtgagtgtagcaagtaatttaagtccaactgctttatttggtaagtttaatgaagaatataatttctacaaacaacaacaagctgaatataaagatttagccttacaaccaccttctgtaactgaatcagaaatgggcaacgcattccaaattgcgaatagcattaacggtttaacgatgaaaattagtgtaccgtcacctaaagaaattacatttttacaaaaatattatatgttgtttggttttgaagtgaatgactataattcatttattgaaccaattaacagtatgactgtttgcaattatttaaaatgtacaggtacgtatactatacgtgacatcgaccccatgttaatggaacaattaaaagcaattttagaatctggtgtaagattttggcataatgacggttcaggtaatccaatgttacaaaatccattaaataacaaatttagagagggggtataatatgaacgaagtaaaattcagatttacagactcagaagcgtttcacatgtttatatacgctggggatttaaaattactctactttttatttgtattaatgttcgttgatattattacaggtatttcaaaagcaattaaaaataataacttatggtcaaaaaaatcaatgagaggattttctaaaaaattattgatattctgtattatcattttagcaaacatcattgaccagattttacaattaaaaggtggtctactcatgattacaatattttattatattgcaaatgagggactttctattgtagaaaattgtgcagaaatggacgtattagtaccagaacaaattaaagataaattaagagtcattaaaaatgatactgaaaagagtgataacaatgaacgatcaagagaagatagataaatttacgcattcctatattaatgatgattttggtttaacgatagaccagttagtccctaaagtaaaaggatatgggcgctttaatgtatggcttggtggtaatgaaagtaaaatcagacaagtattaaaagcagtaaaagagataggtgtttcacctactctttttgccgtatatgaaaaaaatgagggttttagttctggacttggttggttaaaccatacgtctgcacgtggtgattatttaacagatgctaaattcatagcaagaaagttagtatcacaatcaaaacaagctggacaaccgtcttggtatgacgcaggtaacatcgtccactttgtaccacaagacgtacaaagaaaaggtaatgcagattttgcaaaaaatatgaaagcaggtacaattggacgtgcatatattccattaacagcagctgctacttgggcggcatattatcctttaggtttgaaagcatcatataacaaagtacaaaactatggtaatccatttttagacggtgcgaatactattctagcttggggtggtaaattagacggtaaaggtggatcacctagtgattcgtctgacagtggtagtagtggtgacagtggtagttcactactcgctttagcaaaacaagccatgcaagaattattaaaaaaaatacaagacgcattacaatgggacgttcatagtattggtagtgataaattttttagtaatgattattttacattagaaaaaacatttaacaacacatatcatattaaaatgacgattggtttacttgattcattaaaaaaactgattgatagcgttcaagtagatagtgggagtagtagttctaatcctactgatgatgacggagaccataaaccaattagtggtaaatcagtcaagccaaatggaaaaagtggtcgtgtgattggtggtaactggacatatgcacagttaccagaaaaatataaaaaagcaattggtgtacctttattcaaaaaagaatacttatacaaaccaggtaacatatttcctcaaacgggtaatgcaggacaatgtacagaattaacatgggcgtatatgtcacaactacatggtaaaagacaacctaccgacgacggtcaaataacaaacggtcagcgtgtatggtacgtctataaaaagttaggtgcaaaaacaacacataatccaacagtaggttatggtttctctagtaaaccaccatacttacaagcaactgcatatggtattggtcacacaggtgttgttgtagcagtttttgaagatggttcgtttttagttgcaaactataatgtaccaccatatgttgcaccatcacgtgtggtattgtatacactcattaatggcgtaccaaataatgctggtgataatattgtattctttagtggtattgcttaattaactatgctataatgaacacatgctagtaatgctagtaaataaaatacaaaacataatcaattttcgtacacatttttcatgttatctcaaaaagaaaaggagactgttattttaacagttgcctttttttatttcatcatgttcacgttttaatatatgcaaatcagatttgttatgtactgaacgttcaactggaaataagtcgttaagtgaaaatgaaccgatgtcactttcaatataaagaatatcatcaaattgactatggtcgaaattttctctagcgtcttttaatataaattcacgtttcatattaagttcatcagtaaaatattcatcatatacattaccacatacaatttcagttttagacggatatatcgatattgtaccttgctcattatagatacttttattgttttcaataatggcaccgtcaaagaattgttcacgtacaaaggtttcaaaatcgacgcttgtatcaaaggcgtttttcggtataccagcagaagcaattttaatctttccattcacttcatatgcatatttcttatgattcagtacaaacatcttatctatctgttcgttttcaatatcccatttacctaaggctatcgggtcgaataaactggggttcaataagggtttaacaacggatttcatatacaaactatcagtatcgcaataaataaaattgtcgtcaatttcactttccgttaagtattggaaaggaaccaataagttatacaatgaacgtgatgtgacaaatgtagagaataatatattacgttcagtgtttttgtaaccgttaatgatattgtatagttcattgttatcatctaaacggaataagttaaaatgtgaacgtaatgcaggtatgccatataatccatttaaaacgactttagataacataacctcctcatttgagtatgggtgttcgttgatatcatcagtaatgtgatagtcgtaaggtgatgtcatattgattttgttttttaacttaccttgtgttttaataaaatagttttgaaaaataatatcacgtgcatgaaagtattcacattcatatataacaaacgaattaacacgtatatgcatgcaatcaatacccgtaatgtcttgaatcattcttaatgtatttgtattgatattaacgtaatcattatcattattatagtattttacaatcatttgacgtaatacacgtgatttaattttaattaataaatcatcgttaaatacatctttatcaatcttatataatgaaaaataattgtcatcatctaaaaaagtagggattaacgttggttctgaatagtgttcgtaaaagtataaccatgttggaattttttcatgatacatcacataaggataactcgaattgatgtcaatagaaaaacaaggctcatcaattagtttgtttatgtatttggtgttatacatatttaaaccaccacgatagaatgatttaatatagtcataaaaattcatatcatggaaatgataatgtgtataagatattttaatatcttgatattggttgagtaactgaaaacgtgtcatttcattattcaagtaagattccataatattcaatgaaaatgttaatttgttatagtcaaaatttggaaatatatcactataatgaatatggcacatacctaatataatcacgtcattatgaatgtatgtaagttgttcaggtgtgagttttgcaaaacatttcacagcatagtcataggcttcactatcattcatatcattatctttatcaaaaatcgtataattaaaatctgttttaagttgtgattctgttaaataaccaccatcaagtaatttcttacctaatgttgcaattgatgtattggttttcataaagttatcaataatattaaatttaaaaccatttaaaaacattgttaaatctaaattgattgaagatttaacacgtttttctaaaattacattttgatttttggctaaaatagtagcctctttcatttttaatgtgtgttcattttcttctgcagattttaaatatatattttcgcgtgtaatattatcaaaataacgcatggtgtctttaagtaaaaaatgattatcgtatttattacagttatgtgcaatcatgataatatctgtttttgattttgtgattgtatcacgtcttttcacatacgtataaaatgcgtcataaaaagattcgaaactcggaaatacttcaacatcaatttcataaccattaaaccaaccaattgctacagaataagtaacgtttttatatttggttggtttttttcgtccgttaactttattgtacgctaatgtttctatatcccagtataaaatcattcgacgttcatgtttatgatattgcatgcattctagtaatcccataatcttacacaccttttataagccatattgtttcattagatactttttcgtattctctatatagttatcttcgtatattttttcttttctttcaaactcactcatatttttcttcatttcattttttatatgaaattttataattttattcatatctaaatataaatatctatcattatcaaccacgtaatttttagagtaagcattgtcaaaatgtaaattgcttggattgtagtaataacgttccatgttttctttataaaacatatcatcacgtaaataggtaacatgattgtctatatccctaattttagtacaaaattcatattgttttgtatatggtacaacgataatatttgtcataaaagtagttacattatacatgactttaatatatttatcatcagttttgatatagaagaaatcaccgttttgattgatgtgatttcttaaattatcatccgccaaattatattcgttaaattcaaattctccagttgtcatagcgtcgtcatttgaattaaacgcacgtgtgttacgtttttcattcacgtaatcgtttcgtcgcatttctaaaaaaatgtttttgtaaagtcttgatgtattcattttatgcttttgtaataaattgtatatatttaaattggataatataggacttgaaaagttgactgcattacctagtaaaaacattttagggaatccaatataatcaacgttaccatggttacggtcgattgattcatatattgtttttaacttatcccactcatcaattaaataatcatcttcaagtgctaaaaactcatcatatataataataggatagtgttttaaaaagttagaatgatattttaaatcagtggcactattcaaatctgtaatcacaccaatttctttatcttgatagataatagctaaatagtccctagcacttctgaacgtgacacgttttgatttaaatagtggattttcatctatgatttcttcaataaaatcacggtaagcgtcacgtaatgtataatgacgtgataataaagtaaattttatatcaagtttaatagctaaataaataaaaaatgaaacatagttgaacgattttccatcagaacggtttgaaatagatatataataatctatatcatcattcataagttcatcaactaattctatttgattatacttatctgggattttttttctgacatgattgacagcattttgataatctcttaccatgtctaaacgattttgttttaccatgtttttgctccttgtaatagtttatgatgtcgtttacagtgttaaatttattcgtcaaatgttgcataatataaaaagttatacctcacatcttcatcatcaatatttgtcactggtctatctgatttaccaatttctttatataaagtatcgatttctttaatatatttatacattgaagaattattatttttagcttgtaaattatataaagcgtatttatgctttttagcgtttttattattagaatcatcattacggttatatatttcaagaatataatttaattttttatgtcttgaacctcttaccaatgatacagcatttacatatgatacgtttctttctttaggaaaatagggcagatgtgcaaaatgtttccatgtgtcaatgtacgcctcttgtaaatctttatcatcaaatttaaaattaacattactaaaatcatttaaaaataaatctttttcttgctcttttctagcttctctttcttttttccatctatccatttcagacgtatgtctaaccaatgttatcaacctccatataaagcataaataaccattaaaaagataatatagaatataatcaatgtagtgaataaaacaccaaatgacacgcgtatatgcagtgtcataagtatgataagtgtaattaaaaatgctaaaaggaaaacaatggctatgtttaataggttattcatggtcaatcactttcccattatcgtatatgactttgttttgataaataatcattaattcgctttcaagaggtttatcaaaatttgataatacgtcgtcaattgtaacgtttaataaaatttctcttattaattcattacttaaataatttctataataaaatacaagtatattaaaaacatgttttttaatatcaatgtcgatatctaacgtaaataactctttttcaatttcaaaatcatcatattgtttgtcaaactcaatatacacatcacccatatttatttttactatacattttttattagatgaagtaaatttttcaaatttatcattataataatctctatttgttaaaaggtaataaattaaattatttaatctaaaagtagttttaattttcatttttatatctccttaatgtattctatgatatacgcgtattttttagtgaacaggttatattcataatatgaatatacaactttagcgtcatataaatcttcaaacattgagatttgatgtggaaaatgtcctttaatctcatcgcaatataataataccgttttgtatttacgttccatttaaacacctcataaaaaataggggataagtatcccctatgaaattgtattaaaatgatacttgaccaaaattgattgagtaacctttttgaccttttttgttttcatattcataaattgtgaattgaacttctccagcattgataatgtcaacaacgtcctcatctgctctcatttctttaattaattctgttaagtggttcggtaagtttacgttatagtcatcagtgacgataacaccttgttcaccgaattttgattctttgtttgtgaataatgctctaacgatatactcttttttcataccgtatttttctactaattctgatagtttgataaattctctttctttttcctcaaattcaaatctcgctaatgtgttttggtgtcttgataaaatatcttttacgtttgtcattttatttctcctcttatttaaattatttgctttctgcaattgcgatttgtagtaaatcattgtaataaacttgaattgttttcgttgtgcgtgtagtggacaatagtttacatgtgtctggtaataattcttttgcttgtgttttggttaaatgatactcgtgaagtggtaaaaattcctcaatgtattcattatcatcatctaagtaatgaagtatataacctttgacacgtaaggtaacaatgtcgtcaactttcattattatatcactcctttctaaaaaacgtaaacgttatacgtttcataaaatcctttatgcatattccattgttctattgggtcatcaccagcaatataagacaatattgattctggtttagtttcgttgtttagttcatcatttaagaattgaacaacagaactattatagtttaataatagttgttggcaagccgataataagttaattgcattgtcaaatgtataagctggattccattgaatcagtttattgaatagttgcaacatttcagtataggcttgtcctttttcttctggtgcattatcaacattaaccattattatcacttcctaataaagttgaaattacgcgtaaaacagaattatgatttaaatcttcaatttcatcaatgtcaacatcataaaatgaaatttcattttctgttctatcaaataacgctatacataaacttccattcttaaaacgaaaaacatgcttcaactcaatgttttttgtttcattttccatttttgttactccttgttttgattacatacttagtatagcaaacgtttaaaagttttgtcaatagtttttcttaaaaaagtttaaataattttaaaactactatttaatagaagaaataagattttaagttcaaatcataattttgaataaaagtcaatagatacataaattttgtatttgatgaatatgtaataggttagataagttggttaagttgttgcacagtatttttaagtttagtaaagaaatgataagtaaatttataagttttgatttgtataatcgtttattttaaaccggtggggt

SEQ ID NO: 2 - DNA, 2286 bp, Staphylococcus aureus Bacteriophage 44 AHJD
atgggattactagaatgcatgcaatatcataaacatgaacgtcgaatgattttatactgggatatagaaacattagcgtacaataaagttaacggacgaaaaaaaccaaccaaatataaaaacgttacttattctgtagcaattggttggtttaatggttatgaaattgatgttgaagtatttccgagtttcgaatctttttatgacgcattttatacgtatgtgaaaagacgtgatacaatcacaaaatcaaaaacagatattatcatgattgcacataactgtaataaatacgataatcattttttacttaaagacaccatgcgttattttgataatattacacgcgaaaatatatatttaaaatctgcagaagaaaatgaacacacattaaaaatgaaagaggctactattttagccaaaaatcaaaatgtaattttagaaaaacgtgttaaatcttcaatcaatttagatttaacaatgtttttaaatggttttaaatttaatattattgataactttatgaaaaccaatacatcaattgcaacattaggtaagaaattacttgatggtggttatttaacagaatcacaacttaaaacagattttaattatacgatttttgataaagataatgatatgaatgatagtgaagcctatgactatgctgtgaaatgttttgcaaaactcacacctgaacaacttacatacattcataatgacgtgattatattaggtatgtgccatattcattatagtgatatatttccaaattttgactataacaaattaacattttcattgaatattatggaatcttacttgaataatgaaatgacacgttttcagttactcaaccaatatcaagatattaaaatatcttatacacattatcatttccatgatatgaatttttatgactatattaaatcattctatcgtggtggtttaaatatgtataacaccaaatacataaacaaactaattgatgagccttgtttttctattgacatcaattcgagttatccttatgtgatgtatcatgaaaaaattccaacatggttatacttttacgaacactattcagaaccaacgttaatccctacttttttagatgatgacaattatttttcattatataagattgataaagatgtatttaacgatgatttattaattaaaattaaatcacgtgtattacgtcaaatgattgtaaaatactataataatgataatgattacgttaatatcaatacaaatacattaagaatgattcaagacattacgggtattgattgcatgcatatacgtgttaattcgtttgttatatatgaatgtgaatactttcatgcacgtgatattatttttcaaaactattttattaaaacacaaggtaagttaaaaaacaaaatcaatatgacatcaccttacgactatcacattactgatgatatcaacgaacacccatactcaaatgaggaggttatgttatctaaagtcgttttaaatggattatatggcatacctgcattacgttcacattttaacttattccgtttagatgataacaatgaactatacaatatcattaacggttacaaaaacactgaacgtaatatattattctctacatttgtcacatcacgttcattgtataacttattggttcctttccaatacttaacggaaagtgaaattgacgacaattttatttattgcgatactgatagtttgtatatgaaatccgttgttaaacccttattgaaccccagtttattcgacccgatagccttaggtaaatgggatattgaaaacgaacagatagataagatgtttgtactgaatcataagaaatatgcatatgaagtgaatggaaagattaaaattgcttctgctggtataccgaaaaacgcctttgatacaagcgtcgattttgaaacctttgtacgtgaacaattctttgacggtgccattattgaaaacaataaaagtatctataatgagcaaggtacaatatcgatatatccgtctaaaactgaaattgtatgtggtaatgtatatgatgaatattttactgatgaacttaatatgaaacgtgaatttatattaaaagacgctagagaaaatttcgaccatagtcaatttgatgatattctttatattgaaagtgacatcggttcattttcacttaacgacttatttccagttgaacgttcagtacataacaaatctgatttgcatatattaaaacgtgaacatgatgaaataaaaaaaggcaactgttaa

SEQ ID NO: 3 - DNA, 1944 bp, Staphylococcus aureus Bacteriophage 44 AHJD
atggcatataatgaaaacgattttaaatattttgatgacattcgtccatttttagacgaaatttataaaacgagagaacgttatacaccgttttacgatgatagagcagattataatactaattcaaaatcatattatgattatatttcaagattatcaaaactaattgaagtattagcacgtcgtatttgggactatgacaatgaattaaaaaaacgtttcaaaaattgggacgacttaatgaaagcatttccagagcaagcgaaagacttatttagaggttggttaaacgacggtacgattgacagtattattcatgacgagtttaaaaaatatagcgcaggattaacatcggcatttgctttatttaaagttactgaaatgaaacaaatgaatgactttaaatcagaagttaaagacttaattaaagatattgaccgtttcgttaatgggtttgaattaaatgagcttgaaccaaagtttgtgatgggctttggtggtattcgcaacgcagttaaccaatctattaatattgataaagaaacaaatcacatgtactctacacaatccgattctcaaaaacctgaaggtttttggataaataaattaacacctagtggtgacttaatttcaagcatgcgtattgtacagggtggtcatggtacaacaatcggattagaacgtcaatccaatggtgaaatgaaaatctggttacatcacgatggtgttgcaaaactgttacaagtcgcatataaagataattatgtattagatttagaagaggctaaaggtttaacagattatacaccacagtcacttttaaacaaacacacatttacaccgttaattgatgaagcaaatgacaaactcattttaagattcggtgacggaacaatacaggttcgttcaagagcagacgtaaaaaatcacattgataatgtagaaaaagaaatgacaattgataattcagaaaacaatgataatcgttggatgcaaggcattgctgttgatggtgatgatttatactggttaagtggtaacagttcagttaattcacatgttcaaatcggtaaatattcattaacaacaggtcaaaagatttatgattatccatttaagttatcatatcaagacggtattaatttcccacgtgataactttaaagagcctgagggtatttgcatttatacaaatccaaaaacaaaacgtaaatcgttattacttgctatgacaaacggcggtggtggaaaacgtttccataatttatatggtttcttccaacttggtgagtatgaacactttgaagcattacgcgcaagaggttcacaaaactataaattaacaaaagacgacggtcgtgcattatctattccagaccatatcgacgatttaaatgacttaacgcaagctggtttttattatattgacgggggtactgcagaaaaacttaagaatatgccaatgaatggtagcaagcgtataattgacgctggttgtttcattaatgtataccctacaacacaaacattaggtacggttcaagaattaacacgtttctcaacaggtcgtaaaatggttaaaatggtgcgtggtatgactttagacgtatttacgttaaaatgggattatggattatggacaacaatcaaaactgacgcaccatatcaagaatatttggaagcaagtcaatacaataactggattgcttatgtaacaacagctggtgagtattacattacaggtaaccaaatggaattatttagagacgcgccagaagaaattaaaaaagtgggtgcatggttacgtgtgtcaagtggtaacgcagtcggtgaagtaagacaaacattagaggctaatatatcggaatataaagaattcttcagtaatgttaatgcggaaacaaaacatcgtgaatatggttgggtagcaaaacatcaaaaatag

SEQ ID NO: 4 - DNA, 1764 bp, Staphylococcus aureus Bacteriophage 44 AHJD
atgagaaagttaacgaattttaagtttttctataacacaccgtttacagactatcaaaacacgattcattttaatagtaataaagaacgtgatgattattttttaaatggtcgtcattttaaatcgttagactattcaaaacaaccgtataattttatacgtgatagaatggaaatcaatgttgatatgcagtggcatgacgcacaaggtattaactacatgacgtttttatcagattttgaggatagaagatattacgcttttgtaaaccaaatcgaatacgtgaatgacgttgtggttaaaatatattttgtcattgataccattatgacgtatacacaagggaatgtattagagcaactctcaaacgtcaatattgaacgtcaacatttatcaaaacgcacgtataactatatgttaccaatgttacgtaataatgatgatgtgttaaaagtatcaaataaaaactatgtttataaccaaatgcaacaatatttggaaaatttagtattattccagtcaagcgctgatttatcaaagaaatttggtactaaaaaagagccaaacttagatacgtcaaaaggtacgatttatgacaatatcacatcaccagtcaacttatacgttatggaatatggtgactttattaactttatggataaaatgagtgcctatccatggattacgcaaaactttcaaaaggttcaaatgttacctaaagactttattaatacaaaagacttagaggacgttaaaaccagtgaaaaaattacaggattaaaaacattaaaacagggtggtaaatcaaaagaatggagtctaaaagatttatcattaagtttctcaaatcttcaagagatgatgttatctaaaaaagatgaatttaaacatatgatacgtaatgagtatatgacaattgaattttatgactggaatggaaatacgatgttactcgacgctggtaagatttcacaaaaaactggtgttaagttacgtacaaaatcaattattggttatcataatgaagttcgagtatatccagtagattataacagtgctgaaaacgacagaccaatactcgctaaaaataaagaaatattgattgatacgggttcattcttaaatacaaatataacatttaatagttttgcacaagtaccaatattaatcaataatggtatcttaggacaatcacaacaagccaaccgacaaaaaaatgcagaaagtcaattaattacaaatcgtattgataatgtattaaatggtagcgacccgaaatcacgcttttatgacgctgtgagtgtagcaagtaatttaagtccaactgctttatttggtaagtttaatgaagaatataatttctacaaacaacaacaagctgaatataaagatttagccttacaaccaccttctgtaactgaatcagaaatgggcaacgcattccaaattgcgaatagcattaacggtttaacgatgaaaattagtgtaccgtcacctaaagaaattacatttttacaaaaatattatatgttgtttggttttgaagtgaatgactataattcatttattgaaccaattaacagtatgactgtttgcaattatttaaaatgtacaggtacgtatactatacgtgacatcgaccccatgttaatggaacaattaaaagcaattttagaatctggtgtaagattttggcataatgacggttcaggtaatccaatgttacaaaatccattaaataacaaatttagagagggggtataa

SEQ ID NO: 5 - DNA, 1464 bp, Staphylococcus aureus Bacteriophage 44 AHJD
atgatactgaaaagagtgataacaatgaacgatcaagagaagatagataaatttacgcattcctatattaatgatgattttggtttaacgatagaccagttagtccctaaagtaaaaggatatgggcgctttaatgtatggcttggtggtaatgaaagtaaaatcagacaagtattaaaagcagtaaaagagataggtgtttcacctactctttttgccgtatatgaaaaaaatgagggttttagttctggacttggttggttaaaccatacgtctgcacgtggtgattatttaacagatgctaaattcatagcaagaaagttagtatcacaatcaaaacaagctggacaaccgtcttggtatgacgcaggtaacatcgtccactttgtaccacaagacgtacaaagaaaaggtaatgcagattttgcaaaaaatatgaaagcaggtacaattggacgtgcatatattccattaacagcagctgctacttgggcggcatattatcctttaggtttgaaagcatcatataacaaagtacaaaactatggtaatccatttttagacggtgcgaatactattctagcttggggtggtaaattagacggtaaaggtggatcacctagtgattcgtctgacagtggtagtagtggtgacagtggtagttcactactcgctttagcaaaacaagccatgcaagaattattaaaaaaaatacaagacgcattacaatgggacgttcatagtattggtagtgataaattttttagtaatgattattttacattagaaaaaacatttaacaacacatatcatattaaaatgacgattggtttacttgattcattaaaaaaactgattgatagcgttcaagtagatagtgggagtagtagttctaatcctactgatgatgacggagaccataaaccaattagtggtaaatcagtcaagccaaatggaaaaagtggtcgtgtgattggtggtaactggacatatgcacagttaccagaaaaatataaaaaagcaattggtgtacctttattcaaaaaagaatacttatacaaaccaggtaacatatttcctcaaacgggtaatgcaggacaatgtacagaattaacatgggcgtatatgtcacaactacatggtaaaagacaacctaccgacgacggtcaaataacaaacggtcagcgtgtatggtacgtctataaaaagttaggtgcaaaaacaacacataatccaacagtaggttatggtttctctagtaaaccaccatacttacaagcaactgcatatggtattggtcacacaggtgttgttgtagcagtttttgaagatggttcgtttttagttgcaaactataatgtaccaccatatgttgcaccatcacgtgtggtattgtatacactcattaatggcgtaccaaataatgctggtgataatattgtattctttagtggtattgcttaa

SEQ ID NO: 6 - DNA, 1248 bp, Staphylococcus aureus Bacteriophage 44 AHJD
atggtaaaacaaaatcgtttagacatggtaagagattatcaaaatgctgtcaatcatgtcagaaaaaaaatcccagataagtataatcaaatagaattagttgatgaacttatgaatgatgatatagattattatatatctatttcaaaccgttctgatggaaaatcgttcaactatgtttcattttttatttatttagctattaaacttgatataaaatttactttattatcacgtcattatacattacgtgacgcttaccgtgattttattgaagaaatcatagatgaaaatccactatttaaatcaaaacgtgtcacgttcagaagtgctagggactatttagctattatctatcaagataaagaaattggtgtgattacagatttgaatagtgccactgatttaaaatatcattctaactttttaaaacactatcctattattatatatgatgagtttttagcacttgaagatgattatttaattgatgagtgggataagttaaaaacaatatatgaatcaatcgaccgtaaccatggtaacgttgattatattggattccctaaaatgtttttactaggtaatgcagtcaacttttcaagtcctatattatccaatttaaatatatacaatttattacaaaagcataaaatgaatacatcaagactttacaaaaacatttttttagaaatgcgacgaaacgattacgtgaatgaaaaacgtaacacacgtgcgtttaattcaaatgacgacgctatgacaactggagaatttgaatttaacgaatataatttggcggatgataatttaagaaatcacatcaatcaaaacggtgatttcttctatatcaaaactgatgataaatatattaaagtcatgtataatgtaactacttttatgacaaatattatcgttgtaccatatacaaaacaatatgaattttgtactaaaattagggatatagacaatcatgttacctatttacgtgatgatatgttttataaagaaaacatggaacgttattactacaatccaagcaatttacattttgacaatgcttactctaaaaattacgtggttgataatgatagatatttatatttagatatgaataaaattataaaatttcatataaaaaatgaaatgaagaaaaatatgagtgagtttgaaagaaaagaaaaaatatacgaagataactatatagagaatacgaaaaagtatctaatgaaacaatatggcttataa

SEQ ID NO: 7 - DNA, 1227 bp, Staphylococcus aureus Bacteriophage 44 AHJD
atggcacaacaatctacaaaaaatgaaactgcacttttagtagcaaagtcagctaaatcagcgttacaagattttaatcatgattattcaaaatcttggacatttggcgacaaatgggataattcaaatacaatgttcgaaacatttgtaaataaatatttattccctaagattaatgagactttattaatcgatattgcattaggtaatcgttttaattggttagctaaagagcaagattttattggacaatatagtgaagaatacgtgattatggacacagtaccaattaacatggacttatctaaaaatgaggaattaatgttgaaacgtaattatccacgtatggcaactaagttatatggtaacggaattgtgaagaaacaaaaattcacattaaacaacaatgatacacgtttcaatttccaaacattagcagacgcaactaattacgctttaggtgtatacaaaaagaaaatttctgatattaatgtattagaagaaaaagaaatgcgtgcaatgttagttgattactcattgaatcaattatccgaaacaaatgtacgtaaagcaacatcaaaagaagatttagcaagcaaagtttttgaagcaatcctaaacttacaaaacaacagtgctaaatataatgaagtacatcgtgcatcaggtggtgcaattggacaatatacaactgtatcaaaattaaaagatattgtgattttaacaacagattcattaaaatcttatcttttagatactaagattgcaaacacattccagattgcaggcattgatttcacagatcacgttattagttttgacgacttaggtggcgtgtttaaagtaacaaaagaatttaagttacaaaaccaagattcaattgactttttacgtgcgtatggagattatcaatcacaattaggagatacaattccagttggtgctgtatttacttatgatgtatctaaacttaaagagtttactggcaacgttgaagaaattaaaccaaaatcagatttatatgcgtttattttggatattaattcaattaaatataaacgttacacaaaaggtatgttaaaaccaccattccataaccctgaatttgatgaagttacacactggattcattactattcatttaaagccattagtccattctttaataaaattttaattactgaccaagatgtaaatccaaaaccagaggaagaattacaagaataa

SEQ ID NO: 8 - DNA, 984 bp, Staphylococcus aureus Bacteriophage 44 AHJD
atgaacaacgataaaagaggtttaaacgttgagttatcaaaggaaatcagcaaaagagttgttgaacatcgcaacagatttaaacgtcttatgtttaatcgttatttggaatttttaccgctactaatcaactataccaatcgtgatacggttggtatagattttattcagttagaatcagctttaagacaaaacattaatgtagttgttggtgaagctagaaataagcaaattatgattcttggttatgtaaataacacttactttaatcaagcaccaaatttttcatcaaactttaatttccaatttcaaaaacgattaactaaagaagatatatattttattgtacctgactatttaatacctgatgattgtctacaaattcataagctatatgataactgtatgagtggtaactttgttgtcatgcaaaataaaccaattcaatataatagtgatatagaaattatagaacattatactgatgaattagcagaagttgctttatctcgcttttctttaatcatgcaagcaaaatttagcaagatatttaaatcagaaattaatgacgagtcaatcaatcaacttgtgtccgaaatatataacggtgcaccatttgttaaaatgtcacctatgtttaatgcagatgacgatatcattgatttaacaagtaatagcgtaatcccagcattaactgaaatgaaacgggaatatcaaaacaaaattagtgaattaagtaactatttaggcattaattcattagccgttgataaagaaagcggtgtttcagacgaagaggcaaaaagtaatcgtggatttaccacatcaaacagtaatatctatttaaaaggtcgtgaaccaattacgtttttatcaaagcgttatggtttagatattaaaccgtattacgatgatgaaacaacgtctaaaatatcaatggtagacacactttttaaagatgaaagcagtgatataaatggctag

SEQ ID NO: 9 - DNA, 756 bp, Staphylococcus aureus Bacteriophage 44 AHJD
atggctagatacacaatgactttatacgatttcattaaatcagaattgattaaaaaaggtttcaatgaatttgtaaatgataataaattaacgttttatgatgatgaatttcaattcatgcaaaaaatgctgaagttcgacaaagacgttttagctatcgttaatgaaaaagtatttaaaggtttttcattgaaagatgaattatcagatttactttttaaaaaatcatttacgattcattttttagatagagaaatcaacagacaaacagttgaagcatttggcatgcaagtgattactgtatgtattacacatgaggattatttaaatgtggtttattcatcaagtgaagttgaaaaatacttacaatcacaaggcttcacagaacacaatgaagatacaacaagtaacactgatgaaacatcgaatcaaaatgctacatctttagacaattcaactggcatgactgcaaacagaaacgcttatgtgtcattaccacaaagtgaggttaacattgatgttgataatacaacgttacgattcgctgataataatacgattgataacggtaaaactgtgaataaatcgagtaacgaaagtaatcaaaacgcaaaacgtaatcaaaatcaaaaaggtaatgcaaaaggtacacaattcactaagcagtatttaattgataatattgataaagcgtacgatttaagaaagaaaattttaaatgaatttgataaaaaatgttttttacaaatttggtag

SEQ ID NO: 10 - DNA, 753 bp, Staphylococcus aureus Bacteriophage 44 AHJD
atgaaatcacaacaacaagcaaaagaatggatatataagcatgagggggcaggtgttgactttgatggtgcatatggatttcaatgtatggacttatcagttgcttatgtgtattacattactgacggtaaagttcgcatgtggggtaatgctaaagacgcgataaataatgactttaaaggtttagcgacggtgtataaaaatacaccgagctttaaacctcaattaggggacgttgctgtatatacaaatggacaatatggacatattcaatgtgtgttaagtggaaatcttgattattatacatgcttagaacaaaactggttaggcggcggttttgacggttgggaaaaagcaaccattagaacacattattatgacggtgtaactcactttattagacctaaattttcaggtagtaatagcaaagcattagaaacatcaaaagtaaatacatttggaaaatggaaacgaaaccaatacggcacatattatagaaatgaaaatggtacatttacatgtggttttttaccaatatttgcacgtgtcggtagtccaaaattatcagaacctaatggctattggttccaaccaaacggttatacaccatataacgaagtttgtttatcagatggttacgtatggattggttataactggcaaggcacacgttattatttaccagtgcgccaatggaatggaaaaacaggtaatagttacagtgttggtattccttggggggtgttctcataa

SEQ ID NO: 11 - DNA, 483 bp, Staphylococcus aureus Bacteriophage 44 AHJD
ttggttagacatacgtctgaaatggatagatggaaaaaagaaagagaagctagaaaagagcaagaaaaagatttatttttaaatgattttagtaatgttaattttaaatttgatgataaagatttacaagaggcgtacattgacacatggaaacattttgcacatctgccctattttcctaaagaaagaaacgtatcatatgtaaatgctgtatcattggtaagaggttcaagacataaaaaattaaattatattcttgaaatatataaccgtaatgatgattctaataataaaaacgctaaaaagcataaatacgctttatataatttacaagctaaaaataataattcttcaatgtataaatatattaaagaaatcgatactttatataaagaaattggtaaatcagatagaccagtgacaaatattgatgatgaagatgtgaggtataactttttatattatgcaacatttgacgaataa

SEQ ID NO: 12 - DNA, 369 bp, Staphylococcus aureus Bacteriophage 44 AHJD
atgacaaacgtaaaagatattttatcaagacaccaaaacacattagcgagatttgaatttgaggaaaaagaaagagaatttatcaaactatcagaattagtagaaaaatacggtatgaaaaaagagtatatcgttagagcattattcacaaacaaagaatcaaaattcggtgaacaaggtgttatcgtcactgatgactataacgtaaacttaccgaaccacttaacagaattaattaaagaaatgagagcagatgaggacgttgttgacattatcaatgctggagaagttcaattcacaatttatgaatatgaaaacaaaaaaggtcaaaaaggttactcaatcaattttggtcaagtatcattttaa

SEQ ID NO: 13 - DNA, 423 bp, Staphylococcus aureus Bacteriophage 44 AHJD
atgaacgaagtaaaattcagatttacagactcagaagcgtttcacatgtttatatacgctggggatttaaaattactctactttttatttgtattaatgttcgttgatattattacaggtatttcaaaagcaattaaaaataataacttatggtcaaaaaaatcaatgagaggattttctaaaaaattattgatattctgtattatcattttagcaaacatcattgaccagattttacaattaaaaggtggtctactcatgattacaatattttattatattgcaaatgagggactttctattgtagaaaattgtgcagaaatggacgtattagtaccagaacaaattaaagataaattaagagtcattaaaaatgatactgaaaagagtgataacaatgaacgatcaagagaagatagataa

SEQ ID NO: 14 - DNA, 411 bp, Staphylococcus aureus Bacteriophage 44 AHJD
atgaaaattaaaactacttttagattaaataatttaatttattaccttttaacaaatagagattattataatgataaatttgaaaaatttacttcatctaataaaaaatgtatagtaaaaataaatatgggtgatgtgtatattgagtttgacaaacaatatgatgattttgaaattgaaaaagagttatttacgttagatatcgacattgatattaaaaaacatgtttttaatatacttgtattttattatagaaattatttaagtaatgaattaataagagaaattttattaaacgttacaattgacgacgtattatcaaattttgataaacctcttgaaagcgaattaatgattatttatcaaaacaaagtcatatacgataatgggaaagtgattgaccatgaataa

SEQ ID NO: 15 - DNA, 279 bp, Staphylococcus aureus Bacteriophage 44 AHJD
atgaaaatggtacatttacatgtggttttttaccaatatttgcacgtgtcggtagtccaaaattatcagaacctaatggctattggttccaaccaaacggttatacaccatataacgaagtttgtttatcagatggttacgtatggattggttataactggcaaggcacacgttattatttaccagtgcgccaatggaatggaaaaacaggtaatagttacagtgttggtattccttggggggtgttctcataatgggtattttagcctttttctttga

SEQ ID NO: 16 - DNA, 243 bp, Staphylococcus aureus Bacteriophage 44 AHJD
gtgacgataacaccttgttcaccgaattttgattctttgtttgtgaataatgctctaacgatatactcttttttcataccgtatttttctactaattctgatagtttgataaattctctttctttttcctcaaattcaaatctcgctaatgtgttttggtgtcttgataaaatatcttttacgtttgtcattttatttctcctcttatttaaattatttgctttctgcaattgcgatttgtag

SEQ ID NO: 17 - DNA, 237 bp, Staphylococcus aureus Bacteriophage 44 AHJD
atgaaagttgacgacattgttaccttacgtgtcaaaggttatatacttcattacttagatgatgataatgaatacattgaggaatttttaccacttcacgagtatcatttaaccaaaacacaagcaaaagaattattaccagacacatgtaaactattgtccactacacgcacaacgaaaacaattcaagtttattacaatgatttactacaaatcgcaattgcagaaagcaaataa

SEQ ID NO: 18 - DNA, 222 bp, Staphylococcus aureus Bacteriophage 44 AHJD
atggaaagattaaaattgcttctgctggtataccgaaaaacgcctttgatacaagcgtcgattttgaaacctttgtacgtgaacaattctttgacggtgccattattgaaaacaataaaagtatctataatgagcaaggtacaatatcgatatatccgtctaaaactgaaattgtatgtggtaatgtatatgatgaatattttactgatgaacttaatatga

SEQ ID NO: 19 - DNA, 213 bp, Staphylococcus aureus Bacteriophage 44 AHJD
atgttaattggtactgtgtccataatcacgtattcttcactatattgtccaataaaatcttgctctttagctaaccaattaaaacgattacctaatgcaatatcgattaataaagtctcattaatcttagggaataaatatttatttacaaatgtttcgaacattgtatttgaattatcccatttgtcgccaaatgtccaagattttgaataa

SEQ ID NO: 20 - DNA, 207 bp, Staphylococcus aureus Bacteriophage 44 AHJD
atgttacctggtttgtataagtattcttttttgaataaaggtacaccaattgcttttttatatttttctggtaactgtgcatatgtccagttaccaccaatcacacgaccactttttccatttggcttgactgatttaccactaattggtttatggtctccgtcatcatcagtaggattagaactactactcccactatctacttga

SEQ ID NO: 21 - DNA, 189 bp, Staphylococcus aureus Bacteriophage 44 AHJD
atgtctaaacgattttgttttaccatgtttttgctccttgtaatagtttatgatgtcgtttacagtgttaaatttattcgtcaaatgttgcataatataaaaagttatacctcacatcttcatcatcaatatttgtcactggtctatctgatttaccaatttctttatataaagtatcgatttctttaa

SEQ ID NO: 22 - DNA, 183 bp, Staphylococcus aureus Bacteriophage 44 AHJD
atgcaccatcaaagtcaacacctgccccctcatgcttatatatccattcttttgcttgttgttgtgatttcatttatatcactcctatttttgatgttttgctacccaaccatattcacgatgttttgtttccgcattaacattactgaagaattctttatattccgatatattagcctctaa

SEQ ID NO: 23 - DNA, 144 bp, Staphylococcus aureus Bacteriophage 44 AHJD
atgtttgctaaaatgataatacagaatatcaataattttttagaaaatcctctcattgatttttttgaccataagttattatttttaattgcttttgaaatacctgtaataatatcaacgaacattaatacaaataaaaagtag

SEQ ID NO: 24 - DNA, 180 bp, Staphylococcus aureus Bacteriophage 44 AHJD
atgagaacaccccccaaggaataccaacactgtaactattacctgtttttccattccattggcgcactggtaaataataacgtgtgccttgccagttataaccaatccatacgtaaccatctgataaacaaacttcgttatatggtgtataaccgtttggttggaaccaatagccattag

SEQ ID NO: 25 - DNA, 177 bp, Staphylococcus aureus Bacteriophage 44 AHJD
gtgtcaatgtacgcctcttgtaaatctttatcatcaaatttaaaattaacattactaaaatcatttaaaaataaatctttttcttgctcttttctagcttctctttcttttttccatctatccatttcagacgtatgtctaaccaatgttatcaacctccatataaagcataaataa

SEQ ID NO: 26 - DNA, 177 bp, Staphylococcus aureus Bacteriophage 44 AHJD
atggaacgtaaatacaaaacggtattattatattgcgatgagattaaaggacattttccacatcaaatctcaatgtttgaagatttatatgacgctaaagttgtatattcatattatgaatataacctgttcactaaaaaatacgcgtatatcatagaatacattaaggagatataa

SEQ ID NO: 27 - DNA, 168 bp, Staphylococcus aureus Bacteriophage 44 AHJD
atgaataacctattaaacatagccattgttttccttttagcatttttaattacacttatcatacttatgacactgcatatacgcgtgtcatttggtgttttattcactacattgattatattctatattatctttttaatggttatttatgctttatatggaggttga

SEQ ID NO: 28 - DNA, 165 bp, Staphylococcus aureus Bacteriophage 44 AHJD
atgattgtctatatccctaattttagtacaaaattcatattgttttgtatatggtacaacgataatatttgtcataaaagtagttacattatacatgactttaatatatttatcatcagttttgatatagaagaaatcaccgttttgattgatgtgatttcttaa

SEQ ID NO: 29 - DNA, 165 bp, Staphylococcus aureus Bacteriophage 44 AHJD
atggaatatatgcacgtccaattgtacctgctttcatattttttgcaaaatctgcattaccttttctttgtacgtcttgtggtacaaagtggacgatgttacctgcgtcataccaagacggttgtccagcttgttttgattgtgatactaactttcttgctatga

SEQ ID NO: 30 - DNA, 165 bp, Staphylococcus aureus Bacteriophage 44 AHJD
gtgtttaaatggaacgtaaatacaaaacggtattattatattgcgatgagattaaaggacattttccacatcaaatctcaatgtttgaagatttatatgacgctaaagttgtatattcatattatgaatataacctgttcactaaaaaatacgcgtatatcatag

SEQ ID NO: 31 - DNA, 162 bp, Staphylococcus aureus Bacteriophage 44 AHJD
gtgaataaaacaccaaatgacacgcgtatatgcagtgtcataagtatgataagtgtaattaaaaatgctaaaaggaaaacaatggctatgtttaataggttattcatggtcaatcactttcccattatcgtatatgactttgttttgataaataatcattaa

SEQ ID NO: 32 - DNA, 153 bp, Staphylococcus aureus Bacteriophage 44 AHJD
atgatattgtatagttcattgttatcatctaaacggaataagttaaaatgtgaacgtaatgcaggtatgccatataatccatttaaaacgactttagataacataacctcctcatttgagtatgggtgttcgttgatatcatcagtaatgtga

SEQ ID NO: 33 - DNA, 150 bp, Staphylococcus aureus Bacteriophage 44 AHJD
atggcttgttttgctaaagcgagtagtgaactaccactgtcaccactactaccactgtcagacgaatcactaggtgatccacctttaccgtctaatttaccaccccaagctagaatagtattcgcaccgtctaaaaatggattaccatag

SEQ ID NO: 34 - DNA, 150 bp, Staphylococcus aureus Bacteriophage 44 AHJD
atgccattatttaaccacctctaccaaatttgtaaaaaacattttttatcaaattcatttaaaattttctttcttaaatcgtacgctttatcaatattatcaattaaatactgcttagtgaattgtgtaccttttgcattacctttttga

SEQ ID NO: 35 - DNA, 147 bp, Staphylococcus aureus Bacteriophage 44 AHJD
atgatgattctaataataaaaacgctaaaaagcataaatacgctttatataatttacaagctaaaaataataattcttcaatgtataaatatattaaagaaatcgatactttatataaagaaattggtaaatcagatagaccagtga

SEQ ID NO: 36 - DNA, 147 bp, Staphylococcus aureus Bacteriophage 44 AHJD
atgcaacatttgacgaataaatttaacactgtaaacgacatcataaactattacaaggagcaaaaacatggtaaaacaaaatcgtttagacatggtaagagattatcaaaatgctgtcaatcatgtcagaaaaaaaatcccagataa

SEQ ID NO: 37 - DNA, 147 bp, Staphylococcus aureus Bacteriophage 44 AHJD
gtgtatacaataccacacgtgatggtgcaacatatggtggtacattatagtttgcaactaaaaacgaaccatcttcaaaaactgctacaacaacacctgtgtgaccaataccatatgcagttgcttgtaagtatggtggtttactag

SEQ ID NO: 38 - DNA, 144 bp, Staphylococcus aureus Bacteriophage 44 AHJD
atgtcgatatctaacgtaaataactctttttcaatttcaaaatcatcatattgtttgtcaaactcaatatacacatcacccatatttatttttactatacattttttattagatgaagtaaatttttcaaatttatcattataa

SEQ ID NO: 39 - DNA, 144 bp, Staphylococcus aureus Bacteriophage 44 AHJD
gtgtaccttttgcattacctttttgattttgattacgttttgcgttttgattactttcgttactcgatttattcacagttttaccgttatcaatcgtattattatcagcgaatcgtaacgttgtattatcaacatcaatgttaa

SEQ ID NO: 40 - DNA, 141 bp, Staphylococcus aureus Bacteriophage 44 AHJD
gtgctgtatttacttatgatgtatctaaacttaaagagtttactggcaacgttgaagaaattaaaccaaaatcagatttatatgcgtttattttggatattaattcaattaaatataaacgttacacaaaaggtatgttaa

SEQ ID NO: 41 - DNA, 138 bp, Staphylococcus aureus Bacteriophage 44 AHJD
gtggtaactggacatatgcacagttaccagaaaaatataaaaaagcaattggtgtacctttattcaaaaaagaatacttatacaaaccaggtaacatatttcctcaaacgggtaatgcaggacaatgtacagaattaa

SEQ ID NO: 42 - DNA, 138 bp, Staphylococcus aureus Bacteriophage 44 AHJD
atgtcgtcaactttcattattatatcactcctttctaaaaaacgtaaacgttatacgtttcataaaatcctttatgcatattccattgttctattgggtcatcaccagcaatataagacaatattgattctggtttag

SEQ ID NO: 43 - DNA, 138 bp, Staphylococcus aureus Bacteriophage 44 AHJD
atgcacgaccgtcgtcttttgttaatttatagttttgtgaacctcttgcgcgtaatgcttcaaagtgttcatactcaccaagttggaagaaaccatataaattatggaaacgttttccaccaccgccgtttgtcatag

SEQ ID NO: 44 - DNA, 138 bp, Staphylococcus aureus Bacteriophage 44 AHJD
atgcgacttgtaacagttttgcaacaccatcgtgatgtaaccagattttcatttcaccattggattgacgttctaatccgattgttgtaccatgaccaccctgtacaatacgcatgcttgaaattaagtcaccactag

SEQ ID NO: 45 - DNA, 135 bp, Staphylococcus aureus Bacteriophage 44 AHJD
atgttacctatttacgtgatgatatgttttataaagaaaacatggaacgttattactacaatccaagcaatttacattttgacaatgcttactctaaaaattacgtggttgataatgatagatatttatatttag

SEQ ID NO: 46 - DNA, 129 bp, Staphylococcus aureus Bacteriophage 44 AHJD
atggcaccgtcaaagaattgttcacgtacaaaggtttcaaaatcgacgcttgtatcaaaggcgtttttcggtataccagcagaagcaattttaatctttccattcacttcatatgcatatttcttatga

SEQ ID NO: 47 - DNA, 129 bp, Staphylococcus aureus Bacteriophage 44 AHJD
atgattatccatttaagttatcatatcaagacggtattaatttcccacgtgataactttaaagagcctgagggtatttgcatttatacaaatccaaaaacaaaacgtaaatcgttattacttgctatga

SEQ ID NO: 48 - DNA, 129 bp, Staphylococcus aureus Bacteriophage 44 AHJD
atgaatgtatgtaagttgttcaggtgtgagttttgcaaaacatttcacagcatagtcataggcttcactatcattcatatcattatctttatcaaaaatcgtataattaaaatctgttttaagttgtga

SEQ ID NO: 49 - DNA, 129 bp, Staphylococcus aureus Bacteriophage 44 AHJD
atgaggacgttgttgacattatcaatgctggagaagttcaattcacaatttatgaatatgaaaacaaaaaaggtcaaaaaggttactcaatcaattttggtcaagtatcattttaatacaatttcatag

SEQ ID NO: 50 - DNA, 126 bp, Staphylococcus aureus Bacteriophage 44 AHJD
atgagggggcaggtgttgactttgatggtgcatatggatttcaatgtatggacttatcagttgcttatgtgtattacattactgacggtaaagttcgcatgtggggtaatgctaaagacgcgataa

SEQ ID NO: 51 - DNA, 126 bp, Staphylococcus aureus Bacteriophage 44 AHJD
gtgtgttacgtttttcattcacgtaatcgtttcgtcgcatttctaaaaaaatgtttttgtaaagtcttgatgtattcattttatgcttttgtaataaattgtatatatttaaattggataatatag

SEQ ID NO: 52 - DNA, 123 bp, Staphylococcus aureus Bacteriophage 44 AHJD
atgataacaatgaactatacaatatcattaacggttacaaaaacactgaacgtaatatattattctctacatttgtcacatcacgttcattgtataacttattggttcctttccaatacttaa

SEQ ID NO: 53 - DNA, 123 bp, Staphylococcus aureus Bacteriophage 44 AHJD
atgattttagtaatgttaattttaaatttgatgataaagatttacaagaggcgtacattgacacatggaaacattttgcacatctgccctattttcctaaagaaagaaacgtatcatatgtaa

SEQ ID NO: 54 - DNA, 120 bp, Staphylococcus aureus Bacteriophage 44 AHJD
atgtggtttattcatcaagtgaagttgaaaaatacttacaatcacaaggcttcacagaacacaatgaagatacaacaagtaacactgatgaaacatcgaatcaaaatgctacatctttag

SEQ ID NO: 55 - DNA, 120 bp, Staphylococcus aureus Bacteriophage 44 AHJD
atgactggaatggaaatacgatgttactcgacgctggtaagatttcacaaaaaactggtgttaagttacgtacaaaatcaattattggttatcataatgaagttcgagtatatccagtag

SEQ ID NO: 56 - DNA, 117 bp, Staphylococcus aureus Bacteriophage 44 AHJD
atgtgtctggtaataattcttttgcttgtgttttggttaaatgatactcgtgaagtggtaaaaattcctcaatgtattcattatcatcatctaagtaatgaagtatataacctttga

SEQ ID NO: 57 - DNA, 114 bp, Staphylococcus aureus Bacteriophage 44 AHJD
gtgagtattacattacaggtaaccaaatggaattatttagagacgcgccagaagaaattaaaaaagtgggtgcatggttacgtgtgtcaagtggtaacgcagtcggtgaagtaa

SEQ ID NO: 58 - DNA, 111 bp, Staphylococcus aureus Bacteriophage 44 AHJD
atgtaccaccatatgttgcaccatcacgtgtggtattgtatacactcattaatggcgtaccaaataatgctggtgataatattgtattctttagtggtattgcttaattaa

SEQ ID NO: 59 - DNA, 111 bp, Staphylococcus aureus Bacteriophage 44 AHJD
atgcatatttcttatgattcagtacaaacatcttatctatctgttcgttttcaatatcccatttacctaaggctatcgggtcgaataaactggggttcaataagggtttaa

SEQ ID NO: 60 - DNA, 111 bp, Staphylococcus aureus Bacteriophage 44 AHJD
atggattttgtaacattggattacctgaaccgtcattatgccaaaatcttacaccagattctaaaattgcttttaattgttccattaacatggggtcgatgtcacgtatag

SEQ ID NO: 61 - DNA, 111 bp, Staphylococcus aureus Bacteriophage 44 AHJD
atgtaccattttcatttctataatatgtgccgtattggtttcgtttccattttccaaatgtatttacttttgatgtttctaatgctttgctattactacctgaaaatttag

SEQ ID NO: 62 - DNA, 108 bp, Staphylococcus aureus Bacteriophage 44 AHJD
atgtgttttggtgtcttgataaaatatcttttacgtttgtcattttatttctcctcttatttaaattatttgctttctgcaattgcgatttgtagtaaatcattgtaa

SEQ ID NO: 63 - DNA, 105 bp, Staphylococcus aureus Bacteriophage 44 AHJD
gtggtattcgcaacgcagttaaccaatctattaatattgataaagaaacaaatcacatgtactctacacaatccgattctcaaaaacctgaaggtttttggataa

SEQ ID NO: 64 - DNA, 105 bp, Staphylococcus aureus Bacteriophage 44 AHJD
atgcgtcttgtattttttttaataattcttgcatggcttgttttgctaaagcgagtagtgaactaccactgtcaccactactaccactgtcagacgaatcactag

SEQ ID NO: 65 - DNA, 102 bp, Staphylococcus aureus Bacteriophage 44 AHJD
atgacgagtcaatcaatcaacttgtgtccgaaatatataacggtgcaccatttgttaaaatgtcacctatgtttaatgcagatgacgatatcattgatttaa

SEQ ID NO: 66 - DNA, 102 bp, Staphylococcus aureus Bacteriophage 44 AHJD
gtggtggaaaacgtttccataatttatatggtttcttccaacttggtgagtatgaacactttgaagcattacgcgcaagaggttcacaaaactataaattaa

SEQ ID NO: 67 - DNA, 102 bp, Staphylococcus aureus Bacteriophage 44 AHJD
atgatattctttatattgaaagtgacatcggttcattttcacttaacgacttatttccagttgaacgttcagtacataacaaatctgatttgcatatattaa

SEQ ID NO: 68 - DNA, 402 bp, Staphylococcus aureus Bacteriophage 44 AHJD
atgacagaatttgatgaaatcgtaaaaccagacgacaaagaagaaacttcagaatcaactgaagaaaatttagaatcaactgaagaaacttcagaatcaactgaagaatcaactgaagaatcaactgaagaatcaactgaagataaaacagtagaaacaatcgaagaagaaaatgaaaacaaattagaacctactacaacagatgaagatagttcgaaatttgaccctgttgtattagaacaacgtattgcttcattagaacaacaagtgactacttttttatcttcacaaatgcaacaaccacaacaagtacaacaaacacaatcagatgtaacagaatcaaacaaagaagataacgactattcagatgaagaactagttgataagttagatttagattag

SEQ ID NO: 69 - DNA, 303 bp, Staphylococcus aureus Bacteriophage 44 AHJD
atggttaatgttgataatgcaccagaagaaaaaggacaagcctatactgaaatgttgcaactattcaataaactgattcaatggaatccagcttatacatttgacaatgcaattaacttattatcggcttgccaacaactattattaaactataatagttctgttgttcaattcttaaatgatgaactaaacaacgaaactaaaccagaatcaatattgtcttatattgctggtgatgacccaatagaacaatggaatatgcataaaggattttatgaaacgtataacgtttacgttttttag

SEQ ID NO: 70 - DNA, 198 bp, Staphylococcus aureus Bacteriophage 44 AHJD
atggaaaatgaaacaaaaaacattgagttgaagcatgtttttcgttttaagaatggaagtttatgtatagcgttatttgatagaacagaaaatgaaatttcattttatgatgttgacattgatgaaattgaagatttaaatcataattctgttttacgcgtaatttcaactttattaggaagtgataataatggttaa

SEQ ID NO: 71 - DNA, 183 bp, Staphylococcus aureus Bacteriophage 44 AHJD
atgtatgagggaaacaacatgcgttctatgatgggtacatcatatgaagattcaagattaaataaacgaacagaattaaatgaaaacatgtcaattgatacaaataaaagtgaagatagttatggtgtacaaattcattcactttcaaaacaatcatttacaggtgacgttgaggaggaataa

SEQ ID NO: 72 - DNA, 150 bp, Staphylococcus aureus Bacteriophage 44 AHJD
atgaaaacatgtcaattgatacaaataaaagtgaagatagttatggtgtacaaattcattcactttcaaaacaatcatttacaggtgacgttgaggaggaataataaattatggcacaacaatctacaaaaaatgaaactgcacttttag

SEQ ID NO: 73 - DNA, 132 bp, Staphylococcus aureus Bacteriophage 44 AHJD
atgattgttttgaaagtgaatgaatttgtacaccataactatcttcacttttatttgtatcaattgacatgttttcatttaattctgttcgtttatttaatcttgaatcttcatatgatgtacccatcatag

SEQ ID NO: 74 - DNA, 111 bp, Staphylococcus aureus Bacteriophage 44 AHJD
atgttttcatttaattctgttcgtttatttaatcttgaatcttcatatgatgtacccatcatagaacgcatgttgtttccctcatacatgtttaaattcctcctaatctaa

SEQ ID NO: 75 - protein, 761 aa, Staphylococcus aureus Bacteriophage 44 AHJD
MGLLECMQYHKHERRMILYWDIETLAYNKVNGRKKPTKYKNVTYSVAIGWFNGYEIDVEVFPSFESFYDAFYTYVKRRDTITKSKTDIIMIAHNCNKYDNHFLLKDTMRYFDNITRENIYLKSAEENEHTLKMKEATILAKNQNVILEKRVKSSINLDLTMFLNGFKFNIIDNFMKTNTSIATLGKKLLDGGYLTESQLKTDFNYTIFDKDNDMNDSEAYDYAVKCFAKLTPEQLTYIHNDVIILGMCHIHYSDIFPNFDYNKLTFSLNIMESYLNNEMTRFQLLNQYQDIKISYTHYHFHDMNFYDYIKSFYRGGLNMYNTKYINKLIDEPCFSIDINSSYPYVMYHEKIPTWLYFYEHYSEPTLIPTFLDDDNYFSLYKIDKDVFNDDLLIKIKSRVLRQMIVKYYNNDNDYVNINTNTLRMIQDITGIDCMHIRVNSFVIYECEYFHARDIIFQNYFIKTQGKLKNKINMTSPYDYHITDDINEHPYSNEEVMLSKVVLNGLYGIPALRSHFNLFRLDDNNELYNIINGYKNTERNILFSTFVTSRSLYNLLVPFQYLTESEIDDNFIYCDTDSLYMKSVVKPLLNPSLFDPIALGKWDIENEQIDKMFVLNHKKYAYEVNGKIKIASAGIPKNAFDTSVDFETFVREQFFDGAIIENNKSIYNEQGTISIYPSKTEIVCGNVYDEYFTDELNMKREFILKDARENFDHSQFDDILYIESDIGSFSLNDLFPVERSVHNKSDLHILKREHDEIKKGNC

SEQ ID NO: 76 - protein, 647 aa, Staphylococcus aureus Bacteriophage 44 AHJD
MAYNENDFKYFDDIRPFLDEIYKTRERYTPFYDDRADYNTNSKSYYDYISRLSKLIEVLARRIWDYDNELKKRFKNWDDLMKAFPEQAKDLFRGWLNDGTIDSIIHDEFKKYSAGLTSAFALFKVTEMKQMNDFKSEVKDLIKDIDRFVNGFELNELEPKFVMGFGGIRNAVNQSINIDKETNHMYSTQSDSQKPEGFWINKLTPSGDLISSMRIVQGGHGTTIGLERQSNGEMKIWLHHDGVAKLLQVAYKDNYVLDLEEAKGLTDYTPQSLLNKHTFTPLIDEANDKLILRFGDGTIQVRSRADVKNHIDNVEKEMTIDNSENNDNRWMQGIAVDGDDLYWLSGNSSVNSHVQIGKYSLTTGQKIYDYPFKLSYQDGINFPRDNFKEPEGICIYTNPKTKRKSLLLAMTNGGGGKRFHNLYGFFQLGEYEHFEALRARGSQNYKLTKDDGRALSIPDHIDDLNDLTQAGFYYIDGGTAEKLKNMPMNGSKRIIDAGCFINVYPTTQTLGTVQELTRFSTGRKMVKMVRGMTLDVFTLKWDYGLWTTIKTDAPYQEYLEASQYNNWIAYVTTAGEYYITGNQMELFRDAPEEIKKVGAWLRVSSGNAVGEVRQTLEANISEYKEFFSNVNAETKHREYGWVAKHQK

SEQ ID NO: 77 - protein, 587 aa, Staphylococcus aureus Bacteriophage 44 AHJD
MRKLTNFKFFYNTPFTDYQNTIHFNSNKERDDYFLNGRHFKSLDYSKQPYNFIRDRMEINVDMQWHDAQGINYMTFLSDFEDRRYYAFVNQIEYVNDVVVKIYFVIDTIMTYTQGNVLEQLSNVNIERQHLSKRTYNYMLPMLRNNDDVLKVSNKNYVYNQMQQYLENLVLFQSSADLSKKFGTKKEPNLDTSKGTIYDNITSPVNLYVMEYGDFINFMDKMSAYPWITQNFQKVQMLPKDFINTKDLEDVKTSEKITGLKTLKQGGKSKEWSLKDLSLSFSNLQEMMLSKKDEFKHMIRNEYMTIEFYDWNGNTMLLDAGKISQKTGVKLRTKSIIGYHNEVRVYPVDYNSAENDRPILAKNKEILIDTGSFLNTNITFNSFAQVPILINNGILGQSQQANRQKNAESQLITNRIDNVLNGSDPKSRFYDAVSVASNLSPTALFGKFNEEYNFYKQQQAEYKDLALQPPSVTESEMGNAFQIANSINGLTMKISVPSPKEITFLQKYYMLFGFEVNDYNSFIEPINSMTVCNYLKCTGTYTIRDIDPMLMEQLKAILESGVRFWHNDGSGNPMLQNPLNNKFREGV

SEQ ID NO: 78 - protein, 487 aa, Staphylococcus aureus Bacteriophage 44 AHJD
MILKRVITMNDQEKIDKFTHSYINDDFGLTIDQLVPKVKGYGRFNVWLGGNESKIRQVLKAVKEIGVSPTLFAVYEKNEGFSSGLGWLNHTSARGDYLTDAKFIARKLVSQSKQAGQPSWYDAGNIVHFVPQDVQRKGNADFAKNMKAGTIGRAYIPLTAAATWAAYYPLGLKASYNKVQNYGNPFLDGANTILAWGGKLDGKGGSPSDSSDSGSSGDSGSSLLALAKQAMQELLKKIQDALQWDVHSIGSDKFFSNDYFTLEKTFNNTYHIKMTIGLLDSLKKLIDSVQVDSGSSSSNPTDDDGDHKPISGKSVKPNGKSGRVIGGNWTYAQLPEKYKKAIGVPLFKKEYLYKPGNIFPQTGNAGQCTELTWAYMSQLHGKRQPTDDGQITNGQRVWYVYKKLGAKTTHNPTVGYGFSSKPPYLQATAYGIGHTGVVVAVFEDGSFLVANYNVPPYVAPSRVVLYTLINGVPNNAGDNIVFFSGIA

SEQ ID NO: 79 - protein, 415 aa, Staphylococcus aureus Bacteriophage 44 AHJD
MVKQNRLDMVRDYQNAVNHVRKKIPDKYNQIELVDELMNDDIDYYISISNRSDGKSFNYVSFFIYLAIKLDIKFTLLSRHYTLRDAYRDFIEEIIDENPLFKSKRVTFRSARDYLAIIYQDKEIGVITDLNSATDLKYHSNFLKHYPIIIYDEFLALEDDYLIDEWDKLKTIYESIDRNHGNVDYIGFPKMFLLGNAVNFSSPILSNLNIYNLLQKHKMNTSRLYKNIFLEMRRNDYVNEKRNTRAFNSNDDAMTTGEFEFNEYNLADDNLRNHINQNGDFFYIKTDDKYIKVMYNVTTFMTNIIVVPYTKQYEFCTKIRDIDNHVTYLRDDMFYKENMERYYYNPSNLHFDNAYSKNYVVDNDRYLYLDMNKIIKFHIKNEMKKNMSEFERKEKIYEDNYIENTKKYLMKQYGL

SEQ ID NO: 80 - protein, 408 aa, Staphylococcus aureus Bacteriophage 44 AHJD
MAQQSTKNETALLVAKSAKSALQDFNHDYSKSWTFGDKWDNSNTMFETFVNKYLFPKINETLLIDIALGNRFNWLAKEQDFIGQYSEEYVIMDTVPINMDLSKNEELMLKRNYPRMATKLYGNGIVKKQKFTLNNNDTRFNFQTLADATNYALGVYKKKISDINVLEEKEMRAMLVDYSLNQLSETNVRKATSKEDLASKVFEAILNLQNNSAKYNEVHRASGGAIGQYTTVSKLKDIVILTTDSLKSYLLDTKIANTFQIAGIDFTDHVISFDDLGGVFKVTKEFKLQNQDSIDFLRAYGDYQSQLGDTIPVGAVFTYDVSKLKEFTGNVEEIKPKSDLYAFILDINSIKYKRYTKGMLKPPFHNPEFDEVTHWIHYYSFKAISPFFNKILITDQDVNPKPEEELQE

SEQ ID NO: 81 - protein, 327 aa, Staphylococcus aureus Bacteriophage 44 AHJD
MNNDKRGLNVELSKEISKRVVEHRNRFKRLMFNRYLEFLPLLINYTNRDTVGIDFIQLESALRQNINVVVGEARNKQIMILGYVNNTYFNQAPNFSSNFNFQFQKRLTKEDIYFIVPDYLIPDDCLQIHKLYDNCMSGNFVVMQNKPIQYNSDIEIIEHYTDELAEVALSRFSLIMQAKFSKIFKSEINDESINQLVSEIYNGAPFVKMSPMFNADDDIIDLTSNSVIPALTEMKREYQNKISELSNYLGINSLAVDKESGVSDEEAKSNRGFTTSNSNIYLKGREPITFLSKRYGLDIKPYYDDETTSKISMVDTLFKDESSDING

SEQ ID NO: 82 - protein, 251 aa, Staphylococcus aureus Bacteriophage 44 AHJD
MARYTMTLYDFIKSELIKKGFNEFVNDNKLTFYDDEFQFMQKMLKFDKDVLAIVNEKVFKGFSLKDELSDLLFKKSFTIHFLDREINRQTVEAFGMQVITVCITHEDYLNVVYSSSEVEKYLQSQGFTEHNEDTTSNTDETSNQNATSLDNSTGMTANRNAYVSLPQSEVNIDVDNTTLRFADNNTIDNGKTVNKSSNESNQNAKRNQNQKGNAKGTQFTKQYLIDNIDKAYDLRKKILNEFDKKCFLQIW

SEQ ID NO: 83 - protein, 250 aa, Staphylococcus aureus Bacteriophage 44 AHJD
MKSQQQAKEWIYKHEGAGVDFDGAYGFQCMDLSVAYVYYITDGKVRMWGNAKDAINNDFKGLATVYKNTPSFKPQLGDVAVYTNGQYGHIQCVLSGNLDYYTCLEQNWLGGGFDGWEKATIRTHYYDGVTHFIRPKFSGSNSKALETSKVNTFGKWKRNQYGTYYRNENGTFTCGFLPIFARVGSPKLSEPNGYWFQPNGYTPYNEVCLSDGYVWIGYNWQGTRYYLPVRQWNGKTGNSYSVGIPWGVFS

SEQ ID NO: 84 - protein, 160 aa, Staphylococcus aureus Bacteriophage 44 AHJD
LVRHTSEMDRWKKEREARKEQEKDLFLNDFSNVNFKFDDKDLQEAYIDTWKHFAHLPYFPKERNVSYVNAVSLVRGSRHKKLNYILEIYNRNDDSNNKNAKKHKYALYNLQAKNNNSSMYKYIKEIDTLYKEIGKSDRPVTNIDDEDVRYNFLYYATFDE

SEQ ID NO: 85 - protein, 122 aa, Staphylococcus aureus Bacteriophage 44 AHJD
MTNVKDILSRHQNTLARFEFEEKEREFIKLSELVEKYGMKKEYIVRALFTNKESKFGEQGVIVTDDYNVNLPNHLTELIKEMRADEDVVDIINAGEVQFTIYEYENKKGQKGYSINFGQVSF

SEQ ID NO: 86 - protein, 140 aa, Staphylococcus aureus Bacteriophage 44 AHJD
MNEVKFRFTDSEAFHMFIYAGDLKLLYFLFVLMFVDIITGISKAIKNNNLWSKKSMRGFSKKLLIFCIIILANIIDQILQLKGGLLMITIFYYIANEGLSIVENCAEMDVLVPEQIKDKLRVIKNDTEKSDNNERSREDR

SEQ ID NO: 87 - protein, 136 aa, Staphylococcus aureus Bacteriophage 44 AHJD
MKIKTTFRLNNLIYYLLTNRDYYNDKFEKFTSSNKKCIVKINMGDVYIEFDKQYDDFEIEKELFTLDIDIDIKKHVFNILVFYYRNYLSNELIREILLNVTIDDVLSNFDKPLESELMIIYQNKVIYDNGKVIDHE

SEQ ID NO: 88 - protein, 92 aa, Staphylococcus aureus Bacteriophage 44 AHJD
MKMVHLHVVFYQYLHVSVVQNYQNLMAIGSNQTVIHHITKFVYQMVTYGLVITGKAHVIIYQCANGMEKQVIVTVLVFLGGCSHNGYFSLFL

SEQ ID NO: 89 - protein, 80 aa, Staphylococcus aureus Bacteriophage 44 AHJD
VTITPCSPNFDSLFVNNALTIYSFFIPYFSTNSDSLINSLSFSSNSNLANVFWCLDKISFTFVILFLLLFKLFAFCNCDL

SEQ ID NO: 90 - protein, 78 aa, Staphylococcus aureus Bacteriophage 44 AHJD
MKVDDIVTLRVKGYILHYLDDDNEYIEEFLPLHEYHLTKTQAKELLPDTCKLLSTTRTTKTIQVYYNDLLQIAIAESK

SEQ ID NO: 91 - protein, 73 aa, Staphylococcus aureus Bacteriophage 44 AHJD
MERLKLLLLVYRKTPLIQASILKPLYVNNSLTVPLLKTIKVSIMSKVQYRYIRLKLKLYVVMYMMNILLMNLI

SEQ ID NO: 92 - protein, 70 aa, Staphylococcus aureus Bacteriophage 44 AHJD
MLIGTVSIITYSSLYCPIKSCSLANQLKRLPNAISINKVSLILGNKYLFTNVSNIVFELSHLSPNVQDFE

SEQ ID NO: 93 - protein, 68 aa, Staphylococcus aureus Bacteriophage 44 AHJD
MLPGLYKYSFLNKGTPIAFLYFSGNCAYVQLPPITRPLFPFGLTDLPLIGLWSPSSSVGLELLLPLST

SEQ ID NO: 94 - protein, 62 aa, Staphylococcus aureus Bacteriophage 44 AHJD
MSKRFCFTMFLLLVIVYDVVYSVKFIRQMLHNIKSYTSHLHHQYLSLVYLIYQFLYIKYRFL

SEQ ID NO: 95 - protein, 60 aa, Staphylococcus aureus Bacteriophage 44 AHJD
MHHQSQHLPPHAYISILLLVVVISFISLLFLMFCYPTIFTMFCFRINITEEFFIFRYISL

SEQ ID NO: 96 - protein, 47 aa, Staphylococcus aureus Bacteriophage 44 AHJD
MFAKMIIQNINNFLENPLIDFFDHKLLFLIAFEIPVIISTNINTNKK

SEQ ID NO: 97 - protein, 59 aa, Staphylococcus aureus Bacteriophage 44 AHJD
MRTPPKEYQHCNYYLFFHSIGALVNNNVCLASYNQSIRNHLINKLRYMVYNRLVGTNSH

SEQ ID NO: 98 - protein, 58 aa, Staphylococcus aureus Bacteriophage 44 AHJD
VSMYASCKSLSSNLKLTLLKSFKNKSFSCSFLASLSFFHLSISDVCLTNVINLHIKHK

SEQ ID NO: 99 - protein, 58 aa, Staphylococcus aureus Bacteriophage 44 AHJD
MERKYKTVLLYCDEIKGHFPHQISMFEDLYDAKVVYSYYEYNLFTKKYAYIIEYIKEI

SEQ ID NO: 100 - protein, 55 aa, Staphylococcus aureus Bacteriophage 44 AHJD
MNNLLNIAIVFLLAFLITLIILMTLHIRVSFGVLFTTLIIFYIIFLMVIYALYGG

SEQ ID NO: 101 - protein, 54 aa, Staphylococcus aureus Bacteriophage 44 AHJD
MIVYIPNFSTKFILFCIWYNDNICHKSSYIIHDFNIFIISFDIEEITVLIDVIS

SEQ ID NO: 102 - protein, 54 aa, Staphylococcus aureus Bacteriophage 44 AHJD
MEYMHVQLYLLSYFLQNLHYLFFVRLVVQSGRCYLRHTKTVVQLVLIVILTFLL

SEQ ID NO: 103 - protein, 54 aa, Staphylococcus aureus Bacteriophage 44 AHJD
VFKWNVNTKRYYYIAMRLKDIFHIKSQCLKIYMTLKLYIHIMNITCSLKNTRIS

SEQ ID NO: 104 - protein, 53 aa, Staphylococcus aureus Bacteriophage 44 AHJD
VNKTPNDTRICSVISMISVIKNAKRKTMAMFNRLFMVNHFPIIVYDFVLINNH

SEQ ID NO: 105 - protein, 50 aa, Staphylococcus aureus Bacteriophage 44 AHJD
MILYSSLLSSKRNKLKCERNAGMPYNPFKTTLDNITSSFEYGCSLISSVM

SEQ ID NO: 106 - protein, 49 aa, Staphylococcus aureus Bacteriophage 44 AHJD
MACFAKASSELPLSPLLPLSDESLGDPPLPSNLPPQARIVFAPSKNGLP

SEQ ID NO: 107 - protein, 49 aa, Staphylococcus aureus Bacteriophage 44 AHJD
MPLFNHLYQICKKHFLSNSFKIFFLKSYALSILSIKYCLVNCVPFALPF

SEQ ID NO: 108 - protein, 48 aa, Staphylococcus aureus Bacteriophage 44 AHJD
MMILIIKTLKSINTLYIIYKLKIIILQCINILKKSILYIKKLVNQIDQ

SEQ ID NO: 109 - protein, 48 aa, Staphylococcus aureus Bacteriophage 44 AHJD
MQHLTNKFNTVNDIINYYKEQKHGKTKSFRHGKRLSKCCQSCQKKNPR

SEQ ID NO: 110 - protein, 48 aa, Staphylococcus aureus Bacteriophage 44 AHJD
VYTIPHVMVQHMVVHYSLQLKTNHLQKLLQQHLCDQYHMQLLVSMVVY

SEQ ID NO: 111 - protein, 47 aa, Staphylococcus aureus Bacteriophage 44 AHJD
MSISNVNNSFSISKSSYCLSNSIYTSPIFIFTIHFLLDEVNFSNLSL

SEQ ID NO: 112 - protein, 47 aa, Staphylococcus aureus Bacteriophage 44 AHJD
VYLLHYLFDFDYVLRFDYFRYSIYSQFYRYQSYYYQRIVTLYYQHQC

SEQ ID NO: 113 - protein, 46 aa, Staphylococcus aureus Bacteriophage 44 AHJD
VLYLLMMYLNLKSLLATLKKLNQNQIYMRLFWILIQLNINVTQKVC

SEQ ID NO: 114 - protein, 45 aa, Staphylococcus aureus Bacteriophage 44 AHJD
VVTGHMHSYQKNIKKQLVYLYSKKNTYTNQVTYFLKRVMQDNVQN

SEQ ID NO: 115 - protein, 45 aa, Staphylococcus aureus Bacteriophage 44 AHJD
MSSTFIIISLLSKKRKRYTFHKILYAYSIVLLGHHQQYKTILILV

SEQ ID NO: 116 - protein, 45 aa, Staphylococcus aureus Bacteriophage 44 AHJD
MHDRRLLLIYSFVNLLRVMLQSVHTHQVGRNHINYGNVFHHRRLS

SEQ ID NO: 117 - protein, 45 aa, Staphylococcus aureus Bacteriophage 44 AHJD
MRLVTVLQHHRDVTRFSFHHWIDVLIRLLYHDHPVQYACLKLSHH

SEQ ID NO: 118 - protein, 44 aa, Staphylococcus aureus Bacteriophage 44 AHJD
MLPIYVMICFIKKTWNVITTIQAIYILTMLTLKITWLIMIDIYI

SEQ ID NO: 119 - protein, 42 aa, Staphylococcus aureus Bacteriophage 44 AHJD
MAPSKNCSRTKVSKSTLVSKAFFGIPAEAILIFPFTSYAYFL

SEQ ID NO: 120 - protein, 42 aa, Staphylococcus aureus Bacteriophage 44 AHJD
MIIHLSYHIKTVLISHVITLKSLRVFAFIQIQKQNVNRYYLL

SEQ ID NO: 121 - protein, 42 aa, Staphylococcus aureus Bacteriophage 44 AHJD
MNVCKLFRCEFCKTFHSIVIGFTIIHIIIFIKNRIIKICFKL

SEQ ID NO: 122 - protein, 42 aa, Staphylococcus aureus Bacteriophage 44 AHJD
MRTLLTLSMLEKFNSQFMNMKTKKVKKVTQSILVKYHFNTIS

SEQ ID NO: 123 - protein, 41 aa, Staphylococcus aureus Bacteriophage 44 AHJD
MRGQVLTLMVHMDFNVWTYQLLMCITLLTVKFACGVMLKTR

SEQ ID NO: 124 - protein, 41 aa, Staphylococcus aureus Bacteriophage 44 AHJD
VCYVFHSRNRFVAFLKKCFCKVLMYSFYAFVINCIYLNWII

SEQ ID NO: 125 - protein, 40 aa, Staphylococcus aureus Bacteriophage 44 AHJD
MITMNYTISLTVTKTLNVIYYSLHLSHHVHCITYWFLSNT

SEQ ID NO: 126 - protein, 40 aa, Staphylococcus aureus Bacteriophage 44 AHJD
MILVMLILNLMIKIYKRRTLTHGNILHICPIFLKKETYHM

SEQ ID NO: 127 - protein, 39 aa, Staphylococcus aureus Bacteriophage 44 AHJD
MWFIHQVKLKNTYNHKASQNTMKIQQVTLMKHRIKMLHL

SEQ ID NO: 128 - protein, 39 aa, Staphylococcus aureus Bacteriophage 44 AHJD
MTGMEIRCYSTLVRFHKKLVLSYVQNQLLVIIMKFEYIQ

SEQ ID NO: 129 - protein, 38 aa, Staphylococcus aureus Bacteriophage 44 AHJD
MCLVIILLLVFWLNDTREVVKIPQCIHYHHLSNEVYNL

SEQ ID NO: 130 - protein, 37 aa, Staphylococcus aureus Bacteriophage 44 AHJD
VSITLQVTKWNYLETRQKKLKKWVHGYVCQVVTQSVK

SEQ ID NO: 131 - protein, 36 aa, Staphylococcus aureus Bacteriophage 44 AHJD
MYHHMLHHHVWYCIHSLMAYQIMLVIILYSLVVLLN

SEQ ID NO: 132 - protein, 36 aa, Staphylococcus aureus Bacteriophage 44 AHJD
MHISYDSVQTSYLSVRFQYPIYLRLSGRINWGSIRV

SEQ ID NO: 133 - protein, 36 aa, Staphylococcus aureus Bacteriophage 44 AHJD
MDFVTLDYLNRHYAKILHQILKLLLIVPLTWGRCHV

SEQ ID NO: 134 - protein, 36 aa, Staphylococcus aureus Bacteriophage 44 AHJD
MYHFHFYNMCRIGFVSIFQMYLLLMFLMLCYYYLKI

SEQ ID NO: 135 - protein, 35 aa, Staphylococcus aureus Bacteriophage 44 AHJD
MCFGVLIKYLLRLSFYFSSYLNYLLSAIAICSKSL

SEQ ID NO: 136 - protein, 34 aa, Staphylococcus aureus Bacteriophage 44 AHJD
VVFATQLTNLLILIKKQITCTLHNPILKNLKVFG

SEQ ID NO: 137 - protein, 34 aa, Staphylococcus aureus Bacteriophage 44 AHJD
MRLVFFLIILAWLVLLKRVVNYHCHHYYHCQTNH

SEQ ID NO: 138 - protein, 33 aa, Staphylococcus aureus Bacteriophage 44 AHJD
MTSQSINLCPKYITVHHLLKCHLCLMQMTISLI

SEQ ID NO: 139 - protein, 33 aa, Staphylococcus aureus Bacteriophage 44 AHJD
VVENVSIIYMVSSNLVSMNTLKHYAQEVHKTIN

SEQ ID NO: 140 - protein, 33 aa, Staphylococcus aureus Bacteriophage 44 AHJD
MIFFILKVTSVHFHLTTYFQLNVQYITNLICIY

SEQ ID NO: 141 - protein, 133 aa, Staphylococcus aureus Bacteriophage 44 AHJD
MTEFDEIVKPDDKEETSESTEENLESTEETSESTEESTEESTEESTEDKTVETIEEENENKLEPTTTDEDSSKFDPVVLEQRIASLEQQVTTFLSSQMQQPQQVQQTQSDVTESNKEDNDYSDEELVDKLDLD

SEQ ID NO: 142 - protein, 100 aa, Staphylococcus aureus Bacteriophage 44 AHJD
MVNVDNAPEEKGQAYTEMLQLFNKLIQWNPAYTFDNAINLLSACQQLLLNYNSSVVQFLNDELNNETKPESILSYIAGDDPIEQWNMHKGFYETYNVYVF

SEQ ID NO: 143 - protein, 65 aa, Staphylococcus aureus Bacteriophage 44 AHJD
MENETKNIELKHVFRFKNGSLCIALFDRTENEISFYDVDIDEIEDLNHNSVLRVISTLLGSDNNG

SEQ ID NO: 144 - protein, 60 aa, Staphylococcus aureus Bacteriophage 44 AHJD
MYEGNNMRSMMGTSYEDSRLNKRTELNENMSIDTNKSEDSYGVQIHSLSKQSFTGDVEEE

SEQ ID NO: 145 - protein, 49 aa, Staphylococcus aureus Bacteriophage 44 AHJD
MKTCQLIQIKVKIVMVYKFIHFQNNHLQVTLRRNNKLWHNNLQKMKLHF

SEQ ID NO: 146 - protein, 43 aa, Staphylococcus aureus Bacteriophage 44 AHJD
MIVLKVNEFVHHNYLHFYLYQLTCFHLILFVYLILNLHMMYPS

SEQ ID NO: 147 - protein, 36 aa, Staphylococcus aureus Bacteriophage 44 AHJD
MFSFNSVRLFNLESSYDVPIIERMLFPSYMFKFLLI

SEQ ID NO: 148 - DNA, 53 bp, Staphylococcus aureus Bacteriophage 44 AHJD
gatcccggtcgaccaagctttacccatacgacgtcccagactacgccagctga

SEQ ID NO: 149 - DNA, 53 bp, Staphylococcus aureus Bacteriophage 44 AHJD
agcttcagctggcgtagtctgggacgtcgtatgggtaaagcttggtcgaccgg

SEQ ID NO: 150 - DNA, 21 bp, Staphylococcus aureus Bacteriophage 44 AHJD
aattctcgagtaaaataacat

SEQ ID NO: 151 - DNA, 37 bp, Staphylococcus aureus Bacteriophage 44 AHJD
cgggatccgcctccttttctcaacagtcacctgattt

SEQ ID NO: 152 - DNA, 27 bp, Staphylococcus aureus Bacteriophage 44 AHJD
cgggatccatgaggggttccgaagacg

SEQ ID NO: 153 - DNA, 24 bp, Staphylococcus aureus Bacteriophage 44 AHJD
cccaagcttacaatttggactttc

SEQ ID NO: 154 - DNA, 27 bp, Staphylococcus aureus Bacteriophage 44 AHJD
ccgctcgagctccaaattccaaaacag

SEQ ID NO: 155 - DNA, 26 bp, Staphylococcus aureus Bacteriophage 44 AHJD
cgggatccaataagactcctttttac

SEQ ID NO: 156 - DNA, 10 bp, Staphylococcus aureus Bacteriophage 44 AHJD
gcgtcgaccg

SEQ ID NO: 157 - DNA, 20 bp, Staphylococcus aureus Bacteriophage 44 AHJD
tattatccaaaacttgaaca

SEQ ID NO: 158 - DNA, 20 bp, Staphylococcus aureus Bacteriophage 44 AHJD
cggtggtatatccagtgatt

SEQ ID NO: 159 - protein, 133 aa, Staphylococcus aureus Bacteriophage 44 AHJD
MKIKVKKEMRLDELIKWARENPDLSQCKIFFSTGFSDGFVRFHPNTNKCSTSSFIPIDIPFIVDIEKEVTEETKVDRLIELFEIQEGDYNSTLYENTSIKECLYGRCVPTKAFYILNDDLTMTLIWKDGELLV

(19) United States
(12) Patent Application Publication
(10) Pub. No.: US 2004/0091856 A1
(43) Pub. Date: May 13, 2004

(54) DNA sequences from staphylococcus aureus bacteriophage 44AHJD that encode anti-microbial polypeptides

(76) Inventors: Jerry Pelletier, Baie-D'Urfe (CA); Philippe Gros, Lambert (CA); Michael Dubow, Montreal (CA)

Correspondence Address: FOLEY & LARDNER, P.O. BOX 80278, SAN DIEGO, CA, 92138-0278, US

(21) Appl. No.: 09/727,892

(22) Filed: Dec. 1, 2000

Related U.S. Application Data
(60) Provisional application No. 60/168,777, filed on Dec. 1, 1999.

(51) Int. Cl. C12Q 1/68; C12Q 1/70

(52) U.S. Cl. 435/6; 435/5

(57) ABSTRACT

The disclosure concerns particular bacteriophage open reading frame, and portions and products of those open reading frames which have antimicrobial activity Also disclosed is an S. aureus protein that interacts with an inhibitory phage protein. Methods of using such products are also described.

### RELATED APPLICATIONS

This application claims the benefit of U.S. Provisional Application Pelletier, et al., No. 60/168,777 filed Dec. 1, 1999, which is hereby incorporated by reference in its entirety, including drawings.

### BACKGROUND OF THE INVENTION

This invention relates to the identification of antimicrobial agents and of microbial targets of such agents, and in particular to the isolation of bacteriophage DNA sequences, and their translated protein products, showing anti-microbial activity. The DNA sequences can be expressed in expression vectors. These expression constructs and the proteins produced therefrom can be used for a variety of purposes including therapeutic methods and identification of microbial targets.

The following description is provided to assist the understanding of the reader. None of the information provided or references cited is admitted to be prior art to the present invention.

The frequency and spectrum of antibiotic-resistant infections have, in recent years, increased in both the hospital and community. Certain infections have become essentially untreatable and are growing to epidemic proportions in the developing world as well as in institutional settings in the developed world. The staggering spread of antibiotic resistance in pathogenic bacteria has been attributed to microbial genetic characteristics, widespread use of antibiotic drugs and changes in society that enhance the transmission of drug-resistant organisms (for a review, see Cohen, 1992). This spread of drug resistant microbes is leading to ever-increasing morbidity, mortality and health-care costs.

There are over 160 antibiotics currently available for treatment of microbial infections, all based on a few basic chemical structures and targeting a small number of metabolic pathways: bacterial cell wall synthesis, protein synthesis, and DNA replication. Despite all these antibiotics, a person could succumb to an infection as a result of a resistant bacterial infection. Resistance now reaches all classes of antibiotics currently in use, including: β-lactams, fluoroquinolones, aminoglycosides, macrolide peptides, chloramphenicol, tetracyclines, rifampicin, folate inhibitors, glycopeptides, and mupirocin. There is thus a need for new antibiotics, and this need will not subside given the ability bacteria have to overcome each new agent synthesized. It is also likely that targeting new pathways will play an important role in discovery of these new antibiotics. In fact, a number of crucial cellular pathways, such as secretion, cell division, and many metabolic functions, remain untargeted today.

Most major pharmaceutical companies have on-going drug discovery programs for novel anti-microbials. These are based on screens for small molecule inhibitors (e.g., natural products, bacterial culture media, libraries of small molecules, combinatorial chemistry) of crucial metabolic pathways of the micro-organism of interest. The screening process is largely for cytotoxic compounds and in most cases is not based on a known mechanism of action of the compounds. Classical drug screening programs are being exhausted and many of these pharmaceutical companies are looking towards rational drug design programs. Several small to mid-size biotechnology companies, as well as large pharmaceutical companies, have developed systematic high-throughput sequencing programs to decipher the genetic code of specific micro-organisms of interest. The goal is to identify, through sequencing, unique biochemical pathways or intermediates that are unique to the microorganism. Knowledge of the function of these bacterial genes, may form the rationale for a drug discovery program based on the mechanism of action of the identified enzymes/proteins. However, one of the most critical steps in this approach is the ascertainment that the identified proteins and biochemical pathways are 1) non-redundant and essential for bacterial survival, and 2) constitute suitable and accessible targets for drug discovery. These two issues are not easily addressed since to date, 18 prokaryotic genomes have been sequenced and 200 sequenced genomes are expected by the year 2000. For a majority of the sequenced genomes, less than 50% of the open reading frames (ORFs) have been linked to a known function. Even with the genome of Escherichia coli (E. coli), the most extensively studied bacterium, less than two-thirds of the annotated protein coding genes showed significant similarity to genes with ascribed functions (Rusterholtz and Pohlschroder, 1999). Thus considerable work must be undertaken to identify appropriate bacterial targets for drug screening.

### SUMMARY OF THE INVENTION

The present invention is based on the identification of, and demonstration that, specific DNA sequences of a bacteriophage, when introduced into a host bacterium can kill, or inhibit growth, of the host. Thus, these DNA sequences are anti-microbial agents. Information based on these DNA sequences can be utilized to develop peptide mimetics that can also function also as anti-microbials. The identification of the host bacterial proteins, targeted by the anti-microbial bacteriophage DNA sequences, can provide novel targets for drug design, compound screening or determination of new domains on an already known target.

In this regard, the terms “inhibit”, “inhibition”, “inhibitory”, and “inhibitor” all refer to a function of reducing a biological activity or function. Such reduction in activity or function can, for example, be in connection with a cellular component (e.g., an enzyme), or in connection with a cellular process (e.g., synthesis of a particular protein), or in connection with an overall process of a cell (e.g., cell growth). In reference to cell growth, the inhibitory effects may be bactericidal (killing of bacterial cells) or bacteriostatic (i.e., stopping or at least slowing bacterial cell growth). The latter slows or prevents cell growth such that fewer cells of the strain are produced relative to uninhibited cells over a given time period. From a molecular standpoint, such inhibition may equate with a reduction in the level of, or elimination of, the transcription and/or translation of a specific bacterial target(s), or reduction or elimination of activity of a particular target biomolecule.

In a first aspect the invention provides methods for identifying a target for antibacterial agents by identifying the bacterial target(s) of at least one inhibitory gene product, e.g., protein from ORFs 12 and 25, of bacteriophage 44AHJD or a homologous product. Such identification allows the development of antibacterial agents active on such targets. Preferred embodiments for identifying such targets involve the identification of binding of target and phage ORF products to one another. The target molecule may be a bacterial protein or other bacterial biomolecule, e.g., a nucleoprotein, a nucleic acid, a lipid or lipid-containing molecule, a nucleoside or nucleoside derivative, a polysaccharide or polysaccharide-containing molecule, or a peptidoglycan. The phage ORF products may be subportions of a larger ORF product that also binds the host target. Exemplary approaches are described below in the Detailed Description.

Additionally, the invention provides methods for identifying targets for antibacterial agents by identifying homologs of a Staphylococcus aureus target of a bacteriophage 44 AHJD ORFs 12 or 25 product. Such homologs may be utilized in the various aspects and embodiments described herein.

The term “fragment” refers to a portion and/or a segment of a larger molecule or assembly. For proteins, the term “fragment” refers to a molecule which includes at least 5 contiguous amino acids from the reference polypeptide or protein, preferably at least 6, 8, 10, 12, 15, 20, 30, 50 or more contiguous amino acids. In connection with oligo- or polynucleotides, the term “fragment” refers to a molecule which includes at least 15 contiguous nucleotides from a reference polynucleotide, preferably at least 18, 21, 24, 30, 36, 45, 60, 90, 150, or more contiguous nucleotides. Also in preferred embodiments, the fragment has a length in a range with the minimum as described above and a maximum which is no more than 90% of the length (or contains that percent of the contiguous amino acids or nucleotides) of the larger molecule (e.g., of the specified ORF), in other embodiments, the upper limit is no more than 60, 70, or 80% of the length of the larger molecule.

Stating that an agent, compound or test compound is “active on” a particular cellular target, such as the product of a particular gene, means that the target is an important part of a cellular pathway which includes that target and that the agent acts on that pathway. Thus, in some cases the agent may act on a component upstream or downstream of the stated target, including a regulator of that pathway or a component of that pathway. In general, an antibacterial agent is active on an essential cellular function, often on a product of an essential gene.

By “essential”, in connection with a gene or gene product, is meant that the host cannot survive without, or is significantly growth compromised, in the absence or depletion of functional product. An “essential gene” is thus one that encodes a product that is beneficial, or preferably necessary, for cellular growth in vitro in a medium appropriate for growth of a strain having a wild-type allele corresponding to the particular gene in question. Therefore, if an essential gene is inactivated or inhibited, that cell will grow significantly more slowly or even not at all. Preferably growth of a strain in which such a gene has been inactivated will be less than 20%, more preferably less than 10%, most preferably less than 5% of the growth rate of the wild-type, or not at all, in the growth medium. Preferably, in the absence of activity provided by a product of the gene, the cell will not grow at all or will be non-viable, at least under culture conditions similar to normal in vivo growth conditions. For example, absence of the biological activity of certain enzymes involved in bacterial cell wall synthesis can result in the lysis of cells under normal osmotic conditions, even though protoplasts can be maintained under controlled osmotic conditions. Preferably, but not necessarily, if such a gene is inhibited, e.g., with an antibacterial agent or a phage product, the growth rate of the inhibited bacteria will be less than 50%, more preferably less than 30%, still more preferably less than 20%, and most preferably less than 10% of the growth rate of the uninhibited bacteria. As recognized by those skilled in the art, the degree of growth inhibition will generally depend on the concentration of the inhibitory agent. In the context of the invention, essential genes are generally the preferred targets of antimicrobial agents. Essential genes can encode target molecules directly or can encode a product involved in the production, modification, or maintenance of a target molecule.

A “target” refers to a biomolecule that can be acted on by an exogenous agent, thereby modulating, preferably inhibiting, growth or viability of a cell. In most cases such a target will be a nucleic acid sequence or molecule, or a polypeptide or protein. However, other types of biomolecules can also be targets, e.g., membrane lipids and cell wall structural components.

The term “bacterium” refers to a single bacterial strain, and includes a single cell, and a plurality or population of cells of that strain unless clearly indicated to the contrary. In reference to bacteria or bacteriophage, the term “strain” refers to bacteria or phage having a particular genetic content. The genetic content includes genomic content as well as recombinant vectors. Thus, for example, two otherwise identical bacterial cells would represent different strains if each contained a vector, e.g., a plasmid, with different phage ORF inserts.

In the context of the phage nucleic acid sequences, e.g., gene sequences, of this invention, the terms “homolog” and “homologous” denote nucleotide sequences from different bacteria or phage strains or species or from other types of organisms that have significantly related nucleotide sequences, and consequently significantly related encoded gene products, preferably having related function. Homologous gene sequences or coding sequences have at least 70% sequence identity (as defined by the maximal base match in a computer-generated alignment of two or more nucleic acid sequences) over at least one sequence window of 48 nucleotides (or at least 99, 150, 200, or even the entire ORF or other sequence of interest), more preferably at least 80 or 85%, still more preferably at least 90%, and most preferably at least 95%. The polypeptide products of homologous genes have at least 35% amino acid sequence identity over at least one sequence window of 18 amino acid residues (or 24, 30, 33, 50, 100, or an entire polypeptide), more preferably at least 40%, still more preferably at least 50% or 60%, and most preferably at least 70%, 80%, or 90%. Preferably, the homologous gene product is also a functional homolog, meaning that the homolog will functionally complement one or more biological activities of the product being compared. For nucleotide or amino acid sequence comparisons where a homology is defined by a % sequence identity, the percentage is determined using BLAST programs (with default parameters (Altschul et al., 1997, “Gapped BLAST and PSI-BLAST: a new generation of protein database search programs, Nucleic Acid Res. 25:3389-3402). Any of a variety of algorithms known in the art which provide comparable results can also be used, preferably using default parameters. Performance characteristics for three different algorithms in homology searching is described in Salamov et al., 1999, “Combining sensitive database searches with multiple intermediates to detect distant homologues.” Protein Eng. 12:95-100. Another exemplary program package is the GCG™ package from the University of Wisconsin.

Homologs may also or in addition be characterized by the ability of two complementary nucleic acid strands to hybridize to each other under appropriately stringent conditions. Hybridizations are typically and preferably conducted with probe-length nucleic acid molecules, preferably 20-100 nucleotides in length. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g.,. Maniatis, T. et al. (1989) Molecular Cloning: A Laboratory Manual, Cold Spring Harbor University Press, Cold Spring, N.Y.; Ausubel, F. M. et al. (1994) Current Protocols in Molecular Biology. John Wiley & Sons, Secaucus, N.J. Homologs and homologous gene sequences may thus be identified using any nucleic acid sequence of interest, including the phage ORFs and bacterial target genes of the present invention.

A typical hybridization, for example, utilizes, besides the labeled probe of interest, a salt solution such as 6×SSC (NaCl and Sodium Citrate base) to stabilize nucleic acid strand interaction, a mild detergent such as 0.5% SDS, together with other typical additives such as Denhardt's solution and salmon sperm DNA. The solution is added to the immobilized sequence to be probed and incubated at suitable temperatures to preferably permit specific binding while minimizing nonspecific binding. The temperature of the incubations and ensuing washes is critical to the success and clarity of the hybridization. Stringent conditions employ relatively higher temperatures, lower salt concentrations, and/or more detergent than do non-stringent conditions. Hybridization temperatures also depend on the length, complementarity level, and nature (i.e., “GC content”) of the sequences to be tested. Typical stringent hybridizations and washes are conducted at temperatures of at least 40° C., while lower stringency hybridizations and washes are typically conducted at 37° C. down to room temperature (25° C.). One of ordinary skill in the art is aware that these conditions may vary according to the parameters indicated above, and that certain additives such as formamide and dextran sulphate may also be added to affect the conditions.

By “stringent hybridization conditions” is meant hybridization conditions at least as stringent as the following: hybridization in 50% formamide, 5×SSC, 50 mM NaH2PO4, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

Homologous nucleotide sequences will distinguishably hybridize with a reference sequence with up to three mismatches in ten (i.e., at least 70% base match in two sequences of equal length). Preferably, the allowable mismatch level is up to two mismatches in 10, or up to one mismatch in ten, more preferably up to one mismatch in twenty. (Those ratios can, of course, be applied to larger sequences.) Preferred embodiments involve identification of binding between ORF product and bacterial cellular component that include methods for distinguishing bound molecules, for example, affinity chromatography, immunoprecipitation, crosslinking, and/or genetic screen methods that permit protein:protein interactions to be monitored. One of skill in the art is familiar with these techniques and common materials utilized (see, e.g., Coligan, J. et al. (eds.) (1995) Current Protocols in Protein Science, John Wiley & Sons, Secaucus, N.J.).

Genetic screening for the identification of protein:protein interactions typically involves the co-introduction of both a chimeric bait nucleic acid sequence (here, the phage ORF to be tested) and a chimeric target nucleic acid sequence that, when co-expressed and having affinity for one another in a host cell, stimulate reporter gene expression to indicate the relationship. A “positive” can thus suggest a potential inhibitory effect in bacteria. This is discussed in further detail in the Detailed Description section below. In this way, new bacterial targets can be identified that are inhibited by specific phage ORF products or derivatives, fragments, mimetics, or other molecules.

Other embodiments involve the identification and/or utilization of a target which is mutated at the site of phage 44AHJD protein interaction but still functional in the cell by virtue of their host's relatively unresponsive nature in the presence of expression of ORFs previously identified as inhibitory to the non-mutant or wild-type strain. Such mutants have the effect of protecting the host from an inhibition that would otherwise occur (e.g., by competing for binding with the phage ORF product) and indirectly allow identification of the precise responsible target. The identified target can then be used, e.g., for follow-up studies and anti-microbial development. In certain embodiments, rescue from inhibition occurs under conditions in which a bacterial target or mutant target is highly expressed. This is performed, for example, through coupling of the sequence with regulatory element promoters, e.g., as known in the art, which regulate expression at levels higher than wild-type, e.g., at a level sufficiently higher that the inhibitor can be competitively bound to the highly expressed target such that the bacterium is detectably less inhibited.

Identification of the bacterial target can involve identification of a phage-specific site of action. This can involve a newly identified target, or a target where the phage site of action differs from the site of action of a previously known antibacterial agent or inhibitor. For example, phage T7 genes 0.7 and 2.0 target the host RNA polymerase, which is also the cellular target for the antibacterial agent, rifampin. To the extent that a phage product is found to act at a different site than previously described inhibitors, aspects of the present invention can utilize those new, phage-specific sites for identification and use of new agents. The site of action can be identified by techniques known to those skilled in the art, for example, by mutational analysis, binding competition analysis, and/or other appropriate techniques.

Once a bacterial host target or mutant target sequence has been identified, it too can be conveniently sequenced, sequence analyzed (e.g., by computer), and the underlying gene(s), and corresponding translated product(s) further characterized. Preferred embodiments include such analysis and identification. Preferably such a target has not previously been identified as an appropriate target for antibacterial action.

Also in preferred embodiments in which the bacterial target is a polypeptide or nucleic acid molecule, the identification of a bacterial target of a phage ORF product or fragment includes identification of a cellular and/or biochemical function of the bacterial target. As understood by those skilled in the art, this can, for example, include identification of function by identification of homologous polypeptides or nucleic acid molecules having known function, or identification of the presence of known motifs or sequences corresponding to known function. Such identifications can be readily performed using sequence comparison computer software, such as the BLAST programs and similar other programs and sequence and motif databases.

In embodiments involving expression of a phage ORF in a bacterial strain, in preferred embodiments that expression is inducible. By “inducible” is meant that expression is absent or occurs at a low level until the occurrence of an appropriate environmental stimulus provides otherwise. For the present invention such induction is preferably controlled by an artificial environmental change, such as by contacting a bacterial strain population with an inducing compound (i.e., an inducer). However, induction could also occur, for example, in response to build-up of a compound produced by the bacteria in the bacterial culture, e.g., in the medium. As uncontrolled or constitutive expression of inhibitory ORFs can severely compromise bacteria to the point of eradication, such expression is therefore undesirable in many cases because it would prevent effective evaluation of the strain and inhibitor being studied. For example, such uncontrolled expression could prevent any growth of the strain following insertion of a recombinant ORF, thus preventing determination of effective transfection or transformation. A controlled or inducible expression is therefore advantageous and is generally provided through the provision of suitable regulatory elements, e.g., promoter/operator sequences that can be conveniently transcriptionally linked to a coding sequence to be evaluated. In most cases, the vector will also contain sequences suitable for efficient replication of the vector in the same or different host cells and/or sequences allowing selection of cells containing the vector, i.e., “selectable markers.” Further, preferred vectors include convenient primer sequences flanking the cloning region from which PCR and/or sequencing may be performed. In preferred embodiments where the purification of phage product is desired, preferably the bacterium or other cell type does not produce a target for the inhibitory product, or is otherwise resistant to the inhibitory product.

In preferred embodiments, the target of the phage ORF product or fragment is identified from a bacterial animal pathogen, preferably a mammalian pathogen, more preferably a human pathogen, and is preferably a gene or gene product of such a pathogen. Also in preferred embodiments, the target is a gene or gene product, where the sequence of the target is homologous to a gene or gene product from such a pathogen as identified above.

As used herein, the term “mammal” has its usual biological meaning, and particularly includes bovines, swine, dogs, cats, and humans.

Other aspects of the invention provide isolated, purified, or enriched specific phage nucleic acid and amino acid sequences, subsequences, and homologs thereof preferably from or corresponding to ORFs 12 and 25, from bacteriophage 44AHJD (Staphylococcus aureus host bacterium). Such nucleotide sequences are at least 15 nucleotides in length, preferably at least 18, 21, 24, or 27 nucleotides in length, more preferably at least 30, 50, or 90 nucleotides in length. In certain embodiments, longer nucleic acids are preferred, for example those of at least 120, 150, 200, 300, 600, 900 or more nucleotides. Such sequences can, for example, be amplification oligonucleotides (e.g., PCR primers), oligonucleotide probes, sequences encoding a portion or all of a phage-encoded protein, or a fragment or all of a phage-encoded protein. In preferred embodiments, the nucleic acid sequence or amino acid sequence contains a sequence which has a lower length as specified above, and an upper-length limit which is no more than 50, 60, 70, 80, or 90% of the length of the full-length ORF or ORF product. The upper-length limit can also be expressed in terms of the number of base pairs of the ORF (coding region).

As it is recognized that alternate codons will encode the same amino acid for most amino acids due to the degeneracy of the genetic code, the sequences of this aspect includes nucleic acid sequences utilizing such alternate codon usage for one or more codons of a coding sequence. For example, all four nucleic acid sequences GCT, GCC, GCA, and GCG encode the amino acid, alanine. Therefore, if for an amino acid there exists an average of three codons, a polypeptide of 100 amino acids in length will, on average, be encoded by 3¹⁰⁰, or 5×10⁴⁷, nucleic acid sequences. Thus, a nucleic acid sequence can be modified (e.g., a nucleic acid sequence from a phage as specified above) to form a second nucleic acid sequence encoding the same polypeptide as encoded by the first nucleic acid sequence using routine procedures and without undue experimentation. Thus, all possible nucleic acid sequences that encode the amino acid sequences encoded by the phage 44AHJD ORFs 12 and 25, as if all were written out in full, taking into account the codon usage, especially that preferred in the host bacterium.

The alternate codon descriptions are available in common textbooks, for example, Stryer, BIOCHEMISTRY 3rd ed., and Lehninger, BIOCHEMISTRY 3rd ed. Codon preference tables for various types of organisms are available in the literature. Because of the number of sequence variations involving alternate codon usage, for the sake of brevity, individual sequences are not separately listed herein. Instead the alternate sequences are described by reference to the natural sequence with replacement of one or more (up to all) of the degenerate codons with alternate codons from the alternate codon table (Table 2), preferably with selection according to preferred codon usage for the normal host organism or a host organism in which a sequence is intended to be expressed. Those skilled in the art also understand how to alter the alternate codons to be used for expression in organisms where certain codons code differently than shown in the “universal” codon table.

For amino acid sequences, sequences contain at least 5 peptide-linked amino acid residues, and preferably at least 6, 7, 10, 15, 20, 30, or 40, amino acids having identical amino acid sequence as the same number of contiguous amino acid residues in a phage ORF 12 or 25 product. In some cases longer sequences may be preferred, for example, those of at least 50, 70, or 100 amino acids in length. In preferred embodiments, the sequence has bacteria-inhibiting function when expressed or otherwise present in a bacterial cell which is a host for the bacteriophage from which the sequence was derived.

By “isolated” in reference to a nucleic acid is meant that a naturally occurring sequence has been removed from its normal cellular (e.g., chromosomal) environment or is synthesized in a non-natural environment (e.g., artificially synthesized). Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

The term “enriched” means that the specific DNA or RNA sequence constitutes a significantly higher fraction (2-5 fold) of the total DNA or RNA present in the cells or solution of interest than in normal or diseased cells or in cells from which the sequence was originally taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased.

The term “significant” is used to indicate that the level of increase is useful to the person making such an increase and an increase relative to other nucleic acids of about at least 2-fold, more preferably at least 5- to 10-fold or even more. The term also does not imply that there is no DNA or RNA from other sources. The other source DNA may, for example, comprise DNA from a yeast or bacterial genome, or a cloning vector such as pUC 19. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation). Instead, it represents an indication that the sequence is relatively more pure than in the natural environment (compared to the natural level, this level should be at least 2-5 fold greater, e.g., in terms of mg/mL). Individual clones isolated from a genomic or cDNA library may be purified to electrophoretic homogeneity. The claimed DNA molecules obtained from these clones could be obtained directly from total DNA or from total RNA. cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from MRNA and isolation of distinct cDNA clones yields an approximately 106-fold purification of the native message. Thus, purification of at least one order of magnitude, preferably two or three orders, and more preferably four or five orders of magnitude is expressly contemplated. A genomic library can be used in the same way and yields the same approximate levels of purification.

The terms “isolated”, “enriched”, and “purified” with respect to the nucleic acids, above, may similarly be used to denote the relative purity and abundance of polypeptides (multimers of amino acids joined one to another by a-carboxyl:a-amino group (peptide) bonds). These, too, may be stored in, grown in, screened in, and selected from libraries using biochemical techniques familiar in the art. Such polypeptides may be natural, synthetic or chimeric and may be extracted using any of a variety of methods, such as antibody immunoprecipitation, other “tagging” techniques, conventional chromatography and/or electrophoretic methods. Some of the above utilize the corresponding nucleic acid sequence.

As indicated above, aspects and embodiments of the invention are not limited to entire genes and proteins. The invention also provides and utilizes fragments and portions thereof, preferably those which are “active” in the inhibitory sense described above. Such peptides or oligopeptides and oligo or polynucleotides have preferred lengths as specified above for nucleic acid and amino acid sequences from phage; corresponding recombinant constructs can be made to express the encoded same. Also included are homologous sequences and fragments thereof.

The nucleotide and amino acid sequences identified herein are believed to be correct, however, certain sequences may contain a small percentage of errors, e.g., 1-5%. In the a event that any of the sequences have errors, the corrected sequences can be readily provided by one skilled in the art using routine methods. For example, the nucleotide sequences can be confirmed or corrected by obtaining and culturing the relevant phage, and purifying phage genomic nucleic acids. A region or regions of interest can be amplified, e.g., by PCR from the appropriate genomic template, using primers based on the described sequence. The amplified regions can then be sequenced using any of the available methods (e.g., a dideoxy termination method, for example, using commercially available products). This can be done redundantly to provide the corrected sequence or to confirm that the described sequence is correct. Alternatively, a particular sequence or sequences can be identified and isolated as an insert or inserts in a phage genomic library and isolated, amplified, and sequenced by standard methods. Confirmation or correction of a nucleotide sequence for a phage gene provides an amino acid sequence of the encoded product by merely reading off the amino acid sequence according to the normal codon relationships and/or expressed in a standard expression system and the polypeptide product sequenced by standard techniques. The sequences described herein thus provide unique identification of the corresponding genes and other sequences, allowing those sequences to be used in the various aspects of the present invention. Confirmation of a phage ORF encoded amino acid sequence can also be confirmed by constructing a recombinant vector from which the ORF can be expressed in an appropriate host (e.g., E. coli), purified, and sequenced by conventional protein sequencing methods.

In other aspects the invention provides recombinant vectors and cells harboring, one or more phage 44AHJD ORFs, preferably ORF 12 or 25 or portions thereof, or bacterial target sequences described herein, preferably where the phage or bacterial sequence is inserted in a recombinant vector. As understood by those skilled in the art, vectors may assume different forms, including, for example, plasmids, cosmids, and virus-based vectors. See, e.g., Maniatis, T. et al. (1989) Molecular Cloning: A Laboratory Manual, Cold Spring Harbor University Press, Cold Spring, N.Y.; See also, Ausubel, F. M. et al. (eds.) (1994) Current Protocols in Molecular Biology. John Wiley & Sons, Secaucus, N.J.

In preferred embodiments, the vectors will be expression vectors, preferably shuttle vectors that permit cloning, replication, and expression within bacteria. An “expression vector” is one having regulatory nucleotide sequences containing transcriptional and translational regulatory information that controls expression of the nucleotide sequence in a host cell. Preferably the vector is constructed to allow amplification from vector sequences flanking an insert locus. In certain embodiments, the expression vectors may additionally or alternatively support expression, and/or replication in animal, plant and/or yeast cells due to the presence of suitable regulatory sequences, e.g., promoters, enhancers, 3′ stabilizing sequences, primer sequences, etc. In preferred embodiments, the promoters are inducible and specific for the system in which expression is desired, e.g., bacteria, animal, plant, or yeast. The vectors may optionally encode a “tag” sequence or sequences to facilitate protein purification or protein detection. Convenient restriction enzyme cloning sites and suitable selective marker(s) are also optionally included. Such selective markers can be, for example, antibiotic resistance markers or markers which supply an essential nutritive growth factor to an otherwise deficient mutant host, e.g., tryptophan, histidine, or leucine in the Yeast Two-Hybrid systems described below.

The term “recombinant vector” relates to a single- or double-stranded circular nucleic acid molecule that can be transfected into cells and replicated within or independently of a cell genome. A circular double-stranded nucleic acid molecule can be cut and thereby linearized upon treatment with appropriate restriction enzymes. An assortment of nucleic acid vectors, restriction enzymes, and the knowledge of the nucleotide sequences cut by restriction enzymes are readily available to those skilled in the art. A nucleic acid molecule encoding a desired product can be inserted into a vector by cutting the vector with restriction enzymes and ligating the two pieces together. Preferably the vector is an expression vector, e.g., a shuttle expression vector as described above.

By “recombinant cell” is meant a cell possessing introduced or engineered nucleic acid sequences, e.g., as described above. The sequence may be in the form of or part of a vector or may be integrated into the host cell genome. Preferably the cell is a bacterial cell.

In preferred embodiments, the inserted nucleic acid sequence corresponding to at least a portion of a bacteriophage 44AHJD ORF, for example ORFs 12 and 25, gene product has a length as specified for the isolated purified or enriched nucleic acid sequences in an aspect above.

In another aspect, the invention also provides methods for identifying and/or screening compounds “active on” at least one bacterial target of a bacteriophage inhibitor protein or RNA, for example, corresponding to ORFs 12 or 25. Preferred embodiments involve contacting bacterial target proteins with a test compound, and determining whether the compound binds to or reduces the level of activity of the bacterial target, e.g., a bacterial protein. Preferably this is done in vivo under approximately physiological conditions. The compounds that can be used may be large or small, synthetic or natural, organic or inorganic, proteinaceous or non-proteinaceous. In preferred embodiments, the compound is a peptidomimetic, as described herein, a bacteriophage inhibitor protein or fragment or derivative thereof, preferably an “active portion”, or a small molecule. In particular embodiments, the methods include the identification of bacterial targets as described above or otherwise described herein. Preferably the fragment of a bacteriophage inhibitor protein includes less than 80% of an intact bacteriophage inhibitor protein. Preferably, the at least one target includes a plurality of different targets of bacteriophage inhibitor proteins, preferably a plurality of different targets. The plurality of targets can be in or from a plurality of different bacteria, but preferably is from a single bacterial species.

In embodiments involving binding assays, preferably binding is to a fragment or portion of a bacterial target protein, where the fragment includes less than 90%, 80%, 70%, 60%, 50%, 40%, or 30% of an intact bacterial target protein. Preferably, the at least one bacterial target includes a plurality of different targets of bacteriophage inhibitor proteins, preferably a plurality of different targets. The plurality of targets can be in or from a plurality of different bacteria, but preferably is from a single bacterial species.

A “method of screening” refers to a method for evaluating a relevant activity or property of a large plurality of compounds, rather than just one or a few compounds. For example, a method of screening can be used to conveniently test at least 100, more preferably at least 1000, still more preferably at least 10,000, and most preferably at least 100,000 different compounds, or even more.

In the context of this invention, the term “small molecule” refers to compounds having molecular mass of less than 3000 Daltons, preferably less than 2000 or 1500, still more preferably less than 1000, and most preferably less than 600 Daltons. Preferably but not necessarily, a small molecule is not an oligopeptide.

In a related aspect or in preferred embodiments, the invention provides a method of screening for potential antibacterial agents by determining whether any of a plurality of compounds, preferably a plurality of small molecules, is active on at least one target of a bacteriophage inhibitor protein or RNA, for example, a target of ORF 12 or 25 gene product. Preferred embodiments include those described for the above aspect, including embodiments which involve determining whether one or more test compounds bind to or reduce the level of activity of a bacterial target, and embodiments which utilize a plurality of different targets as described above.

The identification of bacteria-inhibiting phage ORFs and their encoded products also provides a method for identifying an active portion of such an encoded product. This also provides a method for identifying a potential antibacterial agent by identifying such an active portion of a phage ORF or ORF product. In preferred embodiments, the identification of an active portion involves one or more of mutational analysis, deletion analysis, or analysis of fragments of such products. The method can also include determination of a 3-dimensional structure of an active portion, such as by analysis of crystal diffraction patterns. In further embodiments, the method involves constructing or synthesizing a peptidomimetic compound, where the structure of the peptidomimetic compound corresponds to the structure of the active portion.

In this context, “corresponds” means that the peptidomimetic compound structure has sufficient similarities to the structure of the active portion that the peptidomimetic will interact with the same molecule as the phage protein and preferably will elicit at least one cellular response in common which relates to the inhibition of the cell by the phage protein.

The methods for identifying or screening for compounds or agents active on a bacterial target of a phage-encoded inhibitor can also involve identification of a phage-specific site of action on the target.

An “active portion” as used herein denotes an epitope, a catalytic or regulatory domain, or a fragment of a bacteriophage inhibitor protein that is responsible for, or a significant factor in, bacterial target inhibition. The active portion preferably may be removed from its contiguous sequences and, in isolation, still effect inhibition.

By “mimetic” is meant a compound structurally and functionally related to a reference compound that can be natural, synthetic, or chimeric. In terms of the present invention, a “peptidomimetic,” for example, is a compound that mimics the activity-related aspects of the 3-dimensional structure of a peptide or polypeptide in a non-peptide compound, for example mimics the structure of a peptide or active portion of a phage- or bacterial ORF-encoded polypeptide.

A related aspect provides a method for inhibiting a bacterial cell by contacting the bacterial cell with a compound active on a bacterial target of a bacteriophage 44 AHJD inhibitor protein or RNA, preferably encoded by or corresponding to bacteriophage 44 AHJD ORF 12 or 25, where the target was uncharacterized. In preferred embodiments, the compound is such a protein, or a fragment or derivative thereof; a structural mimetic, e.g., a peptidomimetic, of such a protein or fragment; a small molecule; the contacting is performed in vitro, the contacting is performed in vivo in an infected or at risk organism, e.g., an animal such as a mammal or bird, for example, a human, or other mammal described herein, or in a plant.

In the context of this invention, the term “bacteriophage inhibitor protein” refers to a protein encoded by a bacteriophage nucleic acid sequence which inhibits bacterial function in a host bacterium. Thus, it is a bacteria-inhibiting phage product.

In the context of this invention, the phrase “contacting the bacterial cell with a compound active on a bacterial target of a bacteriophage inhibitor protein” or equivalent phrases refer to contacting with an isolated, purified, or enriched compound or a composition including such a compound, but specifically does not rely on contacting the bacterial cell with an intact naturally occurring phage which encodes the compound. Preferably no intact phage are involved in the contacting.

Related aspects provide methods for prophylactic or therapeutic treatment of a bacterial infection by administering to an infected, challenged or at risk organism a therapeutically or prophylactically effective amount of a compound active on a target of a bacteriophage 44AHJD product, preferably an ORF 12 or 25 product, e.g., as described for the previous aspect. Preferably the bacterium involved in the infection or risk of infection produces the identified target of the bacteriophage inhibitor protein or alternatively produces a homologous target compound. In preferred embodiments, the host organism is a plant or animal, preferably a mammal or bird, and more preferably, a human or other mammal described herein. Preferred embodiments include, without limitation, those as described for the preceding aspect.

Compounds useful for the methods of inhibiting, methods of treating, and pharmaceutical compositions can include novel compounds, but can also include compounds which had previously been identified for a purpose other than inhibition of bacteria. Such compounds can be utilized as described and can be included in pharmaceutical compositions.

By “treatment” or “treating” is meant administering a compound or pharmaceutical composition for prophylactic and/or therapeutic purposes. The term “prophylactic treatment” refers to treating a patient or animal that is not yet infected but is susceptible to or otherwise at risk of a bacterial infection. The term “therapeutic treatment” refers to administering treatment to a patient already suffering from infection.

The term “bacterial infection” refers to the invasion of the host organism, animal or plant, by pathogenic bacteria. This includes the excessive growth of bacteria which are normally present in or on the body of the organism, but more generally, a bacterial infection can be any situation in which the presence of a bacterial population(s) is damaging to a host organism. Thus, for example, an organism suffers from a bacterial infection when excessive numbers of a bacterial population are present in or on the organism's body, or when the effects of the presence of a bacterial population(s) is damaging to the cells, tissue, or organs of the organism.

The terms “administer”, “administering”, and “administration” refer to a method of giving a dosage of a compound or composition, e.g., an antibacterial pharmaceutical composition, to an organism. Where the organism is a mammal, the method is, e.g., topical, oral, intravenous, transdermal, intraperitoneal, intramuscular, or intrathecal. The preferred method of administration can vary depending on various factors, e.g., the components of the pharmaceutical composition, the site of the potential or actual bacterial infection, the bacterium involved, and the infection severity.

The term “mammal” has its usual biological meaning, referring to any organism of the Class Mammalia of higher vertebrates that nourish their young with milk secreted by mammary glands, e.g., mouse, rat, and, in particular, human, bovine, sheep, swine, dog, and cat.

In the context of treating a bacterial infection a “therapeutically effective amount” or “pharmaceutically effective amount” indicates an amount of an antibacterial agent, e.g., as disclosed for this invention, which has a therapeutic effect. This generally refers to the inhibition, to some extent, of the normal cellular functioning of bacterial cells that renders or contributes to bacterial infection.

The dose of antibacterial agent that is useful as a treatment is a “therapeutically effective amount.” Thus, as used herein, a therapeutically effective amount means an amount of an antibacterial agent that produces the desired therapeutic effect as judged by clinical trial results and/or animal models. This amount can be routinely determined by one skilled in the art and will vary depending on several factors, such as the particular bacterial strain involved and the particular antibacterial agent used.

In connection with claims to methods of inhibiting bacteria and therapeutic or prophylactic treatments, “a compound active on a target of a bacteriophage inhibitor protein” or terms of equivalent meaning differ from administration of or contact with an intact phage naturally encoding the full-length inhibitor compound. While an intact phage may conceivably be incorporated in the present methods, the method at least includes the use of an active compound as specified different from a full length inhibitor protein naturally encoded by a bacteriophage and/or a delivery or contacting method different from administration of or contact with an intact phage naturally encoding the full-length protein. Similarly, pharmaceutical compositions described herein at least include an active compound or composition different from a phage naturally coding the full-length inhibitor protein, or such a full-length protein is provided in the composition in a form different from being encoded by an intact phage. Preferably the methods and compositions do not include an intact phage.

In accordance with the above aspects, the invention also provides antibacterial agents and compounds active on a bacterial target of a bacteriophage 44AHJD inhibitory, preferably ORF 12 or 25, where the target was uncharacterized as indicated above. As previously indicated, such active compounds include both novel compounds and compounds which had previously been identified for a purpose other than inhibition of bacteria. Such previously identified biologically active compounds can be used in embodiments of the above methods of inhibiting and treating. In preferred embodiments, the targets, bacteriophage, and active compound are as described herein for methods of inhibiting and methods of treating. Preferably the agent or compound is formulated in a pharmaceutical composition which includes a pharmaceutically acceptable carrier, excipient, or diluent. In addition, the invention provides agents, compounds, and pharmaceutical compositions where an active compound is active on an uncharacterized phage-specific site on the target.

In preferred embodiments, the target is as described for embodiments of aspects above.

Likewise, the invention provides a method of making an antibacterial agent. The method involves identifying a target of a bacteriophage 44AHJD ORF 12 or 25 product, screening a plurality of compounds to identify a compound active on the target, and synthesizing the compound in an amount sufficient to provide a therapeutic effect when administered to an organism infected by a bacterium naturally producing the target.

In preferred embodiments, the identification of the target and identification of active compounds include steps or methods and/or components as described above (or otherwise herein) for such identification. Likewise, the active compound can be as described above, including fragments and derivatives of phage inhibitor proteins, peptidomimetics, and small molecules. As recognized by those skilled in the art, peptides can be synthesized by expression systems and purified, or can be synthesized artificially by methods well known in the art.

In the context of nucleic acid and/or amino acid sequences of this invention, the terms “correspond” and “corresponding” indicate that the sequence is at least 95% identical, preferably at least 97% identical, and more preferably at least 99% identical to a sequence from the specified phage genome or bacterial genome, a ribonucleotide equivalent, a degenerate equivalent (utilizing one or more degenerate codons), the translated product of a nucleic acid sequence, nucleic acid sequence(s) encoding for a specific polypeptide, or a homologous sequence, where the homolog provides functionally equivalent biological function. It is also understood that the terms “correspond” and “corresponding” indicate that a nucleic acid sequence corresponds to the polypeptide which corresponds to a protein encoded by a bacteriophage ORF, such as for example S. aureus bacteriophage 44AHJD, and a protein can correspond to the nucleic acid sequence(s) which encode therefor.

In embodiments where the bacterial target of a bacteriophage inhibitor ORF product, e.g., an inhibitory protein or polypeptide, the target is preferably encoded by a S. aureus nucleic acid coding sequence from a host bacterium for bacteriophage 44AHJD. Target sequences are described herein by reference to sequence source sites. The sequence encoding the target preferably corresponds to a S. aureus nucleic acid sequence available from numerous sources including S. aureus sequences deposited in GenBank, S. aureus sequences found in European Patent Application No. 97100110.7 to Human Genome Sciences, Inc. filed Jan. 7, 1997, S. aureus sequences available from The Institute for Genome Research (TIGR) at internet address http://www., where the remainder of the address is tigr.org/tdb/mdb/mdbinprogress.html, S. aureus sequences available from the Oklahoma University S. aureus sequencing project can be obtained by following directions provided on the internet address http://www., where the remainder of the address is .genome.ou.edu/staph.html, and S. aureus sequences available from internet address http://www., where the remainder of the address is .sanger.ac.uk/Projects/S_{—}aureus/.

The amino acid sequence of a polypeptide target is readily provided by translating the corresponding coding region. For the sake of brevity, the sequences are not reproduced herein. Also, in preferred embodiments, a target sequence corresponds to a S. aureus coding sequences corresponding to a sequence listed in Table 6 herein. The listing in Table 6 describes S. aureus sequences currently deposited in GenBank. Again, for the sake of brevity, the sequences are described by reference to the GenBank entries instead of being written out in full herein. In cases where an entry for a coding region is not complete, the complete sequence can be readily obtained by routine methods, e.g., by isolating a clone in a phage 44AHJD host S. aureus genomic library, and sequencing the clone insert to provide the relevant coding region. The boundaries of the coding region can be identified by conventional sequence analysis and/or by expression in a bacterium in which the endogenous copy of the coding region has been inactivated and using subcloning to identify the functional start and stop codons for the coding region.

In an additional aspect, the present invention provides a nucleic acid segment which encodes a protein and corresponds to a segment of the nucleic acid sequence of an ORF (open reading frame) from Staphylococcus aureus bacteriophage 44AHJD corresponding to a sequence provided in Table 1. Preferably, the protein is a functional protein. One of ordinary skill in the art would recognize that bacteriophage possess genes which encode proteins which may be either beneficial or detrimental to a bacterial cell. Such proteins act to replicate DNA, translate RNA, manipulate DNA or RNA, and enable the phage to integrate into the bacterial genome. Proteins from bacteriophage can function as, for example, a polymerase, kinase, phosphatase, helicase, nuclease, topoisomerase, endonuclease, reverse transcriptase, endoribonuclease, dehydrogenase, gyrase, integrase, carboxypeptidase, proteinase, amidase, transcriptional regulators and the like, and/or the protein may be a functional protein such as a chaperon, capsid protein, head and tail proteins, a DNA or RNA binding protein, or a membrane protein, all of which are provided as non-limiting examples. Proteins with functions such as these are useful as tools for the scientific community.

Thus, the present invention provides a group of novel proteins from bacteriophage which can be used as tools for biochemical applications such as, for example, DNA and/or RNA sequencing, polymerase chain reaction and/or reverse transcriptase PCR, cloning experiments, cleavage of DNA and/or RNA, reporter assays and the like. Preferably, the protein is encoded by an open reading frame from the nucleic acid sequence of bacteriophage 44AHJD. Within the scope of the present invention are fragments of proteins and/or truncated portions of proteins which have been either engineered through automated protein synthesis, or prepared from nucleic acid segments which correspond to segments of the nucleic acid sequences of bacteriophage 44AHJD, and which are inserted into cells via plasmid vectors which can be induced to express the protein. It is understood by one of skill in the art that mutational analysis of proteins has been known to help provide proteins which are more stable and which have higher and/or more specific activities. Such mutations to proteins encoded by phage 44AHJD ORFs are also within the scope of the present invention, hence, the present invention also provides a mutated protein and/or the mutated nucleic acid segment from bacteriophage 44AHJD which encodes the protein.

In another aspect, the invention provides antibodies which bind proteins encoded by a nucleic acid segment which corresponds to the nucleic acid sequence of an ORF (open reading frame) from Staphylococcus aureus bacteriophage 44AHJD as provided in Table 1. Bacteriophages are bacterial viruses which contain nucleic acid sequences which encode proteins that can correspond to proteins of other bacteriophages and other viruses.

Antibodies targeted for proteins encoded by nucleic acid segments of phage 44AHJD can serve to bind targets encoded by nucleic acid segments from other viruses which correspond to the sequences provided in Table 1. Furthermore, antibodies to proteins encoded by nucleic acid segments of phage 44AHJD can also bind to proteins from other viruses that share similar functions but may not share corresponding sequences. It is understood in the art that proteins with similar activities/functions from a variety of sources generally share motifs, regions, or domains which correspond. Thus, antibodies to motifs, regions, or domains of functional proteins from phage 44AHJD should be useful in detecting corresponding proteins in other bacteriophages and viruses. Such antibodies can also be used to detect the presence of a virus sharing a similar protein. Preferably the virus to be detected is pathogenic to a mammal, such as a dog, cat, bovine, sheep, swine, or a human.

It has been determined that dnaN is a target for bacteriophage 44AHJD ORF 25 product, which acts as an inhibitory factor. Applicants have recognized the utility of the interaction in the development of antibacterial agents. Polypeptide and/or polynucleotide targets such as dnaN are critical targets for bacterial inhibition. S. aureus bacteriophage 44AHJD ORF 25 or derivatives or functional mimetics thereof are useful for inhibiting bacterial growth and the interaction, binding, inhibition and/or activation which occurs between polypeptides and/or polynucleotides, such as for example dnaN of S. aureus and 44AHJD ORF 25 may be used as a target for the screening and rational design of drugs or antibacterial agents. In addition to methods for directly inhibiting a target such as dnaN activity, methods of inhibiting a target such as dnaN expression are also attractive for antibacterial activity.

In a related aspect or in preferred embodiments, the present invention provides methods for identifying compounds which bind to, interact with, inhibit and/or activate an activity and/or expression of a polypeptide and/or polynucleotide of the invention, e.g., a polypeptide or polynucleotide that binds or interacts with a bacteriophage 44 AHJD inhibitory ORF, preferably ORF 12 or 25. Such methods comprise contacting a polypeptide and/or polynucleotide of the invention, such as for example a target or product of ORF 12 or 25 with a compound to be screened under conditions which permit binding or other interaction between the compound and the polypeptide and/or polynucleotide. The method, preferably allows assessment of the binding or other interaction with the compound being identified by associating the binding or interaction with a second component which is capable of providing a detectable signal in response to the binding or interaction of the polypeptide and/or polynucleotide with the compound. Determination of whether the compound binds to, interacts with, activates and/or inhibits an activity or expression of the polypeptide and/or polynucleotide is by detection of the presence or absence of a signal generated from the binding or interaction of the compound with the polypeptide and/or polynucleotide.

In preferred embodiments, the method involves the interaction of an ORF 12 or 25 product or fragment thereof with the corresponding bacterial target or fragment thereof that maintains the interaction with the ORF product or fragment. Interference with the interaction between the components can be monitored, and such interference is indicative of compounds that will inhibit, activate, or enhance the activity of the target molecule.

Preferably, compounds which are identified by methods of the present invention include, but are not limited to, small organic molecules, peptides, polypeptides and antibodies that bind to a polynucleotide and/or polypeptide of the invention, such as for example ORF 12 or 25 gene product or target thereof, and thereby inhibit or extinguish or enhance its activity or expression. Potential compounds also may be small organic molecules, a peptide, a polypeptide such as a closely related protein or antibody that binds the same sites on a binding molecule, such as a binding molecule, without inducing for example dnaN or dnaN homologues or peptido-mimetic derivatives, induced activities, thereby preventing the action or expression of S. aureus bacteriophage 44AHJD ORF 12 or 25 gene product or target thereof and/or for example dnaN polypeptides and/or polynucleotides by excluding S. aureus 44AHJD ORF 12 or 25 gene product or target thereof and/or for example dnaNpolypeptides and/or polynucleotides from binding.

Potential compounds also include small molecules that bind to and occupy the binding site of a polypeptide thereby preventing binding to cellular binding molecules, such that normal biological activity is prevented. Examples of small molecules include but are not limited to small organic molecules, peptides or peptide-like molecules. Other potential compounds include antisense molecules (see Okano, (1991) J. Neurochem. 56, 560; see also “Oligodeoxynucleotides as Antisense Inhibitors of Gene Expression”, CRC Press, Boca Raton, Fla. (1988), for a description of these molecules). Preferred potential compounds include compounds related to and variants of 44AHJD ORF 12 or 25 and of dnaN and any homologues and/or peptido-mimetics and/or fragments thereof. Other examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypeptide of the present invention but do not elicit a response, so that the activity of the polypeptide is prevented.

Compounds may be identified from a variety of sources, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991). Peptide modulators can also be selected by screening large random libraries of all possible peptides of a certain length.

Compounds derived from the polypeptide sequence of 44AHJD ORF 12 or 25 itself could represent fragments representing small overlapping peptide spanning the entire amino acid sequence of the protein. Fragments of 44AHJD ORF 12 or 25 can be produced by proteolytic digestion of the full-length protein as described above. Alternatively, suitable 44AHJD ORF 12 or 25 derived peptide or polypeptide fragments representative of the complete sequence of the protein can be chemically synthesized. For instance, in the multi-pin approach, peptides are simultaneously synthesized by the assembly of small quantities of peptides on plastic pins derivatized with an ester linker based on glycolate and 4-(hydroxymethyl) benzoate (Maeji et al. (1991) Pept Res, 4:142-6).

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Additional features and embodiments of the present invention will be apparent from the following Detailed Description and from the claims, all within the scope of the present invention.

### BRIEF DESCRIPTION OF THE DRAWINGS

FIG. 1 is a flow schematic showing the manipulations necessary to convert pT0021, an arsenite inducible vector containing the luciferase gene, into pTHA or pTM, two ars inducible vectors, or pTMLac , a lactose-inducible promotor. a) Vector pTHA contains BamH I and Sal I cloning sites and a downstream HA epitope tag. b) and c)Vector pTM and pTMLac contain Bam HI and Hind III cloning sites and no HA epitope tag.

FIG. 2 is a schematic representation of the cloning steps involved to place the DNA segments of any of ORFs 12, 25, or other sequences into vectors to assess inhibitory potential. a) For subdloning into pTHA, individual ORFs e.g. 44AHJD ORF 12 and 25 were amplified by the PCR using oligonucleotides targeting the start codon and the penultimated codon of the ORFs. Using this strategy, BamHI and SalI sites were positioned immediately upstream or downstream, respectively of these two codons. Following digestion with BamHI and SalI, the PCR fragments were subdloned into the same sites of pTHA.

b) For subcloning into pTM or pTMLac, (examplified for pTM in b) individual ORFs were amplified by the PCR using oligonucleotides targeting the ATG and stop codons of the ORFs. Using this strategy, Bam HI and Hind III sites were positioned immediately upstream or downstream, respectively of the start and stop codons of each ORF. Following digestion with Bam HI and Hind III, the PCR fragments were subcloned into the same sites of pTM or pTMLac. Clones were verified by PCR and direct sequencing.

FIG. 3 shows a schematic representation of the functional assays used to characterize the bactericidal and bacteriostatic potential of all predicted ORFs (>33 amino acids) encoded by bacteriophage 44AHJD. FIG. 3A) Functional assay on semi-solid support media. FIG. 3B) Functional assay in liquid culture.

FIG. 4 shows the results of the functional assay on semi-solid support media to identify bacteriophage 44AHJD ORFs with anti-microbial activity. FIG. 4 a) shows the lists of the 31 bacteriophage 44AHJD ORFs that were screened in the functional assay and FIG. 4 b) shows inhibition of bacterial growth following induction of expression of phage 44AHJD ORF 12 and 25 from three clones of Staphylococcus aureus transformants tested at four different concentrations. One clone of Staphylococcus aureus transformed with the non-inhibitory ORF (77 bacteriophage ORF 30 cloned into pT vector) was used as control. From these experiments, it is clear that expression of these two ORFs leads to the inhibition of growth of Staphylococcus aureus.

FIG. 5 A and B are the graphs of OD₅₆₅ values and colony forming units (CFU) over time showing the results of functional assay in liquid media to assess bacteriostatic or bactericidal activity of bacteriophage 44AHJD ORF 12 and 25. Growth inhibition assays were performed as detailed in the Detailed Description. The OD₅₆₅ values and the number of CFU were determined from cultures of Staphylococcus aureus transformants harboring a given bacteriophage inhibitory ORF, in the absence or presence of the inducer. The identity of the expression vector and subdloned ORF harbored by the Staphylococcus aureus is given at the top of the each graph. The value of OD and the number of CFU was also determined from non-induced and induced control cultures of Staphylococcus aureus transformants harboring a non-inhibitory phage ORF cloned into the same vector. Each graph represents the average obtained from three Staphylococcus aureus transformants.

FIG. 6 shows the pattern of protein expression of the inhibitory ORF in S. aureus in the presence or in the absence of induction with sodium arsenite. In individual inhibitory ORF (44AHJD phage ORF 12 and 25) cloned into the pTHA vector, the HA tag is set inframe with the ORF and is positioned at the carboxy terminus of each ORF. An anti-HA tag antibody was used for the detection of the ORF expression. The identity of the subcloned ORF harbored by the Staphylococcus aureus transformants is given at the top of the panel.

FIGS. 7A and 7B depict the results from affinity chromatography using GST and GST/44AHJD ORF 25 as ligands with a S. aureus extract prepared by French pressure cell lysis and sonication. Eluates from affinity columns containing the GST and GST/ORF25 ligands at 0, 0.1, 0.5, 1.0, and 2.0 mg/ml resin were resolved by SDS-12.5% PAGE. Proteins were visualized by silver staining. Micro-columns were eluted with: A) 1 M NaCl ABC (ACB; 20 mM Hepes pH 7.5, 10% glycerol, 1 mM DTT, and 1 mM EDTA); and B) 1% SDS. Each molecular weight marker is approximately 100 ng. The lanes labeled ACB indicate eluates from a 2.0 mg/ml ligand column loaded only with ACB buffer containing 75 mM NaCl. The arrows indicate proteins specifically with GST/ORF25.

FIG. 8 shows results of a tryptic peptide mass spectrum of the PT48 protein that interacted with 44AHJD ORF 25 and that was eluted with 1% SDS and labelled: PT48 in FIG. 7B. The control band excised from the 48 kDA region of the gels containing PT48 did not contain PT48.

FIG. 9 shows the identification of PT48 as S. aureus DNA-directed DNA polymerase III beta subunit protein from the Genbank database (accession number: 1084189).

FIG. 10 shows the nucleotide and amino acid sequences of S. aureus DnaN.

### DETAILED DESCRIPTION OF THE PREFERRED EMBODIMENTS

Preliminarily the tables will be briefly described.

Table 1 shows the complete nucleotide sequence of the genome of Staphylococcus aureus bacteriophage 44AHJD.

Table 2 is a table from Alberts et al., MOLECULAR BIOLOGY OF THE CELL 3rd ed., showing the redundancy of the “universal” genetic code.

Table 3 shows the nucleotide and predicted amino acid sequences of ORFs 12 and 25 from bacteriophage 44AHJD.

Table 4 Shows homology search results. BLAST analysis was performed with 44AHJD ORFs 12 and 25 against NCBI non-redundant nucleotide and Swissprot databases. The results of this search indicate that ORF 12 has significant homology to an hypothetical 15.7 Kd protein of Bacillus subtilis located in the SpoIIIC-CWLA intergenic region.

Table 5 shows the physiochemical parameters of phage 44AHJD ORFs 12 and 25. These include the primary amino acid sequence of the predicted protein, the average molecular weight, amino acid composition, theoretical pI and hydrophobicity properties (Kite-Doolittle scale).

Table 6 shows a list of Staphylococcus aureus sequences which may represent genes coding for target sequences for phage 44 AHJD ORFs 12 and 25 encoded antimicrobial proteins or peptides.

Table 7 shows genetic map and sequence position of the 73 orfs predicted to be encoded by phage 44AHJD that are greater than 33 amino acids.

Table 8 shows nucleotide and predicted amino acid sequence of all 73 orfs identified in phage 44AHJD.

The present invention is based on the identification of naturally-occurring DNA sequence elements encoding RNA or proteins with anti-microbial activity. Bacteriophages or phages, are viruses that infect and kill bacteria. They are natural enemies of bacteria and, over the course of evolution have perfected enzymes (products of DNA sequences) which enable them to infect a host bacteria, replicate their genetic material, usurp host metabolism, and ultimately kill their host. The scientific literature documents well the fact that many known bacteria have a large number of such bacteriophages that can infect and kill them (for example, see the ATCC bacteriophage collection at http://www.atcc.org) (Ackermaun and DuBow, 1987). Although we know that many bacteriophages encode proteins which can significantly alter their host's metabolism, determination of the killing potential of a given bacteriophage gene product can only be assessed by expressing the gene product in the target bacterial strain.

As indicated in the Summary above, the present invention is concerned with the use of bacteriophage 44AHJD coding sequences and the encoded polypeptides or RNA transcripts to identify bacterial targets for potential new antibacterial agents. Thus, the invention concerns the selection of relevant bacteria. Particularly relevant bacteria are those which are pathogens of a complex organism such as an animal, e.g., mammals, reptiles, and birds, and plants. However, the invention can be applied to any bacterium (whether pathogenic or not) for which bacteriophage are available or which are found to have cellular components closely homologous to components targeted by phage 44AHJD ORFs 12 and 25.

Identification of ORFs 12 and 25 and products from the phage which inhibit the host bacterium both provides an inhibitor compound and allows identification of the bacterial target affected by the phage-encoded inhibitor. Such a target is thus identified as a potential target for development of other antibacterial agents or inhibitors and the use of those targets to inhibit those bacteria. As indicated above, even if such a target is not initially identified in a particular bacterium, such a target can still be identified if a homologous target is identified in another bacterium. Usually, but not necessarily, such another bacterium would be a genetically closely related bacterium. Indeed, in some cases, an inhibitor encoded by phage 44AHJD ORF 12 or 25 can also inhibit such a homologous bacterial cellular component.

The demonstration that bacteriophage have adapted to inhibiting a host bacterium by acting on a particular cellular component or target provides a strong indication that that component is an appropriate target for developing and using antibacterial agents, e.g., in therapeutic treatments. Thus, the present invention provides additional guidance over mere identification of bacterial essential genes, as the present invention also provides an indication of accessibility of the target to an inhibitor, and an indication that the target is sufficiently stable over time (e.g., not subject to high rates of mutation) as phage acting on that target were able to develop and persist. Thus, the present invention identifies a particular subset of essential cellular components which are particularly likely to be appropriate targets for development of antibacterial agents.

The invention also, therefore, concerns the development or identification of inhibitors of bacteria, in addition to the phage-encoded inhibitory proteins (or RNA transcripts), which are active on the targets of bacteriophage-encoded inhibitors. As described herein, such inhibitors can be of a variety of different types, but are preferably small molecules.

Target proteins of antibiotics generally provide a critical cell function such as DNA replication or cell wall biosynthesis. A proven approach in the discovery of a new drug is to obtain a target protein and to develop in vitro assays to interfere with the biological function of the protein. As described below for DNA polymerase III, biological machineries are often comprised of multiprotein complexes. Thus, any members of essential multiprotein complexes are hypothetical targets for drug development. However, the fact that a protein can be associated with certain biological function does not imply that it represents suitable intervention for new drugs development(Drews J. 2000, Science 287:1960-1964). For instance, although DNA replication is a well-known and essential process for bacterial growth, only a relatively small number of DNA replication complex proteins are targeted by antibiotics. In addition, screening of compounds that inhibit the function of a target must be selective. This underscores the necessity to develop new target-derived strategies that include the step of identification of the protein domain that should be specifically targeted for drug design.

DNA polymerase III holoenzyme is an essential component of bacterial DNA replication machinery. The holoenzyme contains seven different polypeptide chains. Some of these subunits are essential for normal DNA replication in vivo, as shown by the existence of temperature-sensitive (ts) mutations in genes encoding these polypeptides. Type III polymerases are exemplified by the replicase of the Gram-negative bacterium Escherichia coli, in which there are three separate components: a sliding clamp protein, a clamp loader complex and the DNA polymerase itself (Kelman et al. 1995, Annu. Rev. Biochem. 64: 171-200). The clamp loader is a multiprotein complex which uses ATP to assemble the sliding clamp around DNA. The DNA polymerase then binds to the sliding clamp which tethers the polymerase to the DNA. As described in Biochemistry edited by Mathews and Holde [1995; the Benjamin/Cumming Publishing Company], the three subunits—alpha, epsilon and theta—form the polymerase core enzyme. The binding of the gamma complex, the clamp loader, converts the aggregate to a form refered to as DNA polymerase III star, polIII*. This binds to the beta subunit, the clamp slider, to produce the holoenzyme. The beta subunit is a homodimer and forms the ring shaped sliding clamp associated with DNA.

Although there are several studies on the mechanism of replication in Gram-negative bacteria there is little information about how Gram-positive organisms replicate their genetic material. Bacillus subtilis is the best characterized Gram-positive bacterium with respect to DNA replication (Barnes et al. 1995, Methods in Enzy. 262: 35-42), where many genes involved in DNA replication have been identified through the isolation of ts mutants. Studies in B. subtilis have identified a polymerase that appears to be involved in chromosome replication and is termed PolIII. The polC gene encodes Pol III, a large polypeptide likely corresponding to the alpha and epsilon subunits of the E. coli enzyme. B. subtilis and Staphylococcus aureus each also have a gene encoding a protein with 30% homology to the beta subunit of the E coli enzyme; however, neither protein has been purified or characterized (Alfonso and Fisher 1995, Mol. Gen. Gent. 246: 680-686). The S. aureus gene corresponding to the E. coli beta subunit is dnaN. S. aureus dnaN has been described in an International Patent Application entitled: “DNA REPLICATION PROTEINS AND THEIR USE TO SCREEN FOR CHEMICAL INHIBITORS” WO 99/37661.

The following description provides preferred methods for implementing the various aspects of the invention. However, as those skilled in the art will readily recognize, other approaches can be used to obtain and process relevant information. Thus, the invention is not limited to the specifically described methods. In addition, the following description provides a set of steps in a particular order. That series of steps describes the overall development involved in the present invention. However, it is clear that individual steps or portions of steps may be usefully practiced separately, and, further, that certain steps may be performed in a different order or even bypassed if appropriate information is already available or is provided by other sources or methods.

Bacterial Targets for Antibiotics

The main promise for using a bacteriophage approach to drug discovery lies in the potential to determine the optimal molecular target. The target proteins of antibiotics generally provide a critical cell function such as DNA replication or cell wall biosynthesis. A proven approach in the discovery of a new drug is to obtain a target protein and to develop in vitro assays to interfere with the biological function of the protein. As described below for DNA polymerase III, biological machineries are often comprised of multiprotein complexes. Thus, any members of essential multiprotein complexes are hypothetical targets.

DNA polymerase III holoenzyme is an essential component of the DNA replication machinery. The holoenzyme contains seven different polypeptide chains. Some of these subunits are essential for normal DNA replication in vivo, as shown by the existence of temperature-sensitive (ts) mutations in genes encoding these polypeptides. Type III polymerases are exemplified by the replicase of the Gram-negative bacterium Escherichia coli, in which there are three separate components: a sliding clamp protein, a clamp loader complex and the DNA polymerase itself (Kelman et al., 1995, Annu. Rev. Biochem. 64: 171-200). As described in the literature, the three subunits, Alpha, epsilon and theta, form the polymerase core enzyme. The binding of the gamma complex, the clamp loader, converts the aggregate to a form refered to as DNA polymerase III star, polIII*. This binds to the beta subunit, the clamp slider, to produce the holoenzyme. The beta subunit is a homodimer and forms the ring shaped sliding clamp associated with DNA.

Of the Gram-positive organisms, Bacillus subtilis and Streptococcus pyogenes are the best characterized with respect to DNA replication (Barnes et al., 1995, Methods in Enzy. 262:35-42, Bruck I. and O'Donnell, M. 2000, J.Bio01.Chem. 275:28971-28983), where many genes involved in DNA replication have been identified through the isolation of ts mutants. Studies in B.subtilis have indentified a polymerase that appears to be involved in chromosome replication and is termed PolIII. The polC gene encodes Pol III, a large polypeptide corresponding to the alpha and epsilon subunits of E.coli enzyme. B. subtilis and another Gram positive, Staphylococcus aureus each have a gene encoding a protein with 30% homology to the beta subunit of the E.coli enzyme. The S. aureus gene corresponding to the E.coli beta subunit is dnaN. S. aureus DnaN has been described in international patent application, “Dna Replication Proteins and Their Use To Screen for Chemical Inhibitors” WO 99/37661.

Identification of Inhibitory ORF

The methodology previously described in U.S. Provisional Application Pelletier, et al., No. 60/168,777 filed Dec. 1, 1999 was used to identify and characterize DNA sequences from Staphylococcus aureus bacteriophage 44AHJD that can act as anti-microbials. A nucleic acid segment isolated from Staphylococcus aureus bacteriophage 44AHJD encodes a protein, whose gene is referred to as ORF (open reading frame) 12 or 25.

Thus, the present invention provides a nucleic acid sequence isolated from Staphylococcus aureus (Staph A or S. aureus) bacteriophage 44AHJD comprising at least a portion of the gene encoding ORF 12 or 25 with anti-microbial activity. The nucleic acid sequence can be isolated using a method similar to those described herein, or using another method. In addition, such a nucleic acid sequence can be chemically synthesized. Having the anti-microbial nucleic acid sequence of the present invention, parts thereof or oligonucleotides derived therefrom, other anti-microbial sequences from other bacteriophage sources using methods described herein or other methods can be isolated, including screening methods based on nucleic acid sequence hybridization.

The present invention provides the use of the Staph A bacteriophage 44AHJD anti-microbial DNA segment encoding ORF 12 or 25, as a pharmacological agent—either wholly or in part—as well as the use of peptidomimetics, developed from amino acid or nucleotide sequence knowledge of Staph A bacteriophage 44AHJD ORF 12 or 25. This can be achieved where the structure of the peptidomimetic compound corresponds to the structure of the active portion of ORF 12 or 25. In this analysis, the peptide backbone is transformed into a carbon-based hydrophobic structure that can retain cytostatic or cytocidal activity for the bacterium. This is done by standard medicinal chemistry methods, measuring growth inhibition of the various molecules in liquid cultures or on solid medium. These mimetics also represent lead compounds for the development of novel antibiotics.

In this context, “corresponds” means that the peptidomimetic compound structure has sufficient similarities to the structure of the active portion of ORF 12 or 25 that the peptidomimetic will interact with the same molecule as the product of ORF 12 or 25 and preferably will elicit at least one cellular response in common which relates to the inhibition of the cell by the phage protein.

The invention also provides bacteriophage anti-microbial DNA segments from other phages based on nucleic acids and sequences hybridizing to the presently identified inhibitory ORF under high stringency conditions or sequences which are homologous as described above. The bacteriophage anti-microbial DNA segment from bacteriophage 44AHJD ORF 12 or 25 can be used to identify a related segment from another related or unrelated phage based on conditions of hybridization or sequence comparison.

The methodology previously described (U.S. Provisional Application No. 60/110,992, filed Dec. 3, 1998) is used to identify and characterize DNA sequences from Staphylococcus sp. bacteriophage 44 AHJD that can act as antimicrobials.

The Staphylococcus aureus propagating strain (PS 44A) was obtained from the Felix d'Herelle Reference Centre (#HER 1101) was used as a host to propagate its phage 44AHJD, also obtained from the Felix d'Herelle Reference Centre (#HER 101). We find that bacteriophage 44AHJD consists of 16,668 bp (Table 1) predicted to encode 73 ORFs greater than 33 amino acids (Table 7, Table 8). Computational analysis of the predicted protein products of Staphylococcus aureus bacteriophage 44AHJD, which detected homologs in public databases, are listed in Table 6, along with the accompanying list of related proteins. protein products related to those deposited in public databases.

From this analysis, it is apparent that 3 genes (ORF 3, 7, and 8) are related to structural proteins found in other bacteriophages. These include genes predicted to encode a tail protein (ORF 3), an upper collar/connector protein of the phage virion (ORF 7), and a lower collar protein (ORF 8). Bioinformatics has also identified one gene whose product is likely involved in phage DNA synthesis. One gene (ORF 1) shows significant homology to DNA polymerases of a number of bacteriophages, bacteria and fungi, and the product of this gene is likely responsible for replicating the genetic material of bacteriophage 44AHJD. ORF 2 encodes a protein with homology to the dinC gene of Bacillus subtilis which encodes a protein involved in teichoic acid biosynthesis. Teichoic acid is a polyphosphate polymer found in some, but not all, Gram positive organisms (and not in Gram negative organisms), where it is attached to the peptidoglycan layer. The phage protein may thus be involved in the synthesis of this material for incorporation into the cell wall, allowing enhanced lysis by the phage lysis enzymes or, as many enzymes can function in “reverse reactions”, may be involved in its degradation allowing for penetration of the peptidoglycan and phage genome entry into the cell following adsorption. The similarity between Staphylococcus aureus bacteriophage 44AHJD and E. coli phage T7 indicates that they may share similar mechanisms of replication and growth. Both phages belong to the Pododviridae Family of bacteriophages and are members of the “T7-like” Genus of this Family (Ackermann and DuBow; VIth ICTV Report).

Two genes, ORF 9 and 12, were identified with the potential to encode antimicrobial protein products. The predicted product of ORF 9 is related to a class of genes which encodes lysozyme-like functions, enzymes which cleave linkages in the mucopolysaccharide cell wall structure of a variety of micro-organisms, including that from the Staphylococcus aureus bacteriophage. ORF 12 of Staphylococcus aureus bacteriophage 44AHJD shows homology to a set of lysis proteins from several bacteriophages. These lysis proteins are also referred to as holins, and represent phage encoded lysis functions required for transit of the phage murein hydrolases (lysozyme) to the periplasm, where it can digest the cell wall and thus lyse the bacterium.

Thus, the present invention seeks to provide a nucleic acid sequence isolated from Staphylococcus aureus bacteriophage 44AHJD comprising at least a portion of one of the genes described above with antimicrobial activity. For example, ORF 1 encodes a DNA polymerase function. It is possible that this polymerase utilizes host-derived accessory proteins for its activity when replicating the phage template, sequestering such proteins from use by the bacterial polymerase, resulting in inhibition of DNA replication, cell division, and cell growth. Alternatively, ORF 9 directly encodes a polypeptide with antimicrobial activity. ORF 9 is predicted to encode an amidase, a protein known to act as a cell wall degrading enzyme. ORF 12 likely encodes a holin function required for transit of the phage amidase (gene 9 product) to the periplasm. When this type of gene product from Bacillus phage phi 29 (gene 14), was cloned in Escherichia coli, cell death ensued (Steiner et al., 1993). Thus, production of proteins from Bacillus phage phi 29 gene 14 in E. coli resulted in cell death, whereas production of protein from Bacillus phage phi 29 gene 14 concomitantly with the phi 29 lysozyme or unrelated murein-degrading enzymes led to lysis, suggesting that membrane-bound protein 14 induces a nonspecific lesion in the cytoplasmic membrane (Steiner et al., 1993).

The present invention also provides the use of the Staphylococcus bacteriophage 44 AHJD antimicrobial ORFs or ORF products as pharmacological agents, either wholly or in part and derivatives, as well as the use of corresponding peptidomimetics, developed from amino acid or nucleotide sequence knowledge derived from Staphylococcus bacteriophage 44 AHJD killer ORFs. This can be done where the structure of the peptidomimetic compound corresponds to the structure of the active portion of a product of an ORF. In this analysis, the peptide backbone is transformed into a carbon based hydrophobic structure that can retain cytostatic or cytocidal activity for the bacterium. This is done by standard medicinal chemistry methods, measuring growth inhibition of the various molecules in liquid cultures or on solid medium. These mimetics also represent lead compounds for the development of novel antibiotics. In this context, “corresponds” means that the peptidomimetic compound structure has sufficient similarities to the structure of the active portion of a product of one of the Staphylococcus ORFs listed in Table 7, that the peptidomimetic will interact with the same molecule as the product of the ORF, and preferably will elicit at least one cellular response in common which relates to the inhibition of the cell by the phage protein.

To validate the identity of an ORF as a killer ORF, it is preferably expressed in the host or other test bacterial organism and the effect of this expression on bacterial growth and replication is assessed. Therefore, all individual ORFs identified herein, e.g., those identified above, can be expressed, preferably overexpressed, in a suitable host bacterium e.g., a host Staphylococcus and the effect of this expression or overexpression on host metabolism and viability can be measured.

Individual ORFs can be resynthesized from the phage genomic DNA by the polymerase chain reaction (PCR) using oligonucleotide primers flanking the ORF on either side. Those skilled in the art are familiar with the design and synthesis of appropriate primer sequences. These single ORFs are preferably engineered so that they contain appropriate cloning sites at their extremities to allow their introduction into a new bacterial expression plasmid, allowing propagation in a standard bacterial host such as E. coli, but containing the necessary information for plasmid replication in the target microbe, Staphylococcus sp. (hereafter referred to as a shuttle vector). ORF nucleic acid sequences can also be provided by direct chemical synthesis based on the ORF sequences identified herein using conventional synthesis methods familiar to those skilled in the art.

This shuttle vector also preferably contains regulatory sequences that allow inducible expression of the introduced ORF. As the candidate ORF may encode a killer function that will eliminate the host, it is highly advantageous that it not be expressed (or at least not expressed at a substantial level) prior to testing for activity; thus screening for such sequences in a constitutive fashion is less likely to be successful (lethality). For example, regulatory sequences from the ars operon can be used to direct individual ORF expression in Staphylococcus. The ars operon encodes a series of proteins which normally mediate the extrusion of arsenite and several other trivalent oxyanions from the cells when they are exposed to such toxic substances in their environment. The operon encoding this detoxifying mechanism is normally silent and only induced when arsenite-related compounds are present.

Therefore, individual phage ORFs can be expressed in Staphylococcus or other suitable host in an inducible fashion by adding to the culture medium non-toxic arsenite concentrations during the growth of individual Staphylococcus (or other host cells) clones expressing such individual phage ORFs. Toxicity of the phage killer ORF for the host is monitored by reduction or arrest of growth under induction conditions, as measured by optical density in liquid culture or after plating the induced cultures on solid medium. Subsequently, interference of the phage ORF with the host biochemical pathways ultimately leading to reducing or arresting host metabolism can be measured by pulse chase experiments using radiolabeled precursors of either DNA replication, RNA transcription, or protein synthesis.

Of course, other inducible regulatory sequences (e.g., promoters, operators, etc.) may be used (e.g., systems using positive induction of expression or systems using release of repression). A variety of such systems are known to those skilled in the art and can be utilized in the present invention.

Nucleic acid sequences of the present invention can be isolated using a method similar to those described herein or other methods known to those skilled in the art. In addition, such nucleic acid sequences can be chemically synthesized by well-known methods. Having the phage 44 AHJD ORFs, e.g., anti-bacterial ORFs of the present invention, portions thereof, or oligonucleotides derived therefrom as described, other antimicrobial sequences from other bacteriophage sources can be identified and isolated using methods described here or other methods, including methods utilizing nucleic acid hybridization and/or computer-based sequence alignment methods.

The invention also provides bacteriophage antimicrobial DNA segments from other phages based on nucleic acids and sequences hybridizing to the presently identified inhibitory ORF under high stringency conditions or sequences which are highly homologous. The bacteriophage antimicrobial DNA segment from bacteriophage 44 AHJD can be used to identify a related segment from another unrelated phage based on stringent conditions of hybridization or on being a homolog based on nucleic acid and/or amino acid sequence comparisons. As with the phage 44 AHJD inhibitory sequences, such homologous coding sequences and products can be used as antimicrobials, to construct active portions or derivatives, to construct peptidomimetics, and to identify bacterial targets.

Identification of Bacterial Targets

The present invention provides the use of Staphylococcus bacteriophage 44AHJD ORFs, such as for example ORFs 12 and 25 anti-microbial activity to identify essential host bacterium interacting proteins or other targets that could, in turn, be used for drug design and/or screening of test compounds. Thus, the invention provides a method of screening for antibacterial agents by determining whether test compounds interact with (e.g., bind to) the bacterial target. The invention also provides a method of making an antibacterial agent based on production and purification of the protein or RNA product of bacteriophage 44AHJD ORF 12 or 25. The method involves identifying a bacterial target of the product of ORF 12 or 25, screening a plurality of compounds to identify a compound active on the target, and synthesizing the compound in an amount sufficient to provide a therapeutic effect when administered to an organism infected by a bacterium naturally producing the target. The rationale is that the product of ORFs 12 and 25 can physically interact and/or modify certain microbial host components to block their function.

A variety of methods are known to those skilled in the art for identifying interacting molecules and for identifying target cellular components. Several approaches and techniques are described below which can be used to identify the host bacterial pathway and protein that interact or are inhibited by ORF 12 or 25.

The first approach is a genetic screen for protein:protein interaction, e.g., either some form of two-hybrid screen or some form of suppressor screen. In one form of the two hybrid screen involving the yeast two hybrid system, the nucleic acid segment encoding ORF 12 or 25, or a portion thereof, is fused to the carboxyl terminus of the yeast Gal4 DNA binding domain to create a bait vector. A genomic DNA library of cloned S. aureus sequences which have been engineered into a plasmid where the S. aureus sequences are fused to the carboxyl terminus of the yeast GAL4 activation domain II (amino acids 768-881) is also generated. These plasmids are introduced alone, or in combination, into a yeast strain, e.g., AH109 (Clontech Laboratories, Palo Alto, Calif.), previously engineered with chromosomally integrated copies of the E. coli lacZ and the selectable HIS3 and ADE2 genes, both under Gal4 regulation (Durfee et al., 1993). If the two proteins expressed in yeast interact, the resulting complex will activate transcription from promoters containing Gal4 binding sites. The lacZ, HIS3 and ADE2 genes, each driven by a promoter containing Gal4 binding sites, have been integrated into the genome of the host yeast system and are used for measuring protein-protein interactions. Such a system provides a physiological environment in which to detect potential protein interactions.

This system has been extensively used to identify novel protein-protein interaction partners and to map the sites required for interaction (for example, to identify interacting partners of translation factors (Qui et al., 1998), transcription factors (Katagiri et al., 1998), proteins involved in signal transduction (Endo et al., 1997). Alternatively, a bacterial two-hybrid screen can be utilized to circumvent the need for the interacting proteins to be targeted to the nucleus, as is the case in the yeast system (Karimova et al., 1998).

The protein targets of ORFs 12 and 25 can also be identified using bacterial genetic screens. One approach involves the overexpression of ORF 12 or 25 protein in mutagenized S. aureus followed by plating the cells and searching for colonies that can survive the anti-microbial activity of ORF 12 or 25. These colonies are then grown, their DNA extracted, and cloned into an expression vector that contains a replicon of a different incompatibility group from the plasmid expressing ORF 12 or 25. This library is then introduced into a wild-type Staph A bacterium in conjunction with an expression vector driving synthesis of ORF 12 or 25, followed by selection for surviving bacteria. Thus, Staph A DNA fragments from the survivors presumably contain a DNA fragment from the original mutagenized Staph A genome that can protect the cell from the antimicrobial activity of ORF 12 or 25. This fragment can be sequenced and compared with that of the bacterial host to determine in which gene the mutation lies. This approach enables one to determine the targets and pathways that are affected by the killing function.

Alternatively, the bacterial targets can be determined in the absence of selecting for mutations using the approach known as “multicopy suppression”. In this approach, the DNA from the wild type Staph A host is cloned into an expression vector that can coexist with the one containing ORF 12 or 25. Those plasmids that contain host DNA fragments and genes which protect the host from the anti microbial activity of ORF 12 or 25 can then be isolated and sequenced to identify putative targets and pathways in the host bacteria.

Another approach is based on identifying protein:protein interactions between the product of ORF 12 or 25 and S. aureus host proteins, using a biochemical approach based on affinity chromatography. This approach has been used to identify interactions between lambda phage proteins and proteins from their E. coli host (Sopta et al., 1995). The product of ORF 12 or 25 is fused to a tag (e.g. -glutathione-S-transferase) after insertion in a commercially available plasmid vector which directs high-level expression after induction of the responsive promoter driving the fusion protein. The fusion protein is expressed in E. coli, purified, and immobilized on a solid phase matrix. Total cell extracts from S. aureus are then passed through the affinity matrix containing the immobilized phage ORF fusion protein; host proteins retained on the column are then eluted under different conditions of ionic strength, pH, and detergents and identified by gel electrophoresis. They are recovered from the gel by transfer to a high affinity membrane. The proteins are individually digested to completion with a protease (e.g.-trypsin) and either molecular mass or the amino acid sequence of the tryptic fragments can be determined by mass spectrometry using MALDI-TOF technology (Qin et al., 1997). The sequence of the individual peptides from a single protein are then analyzed by a bioinformatics approach to identify the S. aureus protein interacting with the phage ORF. This is performed by a computer search of the S. aureus genome for the identified sequence. Alternatively, tryptic peptide fragments of the S. aureus genome can be predicted by computer software based on the nucleotide sequence of the genome, and the predicted molecular mass of peptide fragments generated in silico compared to the molecular mass of the peptides obtained from each interacting protein eluted from the affinity matrix.

In addition, an oligonucleotide cocktail can be synthesized based on the primary amino acid sequence determined for an interacting S. aureus protein fragment. This oligonucleotide cocktail would comprise a mixture of oligonucleotides based on the nucleotide sequences of the primary amino acid of the predicted peptide, but in which all possible codons for a particular amino acid sequence are present in a subset of the oligonucleotide pool. This cocktail can then be used as a degenerate probe set to screen, by hybridization to genomic or cDNA libraries, to isolate the corresponding gene.

Alternatively, antibodies raised to peptides which correspond to an interacting S. aureus protein fragment can be used to screen expression libraries (genomic or cDNA) to identify the gene encoding the interacting protein.

Vectors

The invention also provides vectors, preferably expression vectors, harboring the anti-microbial DNA nucleic acid segment of the invention in an expressible form, and cells transformed with the same. Such cells can serve a variety of purposes, such as in vitro models for the function of the anti-microbial nucleic acid segment and screening for downstream targets of the anti-microbial nucleic acid segment, as well as expression to provide relatively large quantities of the inhibitory product.

Thus, an expression vector harboring the anti-microbial nucleic acid segment or parts thereof (Staph A bacteriophage 44AHJD ORF 12 or 25) can also be used to obtain substantially pure protein. Well-known vectors, such as the pGEX series (available from Pharmacia), can be used to obtain large amounts of the protein which can then be purified by standard biochemical methods based on charge, molecular mass, solubility, or affinity selection of the protein by using gene fusion techniques (such as GST fusion, which permits the purification of the protein of interest on a glutathione column). Other types of purification methods or fusion proteins could also be used as recognized by those skilled in the art.

Likewise, vectors containing bacteriophage 44AHJD ORFs 12 and 25 can be used in methods for identifying targets of the encoded antibacterial ORF product, e.g., as described above, and/or for testing inhibition of homologous bacterial targets or other potential targets in bacterial species other than Staphylococcus aureus.

Antibodies

Antibodies, both polyclonal and monoclonal, can be prepared against the protein encoded by a bacteriophage anti-microbial DNA segment of the invention (e.g., Staph A bacteriophage 44AHJD ORF 12 or 25) by methods well known in the art. Protein for preparation of such antibodies can be prepared by purification, usually from a recombinant cell expressing the specified ORF or fragment thereof. Those skilled in the art are familiar with methods for preparing polyclonal or monoclonal antibodies (See, e.g., Antibodies: A Laboratory Manual, Harlow and Lane, Cold Spring Harbor Laboratory, CSHL Press, N.Y., 1988).

Such antibodies can be used for a variety of purposes including affinity purification of the protein encoded by the bacteriophage anti-microbial DNA segment, tethering of the protein encoded by the bacteriophage anti-microbial DNA segment to a solid matrix for purposes of identifying interacting host bacterium proteins, and for monitoring of expression of the protein encoded by the bacteriophage anti-microbial DNA segment.

Recombinant Cells

Bacterial cells containing an inducible vector regulating expression of the bacteriophage anti-microbial DNA segment can be used to generate an animal model system for the study of infection by the host bacterium. The functional activity of the proteins encoded by the bacteriophage anti-microbial DNA segments, whether native or mutated, can be tested in animal in vitro or in vivo models.

While such cells containing inducible expression vectors is preferred, other recombinant cells containing a recombinant phage 44AHJD ORF 12 or 25 sequence or portion thereof are also provided by the present invention.

Also, a recombinant cell may contain a recombinant sequence encoding at least a portion of a protein which is a target of phage 44AHJD ORF 12 or 25 inhibitory ORF product.

In the context of this invention, in connection with nucleic acid sequences, the term “recombinant” refers to nucleic acid sequences which have been placed in a genetic location by intervention using molecular biology techniques, and does not include the relocation of phage sequences during or as a result of phage infection of a bacterium or normal genetic exchange processes such as bacterial conjugation.

Derivatization of Identified Anti-microbials

In cases where the identified anti-microbials above are peptidic compounds, the in vivo effectiveness of such compounds may be advantageously enhanced by chemical modification using the natural polypeptide as a starting point and incorporating changes that provide advantages for use, for example, increased stability to proteolytic degradation, reduced antigenicity, improved tissue penetration, and/or improved delivery characteristics.

In addition to active modifications and derivative creations, it can also be useful to provide inactive modifications or derivatives for use as negative controls or introduction of immunologic tolerance. For example, a biologically inactive derivative which has essentially the same epitopes as the corresponding natural antimicrobial can be used to induce immunological tolerance in a patient being treated. The induction of tolerance can then allow uninterrupted treatment with the active anti-microbial to continue for a significantly longer period of time.

Modified anti-microbial polypeptides and derivatives can be produced using a number of different types of modifications to the amino acid chain. Many such methods are known to those skilled in the art. The changes can include, for example, reduction of the size of the molecule, and/or the modification of the amino acid sequence of the molecule. In addition, a variety of different chemical modifications of the naturally occurring polypeptide can be used, either with or without modifications to the amino acid sequence or size of the molecule. Such chemical modifications can, for example, include the incorporation of modified or non-natural amino acids or non-amino acid moieties during synthesis of the peptide chain, or the post-synthesis modification of incorporated chain moieties.

The oligopeptides of this invention can be synthesized chemically or through an appropriate gene expression system. Synthetic peptides can include both naturally occurring amino acids and laboratory synthesized, modified amino acids. into Also provided herein are functional derivatives of anti-microbial proteins or polypeptides. By “functional derivative” is meant a “chemical derivative,” “fragment,” “variant,” “chimera,” or “hybrid” of the polypeptide or protein, which terms are defined below. A functional derivative retains at least a portion of the function of the protein, for example, reactivity with a specific antibody, enzymatic activity or binding activity.

A “chemical derivative” of the complex contains additional chemical moieties not normally a part of the protein or peptide. Such moieties may improve the molecule's solubility, absorption, biological half-life, and the like. The moieties may alternatively decrease the toxicity of the molecule, eliminate or attenuate any undesirable side effect of the molecule, and the like. Moieties capable of mediating such effects are disclosed in Genaro 1995, Remington's Pharmaceutical Science. Procedures for coupling such moieties to a molecule are well known in the art. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing primary amine- containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine alpha-amino group.

Tyrosyl residues are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction carbodiimide (R′-N-C-N-R′) such as 1-cyclohexyl-3-(2-morpholinyl(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking component peptides to each other or the complex to a water-insoluble support matrix or to other macromolecular carriers. Commonly used cross-linking agents include, for example, 1,1-bis (diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl) dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the alpha-amino groups of lysine, arginine, and histidine side chains (Creighton, T. E., Proteins: Structure and Molecular Properties, W. H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half-life, and the like. The moieties may alternatively eliminate or attenuate any undesirable side effect of the protein complex. Moieties capable of mediating such effects are disclosed, for example, in Genaro 1995, Remington's Pharmaceutical Science.

The term “fragment” is used to indicate a polypeptide derived from the amino acid sequence of the protein or polypeptide having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence.

Another functional derivative intended to be within the scope of the present invention is a “variant” polypeptide which either lacks one or more amino acids or contains additional or substituted amino acids relative to the native polypeptide. The variant may be derived from a naturally occurring polypeptide by appropriately modifying the protein DNA coding sequence to add, remove, and/or to modify codons for one or more amino acids at one or more sites of the C-terminus, N-terminus, and/or within the native sequence.

A functional derivative of a protein or polypeptide with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183; Sambrook et al., 1989) wherein nucleotides in the DNA coding sequence are modified such that a modified coding sequence is produced, and thereafter expressing this recombinant DNA in a prokaryotic or eukaryotic host cell, using techniques such as those described above. Alternatively, components of functional derivatives of complexes with amino acid deletions, insertions and/or substitutions may be conveniently prepared by direct chemical synthesis, using methods well-known in the art.

Insofar as other anti-microbial inhibitor compounds identified by the invention described herein may not be peptidal in nature, other chemical techniques exist to allow their suitable modification, as well, and according the desirable principles discussed above.

Administration and Pharmaceutical Compositions

For the therapeutic and prophylactic treatment of infection, the preferred method of preparation or administration of anti-microbial compounds will generally vary depending on the precise identity and nature of the anti-microbial being delivered. Thus, those skilled in the art will understand that administration methods known in the art will also be appropriate for the compounds of this invention. Pharmaceutical compositions are prepared, as understood by those skilled in the art, to be appropriate for therapeutic use. Thus, generally the components and composition are prepared to be sterile and free of components or contaminants which would pose an unacceptable risk to a patient. For compositions to be administered internally is is generally important that the composition be pyrogen free, for example.

The particularly desired anti-microbial can be administered to a patient either by itself, or in pharmaceutical compositions where it is mixed with suitable carriers or excipient(s). In treating an infection, a therapeutically effective amount of an agent or agents is administered. A therapeutically effective dose refers to that amount of the compound that results in amelioration of one or more symptoms of bacterial infection and/or a prolongation of patient survival or patient comfort.

Toxicity, therapeutic and prophylactic efficacy of anti-microbials can be determined by standard pharmaceutical procedures in cell cultures and/or experimental organisms such as animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

For any compound identified and used in the method of the invention, the therapeutically effective dose can be estimated initially from cell culture assays. Such information can be used to more accurately determine useful doses in organisms such as plants and animals, preferably mammals, and most preferably humans. Levels in plasma may be measured, for example, by HPLC or other means appropriate for detection of the particular compound.

The exact formulation, route of administration and dosage can be chosen by the individual physician in view of the patient's condition (see e.g. Fingl et. al., in The Pharmacological Basis of Therapeutics, 1975, Ch. 1 p.1).

It should be noted that the attending physician would know how and when to terminate, interrupt, or adjust administration due to toxicity, organ dysfunction, or other systemic malady. Conversely, the attending physician would also know to adjust treatment to higher levels if the clinical response were not adequate (precluding toxicity). The magnitude of an administered dose in the management of the disorder of interest will vary with the severity of the condition to be treated and the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary or phyto medicine.

Depending on the specific infection target being treated and the method selected, such agents may be formulated and administered systemically or locally, i.e., topically. Techniques for formulation and administration may be found in Genaro 1995, Remington's Pharmaceutical Science. Suitable routes may include , for example, oral, rectal, transdermal, vaginal, transmucosal, intestinal, parenteral, intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate identified anti-microbials of the present invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active anti-microbial compounds in water-soluble form. Alternatively, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The above methodologies may be employed either actively or prophylactically against an infection of interest.

To identify DNA segments of Staph A bacteriophage 44AHJD capable of acting as anti-microbial agents, a strategy described in U.S. Provisional Application Pelletier, et al., No. 60/168,777 filed Dec. 1, 1999 was employed. In essence, the procedure involved sequence characterization of the bacteriophage, identification of protein coding regions (open reading frames or ORFs), subcloning of all ORFs into an appropriate inducible expression vector, transfer of the ORF subclones into Staph. A, followed by induction of ORF expression and assessment of effect on growth. We employed discovery steps as described in the Examples.

There are a number of methods provided for determining if a compound binds to, interacts with, activates or inhibits an activity or expression of a polypeptide and/or polynucleotide target such as dnaN for example. Screening methods that measure the binding of a screened compound to an ORF 12 or 25 target or product, such as for example dnaN polypeptide and/or polynucleotide, or binding of a screened compound to cells or supports bearing an ORF 12 or 25 target or product polypeptide or a fusion protein comprising the target, by direct labeling or indirectly associating a label with a screened compound are within the scope of the present invention. A screening method of the invention may involve competition for binding of a labeled competitor binding molecule, polypeptide and/or polynucleotide, such as bacteriophage 44AHJD ORF 12 or 25 target or product, or a fragment which binds to a target protein such as dnaN.

Accordingly, the present invention provides methods of screening compounds to identify those compounds which modulate, bind to, interact with, inhibit and/or activate activity or expression of a polypeptide or polynucleotide of the invention.

### EXAMPLE I

Growth of Staph A Bacteriophage 44AHJD and Purification of Genomic DNA.

The Staphylococcus aureus propagating strain (PS 44A) (Felix d'Herelle Reference Centre #HER 1101, Ottawa, Canada) was used as a host to propagate its respective phage 44AHJD (Felix d'Herelle Reference Centre #HER101). Two rounds of plaque purification of phage 44AHJD were performed on soft agar essentially as described in Sambrook et al (1989). Briefly, the PS 44AHJD strain was grown overnight at 37° C. in Nutrient broth [NB: 0.3% Bacto beef extract, 0.5% Bacto peptone (Difco Laboratories) and 0.5% NaCl (w/v)]. The culture was then diluted 20× in NB and incubated at 37° C. until the OD₅₄₀= .2 (early log phase) with constant agitation. In order to obtain single plaques, phage 44AHJD was subjected to 10-fold serial dilutions using phage buffer (1 mM MgSO₄, 5 mM MgCl₂, 80 mM NaCl and 0.1% Gelatin (w/v)) and 10 μl of each dilution was used to infect 0.5 ml of the cell suspension in the presence of 400 μg/ml CaCl₂. After incubation of 15 min at room temperature (RT), 2 ml of melted soft agar kept at 45° C. (NB supplemented with 0.6% agar) was added to the mixture and poured onto the surface of 100 mm nutrient agar plates (0.3% Bacto Beef extract, 0.5% Bacto peptone, 0.5% NaCl and 1.5% Bacto agar,(w/v)). After overnight incubation at 30° C., a single plaque was isolated, resuspended in 1 ml of phage buffer by end over end rotation for 2 hrs at 20° C., and the phage suspension was diluted and used for a second infection as described above. After overnight incubation at 30° C., a single plaque was isolated and used as a stock.

The propagation procedure for bacteriophage 44AHJD was modified from the agar layer method of Swanstorm and Adams (1951). Briefly, the PS 44A strain was grown to stationary phase overnight at 37° C. in Nutrient broth. The culture was then diluted twenty-fold in NB and incubated at 37° C. until the OD₅₄₀=. 2. The suspension (15×10⁷ Bacteria) was then mixed with 15×10⁵ plaque forming units (pfu) to give a ratio of 100-bacteria/phage particle in the presence of 400 μg/ml of CaCl₂. After incubation for 15 min at 20° C., 7.5 ml of melted soft agar (NB plus 0.6% agar) were added to the mixture and poured onto the surface of 150 mm nutrient agar plates and incubated 16 hrs at 37° C. To collect the phage plate lysate, 20 ml of NB were added to each plate and the soft agar layer was collected by scrapping off with a clean microscope slide followed by shaking of the agar suspension for 5 min to break up the agar. The mixture was then centrifuged for 10 min at 4,000 RPM (2,830 ×g) in a JA-10 rotor (Beckman) and the supernatant fluid (lysate) was collected and subjected to a treatment with 10 μg /ml of DNase I and RNase A for 30 min at 37° C. To precipitate the phage particles, the phage suspension was adjusted to 10% (w/v) PEG 8000 and 0.5 M of NaCl followed by incubation at 4° C. for 16 hrs. The phage was recovered by centrifugation at 4,000 rpm (3,500 ×g) for 20 min at 4° C. on a GS-6R table top centrifuge (Beckman). The pellet was resuspended with 2 ml of phage buffer (1 mM MgSO₄, 5 mM MgCl₂, 80 mM NaCl and 0.1% Gelatin). The phage suspension was extracted with 1 volume of chloroform and further purified by centrifugation on a cesium chloride step gradient as described in Sambrook et al. (1989), using a TLS 55 rotor centrifuged in an Optima TLX ultracentrifuge (Beckman) for 2 hr at 28,000 rpm (67,000 ×g) at 4° C. Banded phage was collected and ultracentrifuged again on an isopycnic cesium chloride gradient (1.45 g/ml) at 40,000 rpm (64,000 ×g) for 24 h at 4° C. using a TLV rotor (Beckman). The phage was harvested and dialyzed for 4 h at room temperature against 4 L of dialysis buffer consisting of 10 mM NaCl, 50 mM Tris-HCl [pH 8] and 10 mM MgCl₂. Phage DNA was prepared from the phage suspension by adding 20 mM EDTA, 50 ug/ml Proteinase K and 0.5% SDS and incubating for 1 hr at 65° C., followed by successive extractions with 1 volume of phenol, 1 volume of phenol-chloroform and 1 volume of chloroform. The DNA was then dialyzed overnight at 4° C. against 4 L of TE (10 mM Tris HCl [pH 8.0], 1 mM EDTA).

### EXAMPLE II

DNA Seguencing of Bacteriophage 44AHJD Genome

Four micrograms of phage DNA was diluted in 200 μl of TE (10 mM Tris, [pH 8.0], 1 mM EDTA) in a 1.5 ml eppendorf tube and sonication was performed (550 Sonic Dismembrator™, Fisher Scientific). Samples were sonicated under an amplitude of 3 μm with bursts of 5 s spaced by 15 s cooling in ice/water for 3 to 4 cycles. The sonicated DNA was then size fractionated by electrophoresis on 1% agarose gels utilizing TAE (1×TAE is: 40 mM Tris-acetate, 1 mM EDTA [pH 8.0]) as the running buffer. Fractions ranging from 1 to 2 kbp were excised from the agarose gel and purified using a commercial DNA extraction system according to the instructions of the manufacturer (Qiagen), with a final elution of 50 μl of 1 mM Tris HCl [pH 8.5].

The ends of the sonicated DNA fragments were repaired with a combination of T4 DNA polymerase and the Klenow fragment of E. coli DNA polymerase I, as follows. Reactions were performed in a reaction mixture (final volume, 100 μl) containing sonicated phage DNA, 10 mM Tris-HCl [pH 8.0], 50 mM NaCl, 10 mM MgCl₂, 1 mM DTT, 50 μg/ml BSA, 100 μM of each dNTP and 15 units of T4 DNA polymerase (New England Biolabs) for 20 min at 12° C. followed by addition of 12.5 units of Klenow large fragment (New England Biolabs) for 15 min at room temperature. The reaction was stopped by two phenol/chloroform extractions and the DNA was precipitated with ethanol and the final DNA pellet was resuspended in 20 μl of H2O.

Blunt-ended DNA fragments were cloned by ligation directly into the Hinc II site of pKSII+vector (Stratagene) dephosphorylated by treatment with calf intestinal alkaline phosphatase (New England Biolabs). A typical ligation reaction contained 100 ng of vector DNA, 2 to 5 μl of repaired sonicated phage DNA (50-100 ng) in a final volume of 20 μl containing 800 units of T4 DNA ligase (New England Biolabs) and was incubated overnight at 16° C. Transformation and selection of bacterial clones containing recombinant plasmids was performed in E. coli DH10β according to standard procedures (Sambrook et al., 1989).

Recombinant clones were picked from agar plates into 96-well plates containing 100 μl LB and 100 μg/ml ampicillin and incubated at 37° C. The presence of phage DNA insert was confirmed by PCR amplification using T3 and T7 primers flanking the Hinc II cloning site of the pKS II+vector. PCR amplification of foreign insert was performed in a 15 μl reaction volume containing 10 mM Tris HCl [pH 8.3], 50 mM KCl, 1.5 mM MgCl₂, 0.02% gelatin, 1 μM primer, 187.5 μM each dNTP, and 0.75 units Taq polymerase (BRL). The thermocycling parameters were as follows: 2 min initial denaturation at 94° C. for 2 min, followed by 20 cycles of 30 sec denaturation at 94° C., 30 sec annealing at 57° C., and 2 min extension at 72° C., followed by a single extension step at 72° C. for 10 min. Clones with insert sizes of 1 to 2 kbp were selected and plasmid DNA was prepared from the selected clones using QIAprep™ spin miniprep kit (Qiagen).

The nucleotide sequence of the extremities of each recombinant clone was determined using an ABI 377-36 automated sequencer with two types of chemistry: ABI prism Big Dye™ primer cycle sequencing (21M13 primer: #403055)(M13REV primer: #403056) or ABI prism Big Dye™ terminator cycle sequencing ready reaction kit (Applied Biosystems, #4303152). To ensure co-linearity of the sequence data and the genome, all regions of phage genome were sequenced at least once from both directions on two separate clones. In areas that this criteria was not initially met, a sequencing primer was selected and phage DNA was used directly as sequencing template employing ABI prism Big Dye™ terminator cycle sequencing ready reaction kit.

### EXAMPLE III

Bioinformatic Management of Primary Nucleotide Sequence.

Sequence contigs were assembled using Sequencher™ 3.1 software (GeneCodes). To close contig gaps, sequencing primers were selected near the edge of the contigs. Phage DNA was used directly as sequencing template employing ABI prism BIG DYE™ terminator cycle sequencing ready reaction kit. The complete sequence of bacteriophage 44AHJD is shown in Table 1.

A software program was developed and used on the assembled sequence of bacteriophage 44AHJD to identify all putative ORFs larger than 33 codons. Other ORF identification software can also be utilized, preferably programs which allow alternative start codons. The software scans the primary nucleotide sequence starting at nucleotide #1 for an appropriate start codon. Three possible selections can be made for defining the nature of the start codon; I) selection of ATG, II) selection of ATG or GTG, and III) selection of either ATG, GTG, TTG, CTG, ATT, ATC, and ATA. This latter initiation codon set corresponds to the one reported by the NCBI (http://www.ncbi.nlm.nih.gov/htbin-post/Taxonomy/wprintgc?mode=c) for the bacterial genetic code.

When an appropriate start codon is encountered, a counting mechanism is employed to count the number of codons (groups of three nucleotides) between this start codon and the next stop codon downstream of it. If a threshold value of 33 is reached, or exceeded, then the sequence encompassed by these two codons (start and stop codons) is defined as an ORF. This procedure is repeated, each time starting at the next nucleotide following the previous stop codon found, in order to identify all the other putative ORFs. The scan is performed on all three reading frames of both DNA strands of the phage sequence.

Sequence homology (BLAST) searches for each ORF are then carried out using an implementation of BLAST programs, although any of a variety of different sequence comparison and matching programs can be utilized as known to those skilled in the art.

Downloaded public databases used for sequence analysis include:

i) non-redundant GenBank (ftp://ncbi.nlm.nih.gov/blast/db/nr.Z),

ii) Swissprot (ftp://ncbi.nlm.nih.gov/blast/db/swissprot.Z);

iii) vector (ftp://ncbi.nlm.nih.gov/blast/db/vector.Z);

iv) pdbaa databases (ftp://ncbi.nlm.nih.gov/blast/db/pdbaa.Z);

v) Staphylococcus aureus NCTC 8325 (ftp://ftp.genome.ou.edu/pub/staph/staph-1k.fa);

vi) streptococcus pyogenes (ftp://ftp.genome.ou.edu/pub/strep/strep-1k.fa);

vii) streptococcus pneumoniae (ftp://fip.tigr.org/pub/data/s_pneumoniae/gsp.contigs. 112197.Z);

viii) mycobacterium tuberculosis CSU#9 (ftp://ftp.tigr.org/pub/data/m_tuberculosis/TB_{—}091097.Z) and ix) pseudomonas aeruginosa (http://www.genome.washington.edu/pseudo/data.html).

The results of the homology searches performed on the ORFs is shown in Table 4.

### Example IV

Subcloning of Bacteriophage 44AHJD ORFs into a Staph A Inducible Expression System.

Preparation of Shuttle Vectors

The shuttle vector pT0021, in which the firefly luciferase (lucFF) expression is controlled by the ars (arsenite) promoter/operator (Tauriainen et al., 1997), was modified in the following fashion. Two oligonucleotides corresponding to a short antigenic peptide derived from the heamaglutinin protein of influenza virus (HA epitope tag) were synthesized (Field et al., 1988). The sense strand HA tag sequence (with BamHI, SalI and HindIII cloning sites) is:

5′-gatcccggtcgaccaagcttTACCCATACGACGTCCCAGACTACGCCAGCTGA-3′ (where upper case letters denote the nucletotide sequence of the HA tag); the antisense strand HA tag sequence (with a HindIII cloning site) is:

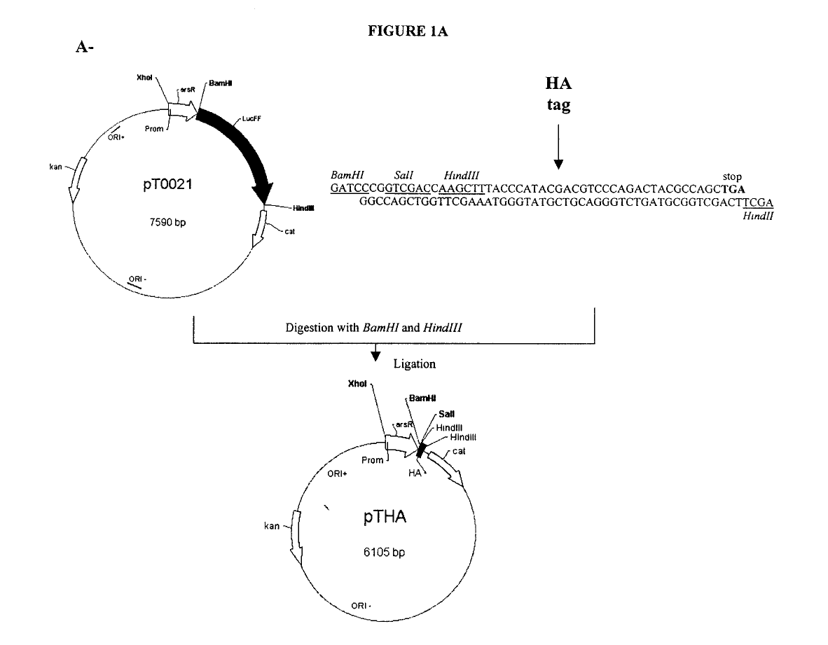

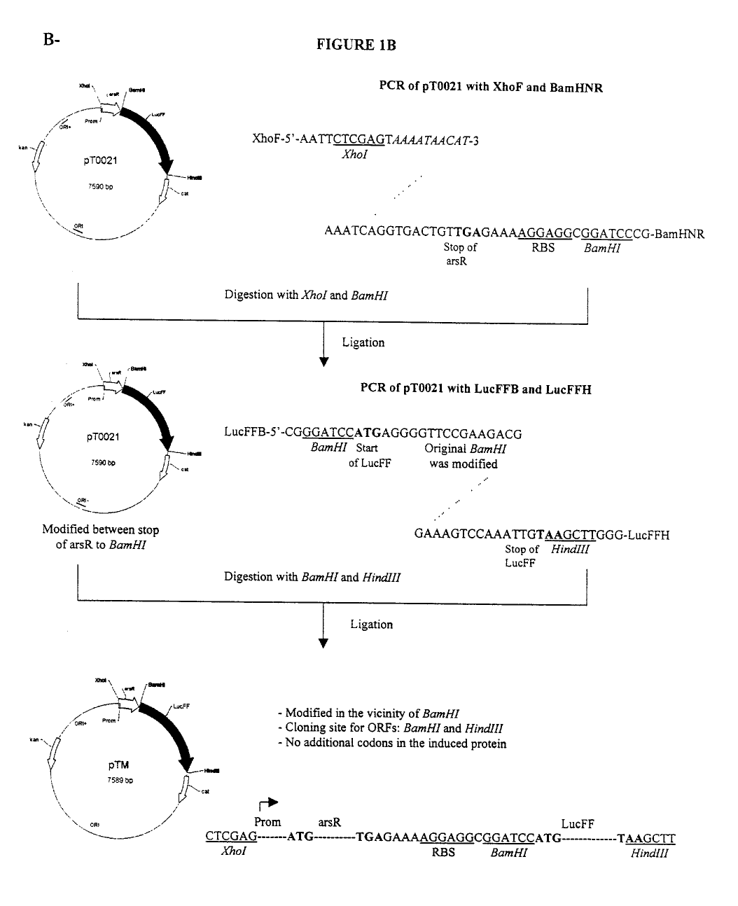

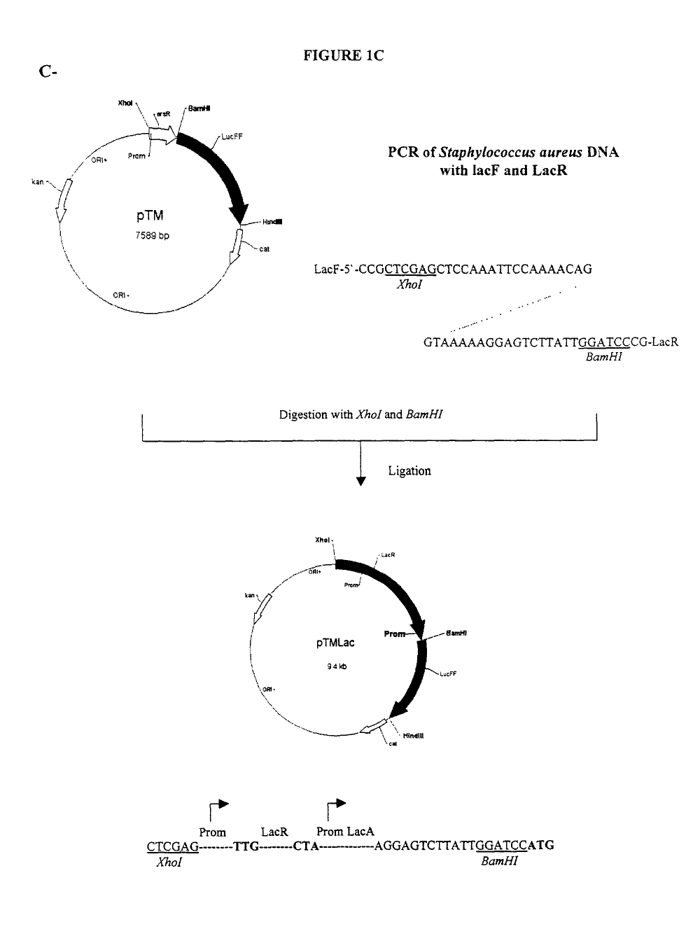

5 ′-agctTCAGCTGGCGTAGTCTGGGACGTCGTATGGGTAaagcttggtcgaccgg-3′ (where upper case letters denote the sequence of the HA tag). The two HA tag oligonucleotides were annealed and ligated into pT0021 vector which had been digested with BamHI and HindIII. This manipulation resulted in replacement of the lucFF gene by the HA tag. This modified shuttle vector containing the arsenite inducible promoter, the arsR gene, and HA tag was named pTHA. A diagram outlining our modification of pT0021 to generate pTHA is shown in FIG. 1A.

The shuttle vector pT0021 was also modified as below to suit our specific application. Two oligonucleotides were synthesized. The sense strand sequence (with XhoI cloning site) is: 5′-AATTCTCGAGTAAAATAACAT-3′; the antisense strand sequence (with a BamHI cloning site) is:

5′-CGGGATCCGCCTCCTTTTCTCAACAGTCACCTGATTT-3′. The two oligonucleotides were used for polymerase chain reaction (PCR) amplification of pT0021 vector. The PCR product was gel purified using the Qiagen kit as described, and digested with XhoI and BamHI. The digested PCR product was again gel purified, ligated into XhoI and BamHI digested pT0021 vector, and used to transform E. coli bacterial strain DH11β (as described above). This manipulation results in the construction of a pT0021-intermediated vector containing a RBS sequence located immediately upstream of the BamHI cloning site. Two other oligonucleotides were synthesized. The sense strand sequence (with BamHI cloning site) is:

5′-CGGGATCCATGAGGGGTTCCGAAGACG-3′; the antisense strand sequence (with a HindIII cloning site) is: 5′-CCCAAGCTTACAATTTGGACTTTC-3′. The two oligonucleotides were used for PCR amplification of pT0021-intermediated vector. The PCR product was gel purified and digested with BamHI and HindIII. The digested PCR product was then gel purified as described, ligated into BamHI and HindIII digested pT0021-intermediated vector, and used to transform E. coli bacterial strain DH10. This modified shuttle vector containing the ATG of the lucFF gene located immediately downstream of the BamHI cloning site was named pTM. A diagram outlining our modification of pT0021 to generate pTM is shown in FIG. 11B.

As another example of inducible promotor, the arsenite-inducible promotor and the asrR gene from the pTM vector were replaced by a lactose-inducible promotor and the lacR gene from Staphylococcus aureus . The S. aureus gene encoding for the repressor of the lac operon (lacR) is found immediately upstream of the promoter-proximal end of the the lacA-G genes. Two oligonucleotides corresponding to a 2.18 kb-DNA region encompassing the lacR and the lac operon promotor region were synthesized. The sense strand sequence is: 5′-ccgctcgagCTCCAAATTCCAAAACAG-3′ (with a XhoI cloning site, ctcgag) ; the antisense strand sequence is: 5′-cgggatccAATAAGACTCCTTTTTAC-3′ (with a BamHI cloning site, ggatcc). These two oligonucleotides were used for the PCR amplification of Staphylococcus aureus DNA. The PCR product was gel purified and digested with XhoI and BamHI. The digested PCR product was also gel purified, ligated into XhoI and BamHI-digested pTM vector, and used to transform E. coli bacterial strain DH10β. In the resulting vector, pTMLac, the firefly luciferase (lucFF) expression is under the control of the S. aureus lac operon promoter/operator. Recombinant pTMLac clones were picked and the sequence integrity of the 2.18 kb-lac operon region (lacR+lac promotor) was verified directly by DNA sequencing. A diagram outlining the pTMSLac vector characteristics is shown in FIG. 1C.

Cloning of ORFs with a Shine-Dalgarno Sequence.

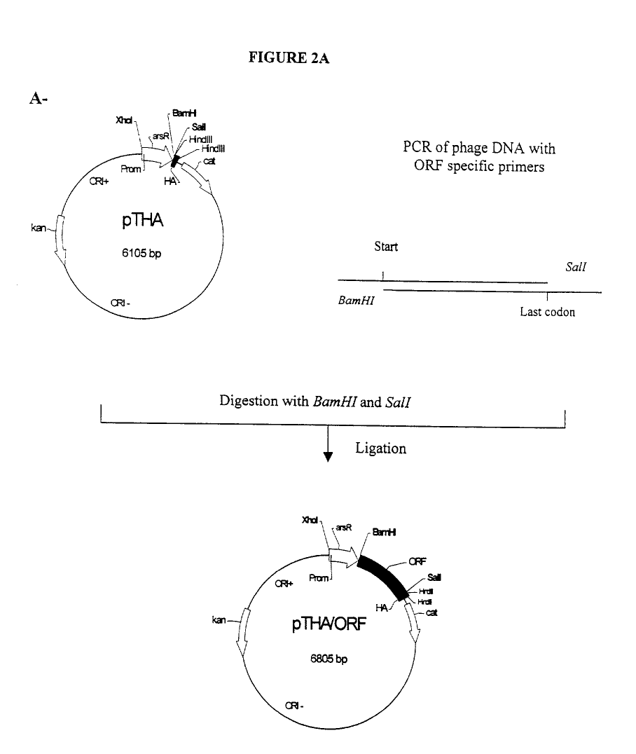

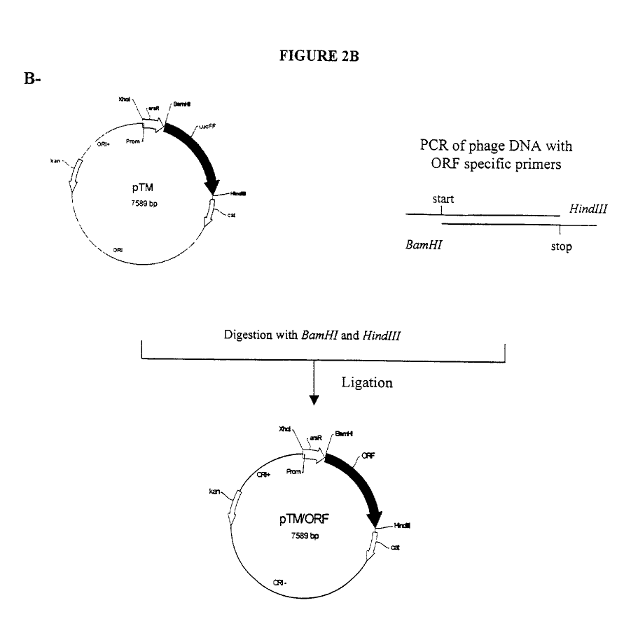

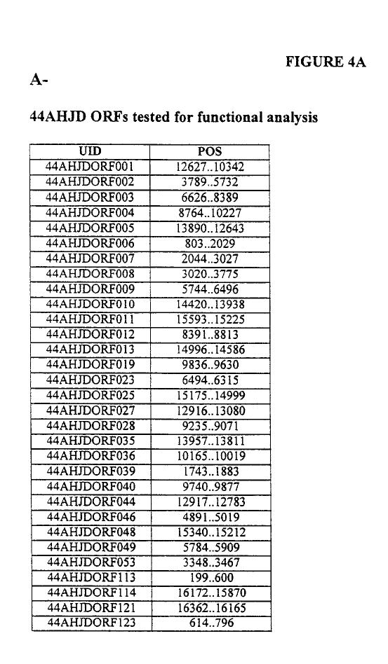

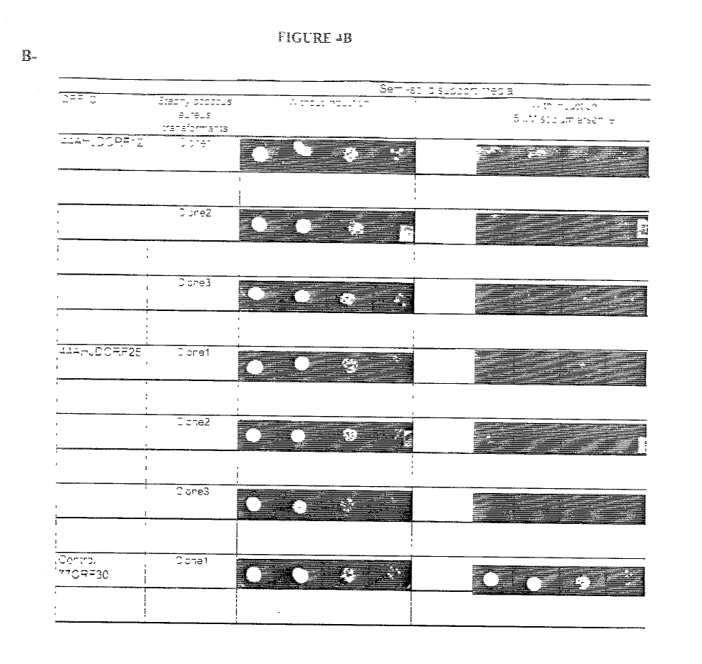

Each ORF, encoded by Bacteriophage 44AHJD, larger than 33 amino acids and having a Shine-Dalgarno sequence upstream of the initiation codon was selected for functional analysis for bacterial inhibition. In total, 31 ORFs were selected and screened as detailed below. A list of these is presented in FIG. 4A. As outlined in FIG. 2A, each individual ORF, from initiation codon to last codon (excluding the stop codon), was amplified from phage genomic DNA using the polymerase chain reaction (PCR). For PCR amplification of ORFs, each sense strand primer targets the initiation codon and is preceded by a BamnHI restriction site (5′cgggatcc3′) and each antisense oligonucleotide targets the pentultimate codon (the one before the stop codon) of the ORF and is preceded by a Sal I restriction site (5′gcgtcgaccg3′). The PCR product of each ORF was gel purified and digested with BamHI and Sall. The digested PCR product was then gel purified using the Qiagen kit as described, ligated into BamHI and SalI digested pTHA vector, and used to transform E. coli bacterial strain DH10β (as described above). As a result of this manipulation, the HA tag is set inframe with the ORF and is positioned at the carboxy terminus of each ORF (pTHA/ORF clones). Recombinant pTHA/ORF clones were picked and their insert sizes were confirmed by PCR analysis using primers flanking the cloning site. The names and sequences of the primers that were used for the PCR amplification were: HAF:

5′TATTATCCAAAACTTGAACA3′; HAR: 5′CGGTGGTATATCCAGTGATT3′. The sequence integrity of cloned ORFs was verified directly by DNA sequencing using primers HAF and HAR. In cases where verification of ORF sequence could not be achieved by one pass with the sequencing primers, additional internal primers were selected and used for sequencing.

Each ORFs cloned into pTHA were also tested following removal of the HA tag. The pT/ORF vectors were obtained by Hind III digestion of individual pTHA/ORFs, gel purification of vector and religation of Hind III ends together.

ORF 12 and ORF 25 were also clones into pTM and pTMLac respectively. Each individual ORF, from initiation codon to stop codon was amplified from phage genomic DNA using the PCR. Each sense strand primer targets the initiation codon and is preceded by a BamHI restriction site (5′-cgggatcc-3′) and each antisense oligonucleotide targets the stop codon of the ORF and is preceded by a HindIII restriction site (5′-cccaagctt-3′). The PCR product of each ORF was purified using the Quiagen kit as described and digested with BamHI and HindIII The digested PCR product was also purified using the Quiagen kit, ligated into BamHI and HindIII digested pTM or pTMLac vector and used to transform E. coli bacterial strain DH10β (as described above). As a result of this manipulation, the ORF is under the control of the arsenite-inducible (pTM) or lactose-inducible (pTMLac) promotors. Recombinant clones were picked and their insert sizes were confirmed by PCR analysis using primers flanking the cloning site. The sequence integrity of cloned ORFs was verified directly by DNA sequencing using primers HAF and HAR.

### EXAMPLE V

Functional Assay for Bacterial Inhibitory Activity of Bacteriophage 44AHJD ORFs.

Transformation of Staphylococcus aureus with Expression Construct Staphylococcus aureus strain RN4220 (Kreiswirth et al., 1983) was used as a recipient for the expression of recombinant plasmids. Electoporation was performed essentially as previously described (Schenk and Laddaga, 1992). Selection of recombinant clones was performed on Luria-Broth agar (LB-agar) plates containing 30 μg/ml of kanamycin.

For each ORF introduced in the pTHA and pT plasmids, 3 independent transformants were isolated and used to individually inoculate cultures in 5 ml of TSB containing 30 μg/ml kanamycin, followed by growth to saturation (16 hrs at 37° C.). An aliquot of this stationary phase culture was used to generate a frozen glycerol stock of the transformant (stored at −80° C.)

The presence of individual phage 44AHJD ORF DNA inserts in the plasmid was verified by PCR amplification using 1.5 μl transformant miniprep DNA in a PCR with primers flanking the cloning site of ORF in pTHA vector (HAF and HAR). The composition of the PCR reaction and the cycling parameters are identical to those employed for library screening described above.

Induction of Gene Expression from the ars- and lac-inducible Promotors

Sodium arsenite (NaAsO₂) was purchased from Sigma (Sigma-Aldrich Canada LTD, Oakville) and was used as heavy metals to induce gene expression from the ars promoter/operator in solid and liquid medium assays.

The lactose (lac) genes of Staphylococcus aureus have been shown to be inducible with the addition of either lactose or galactose to the culture medium (Oskouian & Stewart, 1990, J. Bacteriol. 172: 3804-3812). Galactose (2% w/v) was used to induce the gene expression from the lac promotor/operator in liquid assay.

At pre-determined times, appropriated inducer was added to the culture to induce transcription of the phage ORFs cloned immediately downstream from an arsenite-inducible promoter in the expression plasmids pTHA, pT, or pTM, or a lactose-inducible promotor in the expression plasmid pTMLac. The anti-microbial activity of individual phage 44AHJD ORFs was monitored by two growth inhibitory assays, one on solid agar medium, the other in liquid medium.

The effect of ORF induction on bacterial growth characteristics was then monitored and quantitated.

a Screening on Semi-solid Support Media

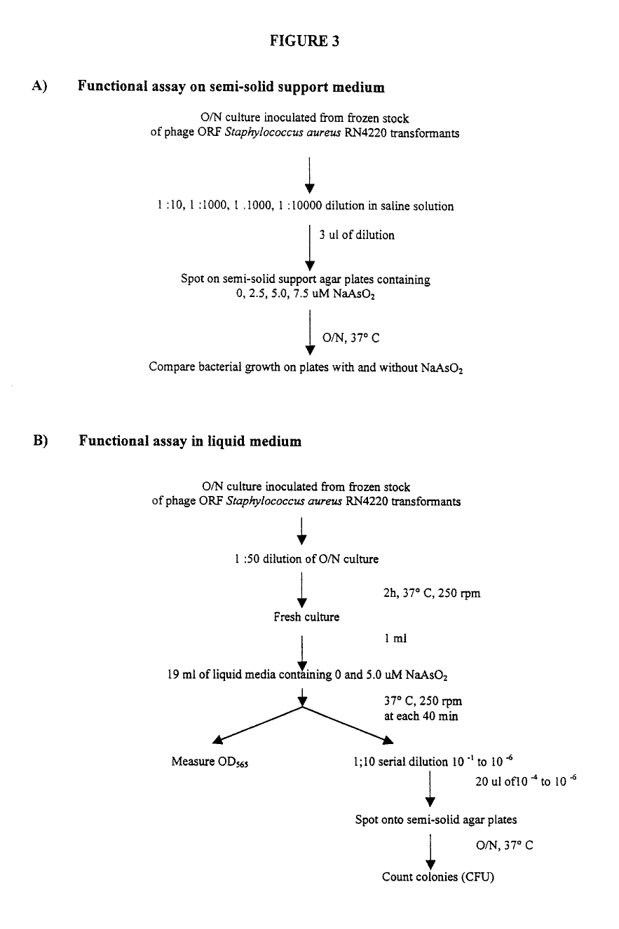

ORFs cloned into pTHA and pT vectors were first screened by the functional assay on semi-solid medium as outlined in FIG. 3A. Cells containing different recombinant plasmids were grown overnight at 37° C. in LB medium supplemented with 30 μg/ml of kanamycin. The cells were then diluted and the identification of inhibitory ORFs was performed by spotting 3 ul of each dilution of S. aureus transformed cells containing phage 44AHJD ORFs onto agar plates containing increasing concentrations of sodium arsenite (0; 2.5; 5; and 7.5 μM) and Kanamycin. The plates were incubated overnight at 37° C., after which a growth inhibition of the ORF transformants on plates that contain arsenite are compared to plates without arsenite. Noninduced and induced cultures of S aureus transformed with a non-inhibitory ORF (77 bacteriophage ORF 30 cloned into pT vector) were included as negative control. The 77 ORF 30 amino acids residue composition from N-terminal to C-terminal is:

MKIKVKKEMRLDELIKWARENPDLSQGKIFFSTGFSDGFVRFHPNTNKCS
TSSFIPIDIPFIVDIEKEVTEETKVDRLIELFEIQEGDYNSTLYENTSIK
ECLYGRCVPTKAFYILNDDL TMTLIWKDGELLV.

Results of the 31 bacteriophage ORFs tested for functional assay on semi-solid media are listed in FIG. 4A. Among them, induction of expression of phage 44AHJD OERF12 and 25 results in the inhibition of growth of the S. aureus transformants. FIG. 4B shows the result of growth inhibition with three clones of S. aureus expressing these inhibitory ORFs or the control non-inhibitory 77 ORF 30.

b Quantification of Growth Inhibition in Liquid Medium

As outlined in FIG. 3B, the effect of ORF induction on bacterial growth inhibition was then further quantitated by functional assay in liquid medium. Cells containing phage 44AHJD ORF 12 or 25 were grown for overnight at 37° C. in LB medium supplemented with the appropriate antibiotic selection. These cultures were 50-fold dilution with fresh media containing kanamycin and the growth was continued for 2 h at 37° C. The same OD565 equivalent of cultures (approximately 1 ml) was added to 19 ml of fresh media containing kanamycin and transferred to a 125 ml-Erlenmeyer flask. The cultures were incubated for an additional 4 hrs at 37° C. in the absence or in the presence of inducer (sodium arsenite at the final concentrations of 5.0 μM or 2.0% galactose). During that period of time, the effect of expression of the phage 44AHJD ORFs on bacterial cell growth was monitored, at each time point intervals, by measuring the OD₅₆₅ and the number of colony forming units (CFU) in the cultures containing or not the inducer. The number of CFU was evaluated as followed. Cultures were serially diluted and aliquots from induced and uninduced cultures were plated out on agar plates containing an appropriate antibiotic selection but lacking inducer. Following incubation overnight at 37° C., the number of colonies was counted. Cultures of S aureus transformed with a non-inhibitory ORF (77 bacteriophage ORF 30 cloned into pT vector or 44AHJD ORF 114 cloned into pTM) were included as control.

As shown in FIG. 5, for each inhibitory ORFs, the number of CFU and OD increased over time under non-induced conditions. Similar growth rates were also observed with transformants harboring non-inhibitory ORF under both induced and non-induced conditions. Cultures of S. aureus transformants harboring the phage ORF 12 or 25 shown a significant lower growth rate compared to their respective parallel cultures grown under noninduced conditions. Induction of expression of ORF 12 or 25 were cytocydal for the bacterial growth. As shown in FIG. 5A, the expression of ORF 12 results in a rapid decrease in the number of CFU. A one log reduction in the number of CFU compared to the number of CFU initially present in the same culture was observed at 1h following induction with sodium arsenite.

As shown in FIG. 5B, the expression of ORF 25 results in a 2 log reduction in the number of CFU compared to the number of CFU initially present in the same culture. The induction of the expression of the same ORF with galactose results in a half log reduction in the number of CFU compared to the number of CFU initially present in the same culture was observed.

### EXAMPLE VI

Phage ORF Protein Expression Analysis in S. aureus

The level of expression of the inhibitory ORFs was measured by performing Western blot analyses. Staphylococcus aureus strain RN4220 was electroporated with each inhibitory ORFs cloned into pTHA vector as described above. Cells containing different recombinant plasmids were grown for overnight at 37° C. in TSB (Tryptic soy broth, DIFCO) medium in the presence of 30 μg/ml kanamycin. The overnight cultures were subjected to a 50-fold dilution with fresh media containing kanamycin and the growth was continued for 2 h at 37° C. At the end, cells were diluted with fresh TSB medium containing or not 5.0 μM of Sodium Arsenite, in the presence of kanamycin and incubated at 37° C. for an additional 3.5 h. The same OD₅₆₅ equivalent of cultures was centrifuged at 3000 g for 5 min and washed with 20 ml of TBS buffer (140 mM NaCl, 25 mM Tris-HCl, pH 7.5) containing protease inhibitors (1 mM of each phenylmethylsulfonyl fluoride (PMSF) and N-ethylmalemyde (NEM)). For lysis, cell pellets were resuspend in 25 μl with TBS buffer containing 1 mM PMSF, 1 mM NEM, 20 μg of each DNAse I and RNase A and 50 Units/ml of lysostaphin, and incubated at 37° C. for 1 h. The reaction was stopped by the addition of 25 μl of 2×SDS buffer (100 mM Tris pH 6.8, 4% SDS, 200 mM DTT, 20% Glycerol and 0.2% Bromophenol blue). Cell lysates were boiled for 10 min, centrifuged for 10 min at 13,000 g and 10-15 μl of the lysates were loaded onto a 15-18% SDS-page using Tris-Glycine-SDS as a running buffer (3.03 g of Tris HCl, 14.4 g of Glycine and 0.1% SDS per liter). After migration, proteins were transferred onto an immobilon-P membrane (PVDF, Millipore) using Tris-Glycin-Methanol as a transfer buffer (3.03 g Tris, 14.4 Glycine and 200 ml Methanol per liter) for 2 hrs at 4° C. at 100 V. PVDF membrane was pretreated in methanol for 30 s, washed 4-5 times with H₂O and soaked in transfer buffer.

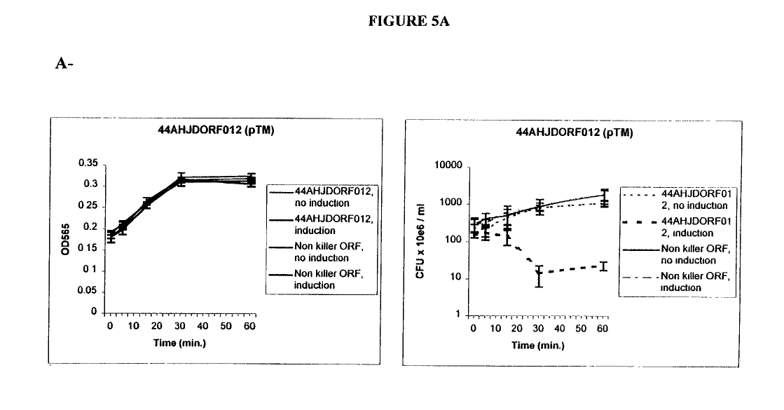

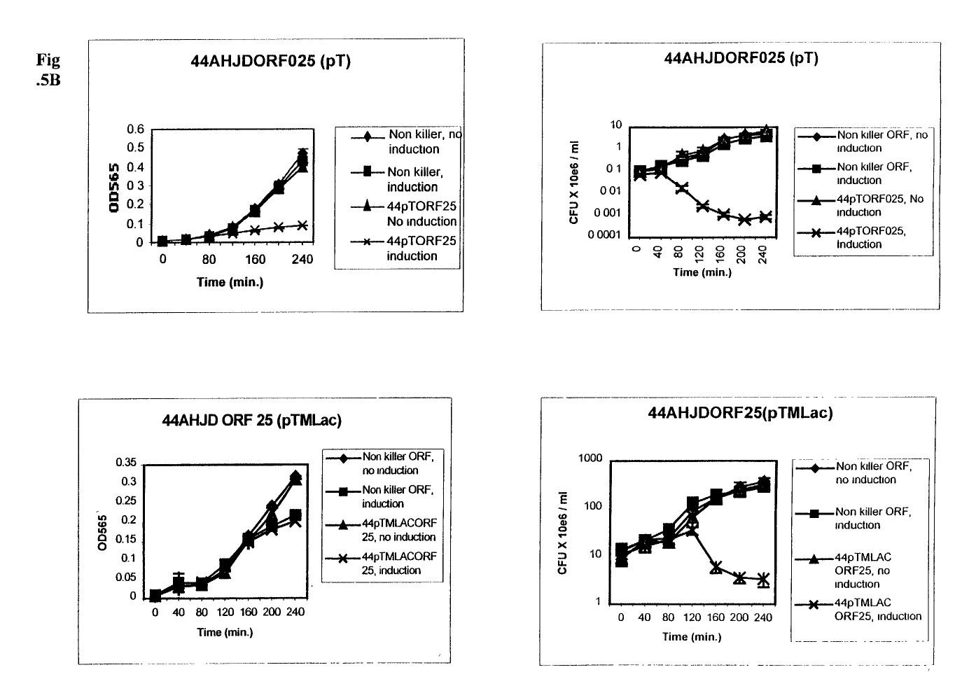

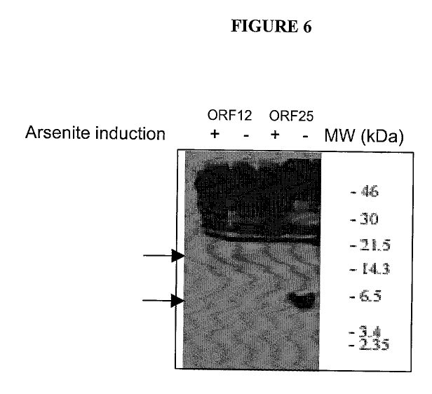

After the transfer, the membrane was blocked in 20 ml of TBS containing 0.05% Tween-20 (TBST), 5% skim milk and 0.5% gelatin for 1 hr at room temperature and then, a pre-blocking antibody (ChromPureRabbit IgG, Jackson immunoResearch lab. # 011-000-003) was added at a dilution of 1/750 and incubated for 1 hr at room temperature or O/N at 4° C. Membrane was washed 6 times for 5 min in TBST at room temperature. The primary antibody (murine mono-HA antibody, Babco # MMS-101 P) directed against the HA epitope tag and diluted 1/1000 was then added and incubated for 3 h at room temperature in the presence of 5% Skim Milk and 0.5% Gelatin. Membrane was washed 6 times for 5 min in TBST at room temperature. A secondary antibody (anti-mouse IgG, peroxidase-linked species-specific whole antibody, Amersham # NA 931) diluted 1/1500 (7.5 μl in 10 ml) was then added and incubated for 1 hr at room temperature. After 6 washes in TBST, the membrane was briefly dried and then, the substrate (Chemiluminescence reagent plus, Mandel # NEL104 ) was added to the membrane and incubated for 1 min at room temperature. The membrane was briefly dried and exposed to x-ray film (Kodak, Biomax MS/MR) for different periods of time (30 s to 10 min). As shows in FIG. 6, the presence of sodium arsenite in the cultures induces the expression of proteins corresponding to the phage 44AHJD ORF 12 and 25.

### EXAMPLE VII

Screening Assays

Phage Display

Phage display is a powerful assay to measure protein:protein interaction. In this scheme, proteins or peptides are expressed as fusions with coat proteins or tail proteins of filamentous bacteriophage. A comprehensive monograph on this subject is Phage Display of Peptides and Proteins. A Laboratory Manual edited by Kay et al. (1996) Academic Press. For phages in the Ff family that include M13 and fd, gene III protein and gene VIII protein are the most commonly-used partners for fusion with foreign protein or peptides. Phagemids are vectors containing origins of replication both for plasmids and for bacteriophage. Phagemids encoding fusions to the gene III or gene VIII can be rescued from their bacterial hosts with helper phage, resulting in the display of the foreign sequences on the coat or at the tip of the recombinant phage.

In the simplest assay, purified recombinant dnaN protein, or a fragment of dnaN, could be immobilized in the wells of a microtitre plate and incubated with phages displaying 44AHJD ORF 25 in fusion with the gene III protein. Washing steps are performed to remove unbound phages and bound phages are detected with monoclonal antibodies directed against phage coat protein (gene VIII protein). Color development by means of an enzyme-linked secondary antibody allows quantitative detection of bound fusion protein. Screening for inhibitors is performed by the incubation of the compound with the immobilized target before the addition of phages. The presence of an inhibitor will specifically reduce the signal in a dose-dependent manner relative to controls without inhibitor.

Identification of the Surface of Interaction on Both Polypeptide Partners.

The invention provides a method for the identification of 44AHJD ORF 25 and DnaN polypeptide fragments which are involved in the interaction between these two proteins. These fragments may include, for example, truncation polypeptides having a portion of an amino acid sequence of any of the two proteins, or variants thereof, such as a continuous series of residues that includes an amino- and/or carboxyl-terminal amino acid sequence.

Partial proteolysis of proteins in solution is one method to delineate the domain boundaries in multi-domain proteins. By subjecting proteins to limited digestion, the most accessible cleavage sites are preferentially hydrolyzed. These cleavage sites preferentially reside in less structured regions which include loops and highly mobile areas typical of the joining amino acids between highly structures domains. Purified 44AHJD ORF 25 or DnaN proteins can be subjected to partial proteolysis. The proteolysis can be performed with low concentrations of proteases (trypsin, chymotrypsin, endoproteinase Glu-C, and Asp-N) with 44AHJD ORF 25 or DnaN in solution, resulting in the generation of defined proteolytic products as observed by SDS-PAGE. An acceptable concentration and reaction time is defined by the near complete conversion of the full-length protein to stable proteolytic products. The proteolytic products are then subjected to affinity chromatography containing the appropriated partner of interaction (44AHJD ORF 25 or DnaN purified proteins) to determine a protein sub-region able to interact. Interacting domains are identified by mass spectrometry to determine both the intact fragment mass and the completely digested with trypsin (by in-gel digestion) to better determine the amino acid residues contained within the partial proteolytic fragment. Using both sets of data, the amino acid sequence of the partial proteolytic fragment can be precisely determined.

Another approach is based on peptide screening using different portions of 44AHJD ORF 25 and DnaN to identify minimal peptides from each polypeptide that are able to disrupt the interaction between the two proteins. It is assumed that fragments able to prevent interaction between 44AHJD ORF 25 and DnaN correspond to domains of interaction located on either of the two proteins. The different peptide fragments can be screened as competitors of interaction in protein: protein binding assays such as the ones described above. Fine mapping of interaction site(s) within a protein can be performed by an extensive screen of small overlapping fragments or peptides spanning the entire amino acid sequence of the protein.

Fragments of 44AHJD ORF 25 or of DnaN can be produced by proteolytic digestion of the full-length proteins as described above. Alternatively, suitable dnaN or 44AHJD ORF 25-derived amino acid fragments representative of the complete sequence of both proteins can be chemical synthesis. For instance, in the multipin approach, peptides are simultaneously synthesis by the assembly of small quantities of peptides (ca. 50 mmol) on plastic pins derivatized with an ester linker based on glycolate and 4-(hydroxymethyl) benzoate (Maeji 1991 Pept Res, 4:142-6).

Functional Assays for Bacterial Growth: OD and CFU Measurement Over Time

Compounds selected for their ability to inhibit the 44AHJD ORF 25-DnaN interaction can be further tested in functional assays on bacterial growth. Cells are grown in the presence of varying concentrations of a candidate compound added directly to the medium. The cultures are then incubated for an additional 4 hrs at 37° C. During that period of time, the effect of inhibitors on bacterial cell growth may be monitored, at 40 min intervals, by measuring the OD565 and the number of colony forming units (CFU) in the cultures. The number of CFU is evaluated as follows: cultures are serially diluted and aliquots from the different cultures are plated out on agar plates. Following incubation overnight at 37° C., the number of colonies are counted. Non-treated cultures of S. aureus are included as control.

Surface Plasmon Resonance

Another powerful assay to screen for inhibitors of a protein: protein interaction is surface plasmon resonance. Surface plasmon resonance is a quantitative method that measures binding between two (or more) molecules by the change in mass near the sensor surface caused by the binding of one protein or other biomolecule from the aqueous phase to a second protein or biomolecule immobilized on the sensor. This change in mass is measured as resonance units versus time after injection or removal of the second protein or biomolecule and is measured using a Biacore Biosensor (Biacore AB). dnaN could be immobilized on a sensor chip (for example, research grade CM5 chip; Biacore AB) using a covalent linkage method (e.g. amine coupling in 10 mM sodium acetate [pH 4.5]). A blank surface is prepared by activating and inactivating a sensor chip without protein immobilization. The binding of 44AHJD ORF 25 to dnaN, or a fragment of dnaN, is measured by injecting purified 44AHJD ORF 25 over the chip surface. Measurements are performed at room temperature. Conditions used for the assay (i.e., those permitting binding) are as follows: 25 mM HEPES-KOH (pH 7.6), 150 mM sodium chloride, 15% glycerol, 1 mM dithiothreitol, and 0.001% Tween 20 with a flow rate of 10 ul/min. Preincubation of the sensor chip with candidate inhibitors will predictably decrease the interaction between 44AHJD ORF 25 and dnaN. A decrease in 44AHJD ORF 25 binding is indicative of competitive binding by the candidate compound.

Fluorescence Resonance Energy Transfer (FRET)

Another method of measuring inhibition of binding of two proteins uses fluorescence resonance energy transfer (FRET; de Angelis, 1999, Physiological Genomics). FRET is a quantum mechanical phenomenon that occurs between a fluorescence donor (D) and a fluorescence acceptor (A) in close proximity (usually <100 A of separation.) if the emission spectrum of D overlaps with the excitation spectrum of A. Variants of the green fluorescent protein (GFP) from the jellyfish Aequorea victoria are fused to a polypeptide or protein and serve as D-A pairs in a FRET scheme to measure protein-protein interaction. Cyan (CFP: D) and yellow (YFP: A) fluorescence proteins are linked with dnaN polypeptide, or a fragment of dnaN and 44AHJD ORF 25 protein respectively. Under optimal proximity, interaction between dnaN, or a fragment of dnaN, and 44AHJD ORF 25 causes a decrease in intensity of CFP concomitant with an increase in YFP fluorescence.

The addition of a candidate modulator to the mixture of appropriately labeled dnaN and 44AHJD ORE 25 protein, will result in an inhibition of energy transfer evidenced by, for example, a decease in YFP fluorescence at a given concentration of 44AHJD ORF 25 relative to a sample without the candidate inhibitor.

Fluorescence Polarization

In addition to the surface plasmon resonance and FRET methods, fluorescence polarization measurement is useful to quantitate protein-protein binding. The fluorescence polarization value for a fluorescently-tagged molecule depends on the rotational correlation time or tumbling rate. Protein complexes, such as those formed by S. aureus dnaN polypeptide, or a fragment of dnaN associating with a fluorescently labeled polypeptide (e.g., 44AHJD ORF 25 or a binding fragment thereof), have higher polarization values than a fluorescently labeled monomeric protein. Inclusion of a candidate inhibitor of the dnaN interaction results in a decrease in fluorescence polarization relative to a mixture without the candidate inhibitor if the candidate inhibitor disrupts or inhibits the interaction of dnaN with its polypeptide binding partner. It is preferred that this method be used to characterize small molecules that disrupt the formation of polypeptide or protein complexes.

Bio Sensor Assay

ICS biosensors have been described by AMBRI (Australian Membrane Biotechnology Research Institute; http//www.ambri.com.au/). In this technology, the self-association of macromolecules such as dnaN, or a fragment of dnaN, and bacteriophage 44AHJD ORF 25, is coupled to the closing of gramacidin-facilitated ion channels in suspended membrane bilayers and hence to a measurable change in the admittance (similar to impedence) of the biosensor. This approach is linear over six order of magnitude of admittance change and is ideally suited for large scale, high through-put screening of small molecule combinatorial libraries.

### EXAMPLE VIII

Identification of Bacterial Target

To identify the S. aureus protein(s) that interact with inhibitory ORF 25 of S. aureus bacteriophage 44AHDK, a GST-fusion of 44AHJD ORF 25 was generated. The recombinant protein was purified and utilized to make a GST/44AHJD ORF 25 affinity column. Cellular extracts prepared from S. aureus cells were incubated with the affinity matrix and the matrix was washed with buffers containing increasing concentrations of salt and different detergents. The protein elution profile was assessed by SDS-polyacrylamide gel electrophoresis (SDS-PAGE). A protein of molecular mass—48 kDa, identified as PT48, was spicifically eluted from the affinity matrix and was not detected in eluates from GST negative control column. Eluted proteins were further characterized to determine the identity of the interacting protein.

Generation of GST/ORF 25 Recombinant Protein Bacteriophage 44AHJD ORF 25 was sub-cloned into pGEX 4T-1 (Pharmacia), an expression vector containing the GST moiety. ORF 25 was obtained by digestion of pTHA/44AHJD ORF 25 (FIG. 2A) with BamHI and SalI. The DNA fragment containing ORF 25 was gel purified by QiaQuick spin columns (Qiagen) and ligated into pGEX 4T-1 (which had been previously digested with Bam HI and Sal I) to generate pGEX 4T/ORF 25. Recombinant expression vectors were identified by restriction enzyme analysis of plasmid minipreps. Large-scale DNA preparations were performed and the resulting insert was sequenced. Test expressions in E. coli BL21 (DE3) Gold cells containing the expression plasmids were performed to identify optimal protein expression conditions. E. coli cells containing the expression constructs were grown in Luria-Bertani Broth at 25° C. to an OD₆₀₀ of 0.4 to 0.6 and induced with 1 mM IPTG for the optimal times and at the optimal temperatures (typically a 2 liter culture of BL21 (DE3) Gold (pGEX 4T/ORF25) grown at 25° C. for 3hrs).

Fusion Protein Purification.

Cells containing GST/ORF 25 fusion protein were suspended in 15 ml lysis buffer/liter of cell culture with GST lysis buffer (20 mM Hepes pH 7.2, 500 mM NaCl, 10% A45 glycerol, 1 mM DTT, 1 mM EDTA, 1 mM benzamidine, and 1 PMSF) and lysed using a French pressure cell followed by three bursts of twenty seconds with an ultra-sonicator at 4° C. Triton X-100 was added to the lysate to a final concentration of 0.1% and mixed for 30 minutes at 4° C. The lysate was centrifuged at 4° C. for 30 minutes at 10,000 rpm in a Sorval SS34 rotor. The supernatant was applied to a 4ml glutathione sepharose column pre-equilibrated with lysis buffer and allowed to flow by gravity. The column was washed with 10 column volumes of lysis buffer and eluted in 1.5 ml fractions with GST elution buffer (20 mM Hepes pH 8.0, 500 mM NaCl, 10% glycerol, 1 mM DTT, 0. 1 mM EDTA, and 25 mM reduced glutathione). The fractions were analyzed by SDS-12.5% PAGE (Laemmli) and proteins were visualized by staining with Coomassie Brilliant Blue R250 stain to assess the amount of eluted GST/ORF 25 protein.

Affinity Column Preparation.

GST and GST/ORF25 were dialyzed overnight against affinity chromatography buffer (ACB; 20 mM Hepes pH 7.5, 10% glycerol, 1 mM DTT, and 1 mM EDTA) containing 1 M NaCl. Protein concentrations were determined by Bio-Rad Protein Assay and crosslinked to Affigel 10 resin (Bio-Rad) at protein/resin concentrations of 0, 0.1, 0.5, 1.0, and 2.0 mg/ml. The crosslinked resin was sequentially incubated in the presence of ethanolamine, and bovine serum albumin (BSA) prior to column packing and equilibration with ACB containing 100 mM NaCl.

S. aureus Extract Preparation.

Two extracts were prepared from S. aureus cell pellets. One lysate was prepared by French pressure cell lysis followed by sonication, and the other by lysostaphin-mediated digestion followed by sonication. The French pressure cell lysate was prepared by suspending 3 g of frozen S. aureus cells in ABC containing 500 mM NaCl, 1 mM PMSF, and 1 mM benzamidine. The suspended cells were subjected to three passes through the French pressure cell followed by 3 sonication bursts of 20 seconds each, made up to 0.1% Triton X-100, stirred for 30 minutes, and centrifuged at 50,000 rpm for 3 hrs in a Ti70 fixed angle Beckman rotor. The efficiency of cell lysis was low and the resulting lysate (7 ml) contained 2.4 mg/ml protein. The pellet after French pressure cell lysis was subjected to cryogenic grinding in liquid nitrogen in the same buffer with a mortar and pestle. The lysate was made up to 0.1% Triton X-100, stirred for 30 minutes, and centrifuged at 50,000 rpm for 3 hrs in a Ti70 fixed angle Beckman rotor yielding a lysate (10 ml) containing 2.0 mg/ml protein. The cell lysates were pooled, concentrated to 8 ml, and dialyzed overnight in a 3000 Mr cut-off dialysis membrane against ACB containing 1 mM PMSF, 1 mM benzamidine, and 75 mM NaCl. The dialyzed protein extract was removed from the dialysis tubing, centrifuged at 10 000 rpm in a Sorval SS34 rotor for 1 hr, and assayed for protein content (Bio-Rad Protein Assay) and salt concentration (conductivity meter).

Affinity Chromatography.

The S. aureus extract was centrifuged at 4° C. in a micro-centrifuge for 15 minutes and 200μl was applied to 200μl columns containing 0, 0.1, 0.5, 1.0, and 2.0 mg/ml ligand. ACB containing 100 mM NaCl (200 μl) was applied to a control column containing 2.0 mg/ml ligand. The columns were washed with 10 column volumes ACB containing 100 mM NaCl and sequentially eluted with ACB containing 1% Triton X-100 and 100 mM NaCl (800 μl), ACB containing 1 M NaCl (800 μl), and 1% SDS (160 μl). 40 μl of each eluate was resolved by SDS-12.5% PAGE (Laemmli) and the protein was visualized by silver stain.

Identification of S. aureus DnaN as an 44AHJD ORF 25 Interacting Protein

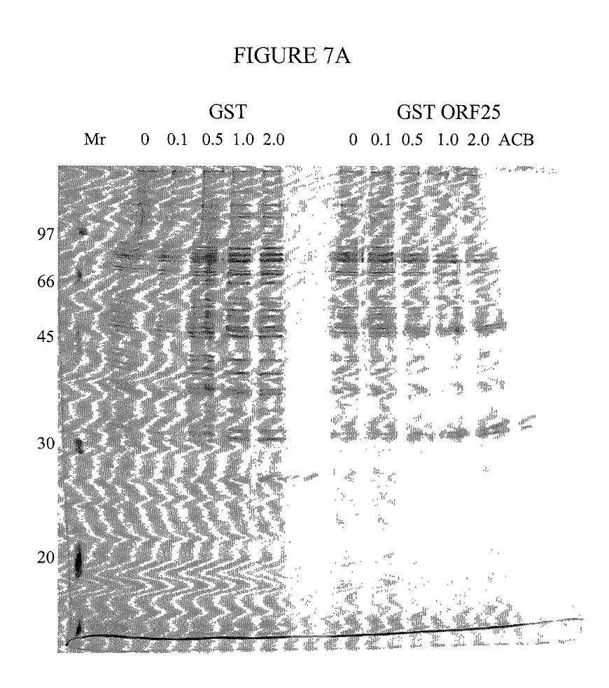

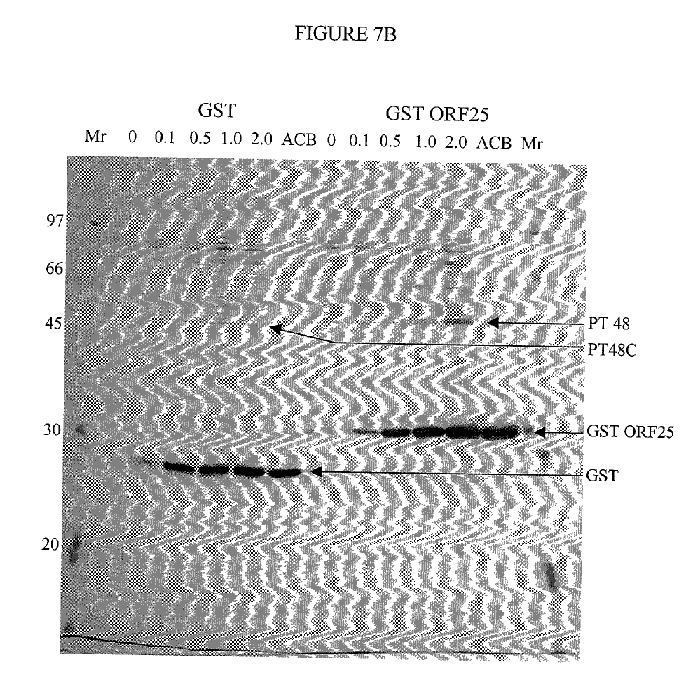

Affinity chromatography was performed using GST and GST ORF25 as ligands coupled to Affigel 10 at protein/resin concentrations of 0, 0.1, 0.5, 1.0, and 2.0 mg/ml. Two S. aureus extracts were used for affinity chromatography with each of the ligands. Two extracts used for affinity chromatography, prepared separately, contained 4.0 and 9.0 mg/ml protein. One candidate interacting protein of 48 kDa (PT48) was observed in the 1% SDS eluates in the initial chromatography experiment (FIG. 7B).

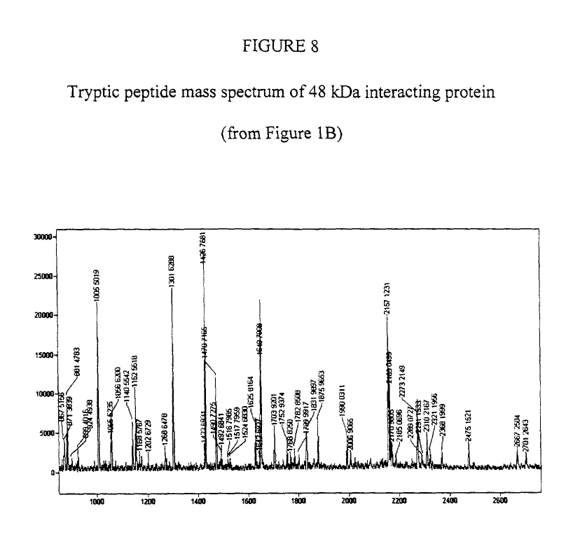

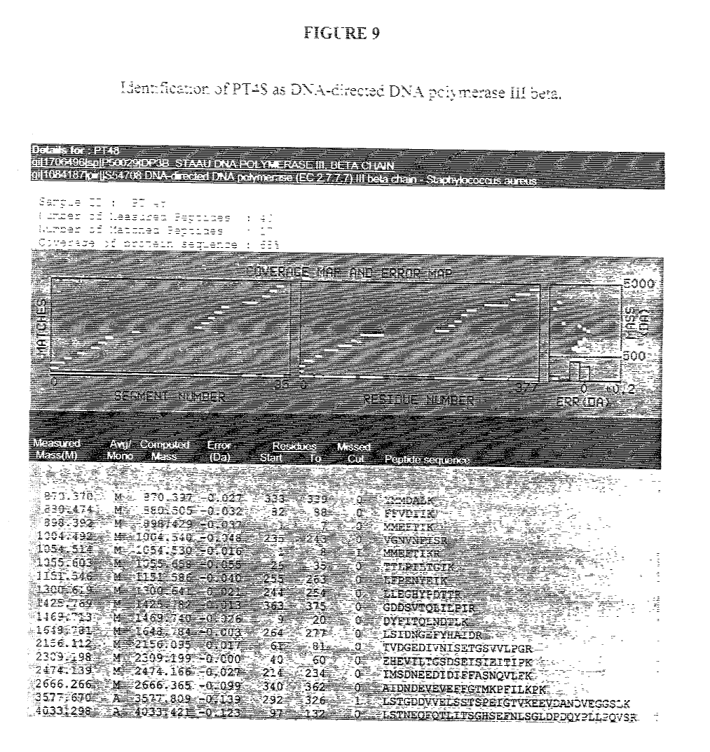

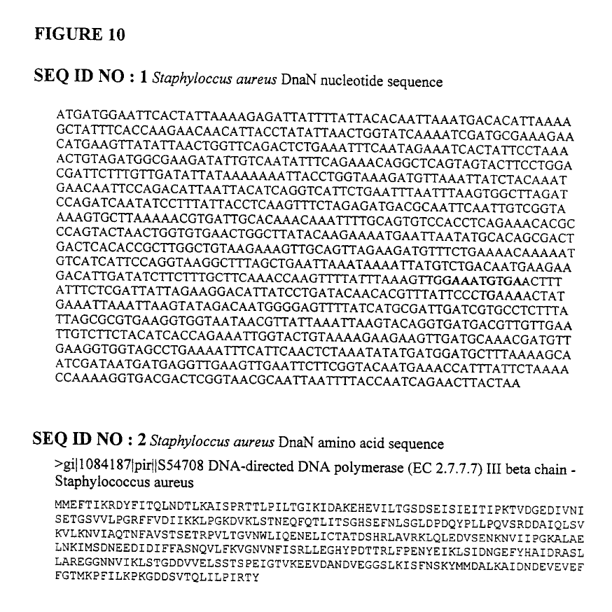

The candidate protein, PT48 was excised from the SDS-PAGE gels and prepared for tryptic peptide mass determination by MALDI-ToF mass spectrometry (Qin, J., Fenyo, D., Zhao, Y., Hall, W. W., Chao, D. M., Wilson, C. J., Young, R. A. and Chait, B. T. (1997) Anal. Chem. 69, 3995-4001). High quality mass spectra were obtained (FIG. 8). The PT48 proteins observed in two affinity chromatography experiments were identical as determined by the masses of the tryptic peptides. Computational analysis (http://prowl.rockfeller.edu/cgi-bin/ProFound) of the mass spectrum obtained identifies the corresponding ORF in the S. aureus nucleotide sequence in the University of Oklahoma S. aureus genomic database (http://www.genome.ou.edu/staph.html). The identity of that protein which binds specifically to GST 0RF25 is the DNA-directed DNA polymerase III beta subunit (Genbank accession #1084187) (FIG. 9 and FIG. 10).

It is important to note that in assays of protein-protein interaction, it is possible that a modulator of the interaction need not necessarily interact directly with the domain(s) of the proteins that physically interact. It is also possible that a modulator will interact at a location removed from the site of protein-protein interaction and cause, for example, a conformational change in the dnaN polypeptide. Modulators (inhibitors or agonists) that act in this manner are of interest since the change they induce may modify the activity of the dnaN polypeptide.

Compounds selected for their ability to bind to dnaN or to inhibit the 44AHJD ORF 25-dnaN interaction can be further tested in functional assays of bacterial growth. Cultures of S. aureus are grown in the presence of varying concentrations of a candidate compound added directly to the medium. The cultures are then incubated for an additional 4 hrs at 37° C. During that period of time, the effect of inhibitors on bacterial cell growth may be monitored at 40 min intervals, by measuring the OD565 and the number of colony forming units (CFU) in the cultures. The number of CFU is evaluated as follows: cultures are serially diluted and aliquots from the different cultures are plated out on agar plates. Following incubation overnight at 37° C., the number of colonies are counted. Non-treated cultures of S. aureus are included as negative control.

REFERENCES

Cohen, M. L. (1992). Science 257:1050-1055.

Rusterholtz, K., and Pohlsclroder, M. (1999). Cell 96, 469-470.

Ackermann, H.-W. and DuBow, M. S. (1987). Viruses of Prokaryotes. CRC Press.

Volumes 1 and 2.

Durfee, T., Becherer, K., Chen, P.-L., Yeh, S.-H., Yang, Y., Kilburn, A. E., Lee, W.-H., and Elledge, S. J. (1993). Genes & Dev. 7: 555-569.

Sopta, M., Carthew, R. W., and Greenblatt, J. (1995) J. Biol. Chem. 260: 10353-10369.

Qin, J., Fenyo, D., Zhao, Y., Hall, W. W., Chao, D. M., Wilson, C. J., Young, R. A. and Chait, B. T. (1997). Anal. Chem. 69: 3995-4001.

Sambrook, J., Fritsch, E. F. and Maniatis, T (1989). Molecular cloning: A laboratory Manual. Cold Spring Harbor Laboratory, New York. Cold Spring Harbor Laboratory Press.

Swanstrom, M. and Adams, M. H. (1951). Agar layer method for production of high titer phage stocks. Proc. Soc. Exptl. Biol. & Med. 78: 372-375.

Tauriainen, S., Karp, M., Chang, W and Virta, M. (1997). Recombinant luminescent bacteria for measuring bioavailable arsenite and antimonite. Appl. Environ. Microbiol. 63:4456-4461.

Field, J., Nikawa, J.-I., Broek, D., MacDonald, B., Rodgers, L., Wilson, I. A., Lemer, R. A., and Wigler, M. (1988). Purification of a RAS-responsive adenylyl cyclase complex from Saccharomyces cerevisiae by use of an epitope addition method. Mol. Cell. Biol. 8: 2159-2165.

Kreiswirth, B N., Lofdahl, S., Belley, M J., O'Reilly, M., Shlievert, P M., Bergdoll, M S. and Novicks, R P. 1983. Nature #305: 709-712.

Schenk, S. and Laddaga, R A. 1992. FEMS Microbiology Letters #94: 133-138.

Oskouian, B. and Stewart, G S. 1990. J. Bacteriol. #172: 3804-3812.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The specific methods and compositions described herein as presently representative of preferred embodiments are exemplary and are not intended as limitations on the scope of the invention. One of ordinary skill in the art would recognize that Bacteriophage 44AHJD ORFs described herein are provided and discussed by way of example, and other the ORFs of Bacteriophage 44AHJD, including amino acid sequences and nucleic acid sequences which encode products, are within the scope of the present invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention. For example, those skilled in the art will recognize that the invention may suitably be practiced using a variety of different expression vectors and sequencing methods within the general descriptions provided.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is not intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group. For example, if there are alternatives A, B, and C, all of the following possibilities are included: A separately, B separately, C separately, A and B, A and C, B and C, and A and B and C.

Thus, additional embodiments are within the scope of the invention and within the following claims.

TABLE 1
Bacteriophage 44AHJD, complete genome sequence.

| | |
|---|---|
| 1 | tccatttctt tactaaactt aaaaatgctg tgcaacaact taaccaactt atctaaccta |
| 61 | ttacatattc atcaaataca aaatttatgt atctattgac ttttattcaa aattatgatt |
| 121 | tcaacatata ataaaattaa tttacttatt taaatattct atgatataat tagttataaa |
| 181 | atatttggag gtgtataaat gacagaattt gatgaaatcg taaaaccaga cgacaaagaa |
| 241 | gaaacttcag aatcaactga agaaaattta gaatcaactg aagaaacttc agaatcaact |
| 301 | gaagaatcaa ctgaagaatc aactgaagaa tcaactgaag ataaaacagt agaaacaatc |
| 361 | gaagaagaaa atgaaaacaa attagaacct actacaacag atgaagatag ttcgaaattt |
| 421 | gaccctgttg tattagaaca acgtattgct tcattagaac aacaagtgac tactttttta |
| 481 | tcttcacaaa tgcaacaacc acaacaagta caacaaacac aatcagatgt aacagaatca |
| 541 | aacaaagaag ataacgacta ttcagatgaa gaactagttg ataagttaga tttagattag |
| 601 | gaggaattta aacatgtatg agggaaacaa catgcgttct atgatgggta catcatatga |
| 661 | agattcaaga ttaaataaac gaacagaatt aaatgaaaac atgtcaattg atacaaataa |
| 721 | aagtgaagat agttatggtg tacaaattca ttcactttca aaacaatcat ttacaggtga |
| 781 | cgttgaggag gaataataaa ttatggcaca acaatctaca aaaaatgaaa ctgcactttt |
| 841 | agtagcaaag tcagctaaat cagcgttaca agattttaat catgattatt caaaatcttg |
| 901 | gacatttggc gacaaatggg ataattcaaa tacaatgttc gaaacatttg taaataaata |
| 961 | tttattccct aagattaatg agactttatt aatcgatatt gcattaggta atcgttttaa |
| 1021 | ttggttagct aaagagcaag attttattgg acaatatagt gaagaatacg tgattatgga |
| 1081 | cacagtacca attaacatgg acttatctaa aaatgaggaa ttaatgttga aacgtaatta |
| 1141 | tccacgtatg gcaactaagt tatatggtaa cggaattgtg aagaaacaaa aattcacatt |
| 1201 | aaacaacaat gatacacgtt tcaatttcca aacattagca gacgcaacta attacgcttt |
| 1261 | aggtgtatac aaaaagaaaa tttctgatat taatgtatta gaagaaaaag aaatgcgtgc |
| 1321 | aatgttagtt gattactoat tgaatcaatt atccgaaaca aatgtacgta aagcaacatc |
| 1381 | aaaagaagat ttagcaagca aagtttttga agcaatccta aacttacaaa acaacagtgc |
| 1441 | taaatataat gaagtacatc gtgcatcagg tggtgcaatt ggacaatata caactgtatc |
| 1501 | aaaattaaaa gatattgtga ttttaacaac agattcatta aaatcttatc ttttagatac |
| 1561 | taagattgca aacacattcc agattgcagg cattgatttc acagatcacg ttattagttt |
| 1621 | tgacgactta ggtggcgtgt ttaaagtaac aaaagaattt aagttacaaa accaagattc |
| 1681 | aattgacttt ttacgtgcgt atggagatta tcaatcacaa ttaggagata caattccagt |
| 1741 | tggtgctgta tttacttatg atgtatctaa acttaaagag tttactggca acgttgaaga |
| 1801 | aattaaacca aaatcagatt tatatgcgtt tattttggat attaattcaa ttaaatataa |
| 1861 | acgttacaca aaaggtatgt taaaaccacc attccataac cctgaatttg atgaagttac |
| 1921 | acactggatt cattactatt catttaaagc cattagtcca ttctttaata aaattttaat |
| 1981 | tactgaccaa gatgtaaatc caaaaccaga ggaagaatta caagaataaa aggagcgtaa |
| 2041 | aatatgaaca acgataaaag aggtttaaac gttgagttat caaaggaaat cagcaaaaga |
| 2101 | gttgttgaac atcgcaacag atttaaacgt cttatgttta atcgttattt ggaattttta |
| 2161 | ccgctactaa tcaactatac caatcgtgat acggttggta tagattttat tcagttagaa |
| 2221 | tcagctttaa gacaaaacat taatgtagtt gttggtgaag ctagaaataa gcaaattatg |
| 2281 | attcttggtt atgtaaataa cacttacttt aatcaagcac caaatttttc atcaaacttt |
| 2341 | aatttccaat ttcaaaaacg attaactaaa gaagatatat attttattgt acctgactat |
| 2401 | ttaatacctg atgattgtct acaaattcat aagctatatg ataactgtat gagtggtaac |
| 2461 | tttgttgtca tgcaaaataa accaattcaa tataatagtg atatagaaat tatagaacat |
| 2521 | tatactgatg aattagcaga agttgcttta tctcgctttt ctttaatcat gcaagcaaaa |
| 2581 | tttagcaaga tatttaaatc agaaattaat gacgagtcaa tcaatcaact tgtgtccgaa |
| 2641 | atatataacg gtgcaccatt tgttaaaatg tcacctatgt ttaatgcaga tgacgatatc |
| 2701 | attgatttaa caagtaatag cgtaatccca gcattaactg aaatgaaacg ggaatatcaa |
| 2761 | aacaaaatta gtgaattaag taactattta ggcattaatt cattagccgt tgataaagaa |
| 2821 | agcggtgttt cagacgaaga ggcaaaaagt aatcgtggat ttaccacatc aaacagtaat |
| 2881 | atctatttaa aaggtcgtga accaattacg tttttatcaa agcgttatgg tttagatatt |
| 2941 | aaaccgtatt acgatgatga aacaacgtct aaaatatcaa tggtagacac actttttaaa |
| 3001 | gatgaaagca gtgatataaa tggctagata cacaatgact ttatacgatt tcattaaatc |
| 3061 | agaattgatt aaaaaaggtt tcaatgaatt tgtaaatgat aataaattaa cgttttatga |
| 3121 | tgatgaattt caattcatgc aaaaaatgct gaagttcgac aaagacgttt tagctatcgt |
| 3181 | taatgaaaaa gtatttaaag gtttttcatt gaaagatgaa ttatcagatt tactttttaa |
| 3241 | aaaatcattt acgattcatt ttttagatag agaaatcaac agacaaacag ttgaagcatt |
| 3301 | tggcatgcaa gtgattactg tatgtattac acatgaggat tatttaaatg tggtttattc |
| 3361 | atcaagtgaa gttgaaaaat acttacaatc acaaggcttc acagaacaca atgaagatac |
| 3421 | aacaagtaac actgatgaaa catcgaatca aaatgctaca tctttagaca attcaactgg |
| 3481 | catgactgca aacagaaacg cttatgtgtc attaccacaa agtgaggtta acattgatgt |
| 3541 | tgataataca acgttacgat tcgctgataa taatacgatt gataacggta aaactgtgaa |
| 3601 | taaatcgagt aacgaaagta atcaaaacgc aaaacgtaat caaaatcaaa aaggtaatgc |
| 3661 | aaaaggtaca caattcacta agcagtattt aattgataat attgataaag cgtacgattt |
| 3721 | aagaaagaaa attttaaatg aatttgataa aaaatgtttt ttacaaattt ggtagaggtg |
| 3781 | gttaaataat ggcatataat gaaaacgatt ttaaatattt tgatgacatt cgtccatttt |
| 3841 | tagacgaaat ttataaaacg agagaaogtt atacaccgtt ttacgatgat agagcagatt |
| 3901 | ataatactaa ttcaaaatca tattatgatt atatttcaag attatcaaaa ctaattgaag |
| 3961 | tattagcacg tcgtatttgg gactatgaca atgaattaaa aaaacgtttc aaaaattggg |
| 4021 | acgacttaat gaaagcattt ccagagcaag cgaaagactt atttagaggt tggttaaacg |
| 4081 | acggtacgat tgacagtatt attcatgacg agtttaaaaa atatagcgca ggattaacat |
| 4141 | cggcatttgc tttatttaaa gttactgaaa tgaaacaaat gaatgacttt aaatcagaag |
| 4201 | ttaaagactt aattaaagat attgaccgtt tcgttaatgg gtttgaatta aatgagcttg |
| 4261 | aaccaaagtt tgtgatgggc tttggtggta ttcgcaacgc agttaaccaa tctattaata |
| 4321 | ttgataaaga aacaaatcac atgtactcta cacaatccga ttctcaaaaa cctgaaggtt |
| 4381 | tttggataaa taaattaaca cctagtggtg acttaatttc aagcatgcgt attgtacagg |
| 4441 | gtggtcatgg tacaacaatc ggattagaac gtcaatccaa tggtgaaatg aaaatctggt |
| 4501 | tacatcacga tggtgttgca aaactgttac aagtcgcata taaagataat tatgtattag |
| 4561 | atttagaaga ggctaaaggt ttaacagatt atacaccaca gtcactttta aacaaacaca |
| 4621 | catttacacc gttaattgat gaagcaaatg acaaactcat tttaagattc ggtgacggaa |
| 4681 | caatacaggt tcgttcaaga gcagacgtaa aaaatcacat tgataatgta gaaaaagaaa |
| 4741 | tgacaattga taattcagaa aacaatgata atcgttggat gcaaggcatt gctgttgatg |
| 4801 | gtgatgattt atactggtta agtggtaaca gttcagttaa ttcacatgtt caaatcggta |
| 4861 | aatattcatt aacaacaggt caaaagattt atgattatcc atttaagtta tcatatcaag |
| 4921 | acggtattaa tttcccacgt gataacttta aagagcctga gggtatttgc atttatacaa |
| 4981 | atccaaaaac aaaacgtaaa tcgttattac ttgctatgac aaacggcggt ggtggaaaac |
| 5041 | gtttccataa tttatatggt ttcttccaac ttggtgagta tgaacacttt gaagcattac |
| 5101 | gcgcaagagg ttcacaaaac tataaattaa caaaagacga cggtcgtgca ttatctattc |
| 5161 | cagaccatat cgacgattta aatgacttaa cgcaagctgg tttttattat attgacgggg |
| 5221 | gtactgcaga aaaacttaag aatatgccaa tgaatggtag caagcgtata attgacgctg |
| 5281 | gttgtttcat taatgtatac cctacaacac aaacattagg tacggttcaa gaattaacac |
| 5341 | gtttctcaac aggtcgtaaa atggttaaaa tggtgcgtgg tatgacttta gacgtattta |
| 5401 | cgttaaaatg ggattatgga ttatggacaa caatcaaaac tgacgcacca tatcaagaat |
| 5461 | atttggaagc aagtcaatac aataactgga ttgcttatgt aacaacagct ggtgagtatt |
| 5521 | acattacagg taaccaaatg gaattattta gagacgcgcc agaagaaatt aaaaaagtgg |
| 5581 | gtgcatggtt acgtgtgtca agtggtaacg cagtcggtga agtaagacaa acattagagg |
| 5641 | ctaatatatc ggaatataaa gaattcttca gtaatgttaa tgcggaaaca aaacatcgtg |
| 5701 | aatatggttg ggtagcaaaa catcaaaaat aggagtgata taaatgaaat cacaacaaca |
| 5761 | agcaaaagaa tggatatata agcatgaggg ggcaggtgtt gactttgatg gtgcatatgg |
| 5821 | atttcaatgt atggacttat cagttgctta tgtgtattac attactgacg gtaaagttcg |
| 5881 | catgtggggt aatgctaaag acgcgataaa taatgacttt aaaggtttag cgacggtgta |
| 5941 | taaaaataca ccgagcttta aacctcaatt aggggacgtt gctgtatata caaatggaca |
| 6001 | atatggacat attcaatgtg tgttaagtgg aaatcttgat tattatacat gcttagaaca |
| 6061 | aaactggtta ggcggcggtt ttgacggttg ggaaaaagca accattagaa cacattatta |
| 6121 | tgacggtgta actcacttta ttagacctaa attttcaggt agtaatagca aagcattaga |
| 6181 | aacatcaaaa gtaaatacat ttggaaaatg gaaacgaaac caatacggca catattatag |
| 6241 | aaatgaaaat ggtacattta catgtggttt tttaccaata tttgcacgtg tcggtagtcc |
| 6301 | aaaattatca gaacctaatg gctattggtt ccaaccaaac ggttatacac catataacga |
| 6361 | agtttgttta tcagatggtt acgtatggat tggttataac tggcaaggca cacgttatta |
| 6421 | tttaccagtg cgccaatgga atggaaaaac aggtaatagt tacagtgttg gtattccttg |
| 6481 | gggggtgttc tcataatggg tattttagcc tttttctttg aatttagttg gaaaagatac |
| 6541 | aaataagagg tgtaaacaat ggctgataga atcgtaagaa gtttaagaca agttgaaaca |
| 6601 | attgaacgtt tattggagga aaaaaatgag aaagttaacg aattttaagt ttttctataa |
| 6661 | cacaccgttt acagactatc aaaacacgat tcattttaat agtaataaag aacgtgatga |
| 6721 | ttatttttta aatggtcgtc attttaaatc gttagactat tcaaaacaac cgtataattt |
| 6781 | tatacgtgat agaatggaaa tcaatgttga tatgcagtgg catgacgcac aaggtattaa |
| 6841 | ctacatgacg tttttatcag attttgagga tagaagatat tacgcttttg taaaccaaat |
| 6901 | cgaatacgtg aatgacgttg tggttaaaat atattttgtc attgatacca ttatgacgta |
| 6961 | tacacaaggg aatgtattag agcaactctc aaacgtcaat attgaacgto aacatttatc |
| 7021 | aaaacgcacg tataactata tgttaccaat gttacgtaat aatgatgatg tgttaaaagt |
| 7081 | atcaaataaa aactatgttt ataaccaaat gcaacaatat ttggaaaatt tagtattatt |
| 7141 | ccagtcaagc gctgatttat caaagaaatt tggtactaaa aaagagccaa acttagatac |
| 7201 | gtcaaaaggt acgatttatg acaatatcac atcaccagtc aacttatacg ttatggaata |
| 7261 | tggtgacttt attaacttta tggataaaat gagtgcctat ccatggatta cgcaaaactt |
| 7321 | tcaaaaggtt caaatgttac ctaaagactt tattaataca aaagacttag aggacgttaa |
| 7381 | aaccagtgaa aaaattacag gattaaaaac attaaaacag ggtggtaaat caaaagaatg |
| 7441 | gagtctaaaa gatttatcat taagtttctc aaatcttcaa gagatgatgt tatctaaaaa |
| 7501 | agatgaattt aaacatatga tacgtaatga gtatatgaca attgaatttt atgactggaa |
| 7561 | tggaaatacg atgttactcg acgctggtaa gatttcacaa aaaactggtg ttaagttacg |
| 7621 | tacaaaatca attattggtt atcataatga agttcgagta tatccagtag attataacag |
| 7681 | tgctgaaaac gacagaccaa tactcgctaa aaataaagaa atattgattg atacgggttc |
| 7741 | attcttaaat acaaatataa catttaatag ttttgcacaa gtaccaatat taatcaataa |
| 7801 | tggtatctta ggacaatcac aacaagccaa ccgacaaaaa aatgcagaaa gtcaattaat |
| 7861 | tacaaatcgt attgataatg tattaaatgg tagcgacccg aaatcacgct tttatgacgc |
| 7921 | tgtgagtgta gcaagtaatt taagtccaac tgctttattt ggtaagttta atgaagaata |
| 7981 | taatttctac aaacaacaac aagctgaata taaagattta gccttacaac caccttctgt |
| 8041 | aactgaatca gaaatgggca acgcattcca aattgcgaat agcattaacg gtttaacgat |
| 8101 | gaaaattagt gtaccgtcac ctaaagaaat tacattttta caaaaatatt atatgttgtt |
| 8161 | tggttttgaa gtgaatgact ataattcatt tattgaacca attaacagta tgactgtttg |
| 8221 | caattattta aaatgtacag gtacgtatac tatacgtgac atcgacccca tgttaatgga |
| 8281 | acaattaaaa gcaattttag aatctggtgt aagattttgg cataatgacg gttcaggtaa |
| 8341 | tccaatgtta caaaatccat taaataacaa atttagagag ggggtataat atgaacgaag |
| 8401 | taaaattcag atttacagac tcagaagcgt ttcacatgtt tatatacgct ggggatttaa |
| 8461 | aattactcta ctttttattt gtattaatgt tcgttgatat tattacaggt atttcaaaag |
| 8521 | caattaaaaa taataactta tggtcaaaaa aatcaatgag aggattttct aaaaaattat |
| 8581 | tgatattctg tattatcatt ttagcaaaca tcattgacca gattttacaa ttaaaaggtg |
| 8641 | gtctactcat gattacaata ttttattata ttgcaaatga gggactttct attgtagaaa |
| 8701 | attgtgcaga aatggacgta ttagtaccag aacaaattaa agataaatta agagtcatta |
| 8761 | aaaatgatac tgaaaagagt gataacaatg aacgatcaag agaagataga taaatttacg |
| 8821 | cattcctata ttaatgatga ttttggttta acgatagacc agttagtccc taaagtaaaa |
| 8881 | ggatatgggc gctttaatgt atggcttggt ggtaatgaaa gtaaaatcag acaagtatta |
| 8941 | aaagcagtaa aagagatagg tgtttcacct actctttttg ccgtatatga aaaaaatgag |
| 9001 | ggttttagtt ctggacttgg ttggttaaac catacgtctg cacgtggtga ttatttaaca |
| 9061 | gatgctaaat tcatagcaag aaagttagta tcacaatcaa aacaagctgg acaaccgtct |
| 9121 | tggtatgacg caggtaacat cgtccacttt gtaccacaag acgtacaaag aaaaggtaat |
| 9181 | gcagattttg caaaaaatat gaaagcaggt acaattggac gtgcatatat tccattaaca |
| 9241 | gcagctgcta cttgggcggc atattatoct ttaggtttga aagcatcata taacaaagta |
| 9301 | caaaactatg gtaatccatt ttta~acggt gcgaatacta ttctagcttg gggtggtaaa |
| 9361 | ttagacggta aaggtggatc acctagtgat tcgtctgaca gtggtagtag tggtgacagt |
| 9421 | ggtagttcac tactcgcttt agcaaaacaa gccatgcaag aattattaaa aaaaatacaa |
| 9481 | gacgcattac aatgggacgt tcatagtatt ggtagtgata aattttttag taatgattat |
| 9541 | tttacattag aaaaaacatt taacaacaca tatcatatta aaatgacgat tggtttactt |
| 9601 | gattcattaa aaaaactgat tgatagcgtt caagtagata gtgggagtag tagttctaat |
| 9661 | cctactgatg atgacggaga ccataaacca attagtggta aatcagtcaa gccaaatgga |
| 9721 | aaaagtggtc gtgtgattgg tggtaactgg acatatgcac agttaccaga aaaatataaa |
| 9781 | aaagcaattg gtgtaccttt attcaaaaaa gaatacttat acaaaccagg taacatattt |
| 9841 | octcaaacgg gtaatgcagg acaatgtaca gaattaacat gggcgtatat gtcacaacta |
| 9901 | catggtaaaa gacaacctac cgacgacggt caaataacaa acggtcagcg tgtatggtac |
| 9961 | gtctataaaa agttaggtgc aaaaacaaca cataatccaa cagtaggtta tggtttctct |
| 10021 | agtaaaccac catacttaca agcaactgca tatggtattg gtcacacagg tgttgttgta |
| 10081 | gcagtttttg aagatggttc gtttttagtt gcaaactata atgtaccacc atatgttgca |
| 10141 | ccatcacgtg tggtattgta tacactcatt aatggcgtac caaataatgc tggtgataat |
| 10201 | attgtattct ttagtggtat tgcttaatta actatgctat aatgaacaca tgctagtaat |
| 10261 | gctagtaaat aaaatacaaa acataatcaa ttttcgtaca catttttcat gttatctcaa |
| 10321 | aaagaaaagg agactgttat tttaacagtt gccttttttt atttcatcat gttcacgttt |
| 10381 | taatatatgc aaatcagatt tgttatgtac tgaacgttca actggaaata agtcgttaag |
| 10441 | tgaaaatgaa ccgatgtcac tttcaatata aagaatatca tcaaattgac tatggtcgaa |
| 10501 | attttctcta gcgtctttta atataaattc acgtttcata ttaagttcat cagtaaaata |
| 10561 | ttcatcatat acattaccac atacaatttc agttttagac ggatatatcg atattgtacc |
| 10621 | ttgctcatta tagatacttt tattgttttc aataatggca ccgtcaaaga attgttcacg |
| 10681 | tacaaaggtt tcaaaatcga cgcttgtatc aaaggcgttt ttcggtatac cagcagaagc |
| 10741 | aattttaatc tttccattca cttcatatgc atatttctta tgattcagta caaacatctt |
| 10801 | atctatctgt tcgttttcaa tatcccattt acctaaggct atcgggtcga ataaactggg |
| 10861 | gttcaataag ggtttaacaa cggatttcat atacaaacta tcagtatcgc aataaataaa |
| 10921 | attgtcgtca atttcacttt ccgttaagta ttggaaagga accaataagt tatacaatga |
| 10981 | acgtgatgtg acaaatgtag agaataatat attacgttca gtgtttttgt aaccgttaat |
| 11041 | gatattgtat agttcattgt tatcatctaa acggaataag ttaaaatgtg aacgtaatgc |
| 11101 | aggtatgcca tataatccat ttaaaacgac tttagataac ataacctcct catttgagta |
| 11161 | tgggtgttcg ttgatatcat cagtaatgtg atagtcgtaa ggtgatgtca tattgatttt |
| 11221 | gttttttaac ttaccttgtg ttttaataaa atagttttga aaaataatat cacgtgcatg |
| 11281 | aaagtattca cattcatata taacaaacga attaacacgt atatgcatgc aatcaatacc |
| 11341 | cgtaatgtct tgaatcattc ttaatgtatt tgtattgata ttaacgtaat cattatcatt |
| 11401 | attatagtat tttacaatca tttgacgtaa tacacgtgat ttaattttaa ttaataaatc |
| 11461 | atcgttaaat acatctttat caatcttata taatgaaaaa taattgtcat catctaaaaa |
| 11521 | agtagggatt aacgttggtt ctgaatagtg ttcgtaaaag tataaccatg ttggaatttt |
| 11581 | ttcatgatac atcacataag gataactcga attgatgtca atagaaaaac aaggctcatc |
| 11641 | aattagtttg tttatgtatt tggtgttata catatttaaa ccaccacgat agaatgattt |
| 11701 | aatatagtca taaaaattca tatcatggaa atgataatgt gtataagata ttttaatatc |
| 11761 | ttgatattgg ttgagtaact gaaaacgtgt catttcatta ttcaagtaag attccataat |
| 11821 | attcaatgaa aatgttaatt tgttatagtc aaaatttgga aatatatcac tataatgaat |
| 11881 | atggcacata cctaatataa tcacgtcatt atgaatgtat gtaagttgtt caggtgtgag |
| 11941 | ttttgcaaaa catttcacag catagtcata ggcttcacta tcattcatat cattatcttt |
| 12001 | atcaaaaatc gtataattaa aatctgtttt aagttgtgat tctgttaaat aaccaccatc |
| 12061 | aagtaatttc ttacctaatg ttgcaattga tgtattggtt ttcataaagt tatcaataat |
| 12121 | attaaattta aaaccattta aaaacattgt taaatctaaa ttgattgaag atttaacacg |
| 12181 | tttttctaaa attacatttt gatttttggc taaaatagta gcctctttca tttttaatgt |
| 12241 | gtgttcattt tcttctgcag attttaaata tatattttcg cgtgtaatat tatcaaaata |
| 12301 | acgcatggtg tctttaagta aaaaatgatt atcgtattta ttacagttat gtgcaatcat |
| 12361 | gataatatct gtttttgatt ttgtgattgt atcacgtctt ttcacatacg tataaaatgc |
| 12421 | gtcataaaaa gattcgaaac tcggaaatac ttcaacatca atttcataac cattaaacca |
| 12481 | accaattgct acagaataag taacgttttt atatttggtt ggtttttttc gtccgttaac |
| 12541 | tttattgtac gctaatgttt ctatatccca gtataaaatc attcgacgtt catgtttatg |
| 12601 | atattgcatg cattctagta atcccataat cttacacacc ttttataagc catattgttt |
| 12661 | cattagatac tttttcgtat tctctatata gttatcttcg tatatttttt cttttctttc |
| 12721 | aaactcactc atatttttct tcatttcatt ttttatatga aattttataa ttttattcat |
| 12781 | atctaaatat aaatatctat cattatcaac cacgtaattt ttagagtaag cattgtcaaa |
| 12841 | atgtaaattg cttggattgt agtaataacg ttccatgttt tctttataaa acatatcatc |
| 12901 | acgtaaatag gtaacatgat tgtctatatc cctaatttta gtacaaaatt catattgttt |
| 12961 | tgtatatggt acaacgataa tatttgtcat aaaagtagtt acattataca tgactttaat |
| 13021 | atatttatca tcagttttga tatagaagaa atcaccgttt tgattgatgt gatttcttaa |
| 13081 | attatcatcc gccaaattat attcgttaaa ttcaaattct ccagttgtca tagcgtcgtc |
| 13141 | atttgaatta aacgcacgtg tgttacgttt ttcattcacg taatcgtttc gtcgcatttc |
| 13201 | taaaaaaatg tttttgtaaa gtcttgatgt attcatttta tgcttttgta ataaattgta |
| 13261 | tatatttaaa ttggataata taggacttga aaagttgact gcattaccta gtaaaaacat |
| 13321 | tttagggaat ccaatataat caacgttacc atggttacgg tcgattgatt catatattgt |
| 13381 | ttttaactta tcccactcat caattaaata atcatcttca agtgctaaaa actcatcata |
| 13441 | tataataata ggatagtgtt ttaaaaagtt agaatgatat tttaaatcag tggcactatt |
| 13501 | caaatctgta atcacaccaa tttctttatc ttgatagata atagctaaat agtccctagc |
| 13561 | acttctgaac gtgacacgtt ttgatttaaa tagtggattt tcatctatga tttcttcaat |
| 13621 | aaaatcacgg taagcgtcac gtaatgtata atgacgtgat aataaagtaa attttatatc |
| 13681 | aagtttaata gctaaataaa taaaaaatga aacatagttg aacgattttc catcagaacg |
| 13741 | gtttgaaata gatatataat aatctatatc atcattcata agttcatcaa ctaattctat |
| 13801 | ttgattatac ttatctggga ttttttttct gacatgattg acagcatttt gataatctct |
| 13861 | taccatgtct aaacgatttt gttttaccat gtttttgctc cttgtaatag tttatgatgt |
| 13921 | cgtttacagt gttaaattta ttcgtcaaat gttgcataat ataaaaagtt atacctcaca |
| 13981 | tcttcatcat caatatttgt cactggtcta tctgatttac caatttcttt atataaagta |
| 14041 | tcgatttctt taatatattt atacattgaa gaattattat ttttagcttg taaattatat |
| 14101 | aaagcgtatt tatgcttttt agcgttttta ttattagaat catcattacg gttatatatt |
| 14161 | tcaagaatat aatttaattt tttatgtctt gaacctctta ccaatgatac agcatttaca |
| 14221 | tatgatacgt ttctttcttt aggaaaatag ggcagatgtg caaaatgttt ccatgtgtca |
| 14281 | atgtacgcct cttgtaaatc tttatcatca aatttaaaat taacattact aaaatcattt |
| 14341 | aaaaataaat ctttttcttg ctcttttcta gcttctcttt cttttttcca tctatccatt |
| 14401 | tcagacgtat gtctaaccaa tgttatcaac ctccatataa agcataaata accattaaaa |
| 14461 | agataatata gaatataatc aatgtagtga ataaaacacc aaatgacacg cgtatatgca |
| 14521 | gtgtcataag tatgataagt gtaattaaaa atgctaaaag gaaaacaatg gctatgttta |
| 14581 | ataggttatt catggtcaat cactttccca ttatcgtata tgactttgtt ttgataaata |
| 14641 | atcattaatt cgctttcaag aggtttatca aaatttgata atacgtcgtc aattgtaacg |
| 14701 | tttaataaaa tttctcttat taattcatta cttaaataat ttctataata aaatacaagt |
| 14761 | atattaaaaa catgtttttt aatatcaatg tcgatatcta acgtaaataa ctctttttca |
| 14821 | atttcaaaat catcatattg tttgtcaaac tcaatataca catcacccat atttattttt |
| 14881 | actatacatt ttttattaga tgaagtaaat ttttcaaatt tatcattata ataatctcta |
| 14941 | tttgttaaaa ggtaataaat taaattattt aatctaaaag tagttttaat tttcattttt |
| 15001 | atatctcctt aatgtattct atgatatacg cgtatttttt agtgaacagg ttatattcat |
| 15061 | aatatgaata tacaacttta gcgtcatata aatcttcaaa cattgagatt tgatgtggaa |
| 15121 | aatgtccttt aatctcatcg caatataata ataccgtttt gtatttacgt tccatttaaa |
| 15181 | cacctcataa aaaatagggg ataagtatcc cctatgaaat tgtattaaaa tgatacttga |
| 15241 | ccaaaattga ttgagtaacc tttttgacct tttttgtttt catattcata aattgtgaat |
| 15301 | tgaacttctc cagcattgat aatgtcaaca acgtcctcat ctgctctcat ttctttaatt |
| 15361 | aattctgtta agtggttcgg taagtttacg ttatagtcat cagtgacgat aacaccttgt |
| 15421 | tcaccgaatt ttgattcttt gtttgtgaat aatgctctaa cgatatactc ttttttcata |
| 15481 | ccgtattttt ctactaattc tgatagtttg ataaattctc tttctttttc Ctcaaattca |
| 15541 | aatctcgcta atgtgttttg gtgtcttgat aaaatatctt ttacgtttgt cattttattt |
| 15601 | ctcctcttat ttaaattatt tgctttctgc aattgcgatt tgtagtaaat cattgtaata |
| 15661 | aacttgaatt gttttcgttg tgcgtgtagt ggacaatagt ttacatgtgt ctggtaataa |
| 15721 | ttcttttgct tgtgttttgg ttaaatgata ctcgtgaagt ggtaaaaatt cctcaatgta |
| 15781 | ttcattatca tcatctaagt aatgaagtat ataacctttg acacgtaagg taacaatgtc |
| 15841 | gtcaactttc attattatat cactcctttc taaaaaacgt aaacgttata cgtttcataa |
| 15901 | aatcctttat gcatattcca ttgttctatt gggtcatcac cagcaatata agacaatatt |
| 15961 | gattctggtt tagtttcgtt gtttagttca tcatttaaga attgaacaac agaactatta |
| 16021 | tagtttaata atagttgttg gcaagccgat aataagttaa ttgcattgtc aaatgtataa |
| 16081 | gctggattcc attgaatcag tttattgaat agttgcaaca tttcagtata ggcttgtcct |
| 16141 | ttttcttctg gtgcattatc aacattaacc attattatca cttcctaata aagttgaaat |
| 16201 | tacgcgtaaa acagaattat gatttaaatc ttcaatttca tcaatgtcaa catcataaaa |
| 16261 | tgaaatttca ttttctgttc tatcaaataa cgctatacat aaacttccat tcttaaaacg |
| 16321 | aaaaacatgc ttcaactcaa tgttttttgt ttcattttcc atttttgtta ctccttgttt |
| 16381 | tgattacata cttagtatag caaacgttta aaagttttgt caatagtttt tcttaaaaaa |
| 16441 | gtttaaataa ttttaaaact actatttaat agaagaaata agattttaag ttcaaatcat |
| 16501 | aattttgaat aaaagtcaat agatacataa attttgtatt tgatgaatat gtaataggtt |
| 16561 | agataagttg gttaagttgt tgcacagtat ttttaagttt agtaaagaaa tgataagtaa |
| 16621 | atttataagt tttgatttgt ataatcgttt attttaaacc ggtggggt |

TABLE 2

| 1st | | | | | 3rd |
|---|---|---|---|---|---|
| position | 2nd position | | | | position |
| (5′ end) | U | C | A | G | (3′ end) |
| U | Phe | Ser | Tyr | Cys | U |
| | Phe | Ser | Tyr | Cys | C |
| | Leu | Ser | Stop | Stop | A |
| | Leu | Ser | Stop | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

TABLE 3
44AHJDORF012, Nucleotides and amino acids sequences

| | |
|---|---|
| 8391 | atgaacgaagtaaaattcagatttacagactcagaagcgtttcac |
| 1 | M N E V K F R F T D S E A F H |
| 8436 | atqtttatatacgctggggatttaaaattactctactttttattt |
| 16 | M F I Y A C D L K L L Y F L F |
| 8481 | gtattaatgttcgttgatattattacaggtatttcaaaagcaatt |
| 31 | V L M F V D I I T G I S K A I |
| 8526 | aaaaataataacttatggtcaaaaaaatcaatgagaggattttct |
| 46 | K N N N L W S K K S M R G F S |
| 8571 | aaaaaattattgatattctgtattatcattttagcaaacatcatt |
| 61 | K K L L I F C I I I L A N I I |
| 8616 | gaccagattttacaattaaaaggtggtctactcatgattacaata |
| 76 | D Q I L Q L K G C L L M I T I |
| 8661 | ttttattatattgcaaatgagggactttctattgtagaaaattgt |
| 91 | F Y Y I A N S C L S I V S N C |
| 8706 | gcagaaatggacgtattagtaccaqaacaaattaaagataaatta |
| 106 | A S M D V L V P E Q I K D K L |
| 8751 | agagtcattaaaaatgatactgaaaagagtgataacaatgaacga |
| 121 | R V I K N D T F K S D N N F R |
| 8796 | tcaagagaaqatagataa 8813 |
| 136 | S R E D R * |

44AHJDORF025, Nucleotides and amino acids sequences

| | |
|---|---|
| 15175 | atggaacgtaaatacaaaacggtattattatattgcgatgagatt |
| 1 | M E R K Y K T V L L Y C D E I |
| 15130 | aaaggacattttccacatcaaatctcaatgtttgaagatttatat |
| 16 | K G H F P H Q I S M F F D L Y |
| 15085 | gacgctaaagttgtatattcatattatgaatataacctgttcact |
| 31 | D A K V V Y S Y Y E Y N L F T |
| 15040 | aaaaaatacgcgtatatcatagaatacattaaggagatataa 14999 |
| 46 | K K Y A Y I I E Y I K E I * |

TABLE 4
Similarities with public sequences

| | |
|---|---|
| Query = | pt\|110882 44AHJDORF012 44AHJD_NT\|8391-8813\|3 1 |
| | (140 letters) |
| Database: | nr |
| | 445,337 sequences; 137,034,979 total letters |

| | Score | E |
|---|---|---|
| Sequences producing significant alignments: | (bits) | Value |
| gi\|140528\|sp\|P24811\|YQXH_BACSU HYPOTHETICAL 15.7 KD PROTEIN IN . . . | 80 | 6e−15 |
| gi\|4126631\|dbj\|BAA36651.1\| (AB016282) ORF45 [bacteriophage phi- . . . | 76 | 1e−13 |
| gi\|141088\|sp\|P26835\|YNGD_CLOPE HYPOTHETICAL 14.9 KD PROTEIN IN . . . | 61 | 5e−09 |
| gi\|2293160 (AF008220) YtkC [Bacillus subtilis] >gi\|2635548\|emb\| . . . | 36 | 0.11 |
| gi\|1181973\|emb\|CAA87743.1\| (Z47794) holin protein [Bacteriophag . . . | 31 | 3.8 |
| gi\|4981272\|gb\|AAD35828.1\|AE001744_18 (AE001744) carboxyl-termin . . . | 30 | 8.5 |

| | |
|---|---|
| Query = | pt\|110882 44AHJDORF012 44AHJD_NT\|8391-8813\|3 1 |
| | (140 letters) |
| Database: | swissprot |
| | 83,367 sequences; 30,300,539 total letters |

| | Score | E |
|---|---|---|
| Sequences producing significant alignments: | (bits) | Value |
| sp\|P24811 YQXH_BACSU HYPOTHETICAL 15.7 KD PROTEIN IN SPOIIIC-C . . . | 80 | 2e−15 |
| sp\|P26835 YNGD_CLOPE HYPOTHETICAL 14.9 KD PROTEIN IN NAGH 3′RE . . . | 61 | 1e−09 |
| sp\|P18015 COP_CLOPE COPY NUMBER PROTEIN (ORF4). | 28 | 7.9 |

| | |
|---|---|
| Query = | pt\|110899 44AHJDORF025 44AHJD_NT\|14999-151751\|-3 1 |
| | (58 letters) |
| Database: | nr |
| | 445,337 sequences; 137,034,979 total letters |

| | Score | E |
|---|---|---|
| Sequences producing significant alignments: | (bits) | Value |
| gi\|1706558\|sp\|P52869\|EAEA_HAFAL INTIMIN (OUTER MEMBRANE PROTEIN . . . | 28 | 8.4 |

| | |
|---|---|
| Query = | pt\|110899 44AHJDORF025 44AHJD_NT\|14999-15175\|-3 1 |
| | (58 letters) |
| Database: | swissprot |
| | 83,367 sequences; 30,300,539 total letters |

| | Score | E |
|---|---|---|
| Sequences producing significant alignments: | (bits) | Value |
| sp\|P52869 EAEA_HAFAL INTIMIN (OUTER MEMBRANE PROTEIN) (ATTACHI . . . | 28 | 1.9 |
| sp\|Q02785 PDRC_YEAST ATP-DEPENDENT PERMEASE PDR12. | 27 | 4.2 |
| sp\|P75252 Y350_MYCPN HYPOTHETICAL PROTEIN MG350 HOMOLOG. | 27 | 5.6 |
| sp\|P41665 Y112_NPVAC HYPOTHETICAL 10.5 KD PROTEIN IN HE65-PK2 . . . | 27 | 5.6 |
| sp\|P26744 VG01_BPP22 PORTAL PROTEIN (PROTEIN GP1). | 26 | 7.3 |
| sp\|P36542 ATPG_HUMAN ATP SYNTHASE GAMMA CHAIN, MITOCHONDRIAL P . . . | 26 | 9.5 |
| sp\|P35435 ATPG_RAT ATP SYNTHASE GAMMA CHAIN, MITOCHONDRIAL (EC . . . | 26 | 9.5 |

TABLE 5
Physico-chemical parameters for 44AHJDORF012

| | |
|---|---|
| 1 | MNEVKFRFTD SEAFHMFIYA GDLKLLYFLF VLMFVDIITG ISKAIKNNNL WSKKSMRGFS |
| 61 | KKLLIFCIII LANIIDQILQ LKGGLLMITI FYYIANEGLS IVBNCAEMDV LVPEQIKDKL |
| 121 | RVTKNDTEKS DNNERSREDR |

| | |
|---|---|
| Number of amino acids: | 140 |
| Average molecular weight (Daltons): | 16294.30 |
| Mean amino acid weight (Daltons): | 116.39 |
| Monoisotopic molecular weight (Daltons): | 16283.58 |
| Mean amino acid monoisotopic weight (Daltons): | 116.31 |

Amino acid composition

| | | | | Average % |
|---|---|---|---|---|
| Acid | Symbol | Number | % | in Swissprot |
| Ala | A | 6 | 4.29% | 7.58% |
| Asp | D | 9 | 6.43% | 5.28% |
| Phe | F | 10 | 7.14% | 4.09% |
| His | H | 1 | 0.71% | 2.24% |
| Lys | K | 13 | 9.29% | 5.95% |
| Met | M | 6 | 4.29% | 2.37% |
| Pro | P | 1 | 0.71% | 4.9% |
| Arg | R | 6 | 4.29% | 5.16% |
| Thr | T | 4 | 2.86% | 5.67% |
| Trp | W | 1 | 0.71% | 1.23% |
| Cys | C | 2 | 1.43% | 1.66% |
| Glu | E | 9 | 6.43% | 6.37% |
| Gly | G | 6 | 4.29% | 6.84% |
| Ile | I | 18 | 12.86% | 5.81% |
| Leu | L | 16 | 11.43% | 9.42% |
| Asn | N | 10 | 7.14% | 4.45% |
| Gln | Q | 3 | 2.14% | 3.97% |
| Ser | S | 8 | 5.71% | 7.12% |
| Val | V | 7 | 5.00% | 6.58% |
| Tyr | Y | 4 | 2.86% | 3.18% |

| | | |
|---|---|---|
| Number of acidic (negative) amino acids (ED): | 18 | 12.86% |
| Number of basic (positive) amino acids (KR): | 19 | 13.57% |
| Total charge (KRED): | 37 | 26.43% |
| Net charge (KR − ED): | 1 | 0.71% |
| Theoritical pI: | 8.16 | |
| Total linear charge density: | 0.28 | |
| Average hydrophobicity: | 1.26 | |
| Ratio of hydrophilicity to hydrophobicity: | 0.92 | |
| Percentage of hydropliilic amino acid: | 45.71% | |
| Percentage of hydrophobic amino acid: | 54.29% | |
| Ratio of % hydrophilic to % hydrophobic: | 0.84 | |

Hydrophobicity plot

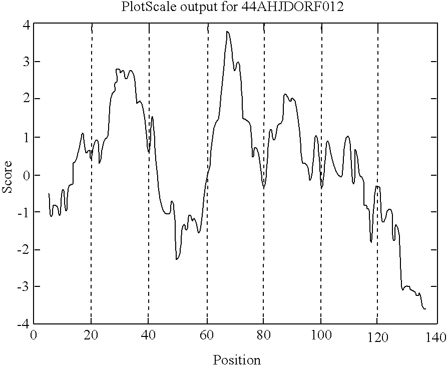

Kyte-Doolittle scale

| | | | | | |
|---|---|---|---|---|---|
| Ala: | 1.800 | Arg: | −4.500 | Asn: | −3.500 |
| Asp: | −3.500 | Cys: | 2.500 | Gly: | −0.400 |
| Gln: | −3.500 | Glu: | −3.500 | His: | −3.200 |
| Ile: | 4.500 | Leu: | 3.800 | Lys: | −3.900 |
| Met: | 1.900 | Phe: | 2.800 | Pro: | −1.600 |
| Ser: | −0.800 | Thr: | −0.700 | Trp: | −0.900 |
| Tyr: | −1.300 | Val: | 4.200 | | |

Physico-chemical parameters for 44AHJDORF025

| | |
|---|---|
| 1 | MERKYKTVLL YCDEIKGHFP HQISMFEDLY DAKVVYSYYE YNLFTKKYAY IIEYTIKEI |

| | |
|---|---|
| Number of amino acids: | 58 |
| Average molecular weight (Daltons): | 7248.41 |
| Mean amino acid weight (Daltons): | 124.97 |
| Monoisotopic molecular weight (Daltons): | 7243.60 |
| Mean amino acid monoisotopic weight (Daltons): | 124.89 |

Amino acid composition

| | | | | Average % |
|---|---|---|---|---|
| Acid | Symbol | Number | % | in Swissprot |
| Ala | A | 2 | 3.45% | 7.58% |
| Asp | D | 3 | 5.17% | 5.28% |
| Phe | F | 3 | 5.17% | 4.09% |
| His | H | 2 | 3.45% | 2.24% |
| Lys | K | 7 | 12.07% | 5.95% |
| Met | M | 2 | 3.45% | 2.37% |
| Pro | P | 1 | 1.72% | 4.9% |
| Arg | R | 1 | 1.72% | 5.16% |
| Thr | T | 2 | 3.45% | 5.67% |
| Trp | W | 0 | 0.00% | 1.23% |
| Cys | C | 1 | 1.72% | 1.66% |
| Glu | E | 6 | 10.34% | 6.37% |
| Gly | G | 1 | 1.72% | 6.84% |
| lIe | I | 6 | 10.34% | 5.81% |
| Leu | L | 4 | 6.90% | 9.42% |
| Asn | N | 1 | 1.72% | 4.45% |
| GIn | Q | 1 | 1.72% | 3.97% |
| Ser | S | 2 | 3.45% | 7.12% |
| Vat | V | 3 | 5.17% | 6.58% |
| Tyr | Y | 10 | 17.24% | 3.18% |

| | | |
|---|---|---|
| Number of acidic (negative) amino acids (ED): | 9 | 15.52% |
| Number of basic (positive) amino acids (KR): | 8 | 13.79% |
| Total charge (KRED): | 17 | 29.31% |
| Net charge (KR − ED): | −1 | −1.72% |
| Theoritical pI: | 6.08 | |
| Total linear charge density: | 0.33 | |
| Average hydrophobicity: | −3.72 | |
| Ratio of hydrophilicity to hydrophobicity: | 1.30 | |
| Percentage of hydrophilic amino acid: | 44.83% | |
| Percentage of hydrophobic amino acid: | 55.17% | |
| Ratio of % hydrophilic to % hydrophobic: | 0.81 | |

Hydrophobicity plot

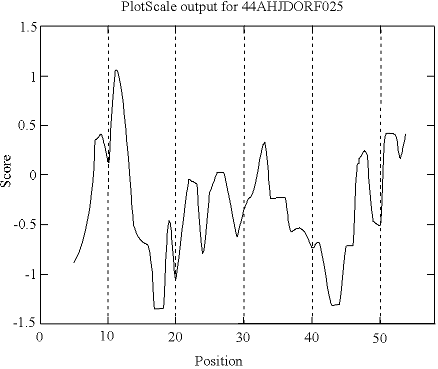

Kyte-Doolittle scale

| | | | | | |
|---|---|---|---|---|---|
| Ala: | 1.800 | Arg: | −4.500 | Asn: | −3.500 |
| Asp: | −3.500 | Cys: | 2.500 | Gly: | −0.400 |
| Gln: | −3.500 | Glu: | −3.500 | His: | −3.200 |
| Ile: | 4.500 | Leu: | 3.800 | Lys: | −3.900 |
| Met: | 1.900 | Phe: | 2.800 | Pro: | −1.600 |
| Ser: | −0.800 | Thr: | −0.700 | Trp: | −0.900 |
| Tyr: | −1.300 | Val: | 4.200 | | |

TABLE 6
>gi|11094395|gb|AAG29618.1| integrase-like protein [Staphylococcus aureus]
>gi|11094394|gb|AAG29617.1|AF217235_20 Orf20 [Staphylococcus aureus]
>gi|11094393|gb|AAG29616.1|AF217235_19 Orf19 [Staphylococcus aureus]
>gi|11094392|gb|AAG29615.1|AF217235_18 Orf18 [Staphylococcus aureus]
>gi|11094391|gb|AAG29614.1|AF217235_17 Orf17 [Staphylococcus aureus]
>gi|11094390|gb|AAG29613.1|AF217235_16 Orf16 [Staphylococcus aureus]
>gi|11094389|gb|AAG29612.1|AF217235_15 Orf15 [Staphylococcus aureus]
>gi|11094388|gb|AAG29611.1|AF217235_14 Orf14 [Staphylococcus aureus]
>gi|11094387|gb|AAG29610.1|AF217235_13 Orf13 [Staphylococcus aureus]
>gi|11094386|gb|AAG29609.1|AF217235_12 Orf12 [Staphylococcus aureus]
>gi|11094385|gb|AAG29608.1|AF217235_11 Orf11 [Staphylococcus aureus]
>gi|11094384|gb|AAG29607.1|AF217235_10 Orf10 [Staphylococcus aureus]
>gi|11094383|gb|AAG29606.1|AF217235_9 Orf9 [Staphylococcus aureus]
>gi|11094382|gb|AAG29605.1|AF217235_8 Orf8 [Staphylococcus aureus]
>gi|11094381|gb|AAG29604.1|AF217235_7 Orf7 [Staphylococcus aureus]
>gi|11094380|gb|AAG29603.1|AF217235_6 Orf6 [Staphylococcus aureus]
>gi|11094379|gb|AAG29602.1|AF217235_5 Orf5 [Staphylococcus aureus]
>gi|11094378|gb|AAG29601.1|AF217235_4 toxic shock syndrome toxin-1 [Staphylococcus aureus]
>gi|11094377|gb|AAG29600.1|AF217235_3 Orf3 [Staphylococcus aureus]
>gi|11094376|gb|AAG29599.1|AF217235_2 staphylococcal enterotoxin C-bovine [Staphylococcus aureus]
>gi|11094375|gb|AAG29598.1|AF217235_1 sel [Staphylococcus aureus]
>gi|9944978|gb|AAG03058.1|AF288215_5 response regulator [Staphylococcus aureus]
>gi|9944977|gb|AAG03057.1|AF288215_4 receptor histidine kinase [Staphylococcus aureus]
>gi|9944976|gb|AAG03056.1|AF288215_3 Agr autoinducing peptide precursor [Staphylococcus aureus]
>gi|9944975|gb|AAG03055.1|AF288215_2 putative AIP processing-secretion protein [Staphylococcus
aureus]
>gi|9944974|gb|AAG03054.1|AF288215_1 delta hemolysin [Staphylococcus aureus]
>gi|10956173|ref|NP_048342.1| ORF64 [Staphylococcus aureus]
>gi|10956172|ref|NP_048341.1| replication protein [Staphylococcus aureus]
>gi|10956170|ref|NP_048340.1| ORF64 [Staphylococcus aureus]
>gi|10956169|ref|NP_048339.1| replication protein [Staphylococcus aureus]
>gi|10956167|ref|NP_052696.1| pot. orfB (aa 1-92) (4557 is 2nd base in codon) [Staphylococcus
aureus]
>gi|10956166|ref|NP_052695.1| pot. orfA [Staphylococcus aureus]
>gi|10956165|ref|NP_052694.1| CAT gene (aa 1-215) [Staphylococcus aureus]
>gi|10956164|ref|NP_052693.1| repD (aa 1-311) [Staphylococcus aureus]
>gi|10956163|ref|NP_052692.1| unidentified reading frame [Staphylococcus aureus]
>gi|10956161|ref|NP_052691.1| kanamycin nucleotidyltransferase (AA 1-253) [Staphylococcus aureus]
>gi|10956160|ref|NP_052690.1| repB polypeptide (AA 1-235) [Staphylococcus aureus]
>gi|10956158|ref|NP_052168.1| recombination protein [Staphylococcus aureus]
>gi|10956157|ref|NP_052167.1| CAT protein [Staphylococcus aureus]
>gi|10956156|ref|NP_052166.1| replication protein [Staphylococcus aureus]
>gi|10956154|ref|NP_053794.1| replication protein [Staphylococcus aureus]
>gi|10956153|ref|NP_053796.1| recombination protein [Staphylococcus aureus]
>gi|10956152|ref|NP_053795.1| tetracycline resistance protein [Staphylococcus aureus]
>gi|10956150|ref|NP_052130.1| beta-lactamase [Staphylococcus aureus]
>gi|10956148|ref|NP_052129.1| beta-lactamase [Staphylococcus aureus]
>gi|10956146|ref|NP_044360.1| tetracycline resistance protein [Staphylococcus aureus]
>gi|10956145|ref|NP_044359.1| replication protein [Staphylococcus aureus]
>gi|10956143|ref|NP_040438.1| reading frame D [Staphylococcus aureus]
>gi|10956142|ref|NP_040437.1| CAT (chloramphenicol resistance) [Staphylococcus aureus]
>gi|10956141|ref|NP_040435.1| reading frame A [Staphylococcus aureus]
>gi|10956140|ref|NP_040436.1| reading frame C (replication) [Staphylococcus aureus]
>gi|10946545|gb|AAG23889.1| TcaB [Staphylococcus aureus]
>gi|10946544|gb|AAG23888.1| TcaA [Staphylococcus aureus]
>gi|10946543|gb|AAG23887.1| TcaR [Staphylococcus aureus]
>gi|2792490|gb|AAB97073.1| coenzyme A disulfide reductase [Staphylococcus aureus]
>gi|10835501|pdb|1D2P|A Chain A, Crystal Structure Of Two B Repeat Units (B1b2) Of The Collagen
Binding Protein (Cna) Of Staphylococcus aureus
>gi|10835500|pdb|1D2O|B Chain B, Crystal Structure Of A Single B Repeat Unit (B1) Of Collagen
Binding Surface Protein (Cna) Of Staphylococcus aureus.
>gi|10835499|pdb|1D2O|A Chain A, Crystal Structure Of A Single B Repeat Unit (B1) Of Collagen
Binding Surface Protein (Cna) Of Staphylococcus aureus.
>gi|1169372|sp|P45555|DNAJ_STAAU CHAPERONE PROTEIN DNAJ (HSP40)
>gi|7672995|gb|AAF66692.1|AF144682_1 immunodominant antigen B [Staphylococcus aureus]
>gi|7672993|gb|AAF66691.1|AF144681_1 immunodominant antigen A [Staphylococcus aureus]
>gi|9955268|pdb|1QE0|B Chain B, Crystal Structure Of Apo S. Aureus Histidyl-Trna Synthetase
>gi|9955267|pdb|1QE0|A Chain A, Crystal Structure Of Apo S. Aureus Histidyl-Trna Synthetase
>gi|9955226|pdb|1F77|B Chain B, Staphylococcal Enterotoxin H Determined To 2.4 A Resolution
>gi|9955225|pdb|1F77|A Chain A, Staphylococcal Enterotoxin H Determined To 2.4 A Resolution
>gi|9954962|pdb|1C79|B Chain B, Staphylokinase (Sak) Dimer
>gi|9954961|pdb|1C79|A Chain A, Staphylokinase (Sak) Dimer
>gi|9954960|pdb|1C78|B Chain B, Staphylokinase (Sak) Dimer
>gi|9954959|pdb|1C78|A Chain A, Staphylokinase (Sak) Dimer
>gi|9954958|pdb|1C77|B Chain B, Staphylokinase (Sak) Dimer
>gi|9954957|pdb|1C77|A Chain A, Staphylokinase (Sak) Dimer
>gi|9954956|pdb|1C76|A Chain A, Staphylokinase (Sak) Monomer
>gi|9954208|gb|AAG08983.1|AF186237_1 ABC protein VgaA variant [Staphylococcus aureus]
>gi|9937366|gb|AAG02426.1|AF290087_3 phosphomevalonate kinase [Staphylococcus aureus]
>gi|9937365|gb|AAG02425.1|AF290087_2 mevalonate diphosphate decarboxylase [Staphylococcus aureus]
>gi|9937364|gb|AAG02424.1|AF290087_1 mevalonate kinase [Staphylococcus aureus]
>gi|9937362|gb|AAG02423.1|AF290086_2 HMG-CoA reductase [Staphylococcus aureus]
>gi|9937361|gb|AAG02422.1|AF290086_1 HMG-CoA synthase [Staphylococcus aureus]
>gi|7415524|dbj|BAA93438.1| FmtB [Staphylococcus aureus]
>gi|7415419|dbj|BAA93431.1| ORF1 [Staphylococcus aureus]
>gi|7415418|dbj|BAA93430.1| FmtB [Staphylococcus aureus]
>gi|10121057|pdb|1FFY|A Chain A, Insights Into Editing From An Ile-Trna Synthetase Structure With
Trna(Ile) And Mupirocin
>gi|110041543|emb|CAC07605.1| unnamed protein product [Staphylococcus aureus]
>gi|1346939|sp|P23215|QACA_STAAU ANTISEPTIC RESISTANCE PROTEIN
>gi|119116|sp|P14319|QACC_STAAU QUATERNARY AMMONIUM COMPOUND-RESISTANCE PROTEIN QACC (QUARTERNARY
AMMONIUM DETERMINANT C) (ETHIDIUM BROMIDE RESISTANCE PROTEIN) (MULTIDRUG RESISTANCE PROTEIN)
>gi|9971595|dbj|BAB12579.1| coagulase [Staphylococcus aureus]
>gi|9971593|dbj|BAB12578.1| coagulase [Staphylococcus aureus]
>gi|9971591|dbj|BAB12577.1| coagulase [Staphylococcus aureus]
>gi|9965494|gb|AAG02249.1| peptide deformylase Pdf1 [Staphylococcus aureus]
>gi|9931634|gb|AAG02239.1|AF295601_1 serine protease-like exoprotein E [Staphylococcus aureus]
>gi|9931632|gb|AAG02238.1|AF295600_1 serine protease-like exoprotein A [Staphylococcus aureus]
>gi|2494147|sp|O05338|PRIM_STAAU DNA PRIMASE
>gi|2492884|sp|Q53634|MENE_STAAU O-SUCCINYLBENZOIC ACID--COA LIGASE (OSB-COA SYNTHETASE) (O-
SUCCINYLBENZOATE-COA SYNTHASE)
>gi|1703465|sp|P52081|ATL_STAAU BIFUNCTIONAL AUTOLYSIN PRECURSOR [INCLUDES: N-ACETYLMURAMOYL-L-
ALANINE AMIDASE ; MANNOSYL-GLYCOPROTEIN ENDO-BETA-N-ACETYLGLUCOSAMIDASE ]
>gi|136130|sp|P19380|T431_STAAU TRANSPOSASE FOR INSERTION SEQUENCE-LIKE ELEMENT IS431MEC
>gi|136126|sp|P14506|T257_STAAU TRANSPOSASE FOR INSERTION SEQUENCE ELEMENT 1S257 IN TRANSPOSON
TN4003
>gi|113675|sp|P24556|ALYS_STAAU AUTOLYSIN (N-ACETYLMURAMOYL-L-ALANINE AMIDASE)
>gi|9801983|gb|AAF99572.1| replication intiation protein [Staphylococcus aureus]
>gi|9801982|gb|AAF99571.1| unknown [Staphylococcus aureus]
>gi|9801981|gb|AAF99570.1| recombinase [Staphylococcus aureus]
>gi|9801980|gb|AAF99569.1| MphBM [Staphylococcus aureus]
>gi|9801979|gb|AAF99568.1| erythromycin resistance protein [Staphylococcus aureus]
>gi|9801978|gb|AAF99567.1| transposase [Staphylococcus aureus]
>gi|9801977|gb|AAF99566.1| unknown [Staphylococcus aureus]
>gi|9801976|gb|AAF99565.1| unknown [Staphylococcus aureus]
>gi|9755015|gb|AAF98155.1| AF251216_3 FhuG [Staphylococcus aureus]
>gi|9755014|gb|AAF98154.1| AF251216_2 FhuB [Staphylococcus aureus]
>gi|9755013|gb|AAF98153.1| AF251216_1 FhuC [Staphylococcus aureus]
>gi|9743649|gb|AAF97986.1| unknown [Staphylococcus aureus]
>gi|9743648|gb|AAD33530.3|AF132117_7 unknown [Staphylococcus aureus]
>gi|9743647|gb|AAD33529.3|AF132117_6 unknown [Staphylococcus aureus]
>gi|9743646|gb|AAD33527.2|AF132117_4 FhuA [Staphylococcus aureus]
>gi|9743645|gb|AAD33526.3|AF132117_3 ferrichrome transport permease [Staphylococcus aureus]
>gi|9743644|gb|AAD33524.3|AF132117_1 ferrichrome transport permease [Staphylococcus aureus]
>gi|9739161|gb|AAF97930.1|AF271715_6 serine protease SplF [Staphylococcus aureus]
>gi|9739160|gb|AAF97929.1|AF271715_5 serine protease SplE [Staphylococcus aureus]
>gi|9739159|gb|AAF97928.1|AF271715_4 serine protease SplD [Staphylococcus aureus]
>gi|9739158|gb|AAF97927.1|AF271715_3 serine protease SplC [Staphylococcus aureus]
>gi|9739157|gb|AAF97926.1|AF271715_2 serine protease SplB [Staphylococcus aureus]
>gi|9739156|gb|AAF97925.1|AF271715_1 serine protease SplA [Staphylococcus aureus]
>gi|9711569|dbj|BAB07846.1| coagulase [Staphylococcus aureus]
>gi|9711565|dbj|BAB07845.1| coagulase [Staphylococcus aureus]
>gi|9711561|dbj|BAB07844.1| coagulase [Staphylococcus aureus]
>gi|9711557|dbj|BAB07843.1| coagulase [Staphylococcus aureus]
>gi|9711553|dbj|BAB07842.1| coagulase [Staphylococcus aureus]
>gi|9711549|dbj|BAB07841.1| coagulase [Staphylococcus aureus]
>gi|9711545|dbj|BAB07840.1| coagulase [Staphylococcus aureus]
>gi|9711541|dbj|BAB07839.1| coagulase [Staphylococcus aureus]
>gi|9711537|dbj|BAB07838.1| coagulase [Staphylococcus aureus]
>gi|9711533|dbj|BAB07837.1| coagulase [Staphylococcus aureus]
>gi|9711528|dbj|BAB07836.1| coagulase [Staphylococcus aureus]
>gi|9711524|dbj|BAB07835.1| coagulase [Staphylococcus aureus]
>gi|9711520|dbj|BAB07834.1| coagulase [Staphylococcus aureus]
>gi|9711516|dbj|BAB07833.1| coagulase [Staphylococcus aureus]
>gi|9711512|dbj|BAB07832.1| coagulase [Staphylococcus aureus]
>gi|9711508|dbj|BAB07831.1| coagulase [Staphylococcus aureus]
>gi|9711504|dbj|BAB07830.1| coagulase [Staphylococcus aureus]
>gi|9711500|dbj|BAB07829.1| coagulase [Staphylococcus aureus]
>gi|9711496|dbj|BAB07828.1| coagulase [Staphylococcus aureus]
>gi|9711492|dbj|BAB07827.1| coagulase [Staphylococcus aureus]
>gi|9711488|dbj|BAB07826.1| coagulase [Staphylococcus aureus]
>gi|9711484|dbj|BAB07825.1| coagulase [Staphylococcus aureus]
>gi|9711480|dbj|BAB07824.1| coagulase [Staphylococcus aureus]
>gi|9622622|gb|AAF89877.1| putative site-specific recombinase XerC [Staphylococcus aureus]
>gi|9622620|gb|AAF89876.1| putative site-specific recombinase XerD [Staphylococcus aureus]
>gi|3806109|gb|AAC69195.1| HsdM-like protein [Staphylococcus aureus]
>gi|3806108|gb|AAC69194.1| exotoxin 4 [Staphylococcus aureus]
>gi|3806107|gb|AAC69193.1| exotoxin 5 [Staphylococcus aureus]
>gi|3806106|gb|AAC69192.1| exotoxin 1 [Staphylococcus aureus]
>gi|3806105|gb|AAC69191.1| exotoxin 3 [Staphylococcus aureus]
>gi|3806104|gb|AAC69190.1| exotoxin 2 [Staphylococcus aureus]
>gi|6176434|gb|AAF05589.1|AF188837_1 exotoxin 1 [Staphylococcus aureus]
>gi|6176433|gb|AAF05588.1|AF188836_1 exotoxin 1 [Staphylococcus aureus]
>gi|6176432|gb|AAF05587.1|AF188835_1 exotoxin 1 [Staphylococcus aureus]
>gi|9501795|dbj|BAB03342.1| Protein A [Staphylococcus aureus]
>gi|9501794|dbj|BAB03341.1| hyothetical protein [Staphylococcus aureus]
>gi|9501793|dbj|BAB03340.1| hypothetical protein [Staphylococcus aureus]
>gi|9501791|dbj|BAB03339.1| ABC transporter [Staphylococcus aureus]
>gi|9501790|dbj|BAB03338.1| ABC transporter [Staphylococcus aureus]
>gi|9501788|dbj|BAB03337.1| ABC transporter [Staphylococcus aureus]
>gi|9501787|dbj|BAB03336.1| ABC transporter [Staphylococcus aureus]
>gi|9501785|dbj|BAB03335.1| hypothetical protein [Staphylococcus aureus]
>gi|9501784|dbj|BAB03334.1| hypothetical protein [Staphylococcus aureus]
>gi|9501783|dbj|BAB03333.1| hypothetical protein [Staphylococcus aureus]
>gi|9501781|dbj|BAB03332.1| fructose specific permease [Staphylococcus aureus]
>gi|9501780|dbj|BAB03331.1| fructose 1-phosphate kinase [Staphylococcus aureus]
>gi|9501779|dbj|BAB03330.1| fru operon repressor [Staphylococcus aureus]
>gi|9501777|dbj|BAB03329.1| hypothetical protein [Staphylococcus aureus]
>gi|9501776|dbj|BAB03328.1| acetyl-CoA c-acetyltransferase [Staphylococcus aureus]
>gi|9501775|dbj|BAB03327.1| long chain fatty acid CoA ligase [Staphylococcus aureus]
>gi|9501774|dbj|BAB03326.1| Pro/Bet transporter homolog [Staphylococcus aureus]
>gi|9501772|dbj|BAB03325.1| response regulator [Staphylococcus aureus]
>gi|9501771|dbj|BAB03324.1| histidine kinase sensor [Staphylococcus aureus]
>gi|9501770|dbj|BAB03323.1| hypothetical protein [Staphylococcus aureus]
>gi|9501769|dbj|BAB03322.1| hypothetical protein [Staphylococcus aureus]
>gi|9501768|dbj|BAB03321.1| methionin aminopeptidase [Staphylococcus aureus]
>gi|9501767|dbj|BAB03320.1| hypothetical protein [Staphylococcus aureus]
>gi|7328282|emb|CAB82465.1| catabolite control protein A [Staphylococcus aureus]
>gi|9408171|emb|CAA71131.1| ORF213 [Staphylococcus aureus]
>gi|9408170|emb|CAA71130.1| ORF133 [Staphylococcus aureus]
>gi|9408169|emb|CAA71129.1| aldehyde dehydrogenase [Staphylococcus aureus]
>gi|8895763|gb|AAF81096.1|AF228662_1 putative undecaprenol kinase [Staphylococcus aureus]
>gi|8777467|dbj|BAA97049.1| 29-kDa cell surface protein [Staphylococcus aureus]
>gi|7328286|emb|CAB82467.1| diaminopimelate decarboxylase [Staphylococcus aureus]
>gi|581546|emb|CAA36783.1| AgrB protein [Staphylococcus aureus]
>gi|581545|emb|CAA36781.1| hypothetical protein [Staphylococcus aureus]
>gi|46600|emb|CAA37901.1| putative transposase [Staphylococcus aureus]
>gi|46599|emb|CAA37900.1| putative transposase [Staphylococcus aureus]
>gi|46513|emb|CAA36786.1| hypothetical protein [Staphylococcus aureus]
>gi|46512|emb|CAA36785.1| hypothetical protein [Staphylococcus aureus]
>gi|46511|emb|CAA36784.1| AgrA protein [Staphylococcus aureus]
>gi|46509|emb|CAA36782.1| hypothetical protein [Staphylococcus aureus]
>gi|46507|emb|CAA36780.1| Hld protein [Staphylococcus aureus]
>gi|46506|emb|CAA36779.1| hypothetical protein [Staphylococcus aureus]
>gi|9256926|pdb|1D6E|C Chain C, Crystal Structure Of Hla-Dr4 Complex With Peptidomimetic And Seb
>gi|9256923|pdb|1D5Z|C Chain C, X-Ray Crystal Structure Of Hla-Dr4 Complexed With Peptidomimetic And
Seb
>gi|9256920|pdb|1D5X|C Chain C, X-Ray Crystal Structure Of Hla-Dr4 Complexed With Dipeptide Mimetic
And Seb
>gi|9256852|pdb|1D5M|C Chain C, X-Ray Crystal Structure Of Hla-Dr4 Complexed With Peptide And Seb
>gi|8569412|pdb|1DEE|H Chain H, Crystal Structure At 2.7a Resolution Of A Complex Between A
Staphylococcus aureus Domain And A Fab Fragment Of A Human Igm Antibody
>gi|8569411|pdb|1DEE|G Chain G, Crystal Structure At 2.7a Resolution Of A Complex Between A
Staphylococcus aureus Domain And A Fab Fragment Of A Human Igm Antibody
>gi|9246437|gb|AAF86053.1|AF210139_1 fmtA-like protein [Staphylococcus aureus]
>gi|9230553|gb|AAF85897.1|AF165314_2 putative protein histidine kinase ArlS [Staphylococcus aureus]
>gi|9230552|gb|AAF85896.1|AF165314_1 putative response regulator ArlR [Staphylococcus aureus]
>gi|9181843|gb|AAF85653.1| orfX [Staphylococcus aureus]
>gi|9181842|gb|AAF85652.1| orf1260 [Staphylococcus aureus]
>gi|9181841|gb|AAF85651.1|AF181950_8 transposase [Staphylococcus aureus]
>gi|9181840|gb|AAF85650.1|AF181950_7 alpha protein [Staphylococcus aureus]
>gi|9181839|gb|AAF85649.1|AF181950_6 beta protein [Staphylococcus aureus]
>gi|9181838|gb|AAF85648.1|AF181950_5 bleomycin resistance protein [Staphylococcus aureus]
>gi|9181837|gb|AAF85647.1|AF181950_4 kanamycin resistance protein [Staphylococcus aureus]
>gi|9181836|gb|AAF85646.1|AF181950_3 transponase [Staphylococcus aureus]
>gi|9181835|gb|AAF85645.1|AF181950_1 low afinity penicillin binding protein [Staphylococcus aureus]
>gi|3603441|gb|AAC35853.1| type b beta-lactamase [Staphylococcus aureus]
>gi|8928563|sp|P81177|AURE_STAAU ZINC METALLOPROTEINASE AUREOLYSIN PRECURSOR (Staphylococcus aureus
NEUTRAL PROTEINASE)
>gi|9049372|dbj|BAA99412.1| exfoliative toxin C [Staphylococcus aureus]
>gi|2495148|sp|Q53727|PCRA_STAAU ATP-DEPENDENT DNA HELICASE PCRA
>gi|115038|sp|P00807|BLAC_STAAU BETA-LACTAMASE PRECURSOR (PENICILLINASE)
>gi|3724158|emb|CAA06500.1| lipoprotein [Staphylococcus aureus]
>gi|3724157|emb|CAA06499.1| ATP binding protein [Staphylococcus aureus]
>gi|3724156|emb|CAA06498.1| membrane protein [Staphylococcus aureus]
>gi|3724155|emb|CAA06497.1| membrane protein [Staphylococcus aureus]
>gi|8885990|gb|AAF80331.1| enterotoxin I [Staphylococcus aureus]
>gi|8648965|emb|CAB94853.1| Map-ND2C protein [Staphylococcus aureus]
>gi|8569359|pdb|1EWC|A Chain A, Crystal Structure Of Zn2+ Loaded Staphylococcal Enterotoxin H
>gi|8134803|sp|Q9ZEH3|UVRC_STAAU EXCINUCLEASE ABC SUBUNIT C
>gi|8134747|sp|Q9Z5C3|TPIS_STAAU TRIOSEPHOSPHATE ISOMERASE (TIM)
>gi|8134611|sp|Q9Z5C4|PGK_STAAU PHOSPHOGLYCERATE KINASE
>gi|8134576|sp|O86491|MURE_STAAU UDP-N-ACETYLMURAMOYLALANYL-D-GLUTAMATE-2,6-DIAMINOPIMELATE LIGASE
(UDP-N-ACETYLNURAMYL-TRIPEPTIDE SYNTHETASE) (MESO-DIAMINOPIMELATE-ADDING ENZYME) (UDP-MURNAC-
TRIPEPTIDE SYNTHETASE)
>gi|7674177|sp|Q9ZAG8|RECU_STAAU RECOMBINATION PROTEIN U HOMOLOG (PENICILLIN-BINDING PROTEIN-RELATED
FACTOR A HOMOLOG) (PBP RELATED FACTOR A HOMOLOG)
>gi|7674147|sp|Q9Z5C9|NRDI_STAAU NRDI PROTEIN
>gi|17388052|sp|O86490|RF3_STAAU PEPTIDE CHAIN RELEASE FACTOR 3 (RF-3)
>gi|7387927|sp|Q9ZEH5|MUS2_STAAU MUTS2 PROTEIN
>gi|7227940|sp|P95842|RSBV_STAAU ANTI-SIGMA B FACTOR ANTAGONIST
>gi|6920067|sp|P81683|EFG_STAAU ELONGATION FACTOR G (EF-G) (85 KDA VITRONECTIN BINDING PROTEIN)
>gi|6686369|sp|O32418|APT_STAAU ADENINE PHOSPHORIBOSYLTRANSFERASE (APRT)
>gi|6685442|sp|P56740|FOLB_STAAU DIHYDRONEOPTERIN ALDOLASE (DHNA)
>gi|6651452|gb|AAF22306.1|AF189239_1 repressor of toxins Rot [Staphylococcus aureus]
>gi|6647733|sp|O32419|RELA_STAAU GTP PYROPHOSPHOKINASE (ATP:GTP 3′-PYROPHOSPHOTRANSFERASE) (PPGPP
SYNTHETASE I) ( (P) PPGPP SYNTHETASE)
>gi|6647411|sp|Q9ZAH5|ALR_STAAU ALANINE RACEMASE
>gi|6226944|sp|O07322|MRAY_STAAU PHOSPHO-N-ACETYLMURAMOYL-PENTAPEPTIDE-TRANSFERASE (UDP-MURNAC-
PENTAPEPTIDE PHOSPHOTRANSFERASE)
>gi|6226498|sp|O05337|YSI3_STAAU HYPOTHETICAL 30.4 KDA PROTEIN IN SIGMA70 OPERON (ORF30)
>gi|6225004|sp|Q9ZAH6|ACPS_STAAU HOLO-[ACYL-CARRIER PROTEIN]SYNTHASE (HOLO-ACP SYNTHASE)
>gi|3913884|sp|Q59801|HYSA_STAAU HYALURONATE LYASE PRECURSOR (HYALURONIDASE) (HYASE)
>gi|3122409|sp|O33595|MURD_STAAU UDP-N-ACETYLMURAMOYLALANINE--D-GLUTAMATE LIGASE (UDP-N-
ACETYLMURAMOYL-L-ALANYL-D-GLUTAMATE SYNTHETASE) (D-GLUTAMIC ACID ADDING ENZYME)
>gi|3122408|sp|O31211|MURC_STAAU UDP-N-ACETYLMURAMATE-- ALANINE LIGASE (UDP-N-ACETYLMURAMOYL-L-
ALANINE SYNTHETASE)
>gi|2500720|sp|O06446|SECA_STAAU PREPROTEIN TRANSLOCASE SECA SUBUNIT
>gi|2499415|sp|Q59821|ODP2_STAAU DIHYDROLIPOAMIDE ACETYLTRANSFERASE COMPONENT OF PYRUVATE
DEHYDROGENASE COMPLEX (E2)
>gi|2498749|sp|Q53726|PCRB_STAAU PCRB PROTEIN
>gi|2494749|sp|Q59812|GLNA_STAAU GLUTAMINE SYNTHETASE (GLUTAMATE SYNTHETASE (GLUTAMATE--AMMONIA LIGASE) (GS)
>gi|2492949|sp|Q59803|AROC_STAAU CHORISMATE SYNTHASE (5-ENOLPYRUVYLSHIKIMATE-3-PHOSPHATE
PHOSPHOLYASE)
>gi|1723227|sp|P52080|YAT3_STAAU HYPOTHETICAL 16.6 KDA PROTEIN IN ATL 5′REGION (ORF3)
>gi|1723225|sp|P52079|YAT2_STAAU HYPOTHETICAL 18.3 KDA PROTEIN IN ATL 5′REGION (ORF2)
>gi|1723223|sp|P52078|YAT1_STAAU HYPOTHETICAL PROTEIN IN ATL 5′REGION (ORF1)
>gi|1709887|sp|P51183|PT1_STAAU PHOSPHOENOLPYRUVATE-PROTEIN PHOSPHOTRANSFERASE (PHOSPHOTRANSFERASE
SYSTEM, ENZYME I)
>gi|1708172|sp|P50915|HEM2_STAAU DELTA-AMINOLEVULINIC ACID DEHYDRATASE (PORPHOBILINOGEN SYNTHASE)
(ALAD) (ALADH)
>gi|1174521|sp|P41972|SYI_STAAU ISOLEUCYL-TRNA SYNTHETASE (ISOLEUCINE--TRNA LIGASE) (ILERS)
>gi|1172890|sp|Q02350|RECA_STAAU RECA PROTEIN
>gi|1170027|sp|P45553|GRPE_STAAU GRPE PROTEIN (HSP-70 COFACTOR) (HSP20)
>gi|729030|sp|P39862|CAPM_STAAU CAPM PROTEIN
>gi|729026|sp|P39858|CAPI_STAAU CAPI PROTEIN
>gi|586026|sp|P02976|SPA1_STAAU IMMUNOGLOBULIN G BINDING PROTEIN A PRECURSOR (IGG BINDING PROTEIN A)
>gi|584922|sp|Q08854|CH60_STAAU 60 KDA CHAPERONIN (PROTEIN CPN60) (GROEL PROTEIN) (HEAT SHOCK
PROTEIN 60)
>gi|461535|sp|Q05615|AROA_STAAU 3-PHOSPHOSHIKIMATE 1-CARBOXYVINYLTRANSFERASE (5-
ENOLPYRUVYLSHIKIMATE-3-PHOSPHATE SYNTHASE) (EPSP SYNTHASE) (EPSPS)
>gi|400202|sp|P31024|LSPA_STAAU LIPOPROTEIN SIGNAL PEPTIDASE (PROLIPOPROTEIN SIGNAL PEPTIDASE)
(SIGNAL PEPTIDASE II) (SPASE II)
>gi|141181|sp|P03861|YP14_STAAU HYPOTHETICAL 14.6 KDA PROTEIN (READING FRAME C) (REPLICATION)
>gi|136616|sp|P13954|TYSY_STAAU THYMIDYLATE SYNTHASE (TS) (TSASE)
>gi|127193|sp|P02979|ERM2_STAAU RRNA ADENINE N-6-METHYLTRANSFERASE (MACROLIDE-LINCOSAMIDE-
STREPTOGRAMIN B RESISTANCE PROTEIN) (ERYTHROMYCIN RESISTANCE PROTEIN)
>gi|125925|sp|P11100|LACD_STAAU TAGATOSE 1,6-DIPHOSPHATE ALDOLASE (TAGATOSE-BISPHOSPHATE ALDOLASE)
(D-TAGATOSE-1,6-BISPHOSPHATE ALDOLASE)
>gi|125922|sp|P11099|LACC_STAAU TAGATOSE-6-PHOSPHATE KINASE (PHOSPHOTAGATOKINASE)
>gi|125919|sp|P26592|LACB_STAAU GALACTOSE-6-PHOSPHATE ISOMERASE LACB SUBUNIT
>gi|123185|sp|P01506|HLD_STAAU DELTA-HEMOLYSIN PRECURSOR (DELTA-TOXIN)
>gi|120457|sp|P14738|FNBA_STAAU FIBRONECTIN-BINDING PROTEIN PRECURSOR (FNBP)
>gi|113525|sp|P21545|AGRB_STAAU ACCESSORY GENE REGULATOR PROTEIN B
>gi|8101860|gb|AAF72664.1|AF259960_1 major cold shock protein CspA [Staphylococcus aureus]
>gi|8099634|gb|AAF72185.1|AF255950_1 AgrD signal peptide precursor [Staphylococcus aureus]
>gi|7959131|dbj|BAA95959.1| secretory protein SAI-B [Staphylococcus aureus]
>gi|7670327|dbj|BAA95014.1| TagG homolog [Staphylococcus aureus]
>gi|7670326|dbj|BAA95013.1| TagH homolog [Staphylococcus aureus]
>gi|7670325|dbj|BAA95012.1| TagA homolog [Staphylococcus aureus]
>gi|7670324|dbj|BAA95011.1| staphylokinase [Staphylococcus aureus]
>gi|7670323|dbj|BAA95010.1| hypothetical protein [Staphylococcus aureus]
>gi|7670322|dbj|BAA95009.1| KdpC homolog [Staphylococcus aureus]
>gi|7670321|dbj|BAA95008.1| KdpB homolog [Staphylococcus aureus]
>gi|7670320|dbj|BAA95007.1| KdpA homolog [Staphylococcus aureus]
>gi|7839534|gb|AAF70313.1|AF260326_2 SrrB [Staphylococcus aureus]
>gi|7839533|gb|AAF70312.1|AF260326_1 SrrA [Staphylococcus aureus]
>gi|7767013|pdb|1ENF|A Chain A, Crystal Structure Of Staphylococcal Enterotoxin H Determined To 1.69
A Resolution
>gi|3401995|pdb|2SPZ|A Chain A, Staphylococcal Protein A, Z-Domain, Nmr, 10 Structures
>gi|1280354|gb|AAA98144.1| ORFA [Staphylococcus aureus]
>gi|7594777|dbj|BAA82240.2| ORF CN050 [Staphylococcus aureus]
>gi|7594776|dbj|BAA82239.2| ORF CN049 [Staphylococcus aureus]
>gi|7594775|dbj|BAA82233.2| ORF N065 [Staphylococcus aureus]
>gi|7594774|dbj|BAA82227.2| ORF CN041 [Staphylococcus aureus]
>gi|7594773|dbj|BAA82226.2| ORF CN040 [Staphylococcus aureus]
>gi|7594772|dbj|BAA82223.2| ORF N060 [Staphylococcus aureus]
>gi|7594771|dbj|BAA82222.2| ORF CN038 [Staphylococcus aureus]
>gi|7594770|dbj|BAA94664.1| ORF N057 [Staphylococcus aureus]
>gi|7594769|dbj|BAA82219.2| methicillin resistance protein MecR1 [Staphylococcus aureus]
>gi|7594768|dbj|BAA82208.2| ORF N051 [Staphylococcus aureus]
>gi|7594767|dbj|BAA82207.2| ORF N050 [Staphylococcus aureus]
>gi|7594766|dbj|BAA82206.2| ORF N049 [Staphylococcus aureus]
>gi|7594765|dbj|BAA94663.1| ORF N043 [Staphylococcus aureus]
>gi|7594764|dbj|BAA94662.1| ORF N042 [Staphylococcus aureus]
>gi|7594763|dbj|BAA94661.1| ORF N041 [Staphylococcus aureus]
>gi|7594762|dbj|BAA94660.1| ORF N039 [Staphylococcus aureus]
>gi|7594761|dbj|BAA94659.1| ORF N038 [Staphylococcus aureus]
>gi|7594760|dbj|BAA94658.1| ORF N033 [Staphylococcus aureus]
>gi|7594759|dbj|BAA94657.1| ORF N032 [Staphylococcus aureus]
>gi|7594758|dbj|BAA94656.1| ORF N031 [Staphylococcus aureus]
>gi|7594757|dbj|BAA94655.1| ORF N030 [Staphylococcus aureus]
>gi|7594756|dbj|BAA94654.1| ORF N029 [Staphylococcus aureus]
>gi|7594755|dbj|BAA82191.2| ORF CN018 [Staphylococcus aureus]
>gi|7594754|dbj|BAA94653.1| ORF N024 [Staphylococcus aureus]
>gi|7594753|dbj|BAA82189.2| ORF CN017 [Staphylococcus aureus]
>gi|7594752|dbj|BAA82178.2| ORF CN007 [Staphylococcus aureus]
>gi|7594751|dbj|BAA94652.1| ORF N010 [Staphylococcus aureus]
>gi|7594750|dbj|BAA94651.1| ORF N009 [Staphylococcus aureus]
>gi|7594749|dbj|BAA94650.1| ORF N008 [Staphylococcus aureus]
>gi|7594748|dbj|BAA94649.1| ORF N007 [Staphylococcus aureus]
>gi|7594747|dbj|BAA82176.2| ORF CN005 [Staphylococcus aureus]
>gi|7594746|dbj|BAA94648.1| ORF N004 [Staphylococcus aureus]
>gi|7594745|dbj|BAA94647.1| ORF CN004 [Staphylococcus aureus]
>gi|7594744|dbj|BAA82175.2| ORF CN003 [Staphylococcus aureus]
>gi|7594743|dbj|BAA82173.2| ORF CN002 [Staphylococcus aureus]
>gi|7594742|dbj|BAA82171.2| ORF CN001 [Staphylococcus aureus]
>gi|7592634|dbj|BAA94340.1| hypothetical protein [Staphylococcus aureus]
>gi|7592633|dbj|BAA94339.1| hypothetical protein [Staphylococcus aureus]
>gi|7592632|dbj|BAA94338.1| hypothetical protein [Staphylococcus aureus]
>gi|7592631|dbj|BAA94337.1| hypothetical protein [Staphylococcus aureus]
>gi|7592630|dbj|BAA94336.1| hypothetical protein [Staphylococcus aureus]
>gi|7592629|dbj|BAA94335.1| hypothetical protein [Staphylococcus aureus]
>gi|7592628|dbj|BAA94334.1| hypothetical protein [Staphylococcus aureus]
>gi|7592627|dbj|BAA94333.1| hypothetical protein [Staphylococcus aureus]
>gi|7592626|dbj|BAA94332.1| hypothetical protein [Staphylococcus aureus]
>gi|7592625|dbj|BAA94331.1| hypothetical protein [Staphylococcus aureus]
>gi|7592624|dbj|BAA94330.1| hypothetical protein [Staphylococcus aureus]
>gi|7592623|dbj|BAA94329.1| hypothetical protein [Staphylococcus aureus]
>gi|7592622|dbj|BAA94328.1| hypothetical protein [Staphylococcus aureus]
>gi|7592621|dbj|BAA94327.1| hypothetical protein [Staphylococcus aureus]
>gi|7592620|dbj|BAA94326.1| hypothetical protein [Staphylococcus aureus]
>gi|7592619|dbj|BAA94325.1| hypothetical protein [Staphylococcus aureus]
>gi|7592618|dbj|BAA86646.3| hypothetical protein [Staphylococcus aureus]
>gi|7592617|dbj|BAA94324.1| hypothetical protein [Staphylococcus aureus]
>gi|7592616|dbj|BAA94323.1| hypothetical protein [Staphylococcus aureus]
>gi|7592615|dbj|BAA94322.1| hypothetical protein [Staphylococcus aureus]
>gi|7592614|dbj|BAA86640.2| hypothetical protein [Staphylococcus aureus]
>gi|7592613|dbj|BAA94321.1| hypothetical protein [Staphylococcus aureus]
>gi|7592612|dbj|BAA94320.1| hypothetical protein [Staphylococcus aureus]
>gi|7592611|dbj|BAA94319.1| hypothetical protein [Staphylococcus aureus]
>gi|7592610|dbj|BAA94318.1| hypothetical protein [Staphylococcus aureus]
>gi|7592609|dbj|BAA94317.1| hypothetical protein [Staphylococcus aureus]
>gi|7592608|dbj|BAA94316.1| hypothetical protein [Staphylococcus aureus]
>gi|7592607|dbj|BAA94315.1| hypothetical protein [Staphylococcus aureus]
>gi|7592606|dbj|BAA94314.1| hypothetical protein [Staphylococcus aureus]
>gi|6332771|dbj|BAA86653.1| open reading frame X [Staphylococcus aureus]
>gi|6332770|dbj|BAA86652.1| transposase [Staphylococcus aureus]
>gi|6332769|dbj|BAA86651.1| glycerophosphoryl diester phosphodiesterase homologue [Staphylococcus
aureus]
>gi|6332768|dbj|BAA86650.1| penicillin-binding protein 2′ [Staphylococcus aureus]
>gi|6332767|dbj|BAA86649.1| hypothetical protein [Staphylococcus aureus]
>gi|6332766|dbj|BAA86648.1| cassette chromosome recombinase A1 [Staphylococcus aureus]
>gi|6332765|dbj|BAA86647.1| hypothetical protein [Staphylococcus aureus]
>gi|6332763|dbj|BAA86645.1| hypothetical protein [Staphylococcus aureus]
>gi|6332762|dbj|BAA86644.1| hypothetical protein [Staphylococcus aureus]
>gi|6332759|dbj|BAA86641.1| hypothetical protein [Staphylococcus aureus]
>gi|6332757|dbj|BAA86639.1| hypothetical protein [Staphylococcus aureus]
>gi|6332756|dbj|BAA86638.1| hypothetical protein [Staphylococcus aureus]
>gi|6332755|dbj|BAA86637.1| hypothetical protein [Staphylococcus aureus]
>gi|6332754|dbj|BAA86636.1| hypothetical protein [Staphylococcus aureus]
>gi|6332753|dbj|BAA86635.1| hypothetical protein [Staphylococcus aureus]
>gi|6332752|dbj|BAA86634.1| hypothetical protein [Staphylococcus aureus]
>gi|6332751|dbj|BAA86633.1| hypothetical protein [Staphylococcus aureus]
>gi|6332750|dbj|BAA86632.1| hypothetical protein [Staphylococcus aureus]
>gi|5360873|dbj|BAA82243.1| orfX [Staphylococcus aureus]
>gi|5360868|dbj|BAA82238.1| transposase for insertion sequence-like element IS431mec [Staphylococcus
aureus]
>gi|5360864|dbj|BAA82234.1| plasmid recombination enzyme [Staphylococcus aureus]
>gi|5360860|dbj|BAA82230.1| bleomycin resistance protein(BRP) [Staphylococcus aureus]
>gi|5360859|dbj|BAA82229.1| kanamycin nucleotidyltransferase [Staphylococcus aureus]
>gi|5360858|dbj|BAA82228.1| transposase for insertion sequence-like element IS431mec [Staphylococcus
aureus]
>gi|5360854|dbj|BAA82224.1| glycerophosphoryldiester phosphodiesterase [Staphylococcus aureus]
>gi|5360851|dbj|BAA82221.1| ORF N059 [Staphylococcus aureus]
>gi|5360850|dbj|BAA82220.1| penicillin binding protein 2′ [Staphylococcus aureus]
>gi|5360848|dbj|BAA82218.1| methicillin resistance protein MecI [Staphylococcus aureus]
>gi|5360847|dbj|BAA82217.1| ORF CN035 [Staphylococcus aureus]
>gi|5360842|dbj|BAA82212.1| ORF CN032 [Staphylococcus aureus]
>gi|5360841|dbj|BAA82211.1| ORF N052 [Staphylococcus aureus]
>gi|5360840|dbj|BAA82210.1| ORF CN031 [Staphylococcus aureus]
>gi|5360839|dbj|BAA82209.1| ORF CN030 [Staphylococcus aureus]
>gi|5360835|dbj|BAA82205.1| rRNA adenine N-6-methyltransferase [Staphylococcus aureus]
>gi|5360834|dbj|BAA82204.1| adenyltransferase(AAD9) [Staphylococcus aureus]
>gi|5360833|dbj|BAA82203.1| transposaseC [Staphylococcus aureus]
>gi|5360832|dbj|BAA82202.1| transposaseB [Staphylococcus aureus]
>gi|5360831|dbj|BAA82201.1| transposaseA [Staphylococcus aureus]
>gi|5360826|dbj|BAA82196.1| site-specific recombinase [Staphylococcus aureus]
>gi|5360824|dbj|BAA82194.1| site-specific recombinase [Staphylococcus aureus]
>gi|5360818|dbj|BAA82188.1| KDP operon transcriptional regulatory protein KdpE [Staphylococcus
aureus]
>gi|5360811|dbj|BAA82181.1| potassium-transporting ATPase (B chanin) [Staphylococcus aureus]
>gi|5360804|dbj|BAA82174.1| ORF N003 [Staphylococcus aureus]
>gi|5360802|dbj|BAA82172.1| ORF N002 [Staphylococcus aureus]
>gi|5360800|dbj|BAA82170.1| ORF N001 [Staphylococcus aureus]
>gi|551990|gb|AAA63529.1| dihydrolipoamide dehydrogenase E2 subunit [Staphylococcus aureus]
>gi|152996|gb|AAA63531.1| dihydrolipoamide acetyltransferase E3 subunit [Staphylococcus aureus]
>gi|152995|gb|AAA63530.1| dihydrolipoamide acetyltransferase E2 subunit [Staphylococcus aureus]
>gi|577647|dbj|BAA07714.1| gamma-hemolysin [Staphylococcus aureus]
>gi|7548684|gb|AAF23273.2|AF101263_3 ABC transporter MreB [Staphylococcus aureus]
>gi|6682106|gb|AAF23274.1|AF101263_4 zinc uptake regulation protein homolog Zur [Staphylococcus
aureus]
>gi|6682104|gb|AAF23272.1|AF101263_2 ABC transporter MreA [Staphylococcus aureus]
>gi|6682103|gb|AAF23271.1|AF101263_1 probable endonuclease IV [Staphylococcus aureus]
>gi|4325247|gb|AAD17309.1| superoxide dismutase SodA [Staphylococcus aureus]
>gi|7532837|gb|AAF63254.1|AF203377_2 replication initiation protein [Staphylococcus aureus]
>gi|7532836|gb|AAF63253.1|AF203377_1 replication-associated protein [Staphylococcus aureus]
>gi|7532834|gb|AAF63252.1|AF203376_2 replication initiation protein [Staphylococcus aureus]
>gi|7532833|gb|AAF63251.1|AF203376_1 replication-associated protein [Staphylococcus aureus]
>gi|7328260|emb|CAA73981.1| hypothetical protein [Staphylococcus aureus]
>gi|7328259|emb|CAA73980.1| hypothetical protein [Staphylococcus aureus]
>gi|7328258|emb|CAA73979.1| protein kinase [Staphylococcus aureus]
>gi|7328257|emb|CAA73978.1| hypothetical protein [Staphylococcus aureus]
>gi|510692|gb|AAA19777.1| enterotoxin H [Staphylococcus aureus]
>gi|7522176|pir||JC7119 Drp35 protein - Staphylococcus aureus
>gi|7470968|pir||S68971 hypothetical protein - Staphylococcus aureus
>gi|7470967|pir||JC5470 hypothetical 29.1 K protein - Staphylococcus aureus
>gi|7470966|pir||T28680 fibrinogen-binding protein homolog - Staphylococcus aureus
>gi|7470965|pir||T28679 fibrinogen-binding protein homolog - Staphylococcus aureus
>gi|7470964|pir||T10908 DNA-directed RNA polymerase (EC 2.7.7.6) beta' chain - Staphylococcus aureus
(fragment)
>gi|7470963|pir||S68731 bleomycin-binding protein - Staphylococcus aureus (fragment)
>gi|7451888|pir||S68970 triacylglycerol lipase (EC 3.1.1.3) precursor - Staphylococcus aureus
>gi|7451316|pir||JC5468 leukocidin chain lukM precursor - Staphylococcus aureus
>gi|7451314|pir||JC5469 Panton-Valentine leukocidin LukF-PV chain precursor - Staphylococcus aureus
>gi|7447124|pir||T10903 acetyltransferase (EC 2.3.1.-) vatB - Staphylococcus aureus
>gi|7443657|pir||JC5607 replication initiation protein dnaA - Staphylococcus aureus
>gi|2145582|pir||S68609 recombinase Sin - Staphylococcus aureus plasmid pSK1
>gi|2145581|pir||S66426 plasmin-sensitive surface protein - Staphylococcus aureus (fragments)
>gi|2126585|pir||PN0638 vgh protein - Staphylococcus aureus
>gi|2126584|pir||JC4555 serine-type D-Ala-D-Ala carboxypeptidase (EC 3.4.16.4) - Staphylococcus
aureus
>gi|2126583|pir||JC4511 pyroglutamyl-peptidase I (EC 3.4.19.3) - Staphylococcus aureus
>gi|2126582|pir||S59955 hypothetical protein 202 - Staphylococcus aureus
>gi|2126581|pir||A55598 hypothetical protein 1 - Staphylococcus aureus (fragment)
>gi|2126580|pir||A53381 hypothetical protein (femC region) - Staphylococcus aureus (fragment)
>gi|2126579|pir||PC4078 hlgC-like protein precursor - Staphylococcus aureus
>gi|2126575|pir||S69209 alpha-toxin precursor - Staphylococcus aureus
>gi|2126574|pir||PQ0040 agrD protein - Staphylococcus aureus
>gi|2126573|pir||S58480 agrC protein - Staphylococcus aureus
>gi|2126572|pir||JC4554 ABC-type transporter homolog - Staphylococcus aureus
>gi|2119993|pir||C40585 recF protein - Staphylococcus aureus
>gi|1363415|pir||S58483 hypothetical protein 8 - Staphylococcus aureus (fragment)
>gi|1363412|pir||JC4282 gamma-hemolysin II precursor - Staphylococcus aureus
>gi|1363410|pir||S49412 fibrinogen-binding protein precursor - Staphylococcus aureus
>gi|1363398|pir||S55767 replication initiator repU - Staphylococcus aureus plasmid pUB110 (fragment)
>gi|1361357|pir||S57202 vitronectin-binding surface protein - Staphylococcus aureus (fragment)
>gi|1361356|pir||B56976 transfer protein complex trsJ - Staphylococcus aureus
>gi|1361355|pir||F56976 transfer complex protein TrsO' - Staphylococcus aureus
>gi|1361354|pir||A36891 transfer complex protein TrsN - Staphylococcus aureus plasmid pGO1
>gi|1361353|pir||E56976 transfer complex protein TrsM - Staphylococcus aureus
>gi|1361352|pir||D56976 transfer complex protein trsL - Staphylococcus aureus
>gi|1361351|pir||C56976 transfer complex protein TrsK - Staphylococcus aureus
>gi|1361349|pir||I36891 transfer complex protein TrsH - Staphylococcus aureus
>gi|1361348|pir||H36891 transfer complex protein TrsG - Staphylococcus aureus
>gi|1361347|pir||G36891 transfer complex protein TrsF - Staphylococcus aureus
>gi|1361346|pir||F36891 transfer complex protein TrsE - Staphylococcus aureus
>gi|1361345|pir||E36891 transfer complex protein TrsD - Staphylococcus aureus
>gi|1361344|pir||D36891 transfer complex protein TrsC - Staphylococcus aureus
>gi|1361343|pir||C36891 transfer complex protein TrsB - Staphylococcus aureus
>gi|1361342|pir||B36891 transfer complex protein TrsA - Staphylococcus aureus
>gi|1361341|pir||S58708 neutral phosphatase - Staphylococcus aureus (ATCC 25923) (fragment)
>gi|1361340|pir||A55856 llm protein - Staphylococcus aureus
>gi|1085937|pir||S42241 hypothetical protein 5 - Staphylococcus aureus plasmid pNS1
>gi|1085933|pir||S42240 hypothetical protein 4 - Staphylococcus aureus plasmid pNS1
>gi|1085928|pir||S42239 hypothetical protein 3 - Staphylococcus aureus plasmid pNS1
>gi|1085919|pir||S42237 hypothetical protein 2 - Staphylococcus aureus plasmid pNS1
>gi|1084189|pir||S54709 hypothetical protein 81 - Staphylococcus aureus
>gi|1075673|pir||S49271 hlgA-like protein precursor - Staphylococcus aureus
>gi|1075672|pir||S52267 DNA polymerase III - Staphylococcus aureus
>gi|1075671|pir||B55548 crtN protein - Staphylococcus aureus
>gi|1075670|pir||A55548 crtM protein - Staphylococcus aureus
>gi|1075668|pir||JC2527 alkaline shock protein - Staphylococcus aureus
>gi|628922|pir||S43693 penicillin-binding protein 2 - Staphylococcus aureus
>gi|543692|pir||S42238 tetracyclin resistance protein - Staphylococcus aureus plasmid pNS1
>gi|541341|pir||S42926 hypothetical membrane spanning protein - Staphylococcus aureus
>gi|541340|pir||S42925 probable transport protein - Staphylococcus aureus
>gi|541339|pir||S39922 pcrB protein - Staphylococcus aureus
>gi|541336|pir||S41539 fibrinogen-binding protein - Staphylococcus aureus
>gi|541335|pir||A48620 adhesin - Staphylococcus aureus (fragment)
>gi|538880|pir||B24362 chloramphenicol O-acetyltransferase leader peptide - Staphylococcus aureus
plasmid pUB112
>gi|484391|pir||JN0627 leukocidin chain F precursor - Staphylococcus aureus
>gi|484390|pir||JN0626 leukocidin chain S precursor - Staphylococcus aureus
>gi|484389|pir||JN0625 gamma-hemolysin chain II precursor - Staphylococcus aureus
>gi|482713|pir||A61069 replication protein REP - Staphylococcus aureus plasmid pOX1000
>gi|482669|pir||A60998 replication protein REP - Staphylococcus aureus plasmid pOX2000
>gi|481955|pir||S40262 hypothetical protein C - Staphylococcus aureus
>gi|481954|pir||S40261 hypothetical protein B - Staphylococcus aureus
>gi|479952|pir||S35697 leukocidin chain F - Staphylococcus aureus
>gi|478296|pir||JN0822 acetyltransferase (EC 2.3.1.-) - Staphylococcus aureus
>gi|478043|pir||C49238 gamma-hemolysin component, HlgC - Staphylococcus aureus
>gi|477912|pir||B49238 gamma-hemolysin gamma 2 component, HlgB - Staphylococcus aureus
>gi|477911|pir||B49234 leucocidin R, component F - Staphylococcus aureus
>gi|477585|pir||A49234 leucocidin R S component - Staphylococcus aureus
>gi|421397|pir||S11782 regulatory protein blaI - Staphylococcus aureus plasmids
>gi|421395|pir||S11780 probable transposase - Staphylococcus aureus transposon Tn552
>gi|421394|pir||S11781 DNA-invertase - Staphylococcus aureus transposon Tn552
>gi|421393|pir||S11779 probable ATP-binding protein - Staphylococcus aureus transposon Tn552
>gi|421390|pir||JN0601 heat shock protein 60 - Staphylococcus aureus
>gi|421389|pir||JN0600 heat shock protein 10 - Staphylococcus aureus
>gi|421388|pir||S32419 gamma-hemolysin chain H gamma II - Staphylococcus aureus
>gi|421387|pir||S34270 fibrinogen-binding protein - Staphylococcus aureus
>gi|421386|pir||S34269 fibrinogen-binding protein - Staphylococcus aureus
>gi|421383|pir||S34444 blaZ protein - Staphylococcus aureus plasmid pI258 (fragment)
>gi|421379|pir||S34447 binR protein - Staphylococcus aureus plasmid pI258 (fragment)
>gi|322083|pir||S32211 leucocidin chain S - Staphylococcus aureus
>gi|322082|pir||S32212 leucocidin chain F - Staphylococcus aureus
>gi|320485|pir||A60633 tetracycline resistance protein - Staphylococcus aureus (strain MRSA101)
>gi|320484|pir||A37389 repN protein - Staphylococcus aureus plasmid pCW7
>gi|320483|pir||C60634 probable transposase - Staphylococcus aureus insertion sequence IS257-3
>gi|320482|pir||B60634 probable transposase - Staphylococcus aureus insertion sequence IS257-2
>gi|320481|pir||A60634 probable transposase - Staphylococcus aureus insertion sequence IS257-1
>gi|320480|pir||A60757 enterotoxin C-1 - Staphylococcus aureus (fragments)
>gi|282254|pir||A41903 recombinase homolog - Staphylococcus aureus (fragment)
>gi|282253|pir||F42721 recombination protein recA - Staphylococcus aureus (fragment)
>gi|282251|pir||S28101 hypothetical protein 2 - Staphylococcus aureus plasmid pC223
>gi|282250|pir||S28102 rlx protein - Staphylococcus aureus plasmid pC223
>gi|282249|pir||S26352 hypothetical protein - Staphylococcus aureus transposon Tn4001
>gi|282243|pir||A42404 collagen adhesin - Staphylococcus aureus
>gi|282241|pir||E41903 recombinase Bin3 - Staphylococcus aureus (fragment)
>gi|282237|pir||S26353 aminoglycoside resistance protein aacA-aphD - Staphylococcus aureus
transposon Tn4001
>gi|282236|pir||A43848 cell surface adhesin for heparan sulfate, 66 K - Staphylococcus aureus
(fragment)
>gi|282235|pir||B43848 cell surface adhesin for heparan sulfate, 60K - Staphylococcus aureus
(fragment)
>gi|280218|pir||A60450 hypothetical protein att155 - Staphylococcus aureus (fragment)
>gi|280217|pir||A44506 lactose operon repressor lacR - Staphylococcus aureus
>gi|97855|pir||JC1204 vgA protein - Staphylococcus aureus
>gi|97854|pir||S12706 type II site-specific deoxyribonuclease (EC 3.1.21.4) Sau96I - Staphylococcus
aureus
>gi|97850|pir||A41511 staphylocoagulase precursor - Staphylococcus aureus (strain BB)
>gi|97847|pir||JQ0759 restriction endonuclease (EC 3.1.-.-) - Staphylococcus aureus
>gi|97846|pir||S09566 repB protein - Staphylococcus aureus plasmid pBD64
>gi|97844|pir||B36242 quinolone resistance protein norA8736 - Staphylococcus aureus plasmid pMR8736
(fragment)
>gi|97843|pir||S12394 probable transport protein qacA - Staphylococcus aureus plasmid pSK1
>gi|97838|pir||S12093 probable transposase - Staphylococcus aureus insertion sequence IS431mec
>gi|97836|pir||A36242 norA209 protein - Staphylococcus aureus plasmid pSA209 (fragment)
>gi|97835|pir||A37838 norA protein - Staphylococcus aureus
>gi|97834|pir||S09565 neomycin resistance protein - Staphylococcus aureus plasmid pBD64
>gi|97831|pir||JQ0773 penicillin-binding protein mecA, low-affinity - Staphylococcus aureus
>gi|97830|pir||S19207 leucocidin R component F precursor - Staphylococcus aureus
>gi|97829|pir||A30471 hypothetical protein rep - Staphylococcus aureus plasmid pSK89
>gi|97828|pir||B30471 hypothetical protein cop - Staphylococcus aureus plasmid pSK89
>gi|97824|pir||S20793 hypothetical protein 5 - Staphylococcus aureus
>gi|97820|pir||S12393 hypothetical protein (qacA 5′ region) - Staphylococcus aureus plasmid pSK1
>gi|97819|pir||S14179 hypothetical protein 140 - Staphylococcus aureus
>gi|97815|pir||B38158 galactose-6-phosphate isomerase 19K chain - Staphylococcus aureus
>gi|97814|pir||A38158 galactose-6-phosphate isomerase (EC 5.1.3.-) 15K chain - Staphylococcus aureus
>gi|97813|pir||S19702 fibronectin-binding protein B - Staphylococcus aureus
>gi|97812|pir||A32192 fibronectin-binding protein - Staphylococcus aureus
>gi|97804|pir||S16509 DNA-invertase - Staphylococcus aureus transposon Tn552
>gi|97795|pir||S09246 coagulase precursor - Staphylococcus aureus (strain 8325-4)
>gi|97793|pir||C34643 cathepsin E (EC 3.4.23.34) - Staphylococcus aureus (fragments)
>gi|97788|pir||S09385 DNA-invertase homolog bin3 - Staphylococcus aureus transposon Tn555
>gi|97786|pir||S15324 beta-hemolysin - Staphylococcus aureus
>gi|97783|pir||B41589 40K elastin-binding protein - Staphylococcus aureus (fragment)
>gi|97782|pir||A41589 25K elastin-binding protein - Staphylococcus aureus (fragment)
>gi|80324|pir||A24456 kanamycin nucleotidyltransferase (EC 2.7.7.-) - Staphylococcus aureus plasmid
pUB110
>gi|79917|pir||S06782 tryptophan synthase (EC 4.2.1.20) - Staphylococcus aureus (fragment)
>gi|79916|pir||A24545 triacylglycerol lipase (EC 3.1.1.3) - Staphylococcus aureus
>gi|79915|pir||C24584 transposition regulatory protein tnpC - Staphylococcus aureus transposon Tn554
>gi|79914|pir||B24584 transposition regulatory protein tnpB - Staphylococcus aureus transposon Tn554
>gi|79913|pir||A24584 transposition regulatory protein tnpA - Staphylococcus aureus transposon Tn554
>gi|79912|pir||S04166 transposase 2 - Staphylococcus aureus transposon Tn4003
>gi|79911|pir||S04162 transposase 1 - Staphylococcus aureus transposon Tn4003
>gi|79910|pir||JS0296 transposase - Staphylococcus aureus
>gi|79906|pir||S06744 staphylocoagulase precursor - Staphylococcus aureus
>gi|79905|pir||A25620 staphylocoagulase - Staphylococcus aureus (fragment)
>gi|79903|pir||S00935 rlx protein - Staphylococcus aureus plasmid pS194
>gi|79902|pir||A29827 replication protein REP - Staphylococcus aureus plasmids
>gi|79901|pir||S00909 replication initiation protein - Staphylococcus aureus plasmid pC223
>gi|79900|pir||A30480 repJ protein - Staphylococcus aureus plasmid pC223
>gi|79899|pir||JT0372 repI protein - Staphylococcus aureus plasmid pUB112
>gi|79898|pir||S00937 repE protein - Staphylococcus aureus plasmid pS194
>gi|79896|pir||A29605 protein A precursor - Staphylococcus aureus (strain Cowan 1)
>gi|79894|pir||S20576 probable regulatory protein mecI - Staphylococcus aureus
>gi|79891|pir||B28474 phosphotransferase system enzyme II (EC 2.7.1.69), lactose-specific, factor II -
Staphylococcus aureus
>gi|79887|pir||JQ1147 N-acetylmuramoyl-L-alanine amidase (EC 3.5.1.28) - Staphylococcus aureus
>gi|79886|pir||JQ1439 multidrug resistance protein - Staphylococcus aureus plasmids
>gi|79884|pir||G29504 hypothetical 20K protein (mer regulatory region) - Staphylococcus aureus
plasmid pI258
>gi|79883|pir||C29504 hypothetical 24K protein (mer operon) - Staphylococcus aureus plasmid pI258
>gi|79882|pir||B29504 hypothetical 18K protein (mer operon) - Staphylococcus aureus plasmid pI258
>gi|79881|pir||A29504 hypothetical 16K protein (mer operon) - Staphylococcus aureus plasmid pI258
>gi|79880|pir||D29504 hypothetical 14K protein (mer operon) - Staphylococcus aureus plasmid pI258
>gi|79879|pir||S20575 mecR1 protein - Staphylococcus aureus
>gi|79877|pir||A31901 lincomycin resistance protein linA' - Staphylococcus aureus (strain BM4611)
>gi|79875|pir||JQ1530 leukocidin chain F precursor - Staphylococcus aureus
>gi|79874|pir||S04359 lacD protein - Staphylococcus aureus
>gi|79871|pir||S00936 hypothetical protein D - Staphylococcus aureus plasmid pS194
>gi|79869|pir||S15767 hypothetical protein 2 (hlb 3′ region) - Staphylococcus aureus (fragment)
>gi|79868|pir||S15765 hypothetical protein 1 (hlb 5′ region) - Staphylococcus aureus (fragment)
>gi|79867|pir||F24584 hypothetical protein - Staphylococcus aureus transposon Tn554
>gi|79866|pir||S04165 hypothetical protein - Staphylococcus aureus plasmid pSK1 transposon Tn4003
>gi|79865|pir||S06784 hypothetical protein (femA 3′ region) - Staphylococcus aureus
>gi|79864|pir||B32561 cadC protein - Staphylococcus aureus plasmid pI258
>gi|79863|pir||A41652 probable glycerophosphodiester phosphodiesterase (EC 3.1.4.46) -
Staphylococcus aureus
>gi|79862|pir||S06783 femA protein - Staphylococcus aureus
>gi|79857|pir||JG0016 epidermal cell differentiation inhibitor precursor - Staphylococcus aureus
>gi|79843|pir||JQ0387 agrB protein - Staphylococcus aureus
>gi|79842|pir||A32357 accessory gene regulatory protein agrA - Staphylococcus aureus
>gi|7437965|pir||JC6560 UDP-N-acetylmuramoylalanine - D-glutamate ligase (EC 6.3.2.9) -
Staphylococcus aureus
>gi|7434769|pir||S34442 transcription initiation factor sigma plaC - Staphylococcus aureus
>gi|2144945|pir||QVSAA protein A precursor - Staphylococcus aureus
>gi|2144683|pir||ENSAC1 enterotoxin C-1 precursor - Staphylococcus aureus
>gi|2144682|pir||ENSAB6 enterotoxin B precursor - Staphylococcus aureus
>gi|2126578|pir||S54426 DNA topoisomerase (ATP-hydrolyzing) (EC 5.99.1.3) chain B - Staphylococcus
aureus
>gi|2126577|pir||S54427 gyrase-like protein alpha chain - Staphylococcus aureus
>gi|2126576|pir||S59956 DNA-directed RNA polymerase (EC 2.7.7.6) beta' chain - Staphylococcus aureus
(fragment)
>gi|2119116|pir||S59954 ribosomal protein L7/L12 - Staphylococcus aureus (fragment)
>gi|2117996|pir||A53641 arsenate reductase (EC 1.-.-.-) - Staphylococcus aureus plasmid pI258
>gi|2117910|pir||S59951 DNA-directed RNA polymerase (EC 2.7.7.6) beta chain - Staphylococcus aureus
>gi|1363411|pir||S58814 cell division protein ftsZ - Staphylococcus aureus
>gi|1361350|pir||A56976 transfer complex protein TrsI - Staphylococcus aureus
>gi|1084187|pir||S54708 DNA-directed DNA polymerase (EC 2.7.7.7) III beta chain - Staphylococcus
aureus
>gi|1075676|pir||S54793 superoxide dismutase (EC 1.15.1.1) (Fe/Mn) - Staphylococcus aureus
(fragment)
>gi|1075669|pir||S52934 alkyl hydroperoxide reductase (EC 1.6.4.-) c22 protein - Staphylococcus
aureus (fragment)
>gi|625852|pir||JP0045 ribosomal protein L30 - Staphylococcus aureus (fragment)
>gi|541338|pir||S39923 DNA helicase pcrA - Staphylococcus aureus
>gi|541337|pir||S40178 isoleucine - tRNA ligase (EC 6.1.1.5) - Staphylococcus aureus
>gi|538884|pir||B46568 ermC protein - Staphylococcus aureus plasmid pT48
>gi|538882|pir||A40585 DNA topoisomerase (ATP-hydrolyzing) (EC 5.99.1.3) chain B - Staphylococcus
aureus
>gi|538881|pir||B40585 DNA topoisomerase (ATP-hydrolyzing) (EC 5.99.1.3) chain A - Staphylococcus
aureus
>gi|538608|pir||A24362 chloramphenicol O-acetyltransferase (EC 2.3.1.28) - Staphylococcus aureus
plasmid pUB112
>gi|482777|pir||A61152 chloramphenicol O-acetyltransferase (EC 2.3.1.28) - Staphylococcus aureus
plasmid pSCS7
>gi|421382|pir||S11783 bla regulator protein blaR1 - Staphylococcus aureus plasmids
>gi|322081|pir||S32014 dihydrofolate reductase (EC 1.5.1.3) - Staphylococcus aureus
>gi|322080|pir||A44849 chloramphenicol O-acetyltransferase (EC 2.3.1.28) - Staphylococcus aureus
plasmid
>gi|282240|pir||B41903 arsenical resistance operon repressor - Staphylococcus aureus plasmid pI258
>gi|282239|pir||D41903 arsenate reductase (EC 1.-.-.-) - Staphylococcus aureus plasmid pI258
>gi|282238|pir||C41903 arsenical pump membrane protein - Staphylococcus aureus
>gi|279459|pir||YXSAT3 thymidylate synthase (EC 2.1.1.45) - Staphylococcus aureus plasmid pSK1
transposon Tn4003
>gi|98263|pir||A36141 cop protein - Staphylococcus aureus plasmid pE194
>gi|97849|pir||S12705 site-specific DNA-methyltransferase (cytosine-specific) (EC 2.1.1.73) Sau96I -
Staphylococcus aureus
>gi|97842|pir||S19721 pyruvate dehydrogenase (lipoamide) (EC 1.2.4.1) chain E1-beta - Staphylococcus
aureus (fragment)
>gi|97837|pir||A31048 phosphotransferase system enzyme II (EC 2.7.1.69), mannitol-specific, factor
III - Staphylococcus aureus (fragments)
>gi|97833|pir||JQ0760 methyltransferase (EC 2.1.1.-) - Staphylococcus aureus
>gi|97832|pir||S20433 lipoprotein signal peptidase (EC 3.4.23.36) - Staphylococcus aureus
>gi|97826|pir||S20799 hypothetical protein 7 - Staphylococcus aureus
>gi|97816|pir||S21758 glutamic acid-specific endopeptidase - Staphylococcus aureus
>gi|97807|pir||A33953 enterotoxin D precursor - Staphylococcus aureus
>gi|97805|pir||S11885 enterotoxin C3 - Staphylococcus aureus
>gi|97798|pir||S19723 dihydrolipoamide dehydrogenase (EC 1.8.1.4) - Staphylococcus aureus
>gi|97797|pir||S19722 dihydrolipoamide S-acetyltransferase (EC 2.3.1.12) chain E2 - Staphylococcus
aureus
>gi|97787|pir||A35001 beta-lactamase (EC 3.5.2.6) PSE-4 precursor - Staphylococcus aureus
>gi|80332|pir||B29827 macrolide/lincosamide/streptogramin B resistance methylase - Staphylococcus
aureus plasmid pE5
>gi|79907|pir||S00938 str protein - Staphylococcus aureus plasmid pS194
>gi|79904|pir||D24584 spectinomycin resistance protein spc - Staphylococcus aureus transposon Tn554
>gi|79893|pir||A32561 probable cadmium-transporting ATPase (EC 3.6.1.-) - Staphylococcus aureus
>gi|79888|pir||F29504 alkylmercury lyase (EC 4.99.1.2) - Staphylococcus aureus plasmid pI258
>gi|79885|pir||E29504 mercury(II) reductase (EC 1.16.1.1) - Staphylococcus aureus plasmid pI258
>gi|79878|pir||A25101 erythromycin resistance protein ermA - Staphylococcus aureus transposon Tn554
>gi|79873|pir||S04358 lacC protein - Staphylococcus aureus
>gi|79861|pir||A26050 exfoliative toxin B precursor - Staphylococcus aureus
>gi|79856|pir||A28179 enterotoxin E precursor - Staphylococcus aureus
>gi|79855|pir||A60114 enterotoxin C-2 precursor - Staphylococcus aureus
>gi|79853|pir||A28664 enterotoxin A precursor - Staphylococcus aureus
>gi|79851|pir||S04164 dihydrofolate reductase (EC 1.5.1.3) - Staphylococcus aureus plasmid pSK1
transposon Tn4003
>gi|79844|pir||A27233 beta-galactosidase (EC 3.2.1.23) - Staphylococcus aureus
>gi|76321|pir||QQSAC2 hypothetical protein C - Staphylococcus aureus plasmid pC221
>gi|76320|pir||QQSAA2 rlx protein - Staphylococcus aureus plasmid pC221
>gi|76319|pir||QQSA8T hypothetical protein B-189 - Staphylococcus aureus plasmid pT181
>gi|76318|pir||QQSAEC hypothetical protein E-229 - Staphylococcus aureus plasmid pC194
>gi|76317|pir||QQSACC hypothetical protein C-120 - Staphylococcus aureus plasmid pC194
>gi|76316|pir||QQSA7C hypothetical protein E-74 - Staphylococcus aureus plasmid pC194
>gi|76315|pir||QQSACE hypothetical protein C-102 - Staphylococcus aureus plasmid pE194
>gi|76314|pir||QQSABE hypothetical protein B-111 - Staphylococcus aureus plasmid pE194
>gi|73155|pir||RQSAD2 repD protein - Staphylococcus aureus plasmid pC221
>gi|73154|pir||RQSACT repC protein - Staphylococcus aureus plasmids
>gi|73152|pir||LFSAP9 ermC leader peptide - Staphylococcus aureus plasmids
>gi|72984|pir||QQSA4E hypothetical protein C-403 - Staphylococcus aureus plasmid pE194
>gi|72843|pir||QQSACT hypothetical protein C-156 - Staphylococcus aureus plasmid pT181
>gi|72842|pir||QQSABT hypothetical protein B-295 - Staphylococcus aureus plasmid pT181
>gi|72420|pir||WPSAHP phosphotransferase system phosphohistidine-containing protein - Staphylococcus
aureus
>gi|69625|pir||XCSAS1 toxic shock syndrome toxin-1 precursor - Staphylococcus aureus
>gi|69556|pir||LESAD delta hemolysin - Staphylococcus aureus
>gi|67766|pir||PNSAP beta-lactamase (EC 3.5.2.6) precursor - Staphylococcus aureus
>gi|67543|pir||PRSAEB epidermolytic toxin B precursor - Staphylococcus aureus plasmid pRW001
>gi|67542|pir||PRSAEA epidermolytic toxin A precursor - Staphylococcus aureus
>gi|67541|pir||PRSASK glutamyl endopeptidase (EC 3.4.21.19) precursor - Staphylococcus aureus
>gi|67305|pir||NCSAF micrococcal nuclease (EC 3.1.31.1) precursor - Staphylococcus aureus
>gi|66882|pir||PKSAF kanamycin kinase (EC 2.7.1.95) - Staphylococcus aureus
>gi|66872|pir||WQSA3L phosphotransferase system enzyme II (EC 2.7.1.69), lactose-specific, factor
III - Staphylococcus aureus
>gi|66524|pir||XXSAC2 chloramphenicol O-acetyltransferase (EC 2.3.1.28) - Staphylococcus aureus
plasmid pC221
>gi|66523|pir||XXSACC chloramphenicol O-acetyltransferase (EC 2.3.1.28) - Staphylococcus aureus
plasmids
>gi|66452|pir||YESA9E rRNA (adenine-N6-)-methyltransferase (EC 2.1.1.48) - Staphylococcus aureus
plasmids
>gi|7381167|gb|AAF61418.1|AF135268_1 ribonuclease P protein component [Staphylococcus aureus]
>gi|6648971|gb|AAF21314.1| site-specific recombinase [Staphylococcus aureus]
>gi|6648970|gb|AAF21313.1|AF118839_1 iron uptake regulatory protein; Fur [Staphylococcus aureus]
>gi|7330783|gb|AAF60251.1| Geh [Staphylococcus aureus]
>gi|7330782|gb|AAF60250.1| IcaC [Staphylococcus aureus]
>gi|7330780|gb|AAF60249.1| Geh [Staphylococcus aureus]
>gi|7330779|gb|AAF60248.1| IcaC [Staphylococcus aureus]
>gi|7330777|gb|AAF60247.1| Geh [Staphylococcus aureus]
>gi|7330776|gb|AAF60246.1| IcaC [Staphylococcus aureus]
>gi|7330774|gb|AAF60245.1| Geh [Staphylococcus aureus]
>gi|7330773|gb|AAF60244.1| IcaC [Staphylococcus aureus]
>gi|7330771|gb|AAF60243.1| Geh [Staphylococcus aureus]
>gi|7330770|gb|AAF60242.1| IcaC [Staphylococcus aureus]
>gi|7330768|gb|AAF60241.1| HprK [Staphylococcus aureus]
>gi|7330767|gb|AAF60240.1| UvrA [Staphylococcus aureus]
>gi|7330765|gb|AAF60239.1| HprK [Staphylococcus aureus]
>gi|7330764|gb|AAF60238.1| UvrA [Staphylococcus aureus]
>gi|7330762|gb|AAF60237.1| HprK [Staphylococcus aureus]
>gi|7330761|gb|AAF60236.1| UvrA [Staphylococcus aureus]
>gi|7330759|gb|AAF60235.1| HprK [Staphylococcus aureus]
>gi|7330758|gb|AAF60234.1| UvrA [Staphylococcus aureus]
>gi|7330756|gb|AAF60233.1| HprK [Staphylococcus aureus]
>gi|7330755|gb|AAF60232.1| UvrA [Staphylococcus aureus]
>gi|7330753|gb|AAF60231.1| HprK [Staphylococcus aureus]
>gi|7330752|gb|AAF60230.1| UvrA [Staphylococcus aureus]
>gi|6671351|gb|AAF23170.1|AF210055_3 AgrC [Staphylococcus aureus]
>gi|6671350|gb|AAF23169.1|AF210055_2 AgrD [Staphylococcus aureus]
>gi|6671349|gb|AAF23168.1|AF210055_1 AgrB [Staphylococcus aureus]
>gi|7328298|emb|CAB82464.1| transketolase, putative [Staphylococcus aureus]
>gi|7328297|emb|CAB82463.1| SOS regulatory LexA protein, putative [Staphylococcus aureus]
>gi|7328295|emb|CAB82462.1| DNA mismatch repair protein [Staphylococcus aureus]
>gi|7328294|emb|CAB82461.1| DNA mismatch repair protein [Staphylococcus aureus]
>gi|7328292|emb|CAB82460.1| hypothetical protein [Staphylococcus aureus]
>gi|7328291|emb|CAB82459.1| hypothetical protein [Staphylococcus aureus]
>gi|7328290|emb|CAB82458.1| hypothetical protein [Staphylococcus aureus]
>gi|7328289|emb|CAB82457.1| hypothetical protein [Staphylococcus aureus]
>gi|7328288|emb|CAB82456.1| hypothetical protein [Staphylococcus aureus]
>gi|7328284|emb|CAB82466.1| MutS protein [Staphylococcus aureus]
>gi|7328280|emb|CAB82478.1| hypothetical protein [Staphylococcus aureus]
>gi|7328279|emb|CAB82477.1| hypothetical protein [Staphylococcus aureus]
>gi|7328278|emb|CAB82476.1| hypothetical protein [Staphylococcus aureus]
>gi|7328277|emb|CAB82475.1| D-alanine aminotransferase [Staphylococcus aureus]
>gi|7328276|emb|CAB82474.1| putative peptidase [Staphylococcus aureus]
>gi|7328275|emb|CAB82473.1| hypothetical protein [Staphylococcus aureus]
>gi|7328274|emb|CAB82472.1| hypothetical protein [Staphylococcus aureus]
>gi|7328272|emb|CAB82471.1| ORF314 [Staphylococcus aureus]
>gi|7328271|emb|CAB82470.1| hypothetical protein [Staphylococcus aureus]
>gi|7328270|emb|CAB82469.1| hypothetical protein [Staphylococcus aureus]
>gi|7328269|emb|CAB82468.1| hypothetical protein [Staphylococcus aureus]
>gi|7106006|emb|CAB75986.1| ornithine carbamoyltransferase [Staphylococcus aureus]
>gi|7272362|gb|AAA26601.2| blaZ [Staphylococcus aureus]
>gi|152968|gb|AAA26604.1| binR [Staphylococcus aureus]
>gi|152967|gb|AAA26603.1| blaI [Staphylococcus aureus]
>gi|152966|gb|AAA26602.1| blaR1 [Staphylococcus aureus]
>gi|7242216|gb|AAB32123.2| porphobilinogen synthase; PBG; HemB [Staphylococcus aureus]
>gi|7239722|gb|AAA71951.2| putative [Staphylococcus aureus]
>gi|7239370|gb|AAF43206.1|AF230358_3 accessory gene regulator protein D [Staphylococcus aureus]
>gi|7239369|gb|AAF43205.1|AF230358_2 accessory gene regulator protein B [Staphylococcus aureus]
>gi|7239368|gb|AAF43204.1|AF230358_1 delta-haemolysin precurser [Staphylococcus aureus]
>gi|310620|gb|AAA71964.1| putative [Staphylococcus aureus]
>gi|310619|gb|AAA71963.1| putative [Staphylococcus aureus]
>gi|310618|gb|AAA71962.1| putative [Staphylococcus aureus]
>gi|310617|gb|AAA71961.1| putative [Staphylococcus aureus]
>gi|310616|gb|AAA71960.1| putative [Staphylococcus aureus]
>gi|310615|gb|AAA71959.1| putative [Staphylococcus aureus]
>gi|310614|gb|AAA71958.1| putative [Staphylococcus aureus]
>gi|310613|gb|AAA71957.1| putative [Staphylococcus aureus]
>gi|310612|gb|AAA71956.1| putative [Staphylococcus aureus]
>gi|310611|gb|AAA71955.1| putative [Staphylococcus aureus]
>gi|310610|gb|AAA71954.1| putative [Staphylococcus aureus]
>gi|310609|gb|AAA71953.1| putative [Staphylococcus aureus]
>gi|310608|gb|AAA71952.1| putative [Staphylococcus aureus]
>gi|4126683|dbj|BAA36693.1| enterotoxin type Gv [Staphylococcus aureus]
>gi|7162103|emb|CAB76672.1| phosphate actyltransferase [Staphylococcus aureus]
>gi|7162101|emb|CAB76671.1| phosphate actyltransferase [Staphylococcus aureus]
>gi|7162099|emb|CAB76670.1| phosphate actyltransferase [Staphylococcus aureus]
>gi|7162097|emb|CAB76669.1| phosphate actyltransferase [Staphylococcus aureus]
>gi|7162095|emb|CAB76668.1| phosphate actyltransferase [Staphylococcus aureus]
>gi|7162093|emb|CAB76667.1| phosphate actyltransferase [Staphylococcus aureus]
>gi|7162091|emb|CAB76666.1| phosphate actyltransferase [Staphylococcus aureus]
>gi|7162089|emb|CAB76665.1| phosphate actyltransferase [Staphylococcus aureus]
>gi|7162087|emb|CAB76664.1| phosphate actyltransferase [Staphylococcus aureus]
>gi|7162085|emb|CAB76663.1| phosphate actyltransferase [Staphylococcus aureus]
>gi|7162083|emb|CAB76662.1| phosphate actyltransferase [Staphylococcus aureus]
>gi|7162081|emb|CAB76661.1| phosphate actyltransferase [Staphylococcus aureus]
>gi|7162079|emb|CAB76660.1| phosphate actyltransferase [Staphylococcus aureus]
>gi|7162077|emb|CAB76659.1| phosphate actyltransferase [Staphylococcus aureus]
>gi|7162075|emb|CAB76658.1| phosphate actyltransferase [Staphylococcus aureus]
>gi|7162073|emb|CAB76657.1| triosephosphate isomerase [Staphylococcus aureus]
>gi|7162071|emb|CAB76656.1| triosephosphate isomerase [Staphylococcus aureus]
>gi|7162069|emb|CAB76655.1| triosephosphate isomerase [Staphylococcus aureus]
>gi|7162067|emb|CAB76654.1| triosephosphate isomerase [Staphylococcus aureus]
>gi|7162065|emb|CAB76653.1| triosephosphate isomerase [Staphylococcus aureus]
>gi|7162063|emb|CAB76652.1| triosephosphate isomerase [Staphylococcus aureus]
>gi|7162061|emb|CAB76651.1| triosephosphate isomerase [Staphylococcus aureus]
>gi|7162059|emb|CAB76650.1| triosephosphate isomerase [Staphylococcus aureus]
>gi|7162057|emb|CAB76649.1| triosephosphate isomerase [Staphylococcus aureus]
>gi|7162055|emb|CAB76648.1| triosephosphate isomarase [Staphylococcus aureus]
>gi|7162053|emb|CAB76647.1| triosephosphate isomerase [Staphylococcus aureus]
>gi|7162051|emb|CAB76646.1| triosephosphate isomerase [Staphylococcus aureus]
>gi|7162049|emb|CAB76645.1| triosephosphate isomerase [Staphylococcus aureus]
>gi|7162047|emb|CAB76644.1| triosephosphate isomerase [Staphylococcus aureus]
>gi|7162045|emb|CAB76643.1| carbamate kinase [Staphylococcus aureus]
>gi|7162043|emb|CAB76642.1| carbamate kinase [Staphylococcus aureus]
>gi|7162041|emb|CAB76641.1| carbamate kinase [Staphylococcus aureus]
>gi|7162039|emb|CAB76640.1| carbamate kinase [Staphylococcus aureus]
>gi|7162037|emb|CAB76639.1| carbamate kinase [Staphylococcus aureus]
>gi|7162035|emb|CAB76638.1| carbamate kinase [Staphylococcus aureus]
>gi|7162033|emb|CAB76637.1| carbamate kinase [Staphylococcus aureus]
>gi|7162031|emb|CAB76636.1| carbamate kinase [Staphylococcus aureus]
>gi|7162029|emb|CAB76635.1| carbamate kinase [Staphylococcus aureus]
>gi|7162027|emb|CAB76634.1| carbamate kinase [Staphylococcus aureus]
>gi|7162025|emb|CAB76633.1| carbamate kinase [Staphylococcus aureus]
>gi|7162023|emb|CAB76632.1| carbamate kinase [Staphylococcus aureus]
>gi|7162021|emb|CAB76631.1| carbamate kinase [Staphylococcus aureus]
>gi|7162019|emb|CAB76630.1| carbamate kinase [Staphylococcus aureus]
>gi|7162017|emb|CAB76629.1| carbamate kinase [Staphylococcus aureus]
>gi|7162015|emb|CAB76628.1| carbamate kinase [Staphylococcus aureus]
>gi|7162013|emb|CAB76627.1| carbamate kinase [Staphylococcus aureus]
>gi|7162011|emb|CAB76626.1| shikimate dehydrogenease [Staphylococcus aureus]
>gi|7162009|emb|CAB76625.1| shikimate dehydrogenease [Staphylococcus aureus]
>gi|7162007|emb|CAB76624.1| shikimate dehydrogenease [Staphylococcus aureus]
>gi|7162005|emb|CAB76623.1| shikimate dehydrogenease [Staphylococcus aureus]
>gi|7162003|emb|CAB76622.1| shikimate dehydrogenease [Staphylococcus aureus]
>gi|7162001|emb|CAB76621.1| shikimate dehydrogenease [Staphylococcus aureus]
>gi|7161999|emb|CAB76620.1| shikimate dehydrogenease [Staphylococcus aureus]
>gi|7161997|emb|CAB76619.1| shikimate dehydrogenease [Staphylococcus aureus]
>gi|7161995|emb|CAB76618.1| shikimate dehydrogenease [Staphylococcus aureus]
>gi|7161993|emb|CAB76617.1| shikimate dehydrogenease [Staphylococcus aureus]
>gi|7161991|emb|CAB76616.1| shikimate dehydrogenease [Staphylococcus aureus]
>gi|7161989|emb|CAB76615.1| shikimate dehydrogenease [Staphylococcus aureus]
>gi|7161987|emb|CAB76614.1| shikimate dehydrogenease [Staphylococcus aureus]
>gi|7161985|emb|CAB76613.1| shikimate dehydrogenease [Staphylococcus aureus]
>gi|7161983|emb|CAB76612.1| shikimate dehydrogenease [Staphylococcus aureus]
>gi|7161981|emb|CAB76611.1| shikimate dehydrogenease [Staphylococcus aureus]
>gi|7161979|emb|CAB76610.1| glycerol kinase [Staphylococcus aureus]
>gi|7161977|emb|CAB76609.1| glycerol kinase [Staphylococcus aureus]
>gi|7161975|emb|CAB76608.1| glycerol kinase [Staphylococcus aureus]
>gi|7161973|emb|CAB76607.1| glycerol kinase [Staphylococcus aureus]
>gi|7161971|emb|CAB76606.1| glycerol kinase [Staphylococcus aureus]
>gi|7161969|emb|CAB76605.1| glycerol kinase [Staphylococcus aureus]
>gi|7161967|emb|CAB76604.1| glycerol kinase [Staphylococcus aureus]
>gi|7161965|emb|CAB76603.1| glycerol kinese [Staphylococcus aureus]
>gi|7161963|emb|CAB76602.1| glycerol kinase [Staphylococcus aureus]
>gi|7161961|emb|CAB76601.1| glycerol kinase [Staphylococcus aureus]
>gi|7161959|emb|CAB76600.1| glycerol kinase [Staphylococcus aureus]
>gi|7161957|emb|CAB76599.1| guanylate kinase [Staphylococcus aureus]
>gi|7161955|emb|CAB76598.1| guanylate kinase [Staphylococcus aureus]
>gi|7161953|emb|CAB76597.1| guanylate kinase [Staphylococcus aureus]
>gi|7161951|emb|CAB76596.1| guanylate kinase [Staphylococcus aureus]
>gi|7161949|emb|CAB76595.1| guanylate kinase [Staphylococcus aureus]
>gi|7161947|emb|CAB76594.1| guanylate kinase [Staphylococcus aureus]
>gi|7161945|emb|CAB76593.1| guanylate kinase [Staphylococcus aureus]
>gi|7161943|emb|CAB76592.1| guanylate kinase [Staphylococcus aureus]
>gi|7161941|emb|CAB76591.1| guanylate kinase [Staphylococcus aureus]
>gi|7161939|emb|CAB76590.1| guanylate kinase [Staphylococcus aureus]
>gi|7161937|emb|CAB76589.1| guanylate kinase [Staphylococcus aureus]
>gi|7161927|emb|CAB76588.1| acetyl coenzyme A acetyltransferase [Staphylococcus aureus]
>gi|7161925|emb|CAB76587.1| acetyl coenzyme A acetyltransferase [Staphylococcus aureus]
>gi|7161923|emb|CAB76586.1| acetyl coenzyme A acetyltransferase [Staphylococcus aureus]
>gi|7161921|emb|CAB76585.1| acetyl coenzyme A acetyltransferase [Staphylococcus aureus]
>gi|7161919|emb|CAB76584.1| acetyl coenzyme A acetyltransferase [Staphylococcus aureus]
>gi|7161917|emb|CAB76583.1| acetyl coenzyme A acetyltransferase [Staphylococcus aureus]
>gi|7161915|emb|CAB76582.1| acetyl coenzyme A acetyltransferase [Staphylococcus aureus]
>gi|7161913|emb|CAB76581.1| acetyl coenzyme A acetyltransferase [Staphylococcus aureus]
>gi|7161911|emb|CAB76580.1| acetyl coenzyme A acetyltransferase [Staphylococcus aureus]
>gi|7161909|emb|CAB76579.1| acetyl coenzyme A acetyltransferase [Staphylococcus aureus]
>gi|7161907|emb|CAB76578.1| acetyl coenzyme A acetyltransferase [Staphylococcus aureus]
>gi|7161905|emb|CAB76577.1| acetyl coenzyme A acetyltransferase [Staphylococcus aureus]
>gi|7161903|emb|CAB76576.1| acetyl coenzyme A acetyltransferase [Staphylococcus aureus]
>gi|7161901|emb|CAB76575.1| acetyl coenzyme A acetyltransferase [Staphylococcus aureus]
>gi|7161899|emb|CAB76574.1| acetyl coenzyme A acetyltransferase [Staphylococcus aureus]
>gi|7161897|emb|CAB76573.1| acetyl coenzyme A acetyltransferase [Staphylococcus aureus]
>gi|7161887|emb|CAB76839.1| Catalase [Staphylococcus aureus]
>gi|7161885|emb|CAB76840.1| Catalase [Staphylococcus aureus]
>gi|7107452|gb|AAF36410.1|AF235026_1 pyruvate dehydrogenase beta subunit PdhB [Staphylococcus
aureus]
>gi|7106008|emb|CAB75987.1| ornithine carbamoyltransferase Otc6850 [Staphylococcus aureus]
>gi|7106004|emb|CAB75985.1| extracellular matrix and plasma binding protein [Staphylococcus aureus]
>gi|7106002|emb|CAB75984.1| extracellular matrix and plasma binding protein [Staphylococcus aureus]
>gi|7019229|emb|CAB75732.1| bone sialoprotein-binding protein [Staphylococcus aureus]
>gi|6180191|gb|AAF05840.1|AF197058_1 trans-2-enoyl-ACP reductase [Staphylococcus aureus]
>gi|6967305|emb|CAB72943.1| hypothetical protein [Staphylococcus aureus]
>gi|6967304|emb|CAB72942.1| hypothetical protein [Staphylococcus aureus]
>gi|6967303|emb|CAB72941.1| hypothetical protein [Staphylococcus aureus]
>gi|6967301|emb|CAB72940.1| hypothetical protein [Staphylococcus aureus]
>gi|6967300|emb|CAB72939.1| hypothetical protein [Staphylococcus aureus]
>gi|6912039|emb|CAB72261.1| penicillin-binding protein 3 [Staphylococcus aureus]
>gi|2506027|dbj|BAA22600.1| NAG [Staphylococcus aureus]
>gi|577649|dbj|BAA07715.1| preLUKM [Staphylococcus aureus]
>gi|216971|dbj|BAA0630.1| glutamic acid specific protease prepropeptide [Staphylococcus aureus]
>gi|6119707|emb|CAB59570.1| aureolysin [Staphylococcus aureus]
>gi|6119705|emb|CAB59569.1| aureolysin [Staphylococcus aureus]
>gi|6119703|emb|CAB59568.1| aureolysin [Staphylococcus aureus]
>gi|6119701|emb|CAB59567.1| aureolysin [Staphylococcus aureus]
>gi|6729657|emb|CAB67709.1| secretory protein [Staphylococcus aureus]
>gi|6729716|pdb|1BQB|A Chain A, Aureolysin, Staphylococcus aureusMetalloproteinase
>gi|5107600|pdb|1KGG|A Chain A, Structure Of Beta-Lactamase Glu166gln:asn170asp Mutant
>gi|6110605|gb|AAF03894.1|AF193842_1 DNA polymerase I [Staphylococcus aureus]
>gi|6690335|gb|AAF24091.1|AF117259_3 ATP binding protein VgA [Staphylococcus aureus]
>gi|6690334|gb|AAF24090.1|AF117259_2 unknown [Staphylococcus aureus]
>gi|6690333|gb|AAF24089.1|AF117259_1 replication protein [Staphylococcus aureus]
>gi|6690331|gb|AAF24088.1|AF117258_5 hydrolase VgB [Staphylococcus aureus]
>gi|6690330|gb|AAF24087.1|AF117258_4 acetyltransferase Vat [Staphylococcus aureus]
>gi|6690329|gb|AAF24086.1|AF11725B_3 resolvase [Staphylococcus aureus]
>gi|6690328|gb|AAF24085.1|AF117258_2 unknown [Staphylococcus aureus]
>gi|6690327|gb|AAF24084.1|AF117258_1 replication protein RepE [Staphylococcus aureus]
>gi|6689210|emb|CAB65404.1| YycJ protein [Staphylococcus aureus]
>gi|6689209|emb|CAB65403.1| YycI protein [Staphylococcus aureus]
>gi|6689208|emb|CAB65402.1| YycH protein [Staphylococcus aureus]
>gi|6689207|emb|CAB65401.1| VicK protein [Staphylococcus aureus]
>gi|6689206|emb|CAB65400.1| TycG protein [Staphylococcus aureus]
>gi|6689205|emb|CAB65399.1| VicR protein [Staphylococcus aureus]
>gi|6681575|dbj|BAA88759.1| cassette chromosome recombinase B [Staphylococcus aureus]
>gi|6681574|dbj|BAA88758.1| cassette chromosome recombinase A [Staphylococcus aureus]
>gi|6681572|dbj|BAA88757.1| cassette chromosome recombinase B [Staphylococcus aureus]
>gi|6681571|dbj|BAA88756.1| cassette chromosome recombinase A [Staphylococcus aureus]
>gi|6681569|dbj|BAA88755.1| cassette chromosome recombinase B [Staphylococcus aureus]
>gi|6681568|dbj|BAA88754.1| cassette chromosome recombinase A [Staphylococcus aureus]
>gi|6644368|gb|AAF21032.1|AF209197_1 UDP-GlcNAc 2-epimerase [Staphylococcus aureus]
>gi|6594284|dbj|BAA88420.1| ATP-binding protein [Staphylococcus aureus]
>gi|6594283|dbj|BAA88419.1| hydrophobic transmembrane protein [Staphylococcus aureus]
>gi|6594281|dbj|BAA88418.1| MsrSA [Staphylococcus aureus]
>gi|6594280|dbj|BAA88417.1| ATP-binding protein [Staphylococcus aureus]
>gi|6594279|dbj|BAA88416.1| hydrophobic transmembrane protein [Staphylococcus aureus]
>gi|6594277|dbj|BAA88415.1| MsrSA [Staphylococcus aureus]
>gi|6594276|dbj|BAA88414.1| ATP-binding protein [Staphylococcus aureus]
>gi|6594275|dbj|BAA88413.1| hydrophobic transmembrane protein [Staphylococcus aureus]
>gi|6578925|gb|AAF18137.1|AF205033_3 glutamyl-tRNAGln amidotransferase subunit B [Staphylococcus
aureus]
>gi|6578924|gb|AAF18136.1|AF205033_2 glutamyl-tRNAGln amidotransferase subunit A [Staphylococcus
aureus]
>gi|6578923|gb|AAF18135.1|AF205033_1 glutamyl-tRNAGln amidotransferase subunit C [Staphylococcus
aureus]
>gi|4185565|gb|AA009131.1| surface protein Pls [Staphylococcus aureus]
>gi|6492112|gb|AAF14183.1| putative transmembrane protein [Staphylococcus aureus]
>gi|6492111|gb|AAF14182.1|AF105976_1 FemX [Staphylococcus aureus]
>gi|6441050|dbj|BAA86894.1| Drp35 [Staphylococcus aureus]
>gi|6434054|emb|CAB60756.1| permease [Staphylococcus aureus]
>gi|6434053|emb|CAB60755.1| ATP-binding protein [Staphylococcus aureus]
>gi|6434052|emb|CAB60754.1| hypothetical protein [Staphylococcus aureus]
>gi|6434050|emb|CAB60753.1| anthranilate synthase component II [Staphylococcus aureus]
>gi|6434049|emb|CAB60752.1| anthranilate phosphoribosyltransferase [Staphylococcus aureus]
>gi|6434048|emb|CAB60751.1| indole-3-glycerol phosphate synthase [Staphylococcus aureus]
>gi|6434047|emb|CAB60750.1| phosphoriborylanthranilate isomerase [Staphylococcus aureus]
>gi|6434045|emb|CAB60749.1| hypothetical protein [Staphylococcus aureus]
>gi|6434044|emb|CAB60748.1| hypothetical protein [Staphylococcus aureus]
>gi|6434042|emb|CAB60747.1| hypothetical protein [Staphylococcus aureus]
>gi|6434041|emb|CAB60746.1| hypothetical protein [Staphylococcus aureus]
>gi|6434040|emb|CAB60745.1| hypothetical protein [Staphylococcus aureus]
>gi|6434038|emb|CAB60744.1| hypothetical protein [Staphylococcus aureus]
>gi|6434037|emb|CAB60743.1| thioredoxine reductase [Staphylococcus aureus]
>gi|6434035|emb|CAB60742.1| hypothetical protein [Staphylococcus aureus]
>gi|6434034|emb|CAB60741.1| hypothetical protein [Staphylococcus aureus]
>gi|6434033|emb|CAB60740.1| thioredoxine reductase [Staphylococcus aureus]
>gi|6434031|emb|CAB60739.1| porphobilinogen synthase [Staphylococcus aureus]
>gi|6434030|emb|CAB60738.1| GSA-1-aminotransferase [Staphylococcus aureus]
>gi|6434029|emb|CAB60737.1| yhjN protein [Staphylococcus aureus]
>gi|6434028|emb|CAB60736.1| DNA-3-methyladenine glycosidase [Staphylococcus aureus]
>gi|6273682|emb|CAA73924.1| transposase [Staphylococcus aureus]
>gi|6273681|emb|CAA73923.1| resolvase [Staphylococcus aureus]
>gi|6273680|emb|CAA73922.1| hypothetical protein [Staphylococcus aureus]
>gi|6273679|emb|CAA73921.1| rRNA methylase [Staphylococcus aureus]
>gi|6273678|emb|CAA73925.1| hypothetical protein [Staphylococcus aureus]
>gi|6166144|sp|Q53665|DPO3_STAAU DNA POLYMERASE III, ALPHA CHAIN POLC-TYPE (POLIII)
>gi|3915801|sp|P50073|PARC_STAAU TOPOISOMERASE IV SUBUNIT A
>gi|2507345|sp|P47770|RPOC_STAAU DNA-DIRECTED RNA POLYMERASE BETA' CHAIN (TRANSCRIPTASE BETA' CHAIN)
(RNA POLYMERASE BETA' SUBUNIT)
>gi|135552|sp|P02983|TCR_STAAU TETRACYCLINE RESISTANCE PROTEIN
>gi|130885|sp|P22490|PRE2_STAAU PLASMID RECOMBINATION ENZYME (MOBILIZATION PROTEIN)
>gi|128852|sp|P00644|NUC_STAAU THERMONUCLEASE PRECURSOR (TNASE) (MICROCOCCAL NUCLEASE)
(STAPHYLOCOCCAL NUCLEASE)
>gi|126941|sp|P26597|MECR_STAEP METHICILLIN RESISTANCE MECR1 PROTEIN
>gi|6137706|pdb|1QTF|A Chain A, Crystal Structure Of Exfoliative Toxin B
>gi|4557981|pdb|1SBB|D Chain D, T-Cell Receptor Beta Chain Complexed With Superantigen Seb
>gi|4557979|pdb|1SBB|B Chain B, T-Cell Receptor Beta Chain Complexed With Superantigen Seb
>gi|3025223|sp|Q53719|YLY1_STAAU HYPOTHETICAL 18.6 KD PROTEIN IN LYTA 3′REGION (ORF1)
>gi|2811052|sp|O07319|YLLB_STAAU HYPOTHETICAL 17.4 KD PROTEIN
>gi|2500373|sp|Q53602|YBXF_STAAU PROBABLE RIBOSOMAL PROTEIN IN RPSL 5′REGION
>gi|2226349|gb|AAB61744.1| CspC [Staphylococcus aureus]
>gi|2226347|gb|AAB61743.1| CspB [Staphylococcus aureus]
>gi|1723202|sp|P55177|YAG5_STAAU HYPOTHETICAL 29.8 KD PROTEIN IN AGR OPERON (ORF 5)
>gi|1176334|sp|P41370|YIL2_STAAU HYPOTHETICAL PROTEIN IN ILES 3′REGION (ORF C)
>gi|1176333|sp|P41369|YIL1_STAAU HYPOTHETICAL PROTEIN IN ILES 5′REGION (ORF B)
>gi|141232|sp|P03860|YPCD_STAAU HYPOTHETICAL 8.7 KD PROTEIN (READING FRAME D)
>gi|141195|sp|P14503|YP2C_STAAU HYPOTHETICAL 27.7 KD PROTEIN
>gi|141194|sp|P12052|YP2B_STAAU HYPOTHETICAL 27.0 KD PROTEIN (ORFD)
>gi|141193|sp|P03866|YP2A_STAAU HYPOTHETICAL 26.9 KD PROTEIN (HYPOTHETICAL PROTEIN C)
>gi|141190|sp|P23217|YP23_STAAU HYPOTHETICAL TRANSCRIPTIONAL REGULATOR IN QACA 5′REGION (ORF 188)
>gi|141183|sp|P13977|YP15_STAAU HYPOTHETICAL 15.5 KD PROTEIN
>gi|141180|sp|P03859|YP12_STAAU HYPOTHETICAL 12.4 KD PROTEIN (READING FRAME C)
>gi|141043|sp|P08655|YMER_STAAU HYPOTHETICAL 19.7 KD PROTEIN IN MERCURIC RESISTANCE OPERON
>gi|140780|sp|P21224|YHLB_STAAU HYPOTHETICAL PROTEIN IN HLB 3′REGION
>gi|1718087|sp|P26839|VATA_STAAU VIRGINIAMYCIN A ACETYLTRANSFERASE
>gi|138137|sp|P17978|VGB_STAAU VIRGINIAMYCIN B HYDROLASE (VGB)
>gi|136457|sp|P06886|TSST_STAAU TOXIC SHOCK SYNDROME TOXIN-1 PRECURSOR (TSST-1)
>gi|6094457|sp|Q53770|TETM_STAAU TETRACYCLINE RESISTANCE PROTEIN TETM (TETA(M))
>gi|6093662|sp|Q53596|PCP_STAAU PYRROLIDONE-CARBOXYLATE PEPTIDASE (5-OXOPROLYL-PEPTIDASE)
(PYROGLUTAMYL-PEPTIDASE I) (PGP-I) (PYRASE)
>gi|3915844|sp|O33276|RRF_STAAU PROBABLE RIBOSOME RECYCLING FACTOR (RIBOSOME RELEASING FACTOR) (RRF)
>gi|3915057|sp|O32422|SYH_STAAU HISTIDYL-TRNA SYNTHETASE (HISTIDINE--TRNA LIGASE) (HISRS)
>gi|3914612|sp|O50581|RECG_STAAU ATP-DEPENDENT DNA HELICASE RECG
>gi|3287914|sp|P81297|STPA_STAAU STAPHOPAIN
>gi|3122859|sp|O08387|SECY_STAAU PREPROTEIN TRANSLOCASE SECY SUBUNIT
>gi|3122722|sp|O06444|RL30_STAAU 50S RIBOSOMAL PROTEIN L30
>gi|3024594|sp|O06442|SECE_STAAU PREPROTEIN TRANSLOCASE SECE SUBUNIT
>gi|3024540|sp|O06443|RL11_STAAU 50S RIBOSOMAL PROTEIN L11
>gi|3024239|sp|O08386|NUSG_STAAU TRANSCRIPTION ANTITERMINATION PROTEIN NUSG
>gi|2501053|sp|P95689|SYS_STAAU SERYL-TRNA SYNTHETASE (SERINE--TRNA LIGASE) (SERRS)
>gi|2501020|sp|Q53638|SYK_STAAU LYSYL-TRNA SYNTHETASE (LYSINE--TRNA LIGASE) (LYSRS)
>gi|2500269|sp|O06445|RL15_STAAU 50S RIBOSOMAL PROTEIN L15
>gi|1710069|sp|P29232|RECF_STAAU RECF PROTEIN
>gi|1709892|sp|P02907|PTHP_STAAU PHOSPHOCARRIER PROTEIN HPR (HISTIDINE-CONTAINING PROTEIN)
>gi|1709733|sp|P51065|PPCK_STAAU PHOSPHOENOLPYRUVATE CARBOXYKINASE [ATP]
>gi|1709584|sp|P50072|PARE_STAAU TOPOISOMERASE IV SUBUNIT B
>gi|1709245|sp|P50588|NDK_STAAU NUCLEOSIDE DIPHOSPHATE KINASE (NDK) (NDP KINASE)
>gi|1351009|sp|P48940|RS7_STAAU 30S RIBOSOMAL PROTEIN S7
>gi|1350927|sp|P48942|RS12_STAAU 30S RIBOSOMAL PROTEIN S12
>gi|1350849|sp|P47768|RPOB_STAAU DNA-DIRECTED RNA POLYMERASE BETA CHAIN (TRANSCRIPTASE BETA CHAIN)
(RNA POLYMERASE BETA SUBUNIT)
>gi|1350771|sp|P48860|RL7_STAAU 50S RIBOSOMAL PROTEIN L7/L12
>gi|1346789|sp|P03864|PRE3_STAAU PLASMID RECOMBINATION ENZYME (MOBILIZATION PROTEIN)
>gi|1346788|sp|P03857|PRE1_STAAU PLASMID RECOMBINATION ENZYME (MOBILIZATION PROTEIN)
>gi|1175774|sp|P45557|PRMA_STAAU PROBABLE METHYLTRANSFERASE
>gi|1174516|sp|P41368|SYIP_STAAU ISOLEUCYL-TRNA SYNTHETASE, MUPIROCIN RESISTANT (ISOLEUCINE--TRNA
LIGASE) (ILERS) (MUPIROCIN RESISTANCE PROTEIN)
>gi|1172527|sp|P45723|PLC_STAAU 1-PHOSPHATIDYLINOSITOL PHOSPHODIESTERASE PRECURSOR
(PHOSPHATIDYLINOSITOL-SPECIFIC PHOSPHOLIPASE C) (PI-PLC)
>gi|586104|sp|P37376|TNPF_STAAU TRANSPOSASE FOR TRANSPOSON TN554 HOMOLOG
>gi|586103|sp|P37375|TNPE_STAAU TRANSPOSASE B (TRANSPOSON TN554 HOMOLOG)
>gi|586027|sp|P38507|SPA2_STAAU IMMUNOGLOBULIN G BINDING PROTEIN A PRECURSOR (IGG BINDING PROTEIN A)
>gi|548620|sp|P17875|PTMA_STAAU PTS SYSTEM, MANNITOL-SPECIFIC IIA COMPONENT (EIIA-MTL) (MANNITOL-
PERMEASE IIA COMPONENT) (PHOSPHOTRANSFERASE ENZYME II, A COMPONENT) (EIII-MTL)
>gi|548619|sp||PTMA_STAAU_2 [Segment 2 of 2] PTS SYSTEM, MANNITOL-SPECIFIC IIA COMPONENT (EIIA-MTL)
(MANNITOL-PERMEASE IIA COMPONENT) (PHOSPHOTRANSFERASE ENZYME II, A COMPONENT) (EIII-MTL)
>gi|548618|sp||PTMA_STAAU_1 [Segment 1 of 2] PTS SYSTEM, MANNITOL-SPECIFIC IIA COMPONENT (EIIA-MTL)
(MANNITOL-PERMEASE IIA COMPONENT) (PHOSPHOTRANSFERASE ENZYME II, A COMPONENT) (EIII-MTL)
>gi|400965|sp|P31337|RADC_STAAU DNA REPAIR PROTEIN RADC HOMOLOG (25 KD PROTEIN)
>gi|136146|sp|P06698|TRAC_STAAU TRANSPOSASE FOR TRANSPOSON TN554
>gi|136133|sp|P18416|TRA3_STAAU TRANSPOSASE FOR TRANSPOSON TN552 (ORF 480)
>gi|135956|sp|P06697|TNPB_STAAU TRANSPOSASE B (TRANSPOSON TN554)
>gi|135955|sp|P06696|TNPA_STAAU TRANSPOSASE A (TRANSPOSON TN554)
>gi|135949|sp|P19775|TRA6_STAAU TRANSPOSASE FOR INSERTION SEQUENCE ELEMENT IS256 IN TRANSPOSON
TN4001
>gi|135248|sp|P23736|T2S9_STAAU TYPE II RESTRICTION ENZYME SAU96I (ENDONUCLEASE SAU96I) (R.SAU96I)
>gi|135247|sp|P16667|T2S3_STAAU TYPE II RESTRICTION ENZYME SAU3AI (ENDONUCLEASE SAU3AI) (R.SAU3AI)
>gi|135003|sp|P04188|STSP_STAAU GLUTAMYL ENDOPEPTIDASE PRECURSOR (STAPHYLOCOCCAL SERINE PROTEINASE)
(V8 PROTEINASE) (ENDOPROTEINASE GLU-C)
>gi|135002|sp|P12055|STR_STAAU STREPTOMYCIN RESISTANCE PROTEIN
>gi|134959|sp|P17855|STC2_STAAU STAPHYLOCOAGULASE PRECURSOR
>gi|134958|sp|P07767|STC1_STAAU STAPHYLOCOAGULASE PRECURSOR
>gi|134189|sp|P00802|SAK_STAAU STAPHYLOKINASE PRECURSOR (NEUTRAL PROTEINASE) (PROTEASE III)
>gi|134150|sp|P04827|S3AD_STAAU STREPTOMYCIN 3′ ′-ADENYLYLTRANSFERASE (AAD(9))
>gi|133479|sp|P26766|RPSD_STAAU RNA POLYMERASE SIGMA FACTOR RPOD
>gi|133112|sp|P14491|RLX3_STAAU RLX PROTEIN
>gi|133111|sp|P03865|RLX2_STAAU RLX PROTEIN
>gi|133109|sp|P12054|RLX1_STAAU RLX PROTEIN
>gi|132380|sp|P08115|REP_STAAU REPLICATION INITIATION PROTEIN
>gi|132374|sp|P03858|REPY_STAAU REPLICATION PROTEIN
>gi|132372|sp|P03862|REPX_STAAU REP PROTEIN (REPLICATION PROTEIN) (READING FRAME A)
>gi|132369|sp|P19529|REPN_STAAU REPLICATION INITIATION PROTEIN
>gi|132368|sp|P14490|REPM_STAAU REPLICATION INITIATION PROTEIN
>gi|132364|sp|P12053|REPE_STAAU REPLICATION INITIATION PROTEIN
>gi|132362|sp|P03065|REPD_STAAU REPLICATION INITIATION PROTEIN
>gi|132361|sp|P03064|REPC_STAAU REPLICATION INITIATION PROTEIN (PROTEIN A)
>gi|132357|sp|P05061|REPB_STAAU REPLICATION PROTEIN
>gi|132322|sp|P13969|REMA_STAAU REPLICATION AND MAINTENANCE PROTEIN (PLASMID REPLICATION PROTEIN)
>gi|131518|sp|P02909|PTLA_STAAU PTS SYSTEM, LACTOSE-SPECIFIC IIA COMPONENT (EIIA-LAC) (LACTOSE-
PERMEASE IIA COMPONENT) (PHOSPHOTRANSFERASE ENZYME II, A COMPONENT) (EIII-LAC)
>gi|131497|sp|P11162|PTLB_STAAU PTS SYSTEM, LACTOSE-SPECIFIC IIBC COMPONENT (EIIBC-LAC) (LACTOSE-
PERMEASE IIBC COMPONENT) (PHOSPHOTRANSFERASE ENZYME II, BC COMPONENT) (EII-LAC)
>gi|130085|sp|P09978|PHLC_STAAU PHOSPHOLIPASE C PRECURSOR (BETA-HEMOLYSIN) (BETA-TOXIN)
(SPHINGOMYELINASE)
>gi|129676|sp|P07944|PBP_STAAU BETA-LACTAM-INDUCIBLE PENICILLIN-BINDING PROTEIN
>gi|129132|sp|P21223|OMP7_STAAU 70 KD OUTER MEMBRANE PROTEIN PRECURSOR
>gi|129123|sp|P21222|NP30_STAAU 30 KD NEUTRAL PHOSPHATASE (NPTASE)
>gi|128511|sp|P21191|NORA_STAAU QUINOLONE RESISTANCE NORA PROTEIN
>gi|127486|sp|P23737|MTS9_STAAU MODIFICATION METHYLASE SAU96I (CYTOSINE-SPECIFIC METHYLTRANSFERASE
SAU96I) (M.SAU96I)
>gi|127485|sp|P16668|MTS3_STAAU MODIFICATION METHYLASE SAU3AI (CYTOSINE-SPECIFIC METHYLTRANSFERASE
SAU3AI) (M.SAU3AI)
>gi|6016606|sp|O68285|MSCL_STAAU LARGE-CONDUCTANCE MECHANOSENSITIVE CHANNEL
>gi|6016162|sp|O34092|GSA_STAAU GLUTAMATE-1-SEMIALDEHYDE 2,1-AMINOMUTASE (GSA) (GLUTAMATE-1-
SEMIALDEHYDE AMINOTRANSFERASE) (GSA-AT)
>gi|4033454|sp|P72364|LEPH_STAAU INACTIVE SIGNAL PEPTIDASE IA
>gi|4033452|sp|P72365|LEP_STAAU SIGNAL PEPTIDASE IB (SPASE IB) (LEADER PEPTIDASE IB)
>gi|3122102|sp|O07325|FTSA_STAAU CELL DIVISION PROTEIN FTSA
>gi|1709003|sp|P50307|METK_STAAU S-ADENOSYLMETHIONINE SYNTHETASE (METHIONINE ADENOSYLTRANSFERASE)
(ADOMET SYNTHETASE)
>gi|1708807|sp|P52282|LGT_STAAU PROLIPOPROTEIN DIACYLGLYCERYL TRANSFERASE
>gi|1707902|sp|P31714|GHM2_STAAU GAMMA-HEMOLYSIN H-GAMMA-II SUBUNIT
>gi|1707901|sp||GHM2_STAAU_2 [Segment 2 of 2] GAMMA-HEMOLYSIN H-GAMMA-II SUBUNIT
>gi|1707900|sp||GHM2_STAAU_1 [Segment 1 of 2] GAMMA-HEMOLYSIN H-GAMMA-II SUBUNIT
>gi|1346584|sp|P80544|MRSP_STAAU METHICILLIN-RESISTANT SURFACE PROTEIN
>gi|1346583|sp||MRSP_STAAU_7 [Segment 7 of 7] METHICILLIN-RESISTANT SURFACE PROTEIN
>gi|1346582|sp||MRSP_STAAU_6 [Segment 6 of 7] METHICILLIN-RESISTANT SURFACE PROTEIN
>gi|1346581|sp||MRSP_STAAU_5 [Segment 5 of 7] METHICILLIN-RESISTANT SURFACE PROTEIN
>gi|1346580|sp||MRSP_STAAU_4 [Segment 4 of 7] METHICILLIN-RESISTANT SURFACE PROTEIN
>gi|1346579|sp||MRSP_STAAU_3 [Segment 3 of 7] METHICILLIN-RESISTANT SURFACE PROTEIN
>gi|1346578|sp||MRSP_STAAU_2 [Segment 2 of 7] METHICILLIN-RESISTANT SURFACE PROTEIN
>gi|1346577|sp||MRSP_STAAU_1 [Segment 1 of 7] METHICILLIN-RESISTANT SURFACE PROTEIN
>gi|1346244|sp|P20832|GYRB_STAAU DNA GYRASE SUBUNIT B
>gi|1346238|sp|P20831|GYRA_STAAU DNA GYRASE SUBUNIT A
>gi|1176137|sp|P45556|HRCA_STAAU HEAT-INDUCIBLE TRANSCRIPTION REPRESSOR HRCA
>gi|1169769|sp|P45498|FTSZ_STAAU CELL DIVISION PROTEIN FTSZ
>gi|547868|sp|P36884|LPCA_STAAU CHLORAMPHENICOL RESISTANCE LEADER PEPTIDE
>gi|400204|sp|P31716|LUKS_STAAU LEUKOCIDIN S SUBUNIT PRECURSOR
>gi|400203|sp|P31715|LUKF_STAAU LEUKOCIDIN F SUBUNIT PRECURSOR (GAMMA-HEMOLYSIN, H-GAMMA-I SUBUNIT)
>gi|127020|sp|P08656|MERT_STAAU MERCURIC TRANSPORT PROTEIN (MERCURY ION TRANSPORT PROTEIN)
>gi|127015|sp|P22874|MERR_STAAU MERCURIC RESISTANCE OPERON REGULATORY PROTEIN
>gi|126999|sp|P08653|MERB_STAAU ALKYLMERCURY LYASE (ORGANOMERCURIAL LYASE)
>gi|126995|sp|P08663|MERA_STAAU MERCURIC REDUCTASE (HG(II) REDUCTASE)
>gi|126940|sp|P26598|MECI_STAEP METHICILLIN RESISTANCE REGULATORY PROTEIN MECI
>gi|126446|sp|P03063|LPRM_STAAU 23S RRNA METHYLASE LEADER PEPTIDE (ERYTHROMYCIN RESISTANCE LEADER
PEPTIDE)
>gi|126333|sp|P10335|LIP_STAAU LIPASE PRECURSOR (GLYCEROL ESTER HYDROLASE)
>gi|125937|sp|P16644|LACR_STAAU LACTOSE PHOSPHOTRANSFERASE SYSTEM REPRESSOR
>gi|125930|sp|P11175|LACG_STAAU 6-PHOSPHO-BETA-GALACTOSIDASE (BETA-D-PHOSPHOGALACTOSIDE
GALACTOHYDROLASE) (PGALASE) (P-BETA-GAL) (PBG)
>gi|125908|sp|P26594|LACA_STAAU GALACTOSE-6-PHOSPHATE ISOMERASE LACA SUBUNIT
>gi|125464|sp|P00554|KKA3_ENTFA AMINOGLYCOSIDE 3′-PHOSPHOTRANSFERASE (KANAMYCIN KINASE, TYPE III) (NEOMYCIN-
KANAMYCIN PHOSPHOTRANSFERASE, TYPE III) (APH(3′)III)
>gi|125191|sp|P05057|KANU_STAAU KANAMYCIN NUCLEOTIDYLTRANSFERASE (NEO(R))
>gi|123184|sp|P09616|HLA_STAAU ALPHA-HEMOLYSIN PRECURSOR (ALPHA-TOXIN) (ALPHA-HL)
>gi|6015099|sp|O69174|ENO_STAAU ENOLASE (2-PHOSPHOGLYCERATE DEHYDRATASE) (2-PHOSPHO-D-GLYCERATE
HYDRO-LYASE) (LAMININ BINDING PROTEIN)
>gi|6014977|sp|Q59822|DLDH_STAAU DIHYDROLIPOAMIDE DEHYDROGENASE (E3 COMPONENT OF PYRUVATE COMPLEX)
(MEMBRANE-BOUND RIBOSOME PROTEIN COMPLEX 50 KD SUBUNIT)
>gi|6014729|sp|P81684|CS40_STAAU 40 KD VITRONECTIN-BINDING CELL SURFACE PROTEIN
>gi|3023644|sp|O05701|DHPS_STAAU DIHYDROPTEROATE SYNTHASE (DIHYDROPTEROATE PYROPHOSPHORYLASE) (DHPS)
>gi|2829402|sp|P49994|DNAA_STAAU CHROMOSOMAL REPLICATION INITIATOR PROTEIN DNAA
>gi|1706496|sp|P50029|DP3B_STAAU DNA POLYMERASE III, BETA CHAIN
>gi|1169381|sp|P45554|DNAK_STAAU DNAK PROTEIN (HEAT SHOCK PROTEIN 70) (HSP70)
>gi|544199|sp|P10167|DYRB_STAAU DIHYDROFOLATE REDUCTASE TYPE I
>gi|462026|sp|P34071|ETC2_STAAU ENTEROTOXIN TYPE C-2 PRECURSOR (SEC2)
>gi|127195|sp|P13978|ERM4_STAAU RRNA ADENINE N-6-METHYLTRANSFERASE (MACROLIDE-LINCOSAMIDE-
STREPTOGRAMIN B RESISTANCE PROTEIN) (ERYTHROMYCIN RESISTANCE PROTEIN)
>gi|127194|sp|P13957|ERM3_STAAU RRNA ADENINE N-6-METHYLTRANSFERASE (MACROLIDE-LINCOSAMIDE-
STREPTOGRAMIN B RESISTANCE PROTEIN) (ERYTHROMYCIN RESISTANCE PROTEIN)
>gi|127191|sp|P06699|ERM1_STAAU RRNA ADENINE N-6-METHYLTRANSFERASE (MACROLIDE-LINCOSAMIDE-
STREPTOGRAMIN B RESISTANCE PROTEIN) (ERYTHROMYCIN RESISTANCE PROTEIN)
>gi|119903|sp|P14305|FEMB_STAAU POSSIBLE PROTEIN FEMB (ORF 419)
>gi|119902|sp|P14304|FEMA_STAAU FACTOR ESSENTIAL FOR EXPRESSION OF METHICILLIN RESISTANCE
>gi|119655|sp|P12993|ETXE_STAAU ENTEROTOXIN TYPE E PRECURSOR (SEE)
>gi|119654|sp|P20723|ETXD_STAAU ENTEROTOXIN TYPE D PRECURSOR (SED)
>gi|119653|sp|P01552|ETXB_STAAU ENTEROTOXIN TYPE B PRECURSOR (SEB)
>gi|119652|sp|P13163|ETXA_STAAU ENTEROTOXIN TYPE A PRECURSOR (SEA)
>gi|119626|sp|P23313|ETC3_STAAU ENTEROTOXIN TYPE C-3 PRECURSOR (SEC3)
>gi|119625|sp|P01553|ETC1_STAAU ENTEROTOXIN TYPE C-1 PRECURSOR (SEC1)
>gi|119624|sp|P09332|ETB_STAAU EXFOLIATIVE TOXIN B PRECURSOR (EPIDERMOLYTIC TOXIN B)
>gi|119621|sp|P09331|ETA_STAAU EXFOLIATIVE TOXIN A PRECURSOR (EPIDERMOLYTIC TOXIN A)
>gi|119131|sp|P24121|EDIN_STAAU EPIDERMAL CELL DIFFERENTIATION INHIBITOR PRECURSOR (EDIN)
>gi|118976|sp|P13955|DYRA_STAAU DIHYDROFOLATE REDUCTASE TYPE I (TN4003)
>gi|5813905|gb|AAD52059.1|AF086783_7 glycerol esther hydrolase [Staphylococcus aureus]
>gi|5813904|gb|AAD52058.1|AF086783_6 IcaC [Staphylococcus aureus]
>gi|5813903|gb|AAD52057.1|AF086783_5 IcaB [Staphylococcus aureus]
>gi|5813902|gb|AAD52056.1|AF086783_4 IcaD [Staphylococcus aureus]
>gi|5813901|gb|AAD52055.1|AF086783_3 IcaA [Staphylococcus aureus]
>gi|5813900|gb|AAD52054.1|AF086783_2 IcaR [Staphylococcus aureus]
>gi|5813899|gb|AAD52053.1|AF086783_1 CapA [Staphylococcus aureus]
>gi|6002652|gb|AAF00080.1|AF095597_1 ferric uptake regulator homolog [Staphylococcus aureus]
>gi|6002650|gb|AAF00079.1|AF055596_1 ferric uptake regulator homolog [Staphylococcus aureus]
>gi|6002648|gb|AAF00078.1|AF095595_1 ferric uptake regulator homolog [Staphylococcus aureus]
>gi|3913259|sp|Q53654|CNA_STAAU COLLAGEN ADHESIN PRECURSOR
>gi|584919|sp|Q08841|CH10_STAAU 10 KD CHAPERONIN (PROTEIN CPN10) (PROTEIN GROES) (HEAT SHOCK PROTEIN
10)
>gi|116920|sp|P25921|COP6_STAAU COP-6 PROTEIN
>gi|1168660|sp|P20384|BIN3_STAAU POTENTIAL DNA-INVERTASE BIN3 (TRANSPOSON TN552)
>gi|729029|sp|P39861|CAPL_STAAU CAPL PROTEIN
>gi|729028|sp|P39860|CAPK_STAAU CAPK PROTEIN
>gi|729027|sp|P39859|CAPJ_STAAU CAPJ PROTEIN
>gi|729025|sp|P39857|CAPH_STAAU CAPH PROTEIN
>gi|729024|sp|P39856|CAPG_STAAU CAPG PROTEIN
>gi|729021|sp|P39855|CAPF_STAAU CAPF PROTEIN
>gi|729020|sp|P39854|CAPE_STAAU CAPE PROTEIN
>gi|729019|sp|P39853|CAPD_STAAU CAPD PROTEIN
>gi|729018|sp|P39852|CAPC_STAAU CAPC PROTEIN
>gi|729017|sp|P39851|CAPB_STAAU CAPB PROTEIN
>gi|729016|sp|P39850|CAPA_STAAU CAPA PROTEIN
>gi|584871|sp|P37374|CADF_STAAU CADMIUM EFFLUX SYSTEM ACCESSORY PROTEIN HOMOLOG
>gi|584870|sp|P37386|CADD_STAAU PROBABLE CADMIUM-TRANSPORTING ATPASE (CADMIUM EFFLUX ATPASE)
>gi|543935|sp|P36883|CAT5_STAAU CHLORAMPHENICOL ACETYLTRANSFERASE
>gi|543934|sp|P36882|CAT4_STAAU CHLORAMPHENICOL ACETYLTRANSFERASE
>gi|231569|sp|P30338|ARSR_STAAU ARSENICAL RESISTANCE OPERON REPRESSOR
>gi|231567|sp|P30330|ARSC_STAAU ARSENATE REDUCTASE (ARSENICAL PUMP MODIFIER)
>gi|231565|sp|P30329|ARSB_STAAU ARSENICAL PUMP MEMBRANE PROTEIN
>gi|140340|sp|P20047|CADC_STAAU CADMIUM EFFLUX SYSTEM ACCESSORY PROTEIN
>gi|115688|sp|P06135|CAT3_STAAU CHLORAMPHENICOL ACETYLTRANSFERASE
>gi|115685|sp|P00486|CAT2_STAAU CHLORAMPHENICOL ACETYLTRANSFERASE
>gi|115680|sp|P00485|CAT1_STAAU CHLORAMPHENICOL ACETYLTRANSFERASE
>gi|115414|sp|P20021|CADA_STAAU PROBABLE CADMIUM-TRANSPORTING ATPASE (CADMIUM EFFLUX ATPASE)
>gi|115052|sp|P22491|BLE2_STAAU BLEOMYCIN RESISTANCE PROTEIN
>gi|115051|sp|P13014|BLE1_BACSP BLEOMYCIN RESISTANCE PROTEIN (BRP)
>gi|115049|sp|P18357|BLAR_STAAU REGULATORY PROTEIN BLAR1
>gi|115044|sp|P18415|BLAI_STAAU PENICILLINASE REPRESSOR (REGULATORY PROTEIN BLAI) (BETA-LACTAMASE
REPRESSOR PROTEIN)
>gi|114996|sp|P19241|BINR_STAAU DNA-INVERTASE BINR (TRANSPOSON TN552)
>gi|114995|sp|P18358|BINL_STAAU TRANSPOSON TN552 RESOLVASE
>gi|114300|sp|P18179|ATBP_STAAU POTENTIAL ATP-BINDING PROTEIN (ORF 271)
>gi|3913011|sp|O05204|AHPF_STAAU ALKYL HYDROPEROXIDE REDUCTASE SUBUNIT F
>gi|113527|sp|P13131|AGRA_STAAU ACCESSORY GENE REGULATOR PROTEIN A
>gi|112954|sp|P14507|ANCA_STAAU BIFUNCTIONAL AAC/APH [INCLUDES: 6′-AMINOGLYCOSIDE N-
ACETYLTRANSFERASE (AAC(6′)); 2″-AMINOGLYCOSIDE PHOSPHOTRANSFERASE (APH(2″))]
>gi|1729798|emb|CAA71069.1| CTORF239 [Staphylococcus aureus]
>gi|1729797|emb|CAA71068.1| sigma-B [Staphylococcus aureus]
>gi|1729796|emb|CAA71067.1| rsbW [Staphylococcus aureus]
>gi|1729795|emb|CAA71066.1| rsbV [Staphylococcus aureus]
>gi|1729794|emb|CAA71065.1| rsbU [Staphylococcus aureus]
>gi|1729793|emb|CAA71064.1| ORF136 [Staphylococcus aureus]
>gi|1729792|emb|CAA71063.1| ORF56 [Staphylococcus aureus]
>gi|5834651|emb|CAB55331.1| putative mannitol-specific IIA component [Staphylococcus aureus]
>gi|5834650|emb|CAB55330.1| putative mannitol-1-phosphate 5-dehydrogenase [Staphylococcus aureus]
>gi|5834649|emb|CAB55329.1| Mrp protein [Staphylococcus aureus]
>gi|5834648|emb|CAA71060.2| phosphoglucosamine mutase, GlmM [Staphylococcus aureus]
>gi|5834647|emb|CAB55328.1| hypothetical protein [Staphylococcus aureus]
>gi|5834646|emb|CAB55327.1| hypothetical protein [Staphylococcus aureus]
>gi|5834645|emb|CAB55326.1| arginase [Staphylococcus aureus]
>gi|4775551|emb|CAA71062.1| CTORF1365 [Staphylococcus aureus]
>gi|4775543|emb|CAA70781.1| arginase [Staphylococcus aureus]
>gi|4775542|emb|CAA70780.1| ORF94 [Staphylococcus aureus]
>gi|46695|emb|CAA25094.1| protein A [Staphylococcus aureus]
>gi|5822514|pdb|3LKF|A Chain A, Leukocidin F (Hlgb) From Staphylococcus aureusWith Phosphocholine
Bound
>gi|5822485|pdb|2LKF|A Chain A, Leukocidin F (Hlgb) From Staphylococcus aureus
>gi|5822083|pdb|1LKF|A Chain A, Leukocidin F (Hlgb) From Staphylococcus aureus
>gi|5822030|pdb|1CQV|A Chain A, Crystal Structure Of Staphylococcal Enterotoxin C2 At 100k
>gi|5739084|gb|AAD50329.1|AF077865_1 beta-lactamase [Staphylococcus aureus]
>gi|5726436|gb|AAD48437.1|AF162687_1 sortase [Staphylococcus aureus]
>gi|5726302|gb|AAD48404.1|AF129010_3 CsbB homolog [Staphylococcus aureus]
>gi|5726301|gb|AAD48403.1|AF129010_2 histidine protein kinase SaeS [Staphylococcus aureus]
>gi|5726300|gb|AAD48402.1|AF129010_1 response regulator SaeR [Staphylococcus aureus]
>gi|5690277|gb|AAD47014.1|AF147744_4 transporter [Staphylococcus aureus]
>gi|5690276|gb|AAD47013.1|AF147744_3 lantibiotic modifying enzyme [Staphylococcus aureus]
>gi|5690275|gb|AAD47012.1|AF147744_2 lantibiotic structural protein beta [Staphylococcus aureus]
>gi|5690274|gb|AAD47011.1|AF147744_1 lantibiotic structural protein alpha [Staphylococcus aureus]
>gi|5672689|dbj|BAA13059.2| D-alanine-D-alanyl carrier protein ligase [Staphylococcus aureus]
>gi|1405338|dbj|BAA13062.1| extramembranal protein [Staphylococcus aureus]
>gi|1405337|dbj|BAA13061.1| D-alanyl carrier protein [Staphylococcus aureus]
>gi|1405336|dbj|BAA13060.1| hypothethecal membrane transporter [Staphylococcus aureus]
>gi|1405334|dbj|BAA13058.1| unknown [Staphylococcus aureus]
>gi|5679714|emb|CAB51807.1| cell surface protein map-w [Staphylococcus aureus]
>gi|5531420|emb|CAB50920.1| map-7 protein [Staphylococcus aureus]
>gi|4558750|gb|AAD22731.1| heat shock protein 60 kDa [Staphylococcus aureus]
>gi|4558748|gb|AAD22730.1| heat shock protein 60 kDa [Staphylococcus aureus]
>gi|4558746|gb|AAD22729.1| heat shock protein 60 kDa [Staphylococcus aureus]
>gi|4558744|gb|AAD22728.1| heat shock protein 60 kDa [Staphylococcus aureus]
>gi|4558742|gb|AAD22727.1| heat shock protein 60 kDa [Staphylococcus aureus]
>gi|4558740|gb|AAD22726.1| heat shock protein 60 kDa [Staphylococcus aureus]
>gi|4558738|gb|AAD22725.1| heat shock protein 60 kDa [Staphylococcus aureus]
>gi|4558736|gb|AAD22724.1| heat shock protein 60 kDa [Staphylococcus aureus]
>gi|4558706|gb|AAD22709.1| heat shock protein 60 [Staphylococcus aureus subsp. aureus]
>gi|4558704|gb|AAD22708.1| heat shock protein 60 [Staphylococcus aureus subsp. anaerobius]
>gi|4205743|gb|AAD11256.1| heat shock protein 60 [Staphylococcus aureus]
>gi|5542332|pdb|1PVL| Structure Of The Panton-Valentine Leucocidin F Component From Staphylococcus
Aureus
>gi|2642659|gb|AAB87090.1| UDP-N-acetylmuramoyl-L-alanine synthetase [Staphylococcus aureus]
>gi|1916729|gb|AAB51227.1| CadD [Staphylococcus aureus]
>gi|5441303|gb|AAD43176.1|AF098801_1 penicillin-binding protein Pbp2b [Staphylococcus aureus]
>gi|581566|emb|CAA45142.1| mecR1 [Staphylococcus aureus]
>gi|46615|emb|CAA45143.1| mecI [Staphylococcus aureus]
>gi|46613|emb|CAA45141.1| mecA [Staphylococcus aureus]
>gi|5391440|dbj|BAA82253.1| orf2 [Staphylococcus aureus]
>gi|5391439|dbj|BAA82252.1| orfX [Staphylococcus aureus]
>gi|5391438|dbj|BAA82251.1| orf3 [Staphylococcus aureus]
>gi|5391437|dbj|BAA82250.1| orf1 [Staphylococcus aureus]
>gi|5327232|emb|CAB46341.1| adenine mathylase [Staphylococcus aureus]
>gi|5327231|emb|CAB46340.1| adenine methaylase [Staphylococcus aureus]
>gi|5327230|emb|CAB46339.1| hypothetical protein [Staphylococcus aureus]
>gi|5114231|gb|AAD40238.1|AF136709_2 histidine kinase YycG [Staphylococcus aureus]
>gi|5114230|gb|AAD40237.1|AF136709_1 response regulator YycF [Staphylococcus aureus]
>gi|3767595|dbj|BAA33858.1| ORF4 [Staphylococcus aureus]
>gi|3767594|dbj|BAA33857.1| Eprh [Staphylococcus aureus]
>gi|3767593|dbj|BAA33856.1| LytN [Staphylococcus aureus]
>gi|3767592|dbj|BAA33855.1| ORF1 [Staphylococcus aureus]
>gi|2605638|gb|AAB84174 .1| staphylokinase [Staphylococcus aureus]
>gi|5031413|gb|AAD38159.1|AF151117_1 replication protein [Staphylococcus aureus]
>gi|4930180|pdb|2DHN| Complex Of 7,8-Dihydroneopterin Aldolase From Staphylococcus aureus With 6-
Hydroxymethyl-7,8-Dihydropterin At 2.2 A Resolution
>gi|4930033|pdb|1DHN| 1.65 Angstrom Resolution Structure Of 7,8-Dihydroneopterin Aldolase From
Staphylococcus aureus
>gi|4929299|gb|AAD33940.1|AF144661_1 factor essential for methicillin resistance [Staphylococcus
aureus subsp. anaerobius]
>gi|3892895|emb|CAA75651.1| phosphoglucosamine-mutase [Staphylococcus aureus]
>gi|3892894|emb|CAA75650.1| hypothetical protein [Staphylococcus aureus]
>gi|3892893|emb|CAA75649.1| hypothetical protein [Staphylococcus aureus]
>gi|3892892|emb|CAA75648.1| arginase [Staphylococcus aureus]
>gi|4115707|dbj|BAA36484.1| NorA [Staphylococcus aureus]
>gi|4582216|emb|CAB40191.1| elongation factor G (EF-G) [Staphylococcus aureus]
>gi|4574238|gb|AAD23963.1|AF106851_2 FmhC [Staphylococcus aureus]
>gi|4574237|gb|AAD23962.1|AF106851_1 LytN [Staphylococcus aureus]
>gi|4574235|gb|AAD23961.1|AF106850_1 FmhB [Staphylococcus aureus]
>gi|4574233|gb|AAD23960.1|AF106849_1 FmhA [Staphylococcus aureus]
>gi|4572581|gb|AAD15142.2| Unknown [Staphylococcus aureus]
>gi|3891901|pdb|1CV8| Staphopain, Cysteine Proteinase From Staphylococcus aureus V8
>gi|2981905|pdb|3NUC| Staphlococcal Nuclease, 1-N-Propane Thiol Disulfide To V23c Variant
>gi|2981899|pdb|2NUC| Staphlococcal Nuclease, Ethane Thiol Disulfide To V23c Variant
>gi|1942332|pdb|1SNQ| Protein Stability In Staphylococcal Nuclease
>gi|1942331|pdb|1SNP| Protein Stability In Staphylococcal Nuclease
>gi|1942330|pdb|1SNO| Protein Stability In Staphylococcal Nuclease
>gi|4139848|pdb|1SSN| Staphylokinase, Sakstar Variant, Nmr, 20 Structures
>gi|4139648|pdb|1TS5|B Chain B, I140t Mutant Of Toxic Shock Syndrome Toxin-1 From S. Aureus
>gi|4139647|pdb|1TS5|A Chain A, I140t Mutant Of Toxic Shock Syndrome Toxin-1 From S. Aureus
>gi|4139646|pdb|1TS4|B Chain B, Q139k Mutant Of Toxic Shock Syndrome Toxin-1 From S. Aureus
>gi|4139645|pdb|1TS4|A Chain A, Q139k Mutant Of Toxic Shock Syndrome Toxin-1 From S. Aureus
>gi|4139644|pdb|1TS3|C Chain C, H135a Mutant Of Toxic Shock Syndrome Toxin-1 From S. Aureus
>gi|4139643|pdb|1TS3|B Chain B, H135a Mutant Of Toxic Shock Syndrome Toxin-1 From S. Aureus
>gi|4139642|pdb|1TS3|A Chain A, H135a Mutant Of Toxic Shock Syndrome Toxin-1 From S. Aureus
>gi|4139641|pdb|1TS2|C Chain C, T128a Mutant Of Toxic Shock Syndrome Toxin-1 From S. Aureus
>gi|4139640|pdb|1TS2|B Chain B, T128a Mutant Of Toxic Shock Syndrome Toxin-1 From S. Aureus
>gi|4139639|pdb|1TS2|A Chain A, T128a Mutant Of Toxic Shock Syndrome Toxin-1 From S. Aureus
>gi|3891592|pdb|1AW7|D Chain D, Q136a Mutant Of Toxic Shock Syndrome Toxin-1 From S. Aureus
>gi|3891591|pdb|1AW7|C Chain C, Q136a Mutant Of Toxic Shock Syndrome Toxin-1 From S. Aureus
>gi|3891590|pdb|1AW7|B Chain B, Q136a Mutant Of Toxic Shock Syndrome Toxin-1 From S. Aureus
>gi|3891589|pdb|1AW7|A Chain A, Q136a Mutant Of Toxic Shock Syndrome Toxin-1 From S. Aureus
>gi|4454324|emb|CAA10802.1| MapN protein [Staphylococcus aureus]
>gi|3970797|emb|CAA74099.1| polynucleotide phosphorylase [Staphylococcus aureus]
>gi|4530244|gb|AAD21960.1| putative exoprotein DltD [Staphylococcus aureus]
>gi|4530243|gb|AAD21959.1| D-alanine carrier protein DltC [Staphylococcus aureus]
>gi|4530242|gb|AAD21958.1| putative membrane protein DltB [Staphylococcus aureus]
>gi|4530241|gb|AAD21957.1| D-alanine-D-alanyl carrier protein ligase DltA [Staphylococcus aureus]
>gi|4530240|gb|AAD21956.1| unknown [Staphylococcus aureus]
>gi|4490615|emb|CAB38647.1| triosephosphate isomerase [Staphylococcus aureus]
>gi|4490614|emb|CAB38646.1| phosphoglycerate kinase [Staphylococcus aureus]
>gi|4490613|emb|CAB38645.1| glyceraldehyde-3-phosphate dehydrogenase [Staphylococcus aureus]
>gi|4490612|emb|CAB38644.1| gap regulator [Staphylococcus aureus]
>gi|4490610|emb|CAB38643.1| ribonucleotide reductase minor subunit [Staphylococcus aureus]
>gi|4490609|emb|CAB38642.1| ribonucelotide reductase major subunit [Staphylococcus aureus]
>gi|4490608|emb|CAB38641.1| NRD1 [Staphylococcus aureus]
>gi|4454322|emb|CAA10788.1| hypothetical protein [Staphylococcus aureus]
>gi|4454321|emb|CAA10787.1| hypothetical protein [Staphylococcus aureus]
>gi|4126675|dbj|BAA36689.1| Hypothetical protein [Staphylococcus aureus]
>gi|4126674|dbj|BAA36688.1| Hypothetical protein [Staphylococcus aureus]
>gi|4126673|dbj|BAA36687.1| CzrA [Staphylococcus aureus]
>gi|4126672|dbj|BAA36686.1| czcD [Staphylococcus aureus]
>gi|4126671|dbj|BAA36685.1| Hypothetical protein [Staphylococcus aureus]
>gi|4001731|dbj|BAA35101.1| MnhG [Staphylococcus aureus]
>gi|4001730|dbj|BAA35100.1| MnhF [Staphylococcus aureus]
>gi|4001729|dbj|BAA35099.1| MnhE [Staphylococcus aureus]
>gi|4001728|dbj|BAA35098.1| MnhD [Staphylococcus aureus]
>gi|4001727|dbj|BAA35097.1| MnhC [Staphylococcus aureus]
>gi|4001726|dbj|BAA35096.1| MnhB [Staphylococcus aureus]
>gi|4001725|dbj|BAA35095.1| MnhA [Staphylococcus aureus]
>gi|4001724|dbj|BAA35094.1| OrfA [Staphylococcus aureus]
>gi|3694944|gb|AAC62498.1| SirC [Staphylococcus aureus]
>gi|3694943|gb|AAC62497.1| SirB [Staphylococcus aureus]
>gi|3694942|gb|AAC62496.1| lipoprotein SirA [Staphylococcus aureus]
>gi|4138456|emb|CAA11555.1| Map protein [Staphylococcus aureus]
>gi|4138445|emb|CAA77018.1| adenine methylase [Staphylococcus aureus]
>gi|3955031|emb|CAA76853.1|PBP2 [Staphylococcus aureus]
>gi|3955030|emb|CAA76852.1| unknown [Staphylococcus aureus]
>gi|3550596|emb|CAA06652.1| sdr E [Staphylococcus aureus]
>gi|3550594|emb|CAA06651.1| sdrD [Staphylococcus aureus]
>gi|3550592|emb|CAA06650.1| sdrc [Staphylococcus aureus]
>gi|809080|emb|CAA24595.1| reading frame [Staphylococcus aureus]
>gi|3320606|gb|AAD09875.1| putative heme A synthase [Staphylococcus aureus]
>gi|4104230|gb|AAD01977.1| phospho-N-acetylmuramoyl-pentapeptide translocase [Staphylococcus aureus]
>gi|4103900|gb|AAD01884.1| 60 kDa heat shock protein [Staphylococcus aureus]
>gi|4097757|gb|AAD00167.1| lytic regulatory protein [Staphylococcus aureus]
>gi|4090655|gb|AAC98834.1| ORF64 [Staphylococcus aureus]
>gi|4090654|gb|AAC98833.1| replication protein [Staphylococcus aureus]
>gi|4090652|gb|AAC98832.1| ORF64 [Staphylococcus aureus]
>gi|4090651|gb|AAC98831.1| replication protein [Staphylococcus aureus]
>gi|2811118|gb|AAC95492.1| unknown [Staphylococcus aureus]
>gi|2811115|gb|AAC95491.1| unknown [Staphylococcus aureus]
>gi|4009497|gb|AAC95464.1| cell division protein DivIVA [Staphylococcus aureus]
>gi|4009496|gb|AAC95463.1| YlmH [Staphylococcus aureus]
>gi|4009495|gb|AAC95462.1| YlmG [Staphylococcus aureus]
>gi|4009494|gb|AAC95461.1| YlmF [Staphylococcus aureus]
>gi|4009493|gb|AAC95460.1| YlmE [Staphylococcus aureus]
>gi|4009492|gb|AAC95459.1| YlmD [Staphylococcus aureus]
>gi|4009491|gb|AAC95458.1| cell division protein FtsZ [Staphylococcus aureus]
>gi|1402771|gb|AAC80254.1| major cold-shock protein [Staphylococcus aureus]
>gi|3372542|gb|AAC78590.1| enterotoxin J [Staphylococcus aureus]
>gi|3372541|gb|AAC78589.1| enterotoxin D [Staphylococcus aureus]
>gi|3892644|dbj|BAA34540.1| MphBM [Staphylococcus aureus]
>gi|3892643|dbj|BAA34539.1| MsrSA [Staphylococcus aureus]
>gi|3892642|dbj|BAA34538.1| leader peptide [Staphylococcus aureus]
>gi|3850852|emb|CAA76222.1| hypothetical protein [Staphylococcus aureus]
>gi|3850851|emb|CAA76221.1| alr protein [Staphylococcus aureus]
>gi|3850850|emb|CAA76220.1| dpj protein [Staphylococcus aureus]
>gi|3850849|emb|CAA76219.1| hypothetical protein [Staphylococcus aureus]
>gi|3850848|emb|CAA76218.1| hypothetical protein [Staphylococcus aureus]
>gi|3850847|emb|CAA76217.1| hypothetical protein [Staphylococcus aureus]
>gi|3850846|emb|CAA76216.1| kpdC protein [Staphylococcus aureus]
>gi|3800828|gb|AAC69846.1| oligopeptide transporter putative ATPase domain [Staphylococcus aureus]
>gi|3800827|gb|AAC69845.1| oligopeptide transporter putative ATPase domain [Staphylococcus aureus]
>gi|3800826|gb|AAC69844.1| oligopeptide transporter putative membrane permease domain
[Staphylococcus aureus]
>gi|3800825|gb|AAC69843.1| oligopeptide transporter putative membrane permease domain
[Staphylococcus aureus]
>gi|3800823|gb|AAC69842.1| unknown [Staphylococcus aureus]
>gi|3800822|gb|AAC69841.1| oligopeptide transporter putative ATPase domain [Staphylococcus aureus]
>gi|3800821|gb|AAC69840.1| oligopeptide transporter putative ATPase domain [Staphylococcus aureus]
>gi|3800820|gb|AAC69839.1| oligopeptide transporter putative membrane permease domain
[Staphylococcus aureus]
>gi|3800819|gb|AAC69838.1| oligopeptide transporter putative membrane permease domain
[Staphylococcus aureus]
>gi|3800818|gb|AAC69837.1| oligopeptide transporter putative substrate binding domain
[Staphylococcus aureus]
>gi|2765304|emb|CAA73668.1| leukotoxin, LukD [Staphylococcus aureus]
>gi|2765303|emb|CAA73667.1| leukotoxin LukE [Staphylococcus aureus]
>gi|3212829|pdb|5NUC| Staphylococcal Nuclease, 1-N-Pentane Thiol Disulfide To V23c Variant
>gi|3212819|pdb|3SEB| Staphylococcal Enterotoxin B
>gi|2982145|pdb|2SAK| Staphylokinase (Sakstar Variant)
>gi|2914575|pdb|7AHL|G Chain G, Alpha-Hemolysin From Staphylococcus aureus
>gi|2914574|pdb|7AHL|F Chain F, Alpha-Hemolysin From Staphylococcus aureus
>gi|2914573|pdb|7AHL|E Chain E, Alpha-Hemolysin From Staphylococcus aureus
>gi|2914572|pdb|7AHL|D Chain D, Alpha-Hemolysin From Staphylococcus aureus
>gi|2914571|pdb|7AHL|C Chain C, Alpha-Hemolysin From Staphylococcus aureus
>gi|2914570|pdb|7AHL|B Chain B, Alpha-Hemolysin From Staphylococcus aureus
>gi|2914569|pdb|7AHL|A Chain A, Alpha-Hemolysin From Staphylococcus aureus
>gi|2914205|pdb|2SEB|D Chain D, X-Ray Crystal Structure Of Hla-Dr4 Complexed With A Peptide From
Human Collagen Ii
>gi|2780937|pdb|5TSS|B Chain B, Toxic Shock Syndrome Toxin-1: Orthorhombic P222(1) Crystal Form
>gi|2780936|pdb|5TSS|A Chain A, Toxic Shock Syndrome Toxin-1: Orthorhombic P222(1) Crystal Form
>gi|2780930|pdb|4TSS| Toxic Shock Syndrome Toxin-1: Tetragonal P4(1)2(1)2 Crystal Form
>gi|2780925|pdb|3TSS| Toxic Shock Syndrome Toxi-1 Tetramutant, P2(1) Crystal Form
>gi|2780919|pdb|2TSS|C Chain C, Toxic Shock Syndrome Toxin-1 From Staphylococcus aureus:
Orthorhombicc222(1) Crystal Form
>gi|2780918|pdb|2TSS|B Chain B, Toxic Shock Syndrome Toxin-1 From Staphylococcus aureus:
Orthorhombicc222(1) Crystal Form
>gi|2780917|pdb|2TSS|A Chain A, Toxic Shock Syndrome Toxin-1 From Staphylococcus aureus:
Orthorhombicc222(1) Crystal Form
>gi|2392714|pdb|2QIL|C Chain C, Toxic Shock Syndrome Toxin-1 At 2.07 A Resolution
>gi|2392713|pdb|2QIL|B Chain B, Toxic Shock Syndrome Toxin-1 At 2.07 A Resolution
>gi|2392712|pdb|2QIL|A Chain A, Toxic Shock Syndrome Toxin-1 At 2.07 A Resolution
>gi|1310952|pdb|2SOB| Sn-Ob, Ob-Fold Sub-Domain Of Staphylococcal Nuclease, Nmr, 10 Structures
>gi|2781288|pdb|1SXT|B Chain B, Staphylococcal Enterotoxin Type A (Sea) Co-Crystallised With Zinc
>gi|2781287|pdb|1SXT|A Chain A, Staphylococcal Enterotoxin Type A (Sea) Co-Crystallised With Zinc
>gi|2624726|pdb|1SE4| Staphylococcal Enterotoxin B Complexed With Lactose
>gi|2392546|pdb|1SE3| Staphylococcal Enterotoxin B Complexed With Gm3 Trisaccharide
>gi|2098291|pdb|1SND|B Chain B, Staphylococcal Nuclease Dimer Containing A Deletion Of Residues 114-119
Complexed With Calcium Chloride And The Competitive Inhibitor Deoxythymidine-3′,5′-Diphosphate
>gi|2098290|pdb|1SND|A Chain A, Staphylococcal Nuclease Dimer Containing A Deletion Of Residues 114-119
Complexed With Calcium Chloride And The Competitive Inhibitor Deoxythymidine-3′,5′-Diphosphate
>gi|1942753|pdb|1STE| Staphylococcal Enterotoxin C2 From Staphylococcus aureus
>gi|1633348|pdb|1SEB|H Chain H, Complex Of The Human Mhc Class Ii Glycoprotein Hla-Dr1 And The
Bacterial Superantigen Seb
>gi|1633344|pdb|1SEB|D Chain D, Complex Of The Human Mhc Class Ii Glycoprotein Hla-Dr1 And The
Bacterial Superantigen Seb
>gi|1431724|pdb|1SE2| Staphylococcal Enterotoxin C2, Monoclinic Form
>gi|3212584|pdb|1OME|B Chain B, Crystal Structure Of The Omega Loop Deletion Mutant (Residues 163-178
Deleted) Of Beta-Lactamase From Staphylococcus aureusPc1
>gi|3212583|pdb|1OME|A Chain A, Crystal Structure Of The Omega Loop Deletion Mutant (Residues 163-178
Deleted) Of Beta-Lactamase From Staphylococcus aureusPc1
>gi|2392515|pdb|1QIL|C Chain C, Inactive Mutant Toxic Shock Syndrome Toxin-1 At 2.5 A
>gi|2392514|pdb|1QIL|B Chain B, Inactive Mutant Toxic Shock Syndrome Toxin-1 At 2.5 A
>gi|2392513|pdb|1QIL|A Chain A, Inactive Mutant Toxic Shock Syndrome Toxin-1 At 2.5 A
>gi|2392479|pdb|1NUC| Staphylococcal Nuclease, V23c Variant
>gi|2624537|pdb|1JCK|D Chain D, T-Cell Receptor Beta Chain Complexed With Sec3 Superantigen
>gi|2624535|pdb|1JCK|B Chain B, T-Cell Receptor Beta Chain Complexed With Sec3 Superantigen
>gi|2098496|pdb|1KGE| Structure Of Beta-Lactamase Asn 170 Met Mutant
>gi|1942204|pdb|1KGF| Structure Of Beta-Lactamase Asn 170 Gln Mutant
>gi|1827772|pdb|1KNY|B Chain B, Kanamycin Nucleotidyltransferase
>gi|1827771|pdb|1KNY|A Chain A, Kanamycin Nucleotidyltransferase
>gi|2982092|pdb|1EXF|A Chain A, Exfoliative Toxin A
>gi|2098519|pdb|1EDL| Staphylococcal Protein A E-Domain (−60), Nmr, 22 Structures
>gi|2098517|pdb|1EDI| Staphylococcal Protein A E-Domain (180), Nmr, Minimized Average Structure
>gi|2098516|pdb|1EDJ| Staphylococcal Protein A E-Domain (180), Nmr, 20 Structures
>gi|2098515|pdb|1EDK| Staphylococcal Protein A E-Domain (−60), Nmr, Minimized Average Structure
>gi|1942144|pdb|1DJC| Structure Of Beta-Lactamase Precursor, S70a Mutant, At 120k
>gi|1942143|pdb|1DJB| Structure Of Beta-Lactamase Precursor, S70a Mutant, At 298k
>gi|1942142|pdb|1DJA| Structure Of Beta-Lactamase Precursor, K73h Mutant, At 298k
>gi|1633233|pdb|1ESF|B Chain B, Staphylococcal Enterotoxin A
>gi|1633232|pdb|1ESF|A Chain A, Staphylococcal Enterotoxin A
>gi|1942696|pdb|1BDD| Staphylococcus aureus Protein A, Immunoglobulin-Binding B Domain, Nmr,
Minimized Average Structure
>gi|1942695|pdb|1BDC| Staphylococcus aureus Protein A, Immunoglobulin-Binding B Domain, Nmr, 10
Structures
>gi|3318765|pdb|1AMX| Collagen-Binding Domain From A Staphylococcus aureus Adhesin
>gi|3212427|pdb|1AD4|B Chain B, Dihydropteroate Synthetase Complexed With Oh-Ch2-Pterin-
Pyrophosphate From Staphylococcus aureus
>gi|3212426|pdb|1AD4|A Chain A, Dihydropteroate Synthetase Complexed With Oh-Ch2-Pterin-
Pyrophosphate From Staphylococcus aureus
>gi|3212425|pdb|1AD1|B Chain B, Dihydropteroate Synthetase (Apo Form) From Staphylococcus aureus
>gi|3212424|pdb|1AD1|A Chain A, Dihydropteroate Synthetase (Apo Form) From Staphylococcus aureus
>gi|2554719|pdb|1AGJ|B Chain B, Epidermolytic Toxin A From Staphylococcus aureus
>gi|2554718|pdb|1AGJ|A Chain A, Epidermolytic Toxin A From Staphylococcus aureus
>gi|2554635|pdb|1ALQ| Circularly Permuted Beta-Lactamase From Staphylococcus aureus Pc1
>gi|2392077|pdb|1AEX| Staphylococcal Nuclease, Methane Thiol Disulfide To V23c Variant
>gi|3212327|pdb|1A3V| Staphylococcal Nuclease, Cyclopentane Thiol Disulfide To V23c Variant
>gi|3212326|pdb|1A3U| Staphylococcal Nuclease, Cyclohexane Thiol Disulfide To V23c Variant
>gi|3212325|pdb|1A3T| Staphylococcal Nuclease, V23c Variant, Complex With 2-Fluoroethane Thiol And
3′,5′-Thymidine Diphosphate
>gi|3212274|pdb|1A2U| Staphylococcal Nuclease, V23c Variant, Complex With 1-N-Butane Thiol And
3′,5′-Thymidine Diphosphate
>gi|3212273|pdb|1A2T| Staphylococcal Nuclease, B-Mercaptoethanol Disulfide To V23c Variant
>gi|3776113|emb|CAA11406.1| succinate dehydrogenase complex, cytochrome b558 subunit [Staphylococcus
aureus]
>gi|3776112|emb|CAA11405.1| excinuclease ABC, subunit C [Staphylococcus aureus]
>gi|3776111|emb|CAA11404.1| thioredoxin [Staphylococcus aureus]
>gi|3776110|emb|CAA11403.1| MutS-like protein [Staphylococcus aureus]
>gi|3747042|gb|AAC64162.1| tyrosine recombinase XerD [Staphylococcus aureus]
>gi|3676411|gb|AAC63227.1| putative transposase TnpE [Staphylococcus aureus]
>gi|3676410|gb|AAC63226.1| thymidylate synthetase ThyE [Staphylococcus aureus]
>gi|3676409|gb|AAC63225.1| trimethoprim resistance protein DfrA [Staphylococcus aureus]
>gi|3676408|gb|AAC63224.1| unknown [Staphylococcus aureus]
>gi|3676407|gb|AAC63223.1| putative transposase TnpD [Staphylococcus aureus]
>gi|3676406|gb|AAC63222.1| replication protein Rep [Staphylococcus aureus]
>gi|3676405|gb|AAC63221.1| putative transposase TnpC [Staphylococcus aureus]
>gi|3676456|gb|AAC61974.1| putative transposase TnpG [Staphylococcus aureus]
>gi|3676455|gb|AAC61973.1| putative transposase TnpF [Staphylococcus aureus]
>gi|3676454|gb|AAC61972.1| bifunctional aminoglycoside modifying enzyme AacA-AphD [Staphylococcus
aureus]
>gi|3676453|gb|AAC61971.1| unknown [Staphylococcus aureus]
>gi|3676452|gb|AAC61970.1| putative transposase TnpE [Staphylococcus aureus]
>gi|3676451|gb|AAC61969.1| multidrug resistance efflux protein Smr [Staphylococcus aureus]
>gi|3676450|gb|AAC61968.1| putative replication initiation protein Rep(RC) [Staphylococcus aureus]
>gi|3676449|gb|AAC61967.1| putative transposase TnpD [Staphylococcus aureus]
>gi|3676448|gb|AAC61966.1| unknown [Staphylococcus aureus]
>gi|3676447|gb|AAC61965.1| putative single-stranded DNA binding protein TraM [Staphylococcus aureus]
>gi|3676446|gb|AAC61964.1| putative membrane protein TraL [Staphylococcus aureus]
>gi|3676445|gb|AAC61963.1| putative membrane protein TraK [Staphylococcus aureus]
>gi|3676444|gb|AAC61962.1| putative membrane protein TraJ [Staphylococcus aureus]
>gi|3676443|gb|AAC61961.1| putative topoisomerase TraI [Staphylococcus aureus]
>gi|3676442|gb|AAC61960.1| lipoprotein TraH [Staphylococcus aureus]
>gi|3676441|gb|AAC61959.1| putative membrane protein TraG [Staphylococcus aureus]
>gi|3676440|gb|AAC61958.1| putative membrane protein TraF [Staphylococcus aureus]
>gi|3676439|gb|AAC61957.1| putative ATPase TraE [Staphylococcus aureus]
>gi|3676438|gb|AAC61956.1| TraD [Staphylococcus aureus]
>gi|3676437|gb|AAC61955.1| putative membrane protein TraC [Staphylococcus aureus]
>gi|3676436|gb|AAC61954.1| putative membrane protein TraB [Staphylococcus aureus]
>gi|3676435|gb|AAC61953.1| TraA [Staphylococcus aureus]
>gi|3676434|gb|AAC61952.1| putative regulator of transfer genes ArtA [Staphylococcus aureus]
>gi|3676433|gb|AAC61951.1| putative transposase TnpC [Staphylococcus aureus]
>gi|3676432|gb|AAC61950.1| aminoglycoside adenyltransferase AadD [Staphylococcus aureus]
>gi|3676431|gb|AAC61949.1| bleomycin resistance protein Ble [Staphylococcus aureus]
>gi|3676430|gb|AAC61948.1| Pre [Staphylococcus aureus]
>gi|3676429|gb|AAC61947.1| putative transposase TnpB [Staphylococcus aureus]
>gi|3676428|gb|AAC61946.1| membrane protein [Staphylococcus aureus]
>gi|3676427|gb|AAC61945.1| putative transposase TnpA [Staphylococcus aureus]
>gi|3676426|gb|AAC61944.1| putative replication initiation protein Rep [Staphylococcus aureus]
>gi|3676425|gb|AAC61943.1| unknown [Staphylococcus aureus]
>gi|3676424|gb|AAC61942.1| unknown [Staphylococcus aureus]
>gi|3676423|gb|AAC61941.1| unknown [Staphylococcus aureus]
>gi|3676422|gb|AAC61940.1| putative membrane protein [Staphylococcus aureus]
>gi|3676421|gb|AAC61939.1| unknown [Staphylococcus aureus]
>gi|3676420|gb|AAC61938.1| oriT nickase Nes [Staphylococcus aureus]
>gi|3676419|gb|AAC61937.1| LtrC-like protein [Staphylococcus aureus]
>gi|3676418|gb|AAC61936.1| unknown [Staphylococcus aureus]
>gi|3676417|gb|AAC61935.1| unknown [Staphylococcus aureus]
>gi|3676416|gb|AAC61934.1| putative resolvase Res [Staphylococcus aureus]
>gi|3676415|gb|AAC61933.1| unknown [Staphylococcus aureus]
>gi|3676414|gb|AAC61932.1| unknown [Staphylococcus aureus]
>gi|3676413|gb|AAC61931.1| unknown [Staphylococcus aureus]
>gi|410007|gb|AAC60446.1| leukocidin F component [Staphylococcus aureus, MRSA No. 4, Peptide, 323
aa]
>gi|410006|gb|AAC60445.1| leukocidin S component [Staphylococcus aureus, MRSA No. 4, Peptide, 315
aa]
>gi|410005|gb|AAC60444.1| gamma-hemolysin II, H gamma II [Staphylococcus aureus, MRSA No. 4,
Peptide, 309 aa]
>gi|2271510|gb|AAC46291.1| UDP-N-acetylmuramoyl-L-alanine : D-glutamate ligase; MurD [Staphylococcus
aureus]
>gi|1773355|gb|AAC46099.1| Cap5P [Staphylococcus aureus]
>gi|1773354|gb|AAC46098.1| Cap5O [Staphylococcus aureus]
>gi|1773353|gb|AAC46097.1| Cap5N [Staphylococcus aureus]
>gi|1773352|gb|AAC46096.1| Cap5M [Staphylococcus aureus]
>gi|1773351|gb|AAC46095.1| Cap5L [Staphylococcus aureus]
>gi|1773350|gb|AAC46094.1| Cap5K [Staphylococcus aureus]
>gi|1773349|gb|AAC46093.1| Cap5J [Staphylococcus aureus]
>gi|1773348|gb|AAC46092.1| Cap5I [Staphylococcus aureus]
>gi|1773347|gb|AAC46091.1| Cap5H [Staphylococcus aureus]
>gi|1773346|gb|AAC46090.1| Cap5G [Staphylococcus aureus]
>gi|1773345|gb|AAC46089.1| Cap5F [Staphylococcus aureus]
>gi|1773344|gb|AAC46088.1| Cap5E [Staphylococcus aureus]
>gi|1773343|gb|AAC46087.1| Cap5D [Staphylococcus aureus]
>gi|1773342|gb|AAC46086.1| Cap5C [Staphylococcus aureus]
>gi|1773341|gb|AAC46085.1| Cap5B [Staphylococcus aureus]
>gi|1773340|gb|AAC46084.1| Cap5A [Staphylococcus aureus]
>gi|1673629|gb|AAC46100.1| O-acetyl transferase [Staphylococcus aureus]
>gi|706922|gb|AAC46354.1| ribosomal protein S7 [Staphylococcus aureus]
>gi|706921|gb|AAC46353.1| ribosomal protein S12 [Staphylococcus aureus]
>gi|706920|gb|AAC46352.1| unknown [Staphylococcus aureus]
>gi|2589184|gb|AAC45836.1| GSA-1-aminotransferase [Staphylococcus aureus]
>gi|2589183|gb|AAC45835.1| d-aminolevulinic acid dehydratase [Staphylococcus aureus]
>gi|2589182|gb|AAC45834.1| uroporphyrinogen III synthase [Staphylococcus aureus]
>gi|2589181|gb|AAC45833.1| porphobilinogen deaminase [Staphylococcus aureus]
>gi|2149898|gb|AAC45629.1| cell division protein [Staphylococcus aureus]
>gi|2149897|gb|AAC45628.1| cell division protein [Staphylococcus aureus]
>gi|2149896|gb|AAC45627.1| cell division protein [Staphylococcus aureus]
>gi|2149895|gb|AAC45626.1| D-glutamic acid adding enzyme [Staphylococcus aureus]
>gi|2149894|gb|AAC45625.1| phospho-N-muramic acid-pentapeptide translocase [Staphylococcus aureus]
>gi|2149893|gb|AAC45624.1| penicillin-binding protein 1 [Staphylococcus aureus]
>gi|2149892|gb|AAC45623.1| cell division protein [Staphylococcus aureus]
>gi|2149891|gb|AAC45622.1| unknown [Staphylococcus aureus]
>gi|2149890|gb|AAC45621.1| unknown [Staphylococcus aureus]
>gi|1314302|gb|AAC45357.1| isoleucyl-tRNA synthetase [Staphylococcus aureus]
>gi|1314301|gb|AAC45356.1| unknown [Staphylococcus aureus]
>gi|1575026|gb|AAC44840.1| LrgB
>gi|1575025|gb|AAC44839.1| holin-like protein LrgA
>gi|710422|gb|AAC44803.1| cmp-binding-factor 1
>gi|710421|gb|AAC44802.1| unknown
>gi|1595810|gb|AAC44435.1| type-I signal peptidase SpsB [Staphylococcus aureus]
>gi|1595809|gb|AAC44434.1| type-I signal peptidase SpsA [Staphylococcus aureus]
>gi|1397239|gb|AAC44135.1| elastin binding protein
>gi|1001961|gb|AAC43470.1| MHC class II analog
>gi|3327949|gb|AAC38785.1| putative recombinase Sin [Staphylococcus aureus]
>gi|3327948|gb|AAC38784.1| multidrug efflux protein QacB [Staphylococcus aureus]
>gi|3327947|gb|AAC38783.1| transcriptional regulator QacR [Staphylococcus aureus]
>gi|3327945|gb|AAC38782.1| putative transposase TnpA [Staphylococcus aureus]
>gi|3327944|gb|AAC38781.1| delta-orf186 [Staphylococcus aureus]
>gi|3327943|gb|AAC38780.1| multidrug efflux protein QacB [Staphylococcus aureus]
>gi|3327942|gb|AAC38779.1| transcriptional regulator QacR [Staphylococcus aureus]
>gi|3135292|gb|AAC38560.1| large conductance mechanosensitive channel [Staphylococcus aureus]
>gi|2827912|gb|AAC38446.1| IgG-binding protein SBI [Staphylococcus aureus]
>gi|2565311|gb|AAC38087.1| high affinity proline permease [Staphylococcus aureus]
>gi|2315995|gb|AAC38146.1| branched-chain amino acid carrier protein [Staphylococcus aureus]
>gi|4379428|emb|CAA11546.1| thioredoxin reductase [Staphylococcus aureus]
>gi|3445567|gb|AAC32485.1| transport protein [Staphylococcus aureus]
>gi|3445566|gb|AAC32484.1| repressor protein [Staphylococcus aureus]
>gi|3411114|gb|AAC31156.1| DNA topoisomerase IV subunit A [Staphylococcus aureus]
>gi|3411112|gb|AAC31155.1| DNA topoisomerase IV subunit A [Staphylococcus aureus]
>gi|3411110|gb|AAC31154.1| DNA topoisomerase IV subunit A [Staphylococcus aureus]
>gi|3411108|gb|AAC31153.1| DNA topoisomerase IV subunit A [Staphylococcus aureus]
>gi|3411106|gb|AAC31152.1| DNA topoisomerase IV subunit A [Staphylococcus aureus]
>gi|3411104|gb|AAC31151.1| DNA topoisomerase IV subunit A [Staphylococcus aureus]
>gi|3411102|gb|AAC31150.1| DNA topoisomerase IV subunit A [Staphylococcus aureus]
>gi|3411100|gb|AAC31149.1| DNA topoisomerase IV subunit A [Staphylococcus aureus]
>gi|3411098|gb|AAC31148.1| DNA topoisomerase IV subunit A [Staphylococcus aureus]
>gi|3411096|gb|AAC31147.1| DNA topoisomerase IV subunit A [Staphylococcus aureus]
>gi|3411092|gb|AAC31144.1| DNA gyrase subunit A; topoisomerase [Staphylococcus aureus]
>gi|3411090|gb|AAC31143.1| DNA gyrase subunit A; topoisomerase [Staphylococcus aureus]
>gi|3411088|gb|AAC31142.1| DNA gyrase subunit A; topoisomerase [Staphylococcus aureus]
>gi|3411086|gb|AAC31141.1| DNA gyrase subunit A; topoisomerase [Staphylococcus aureus]
>gi|3411084|gb|AAC31140.1| DNA gyrase subunit A; topoisomerase [Staphylococcus aureus]
>gi|3411082|gb|AAC31139.1| DNA gyrase subunit A; topoisomerase [Staphylococcus aureus]
>gi|3411080|gb|AAC31138.1| DNA gyrase subunit A; topoisomerase [Staphylococcus aureus]
>gi|3411078|gb|AAC31137.1| DNA gyrase subunit A; topoisomerase [Staphylococcus aureus]
>gi|3411076|gb|AAC31136.1| DNA gyrase subunit A; topoisomerase [Staphylococcus aureus]
>gi|3411074|gb|AAC31135.1| DNA gyrase subunit A; topoisomerase [Staphylococcus aureus]
>gi|2689564|gb|AAC28969.1| integrase [Staphylococcus aureus]
>gi|2689563|gb|AAC28968.1| enterotoxin [Staphylococcus aureus]
>gi|2689562|gb|AAC28967.1| orf15 [Staphylococcus aureus]
>gi|2889561|gb|AAC28966.1| orf14 [Staphylococcus aureus]
>gi|2689560|gb|AAC28965.1| orf13 [Staphylococcus aureus]
>gi|2689559|gb|AAC28964.1| orf12 [Staphylococcus aureus]
>gi|2689558|gb|AAC28963.1| orf11 [Staphylococcus aureus]
>gi|2689557|gb|AAC28962.1| orf10 [Staphylococcus aureus]
>gi|2689556|gb|AAC28961.1| orf9 [Staphylococcus aureus]
>gi|2689555|gb|AAC28960.1| orf8 [Staphylococcus aureus]
>gi|2689554|gb|AAC28959.1| orf7 [Staphylococcus aureus]
>gi|2889553|gb|AAC28958.1| orf6 [Staphylococcus aureus]
>gi|2689552|gb|AAC28957.1| orf5 [Staphylococcus aureus]
>gi|2689551|gb|AAC28956.1| orf4 [Staphylococcus aureus]
>gi|2689550|gb|AAC28955.1| orf3 [Staphylococcus aureus]
>gi|2689549|gb|AAC28954.1| toxic shock syndrome toxin-1 [Staphylococcus aureus]
>gi|2689548|gb|AAC28953.1| orf1 [Staphylococcus aureus]
>gi|3393011|emb|CAA12115.1| Clumping factor B [Staphylococcus aureus]
>gi|3323613|gb|AAC26661.1| extracellular enterotoxin type I precursor [Staphylococcus aureus]
>gi|3323611|gb|AAC26660.1| extracellular enterotoxin type G precursor [Staphylococcus aureus]
>gi|3256224|emb|CAA74741.1| ypfP [Staphylococcus aureus]
>gi|3256223|emb|CAA74740.1| UDP-N-acetylmuramyl-tripeptide synthetase [Staphylococcus aureus]
>gi|3256222|emb|CAA74739.1| peptide chain release factor 3 [Staphylococcus aureus]
>gi|230814|pdb|3BLM| Beta-Lectamase (E.C.3.5.2.6)
>gi|230746|pdb|2SNS| Staphylococcal Nuclease (E.C.3.1.33.1) Complex With 2(Prime)-Deoxy-3(Prime)-
5(Prime)-Diphosphothymidine
>gi|230745|pdb|2SNM| Staphylococcal Nuclease (E.C.3.1.31.1) Mutant With Val 66 Replaced By Lys
(V66k)
>gi|576398|pdb|2ENB| Staphylococcal Nuclease (E.C.3.1.31.1) Mutation With Asp 21 Replaced By Glu
(D21e) complexed With The Inhihitor Thymidine 3′,5′-Diphosphate
>gi|443374|pdb|2DTB| Delta-Toxin (Delta-Haemolysin) (Nmr, 9 Structures)
>gi|1421454|pdb|1ZER| Mol_id: 1; Molecule: Histidine-Containing Phosphocarrier Protein; Chain:
Null; Synonym: Hpr
>gi|576294|pdb|1SYG| Staphylococcal Nuclease (E.C.3.1.31.1) Mutant With Pro 117 Replaced By Ala
(P117a)
>gi|576293|pdb|1SYF| Staphylococcal Nuclease (E.C.3.1.31.1) Mutant With Pro 117 Replaced By Thr
(P117t) Complexed With 2′-Deoxy-3′-5′-Diphosphothymidine And Calcium
>gi|576292|pdb|1SYE| Staphylococcal Nuclease (E.C.3.1.31.1) Mutant With Pro 117 Replaced By Thr
(P117t)
>gi|576291|pdb|1SYD| Staphylococcal Nuclease (E.C.3.1.31.1) Mutant With Pro 117 Replaced By Gly
(P117g) Complexed With 2′-Deoxy-3′-5′-Diphosphothymidine And Calcium
>gi|576290|pdb|1SYC| Staphylococcal Nuclease (E.C.3.1.31.1) Mutant With Pro 117 Replaced By Gly
(P117g)
>gi|576289|pdb|1SYB| Staphylococcal Nuclease (E.C.3.1.31.1) With Residues 27-31 (Tyr-Lys-Gly-Gln-
Pro) Replaced With Residues 160-165 Of Concanavalin A (Ser-Ser-Asn-Gly-Ser-Pro) Complexed With 2′-
Deoxy-3′-5′-Diphosphothymidine And Calcium
>gi|349914|pdb|1STY| Staphylococcal Nuclease (E.C.3.1.31.1) Insertion Mutant With Glycine Residue
Inserted In An Alpha Helix, Between Arg126 And Lys127 (126g127) Complex With Calcium And Inhibitor
Thymidine 3′,5′-Bisphosphate)
>gi|999674|pdb|1STH| Staphylococcal Nuclease (E.C.3.1.31.1) Complexed With Co(Ii) Ion And Thymidine
3′,5′-Bisphosphete (Pdtp)
>gi|999672|pdb|1STG| Staphylococcal Nuclease (E.C.3.1.31.1)
>gi|576287|pdb|1STN| Stephylococcal Nuclease (E.C.3.1.31.1)
>gi|576286|pdb|1STB| Staphylococcal Nuclease (E.C.3.1.31.1) Insertion Mutant With Leu Inserted At
The End Of The Third Beta-Strand Between Leu 36 And Leu 37 (Ins(L36-L)) Complexed With Thymidine
3′,5′-Diphosphate And Calcium
>gi|576285|pdb|1STA| Staphylococcal Nuclease (E.C.3.1.31.1) Double Insertion Mutant With Two
Glycine Residues Inserted In The First Beta Strand Between Pro 11 And Ala 12 (Ins(P11-Gg)) Complexed
With Calcium And The Inhibitor Thymidine 3′,5′-Diphosphate
>gi|230332|pdb|1SNM| Staphylococcal Nuclease (E.C.3.1.31.1) Mutant (Glu 43 Replaced By Asp) (E43D)
Complex With A Calcium Ion And 3-Prime, 5-Prime-Deoxythymidine Bisphosphate
>gi|230331|pdb|1SNC| Staphylococcal Nuclease (E.C.3.1.31.1) Complex With A Calcium Ion And 3-Prime,
5-Prime-Deoxythymidine Bisphosphate
>gi|1431686|pdb|1PIO|B Chain B, Mol_id: 1; Molecule: Beta-Lactemese; chain: A, B; Synonym:
Penicillinase; Ec: 3.5.2.6; Engineered: Yes; Mutation: Ins(Met 30), A238s, Del(I239)
>gi|1431685|pdb|1PIO|A Chain A, Mol_id: 1; Molecule: Beta-Lactamese; Chain: A, B; Synonym:
Penicillinase; Ec: 3.5.2.6; Engineered: Yes; Mutation: Ins(Met 30), A238s, Del(I239)
>gi|11127093|pdb|1NSN|S Chain S, Immunoglobulin, Staphylococcal Nuclease Mol_id: 1; Molecule: Igg Fab
(Igg1, Kappa); Chain: L, H; Domain: Fragment N10; Synonym: N10 Fab Immunoglobulin; Mol_id: 2;
Molecule: Staphylococcal Nuclease; Chain: S; Synonym: Stephylococcal Nuclease Ribonucleate,
(Deoxyribonucleate)-3′-Nucleotidohydrolase; Ec: 3.1.31.1; Engineered: Yes
>gi|999581|pdb|1KDC| Staphylococcal Nuclease (E.C.3.1.31.1) Mutant With Lys 116 Replaced By Asn
(K116n)
>gi|999578|pdb|1KDB| Staphylococcal Nuclease (E.C.3.1.31.1) Mutant With Lys 116 Replaced By Glu
(K116e)
>gi|999577|pdb|1KDA| Staphylococcal Nuclease (E.C.3.1.31.1) Mutant With Lys 116 Replaced By Asp
(K116d)
>gi|640137|pdb|1KAN|B Chain B, Kanamycin Nucleotidyltransferase (E.C.2.7.7.-) Mutant With Asp 80
Replaced By Tyr And Thr 130 Replaced By Lys (D80y,T130k)
>gi|640136|pdb|1KAN|A Chain A, Kanamycin Nucleotidyltransferase (E.C.2.7.7.-) Mutant With Asp 80
Replaced By Tyr And Thr 130 Replaced By Lys (D80y,T130k)
>gi|494228|pdb|1KAB| Staphylococcal Nuclease (E.C.3.1.33.1) Mutant With Lys 116 Replaced By Gly
(K116g)
>gi|494227|pdb|KAA| Staphylococcal Nuclease (E.C.3.1.33.1) Mutant With Lys 116 Replaced By Ala
(K116a)
>gi|229907|pdb|1FC2|C Chain C, Immunoglobulin Fc And Fragment B Of Protein A Complex
>gi|576100|pdb|1ENC| Staphylococcal Nuclease (E.C.3.1.31.1) Mutant With Asp 21 Replaced By Glu
(D21e) Complexed With A Calcium Ion And The Inhibitor Thymidine 3′,5′-Diphosphate
>gi|576099|pdb|1ENA| Staphylococcal Nuclease (E.C.3.1.31.1) Mutation With Asp 21 Replaced By Glu
(D21e) Complexed With A Calcium Ion
>gi|442844|pdb|1DTC| Acetyl-Delta-Toxin (Acetyl-Delta-Haemolysin) (Nmr, 12 Structures)
>gi|640251|pdb|1BLH| Beta-Lactamase (E.C.3.5.2.6) Complexed With [[n-
(Benzyloxycarbonyl) amino]methyl]phosphonate
>gi|515092|pdb|1BLP| Beta-Lactamase (E.C.3.5.2.6) P54 Mutant With Asp 179 Replaced By Asn (D179n)
>gi|493890|pdb|1BLC| Beta-Lactamase (E.C.3.5.2.6) Complex With Degradation Products Of Clavulanate
>gi|3152725|gb|AAC17130.1| enolase [Staphylococcus aureus]
>gi|2463563|dbj|BAA22556.1| MURD [Staphylococcus aureus]
>gi|2463562|dbj|BAA22555.1| MRAY [Staphylococcus aureus]
>gi|2463561|dbj|BAA22554.1| penicillin-binding protein 1 [Staphylococcus aureus]
>gi|2463560|dbj|BAA22553.1| unnamed protein product [Staphylococcus aureus]
>gi|2463559|dbj|BAA22552.1| unnamed protein product [Staphylococcus aureus]
>gi|1835218|emb|CAA71057.1| seryl-trna synthetase [Staphylococcus aureus]
>gi|3122772|sp|O31203|RRF1_STAAU PROBABLE RIBOSOME RECYCLING FACTOR (RIBOSOME RELEASING FACTOR)
(RRF)
>gi|1488695|gb|AAC12901.1| novel antigen; orf-2 [Staphylococcus aureus]
>gi|2826896|dbj|BAA24572.1| RecG [Staphylococcus aureus]
>gi|2791991|emb|CAA74380.1| putative transposase [Staphylococcus aureus]
>gi|2791990|emb|CAA74379.1| hypothetical protein [Staphylococcus aureus]
>gi|2791989|emb|CAA74378.1| hypothetical protein [Staphylococcus aureus]
>gi|2791988|emb|CAA74377.1| hypothetical protein [Staphylococcus aureus]
>gi|2791987|emb|CAA74376.1| PBP2A [Staphylococcus aureus]
>gi|2791986|emb|CAA74375.1| MecR1 protein [Staphylococcus aureus]
>gi|2791985|emb|CAA74374.1| MecI protein [Staphylococcus auraus]
>gi|2791984|emb|CAA74373.1| putative repressor [Staphylococcus aureus]
>gi|2769708|gb|AAB95639.1| pristinamycin resistance protein VgaB [Staphylococcus aureus]
>gi|2736228|gb|AAB94106.1| transducer protein [Staphylococcus aureus]
>gi|2736227|gb|AAB94105.1| mutant sensor protein [Staphylococcus aureus]
>gi|2736226|gb|AAB94104.1| pre-pheromone [Staphylococcus aureus]
>gi|2736225|gb|AAB94103.1| signal transduction protein [Staphylococcus aureus]
>gi|2736223|gb|AAB94102.1| transducer protein [Staphylococcus aureus]
>gi|2736222|gb|AAB94101.1| mutant sensor protein [Staphylococcus aureus]
>gi|2736221|gb|AAB94100.1| pre-pheromone [Staphylococcus aureus]
>gi|2736220|gb|AAB94099.1| signal transduction protein [Staphylococcus aureus]
>gi|2736218|gb|AAB94098.1| transducer protein [Staphylococcus aureus]
>gi|2736217|gb|AAB94097.1| mutant sensor protein [Staphylococcus aureus]
>gi|2736216|gb|AAB94096.1| pre-pheromone [Staphylococcus aureus]
>gi|2736215|gb|AAB94095.1| signal transduction protein [Staphylococcus aureus]
>gi|2696796|dbj|BAA24012.1| Fmt [Staphylococcus aureus]
>gi|2696713|dbj|BAA24009.1| integrase [Staphylococcus aureus]
>gi|2696712|dbj|BAA24008.1| LukF-PV [Staphylococcus aureus]
>gi|2696711|dbj|BAA24007.1| LukS-PV [Staphylococcus aureus]
>gi|216977|dbj|BAA00126.1| staphylocoagulase precursor [Staphylococcus aureus]
>gi|773396|emb|CAA39963.1| QacA protein [Staphylococcus aureus]
>gi|46660|emb|CAA39962.1| ORF188, has identity with known regulators such as tat regulator in Tn10
[Staphylococcus aureus]
>gi|2645713|gb|AAB87473.1| ribosome recycling factor [Staphylococcus aureus]
>gi|2641998|dbj|BAA23610.1| lipophilic protein [Staphylococcus aureus]
>gi|2580436|dbj|BAA23141.1| histidyl-tRNA synthetase [Staphylococcus aureus]
>gi|2580435|dbj|BAA23140.1| N-acetylmuramoyl-L-alanine amidase [Staphylococcus aureus]
>gi|2580434|dbj|BAA23139.1| ORF1 [Staphylococcus aureus]
>gi|2580433|dbj|BAA23138.1| ppGpp hydrolase [Staphylococcus aureus]
>gi|2580432|dbj|BAA23137.1| adenine phosphoribosyltransferase [Staphylococcus aureus]
>gi|1575062|gb|AAB81288.1| lytS [Staphylococcus aureus]
>gi|1575061|gb|AAB81287.1| ScdA [Staphylococcus aureus]
>gi|1694677|dbj|BAA13755.1| DnaA [Staphylococcus aureus]
>gi|2506163|gb|AAB81232.1| AgrD [Staphylococcus aureus]
>gi|1916240|gb|AAB81231.1| AgrA [Staphylococcus aureus]
>gi|1916239|gb|AAB81230.1| AgrC-31 [Staphylococcus aureus]
>gi|1916238|gb|AAB81229.1| AgrB [Staphylococcus aureus]
>gi|2506165|gb|AAB80783.1| AgrD [Staphylococcus aureus]
>gi|2506164|gb|AAB80779.1| AgrD [Staphylococcus aureus]
>gi|1916248|gb|AAB80782.1| AgrA [Staphylococcus aureus]
>gi|1916247|gb|AAB80781.1| AgrC [Staphylococcus aureus]
>gi|1916246|gb|AAB80780.1| AgrB [Staphylococcus aureus]
>gi|1916244|gb|AAB80778.1| AgrA [Staphylococcus aureus]
>gi|1916243|gb|AAB80777.1| AgrC [Staphylococcus aureus]
>gi|1916242|gb|AAB80776.1| AgrB [Staphylococcus aureus]
>gi|2344765|dbj|BAA21889.1| glutamic acid-specific protease [Staphylococcus aureus]
>gi|2302281|emb|CAA03107.1| unnamed protein product [Staphylococcus aureus]
>gi|2302279|emb|CAA03106.1| unnamed protein product [Staphylococcus aureus]
>gi|2258300|gb|AAB63269.1| AgrC [Staphylococcus aureus]
>gi|2258299|gb|AAB63268.1| AgrD [Staphylococcus aureus]
>gi|2258298|gb|AAB63267.1| AgrB [Staphylococcus aureus]
>gi|2258296|gb|AAB63266.1| AgrC [Staphylococcus aureus]
>gi|2258295|gb|AAB63265.1| AgrD [Staphylococcus aureus]
>gi|2258294|gb|AAB63264.1| AgrB [Staphylococcus aureus]
>gi|2239274|gb|AAB62278.1| peptidoglycan hydrolase [Staphylococcus aureus]
>gi|2224842|emb|CAA52098.1| squalene synthase [Staphylococcus aureus]
>gi|2224841|emb|CAA52097.1| squalene desaturase [Staphylococcus aureus]
>gi|437916|emb|CAA52296.1| isoleucyl-tRNA synthetase [Staphylococcus aureus]
>gi|2190507|emb|CAA71446.1| outer surface binding 70 kD protein [Staphylococcus aureus]
>gi|153069|gb|AAB59090.1| sigma factor
>gi|4433370|dbj|BAA22521.1| lipophilic protein which affects bacterial lysis rate and methicillin
resistance level [Staphylococcus aureus]
>gi|1262748|dbj|BAA12148.1| LukF-PV like component [Staphylococcus aureus]
>gi|1230554|dbj|BAA12147.1| LukM component [Staphylococcus aureus]
>gi|725454|dbj|BAA04185.1| autolysin [Staphylococcus aureus]
>gi|725453|dbj|BAA04184.1| ORF3 [Staphylococcus aureus]
>gi|725452|dbj|BAA04183.1| ORF2 [Staphylococcus aureus]
>gi|725451|dbj|BAA04182.1| ORF1 [Staphylococcus aureus]
>gi|540542|dbj|BAA01370.1| DNA gyrase A [Staphylococcus aureus]
>gi|540541|dbj|BAA01369.1| DNA gyrase B [Staphylococcus aureus]
>gi|522106|dbj|BAA06360.1| HSP40 [Staphylococcus aureus]
>gi|487331|dbj|BAA06361.1| ORF35 [Staphylococcus aureus]
>gi|487327|dbj|BAA06357.1| ORF37 [Staphylococcus aureus]
>gi|441211|dbj|BAA06359.1| HSP70 [Staphylococcus aureus]
>gi|441210|dbj|BAA06358.1| HSP20 [Staphylococcus aureus]
>gi|441208|dbj|BAA03533.1| HSP60 [Staphylococcus aureus]
>gi|441207|dbj|BAA03532.1| HSP10 [Staphylococcus aureus]
>gi|216975|dbj|BAA14147.1| ORF for norA [Staphylococcus aureus]
>gi|1483182|dbj|BAA13160.1| DNA polymerase III [Staphylococcus aureus]
>gi|1777321|dbj|BAA11087.1| DNA topoisomerase IV GrlA subunit [Staphylococcus aureus]
>gi|1777320|dbj|BAA11086.1| DNA topoisomerase IV GrlB subunit [Staphylococcus aureus]
>gi|1777318|dbj|BAA11085.1| DNA topoisomerase IV GrlA subunit [Staphylococcus aureus]
>gi|1777317|dbj|BAA11084.1| DNA topoisomerase IV GrlB subunit [Staphylococcus aureus]
>gi|628920|pir||S40421 hypothetical protein - Staphylococcus aureus
>gi|97848|pir||S12902 ricin chain A - Staphylococcus aureus
>gi|97853|pir||S14180 transposase - Staphylococcus aureus (fragment)
>gi|79909|pir||JU0116 transposase (insertion sequence IS431) - Staphylococcus aureus
>gi|79889|pir||S00092 penicillin-binding protein - Staphylococcus aureus
>gi|79872|pir||S04360 lacB protein - Staphylococcus aureus (fragment)
>gi|628921|pir||S40422 hypothetical protein - Staphylococcus aureus
>gi|1084186|pir||S54707 dnaA protein - Staphylococcus aureus
>gi|628917|pir||S27240 enterotoxin B - Staphylococcus aureus (fragments)
>gi|79852|pir||A29566 enterotoxin A - Staphylococcus aureus
>gi|79850|pir||S02008 dihydrofolate reductase (EC 1.5.1.3) - Staphylococcus aureus (fragment)
>gi|79845|pir||S15766 beta-hemolysin - Staphylococcus aureus
>gi|97845|pir||C42295 recf protein - Staphylococcus aureus (fragment)
>gi|2078390|gb|AAB54024.1| SecA [Staphylococcus aureus]
>gi|2078382|gb|AAB54022.1| SecY [Staphylococcus aureus]
>gi|2078381|gb|AAB54021.1| ribosomal protein L15 [Staphylococcus aureus]
>gi|2078380|gb|AAB54020.1| ribosomal protein L30 [Staphylococcus aureus]
>gi|2078378|gb|AAB54019.1| RplK; ribosomal protein L11 [Staphylococcus aureus]
>gi|2078377|gb|AAB54018.1| NusG [Staphylococcus aureus]
>gi|2078376|gb|AAB54017.1| SecE [Staphylococcus aureus]
>gi|2058356|emb|CAB06539.1| dihydropteroate synthase [Staphylococcus aureus]
>gi|1943995|dbj|BAA19494.1| sigA = sigma70 [Staphylococcus aureus]
>gi|1943994|dbj|BAA19493.1| dnaG [Staphylococcus aureus]
>gi|1943993|dbj|BAA19492.1| orf30 [Staphylococcus aureus]
>gi|1943992|dbj|BAA19491.1| orf15 [Staphylococcus aureus]
>gi|1944992|emb|CAA68933.1| ORF6 [Staphylococcus aureus]
>gi|1934991|emb|CAA68932.1| sigma factor B [Staphylococcus aureus]
>gi|1934990|emb|CAA68931.1| rsbW [Staphylococcus aureus]
>gi|1934989|emb|CAA68930.1| rsbV [Staphylococcus aureus]
>gi|1934988|emb|CAA68929.1| rsbU [Staphylococcus aureus]
>gi|1934987|emb|CAA68928.1| ORF1 [Staphylococcus aureus]
>gi|1916317|gb|AAB51152.1| alkyl hydroperoxide reductase subunit F [Staphylococcus aureus]
>gi|1916316|gb|AAB51151.1| alkyl hydroperoxide reductase subunit C [Staphylococcus aureus]
>gi|1913907|gb|AAB51063.1| TagD [Staphylococcus aureus]
>gi|1913906|gb|AAB51062.1| TagX [Staphylococcus aureus]
>gi|1913905|gb|AAB51061.1| TagB [Staphylococcus aureus]
>gi|1864022|gb|AAB50178.1| pencillin binding protein 4
>gi|881631|gb|AAB50179.1| AbcA
>gi|1657655|gb|AAB49445.1| Cap8P [Staphylococcus aureus]
>gi|1657654|gb|AAB49444.1| Cap8O [Staphylococcus aureus]
>gi|1657653|gb|AAB49443.1| Cap8N [Staphylococcus aureus]
>gi|1657652|gb|AAB49442.1| Cap8M [Staphylococcus aureus]
>gi|1657651|gb|AAB49441.1| Cap8L [Staphylococcus aureus]
>gi|1657650|gb|AAB49440.1| Cap8K [Staphylococcus aureus]
>gi|1657649|gb|AAB49439.1| Cap8J [Staphylococcus aureus]
>gi|1657648|gb|AAB49438.1| Cap8I [Staphylococcus aureus]
>gi|1657647|gb|AAB49437.1| Cap8H [Staphylococcus aureus]
>gi|1657646|gb|AAB49436.1| Cap8G [Staphylococcus aureus]
>gi|1657645|gb|AAB49435.1| Cap8F [Staphylococcus aureus]
>gi|1657644|gb|AAB49434.1| Cap8E [Staphylococcus aureus]
>gi|1657643|gb|AAB49433.1| Cap8D [Staphylococcus aureus]
>gi|1657642|gb|AAB49432.1| Cap8C [Staphylococcus aureus]
>gi|1657641|gb|AAB49431.1| Cap8B [Staphylococcus aureus]
>gi|1657640|gb|AAB49430.1| Cap8A [Staphylococcus aureus]
>gi|1854577|gb|AAB48183.1| lytR [Staphylococcus aureus]
>gi|862312|gb|AAB48182.1| lytS [Staphylococcus aureus]
>gi|1731452|gb|AAB48104.1| recombination protein [Staphylococcus aureus]
>gi|1053003|gb|AAB48103.1| CAT protein [Staphylococcus aureus]
>gi|1053002|gb|AAB48102.1| replication protein [Staphylococcus aureus]
>gi|1848269|gb|AAB47993.1| quaternary ammonium compounds resistance protein Qac [Staphylococcus
aureus]
>gi|1848268|gb|AAB47992.1| replication protein Rep [Staphylococcus aureus]
>gi|677847|emb|CAA24593.1| reading frame A [Staphylococcus aureus]
>gi|677846|emb|CAA24590.1| reading frame C [Staphylococcus aureus]
>gi|677845|emb|CAA24589.1| reading frame D [Staphylococcus aureus]
>gi|677844|emb|CAA24588.1| reading frame E [Staphylococcus aureus]
>gi|46746|emb|CAA38969.1| truncated alpha-toxin [Staphylococcus aureus]
>gi|46559|emb|CAA24592.1| reading frame F transl. attenuator [Staphylococcus aureus]
>gi|46558|emb|CAA24591.1| reading frame B mls resistance [Staphylococcus aureus]
>gi|987499|gb|AAB41908.1| 5-dehdroquinate synthase
>gi|987498|gb|AAB41907.1| chorismate synthase
>gi|987497|gb|AAB41906.1| nucleoside diphosphate kinase
>gi|987496|gb|AAB41905.1| geranylgeranyl pyrophosphate synthetase homolog; Method: conceptual
translation supplied by author
>gi|1262138|emb|CAA62900.1| glycerol 3-phosphate cytidyltransferase [Staphylococcus aureus]
>gi|1262137|emb|CAA62899.1| penicillin-binding protein 4 [Staphylococcus aureus]
>gi|1262136|emb CAA62898.1| ATP-binding cassette transporter A [Staphylococcus aureus]
>gi|1045529|gb|AAB39957.1| beta-lactamase
>gi|1045527|gb|AAB39956.1| beta-lactamase
>gi|1045525|gb|AAB39955.1| tetracycline resistance protein [Staphylococcus aureus]
>gi|1045524|gb|AAB39954.1| replication protein [Staphylococcus aureus]
>gi|1684749|emb|CAA70762.1| femD [Staphylococcus aureus]
>gi|1204146|emb|CAA65106.1| fibronectin-binding protein [Staphylococcus aureus]
>gi|1684751|emb|CAA70579.1| DNA directed RNA polymerase beta' chain [Staphylococcus aureus]
>gi|1478385|gb|AAB36169.1| MsrSa = 63 kda MsrA homolog {N-terminal} [Staphylococcus aureus, clinical
isolate, pEP2104, Peptide Plasmid Partial, 31 aa]
>gi|1042046|gb|AAB34958.1| IgG-binding polypeptide = protein A homolog [Staphylococcus aureus, strain
8325-4, Peptide Partial, 84 aa]
>gi|999313|gb|AAB34910.1| 60 kda vitronectin-binding surface protein {N-terminal} [Staphylococcus
aureus, prototype V8, Peptide Partial, 20 aa]
>gi|998765|gb|AAB34258.1| enterotoxin H {N-terminal} [Staphylococcus aureus, FRI-569, Peptide
Partial, 30 aa]
>gi|894289|gb|AAB33482.1| alkaline shock protein 23, ASP23 [Staphylococcus aureus, 912, Peptide, 169
aa]
>gi|619317|gb|AAB32218.1| beta-hemolysin, phospholipase C, PLC [Staphylococcus aureus, 126/89,
Peptide, 331 aa]
>gi|693735|gb|AAB31949.1| NorA {ISP794, quinolone resistance} [Staphylococcus aureus, NCTC 8325,
Peptide Insertion, 388 aa]
>gi|456770|gb|AAB28795.1| Tet(K) = tetracycline efflux protein [Staphylococcus aureus, pT181, Peptide
Plasmid, 459 aa]
>gi|433036|gb|AAB28599.1| adhesin {collagen binding domain} [Staphylococcus aureus, FDA 574, Peptide
Partial, 37 aa]
>gi|1680566|gb|AH004229.1| No definition line found
>gi|1680565|gb|AH004228.1| No definition line found
>gi|299115|gb|AAB26122.1| gamma-hemolysin component II, H gamma II = leukocidin S homolog
[Staphylococcus aureus, 4, RIMD 310925, Peptide Partial, 5 aa, segment 2 of 2]
>gi|299114|gb|AAB26121.1| gamma-hemolysin component II, H gamma II = leukocidin S homolog
[Staphylococcus aureus, 4, RIMD 310925, Peptide Partial, 58 aa, segment 1 of 2]
>gi|299112|gb|AAB26120.1| gamma-hemolysin component I, H gamma I = leukocidin F homolog
[Staphylococcus aureus, 4, RIMD 310925, Peptide Partial, 2 aa, segment 2 of 2]
>gi|299111|gb|AAB26119.1| gamma-hemolysin component I, H gamma I = leukocidin F homolog
[Staphylococcus aureus, 4, RIMD 310925, Peptide Partial, 59 aa, segment 1 of 2]
>gi|265412|gb|AAB25337.1| V8 protease [Staphylococcus aureus, Peptide, 276 aa]
>gi|248665|gb|AAB22051.1| chloramphenicol acetyltransferase, CAT {EC 2.3.1.28} [Staphylococcus
aureus, 4.6 kb chloramphenicol resistance (CmR) plasmid pSCS6, Peptide Plasmid, 215 aa]
>gi|246440|gb|AAB21603.1| 60 kda cell surface adhesin for heparan sulfate [Staphylococcus aureus,
Peptide Partial, 4 aa]
>gi|246439|gb|AAB21602.1| 66 kda cell surface adhesin for heparan sulfate [Staphylococcus aureus,
Peptide Partial, 9 aa]
>gi|239960|gb|AAB20545.1| 25-kda elastin-binding protein [Staphylococcus aureus, Peptide Partial, 14
aa]
>gi|239959|gb|AAB20544.1| 40-kda elastin-binding protein [Staphylococcus aureus, Peptide Partial, 18
aa]
>gi|1673527|gb|AAB18959.1| transposase [Staphylococcus aureus]
>gi|1673525|gb|AAB18958.1| transposase [Staphylococcus aureus]
>gi|1644433|gb|AAB17663.1| D-specific D-2-hydroxyacid dehydrogenase [Staphylococcus aureus]
>gi|581567|emb|CAA37260.1| Sau96I DNA methyltransferase [Staphylococcus aureus]
>gi|46618|emb|CAA37259.1| Sau96I restriction endonuclease [Staphylococcus aureus]
>gi|46597|emb|CAA37902.1| transposase [Staphylococcus aureus]
>gi|1587088|prf||2205353A pheromone [Staphylococcus aureus]
>gi|1583755|prf||2121375A MHC class II-like protein [Staphylococcus aureus]
>gi|229342|prf||710414A nuclease [Staphylococcus aureus]
>gi|229233|prf||670719A nuclease [Staphylococcus aureus]
>gi|1096955|prf||2113202C RNA polymerase:SUBUNIT = beta' [Staphylococcus aureus]
>gi|1096954|prf||2113202B RNA polymerase:SUBUNIT = beta [Staphylococcus aureus]
>gi|1096953|prf||2113202A ORF 202 [Staphylococcus aureus]
>gi|1095875|prf||2110238A lipase [Staphylococcus aureus]
>gi|1094971|prf||2107219C RNA polymerase:SUBUNIT = beta' [Staphylococcus aureus]
>gi|1094970|prf||2107219B RNA polymerase:SUBUNIT = beta [Staphylococcus aureus]
>gi|1094969|prf||2107219A rpoB upstream ORF [Staphylococcus aureus]
>gi|1093504|prf||2104216A LukM protein [Staphylococcus aureus]
>gi|1092377|prf||2023311A exotoxin [Staphylococcus aureus]
>gi|742313|prf||2009360B pcrB protein [Staphylococcus aureus]
>gi|742312|prf||2009360A helicase [Staphylococcus aureus]
>gi|448909|prf||1918210C leukocidin [Staphylococcus aureus]
>gi|448908|prf||1918210B leukocidin [Staphylococcus aureus]
>gi|448907|prf||1918210A gamma hemolysin [Staphylococcus aureus]
>gi|444424|prf||1907159A ethidium bromide resistance gene [Staphylococcus aureus]
>gi|384172|prf||1905282A rep protein [Staphylococcus aureus]
>gi|384170|prf||1905280A protein A [Staphylococcus aureus]
>gi|383540|prf||1903261A toxic shock syndrome toxin [Staphylococcus aureus]
>gi|228896|prf||1814271A Glu-C endoprotease [Staphylococcus aureus]
>gi|228567|prf||1806229B repressor [Staphylococcus aureus]
>gi|228566|prf||1806229A coinducer protein [Staphylococcus aureus]
>gi|228100|prf||1717222A REP protein [Staphylococcus aureus]
>gi|227968|prf||1714238A beta lactamase mutant S-3P [Staphylococcus aureus]
>gi|227467|prf||1704203A enterotoxin A [Staphylococcus aureus]
>gi|226860|prf||1609133A plasmid pOX1000 ORF A [Staphylococcus aureus]
>gi|581544|emb|CAA27142.1| kanamycin nucleotidyltransferase (AA 1-253) [Staphylococcus aureus]
>gi|46496|emb|CAA27141.1| repB polypeptide (AA 1-235) [Staphylococcus aureus]
>gi|1245474|gb|AAB09712.1| nicking enzyme [Staphylococcus aureus]
>gi|1586531|prf||2204232B penicillin-binding protein 4 [Staphylococcus aureus]
>gi|1586530|prf||2204232A ABC transporter-like protein [Staphylococcus aureus]
>gi|1585878|prf||2202209C ORF 3 [Staphylococcus aureus]
>gi|1585877|prf||2202209B ORF 2 [Staphylococcus aureus]
>gi|1585876|prf||2202209A ORF 1 [Staphylococcus aureus]
>gi|1053140|gb|AAB09660.1| tetracycline resistance protein [Staphylococcus aureus]
>gi|1575125|gb|AAB09464.1| beta-lactamase
>gi|226340|prf||1507213A transposase, insertion seq IS257 [Staphylococcus aureus]
>gi|225999|prf||1405331D repE gene [Staphylococcus aureus]
>gi|225998|prf||1405331C ORF D [Staphylococcus aureus]
>gi|225997|prf||1405331B rlx gene [Staphylococcus aureus]
>gi|359739|prf||1313299A staphylocoagulase [Staphylococcus aureus]
>gi|225442|prf||1303274B gene B [Staphylococcus aureus]
>gi|224810|prf||1202257F ORF [Staphylococcus aureus]
>gi|224809|prf||1202257E gene tnpC [Staphylococcus aureus]
>gi|224808|prf||1202257D gene tnpB [Staphylococcus aureus]
>gi|224806|prf||1202257B gene spc [Staphylococcus aureus]
>gi|224805|prf||1202257A gene ermA [Staphylococcus aureus]
>gi|1567208|emb|CAA02065.1| Ble [Staphylococcus aureus]
>gi|1860732|gb|AAB07805.1| phosphoenolpyruvate carboxykinase [Staphylococcus aureus]
>gi|1272327|gb|AAB07765.1| 3′5′-aminoglycoside phosphotransferase [Staphylococcus aureus]
>gi|1272326|gb|AAB07764.1| truncated streptothricin acetyl transferase [Staphylococcus aureus]
>gi|1236640|gb|AAB07747.1| multidrug resistance protein [Staphylococcus aureus]
>gi|1236639|gb|AAB07746.1| partial duplication of the qacC gene [Staphylococcus aureus]
>gi|1236638|gb|AAB07745.1| replication protein [Staphylococcus aureus]
>gi|1053000|gb|AAB07714.1| replication protein [Staphylococcus aureus]
>gi|1052999|gb|AAB07713.1| recombination protein [Staphylococcus aureus]
>gi|1052998|gb|AAB07712.1| tetracycline resistance protein [Staphylococcus aureus]
>gi|1125687|emb|CAA60583.1| glycerol-3-phosphate cytidyltransferase [Staphylococcus aureus]
>gi|1125686|emb|CAA60582.1| penicillin binding protein 4 [Staphylococcus aureus]
>gi|1125685|emb|CAA60581.1| mdr [Staphylococcus aureus]
>gi|1125683|emb|CAA60586.1| glycerol-3-phosphate cytidyltransferase [Staphylococcus aureus]
>gi|1125682|emb|CAA60585.1| penicillin binding protein 4 [Staphylococcus aureus]
>gi|1125681|emb|CAA60584.1| mdr [Staphylococcus aureus]
>gi|1495791|emb|CAA61517.1| DNA-directed RNA polymerase [Staphylococcus aureus]
>gi|758691|gb|AAB06195.1| enterotoxin D
>gi|1480567|gb|AAB05743.1| protein A
>gi|1477533|gb|AAB05396.1| sarA
>gi|1477532|gb|AAB05395.1| ORF3
>gi|225996|prf||1405331A str gene [Staphylococcus aureus]
>gi|225995|prf||1405330A repM gene [Staphylococcus aureus]
>gi|225821|prf||1314205A protein A [Staphylococcus aureus]
>gi|225444|prf||1303274D gene D [Staphylococcus aureus]
>gi|225443|prf||1303274C gene C [Staphylococcus aureus]
>gi|225441|prf||1303274A gene A [Staphylococcus aureus]
>gi|224812|prf||1202257H peptide 2 [Staphylococcus aureus]
>gi|224811|prf||1202257G peptide 1 [Staphylococcus aureus]
>gi|224807|prf||1202257C gene tnpA [Staphylococcus aureus]
>gi|224650|prf||1109959A nuclease,staphylococcal [Staphylococcus aureus]
>gi|223937|prf||1005204A hemolysin delta [Staphylococcus aureus]
>gi|1408063|gb|AAB03636.1| methicillin-resistance protein
>gi|295162|gb|AAB03637.1| unknown ORF1; putative
>gi|1407784|gb|AAB03613.1| orf-1; novel antigen
>gi|642270|emb|CAA88043.1| DNA polymerase III [Staphylococcus aureus]
>gi|1311537|gb|AAB02113.1| cop protein
>gi|153094|gb|AAB02114.1| resistance protein
>gi|153092|gb|AAB02112.1| replication protein
>gi|673492|emb|CAA24594.1| nuclease [Staphylococcus aureus]
>gi|1340131|emb|CAA66627.1| ORF4 [Staphylococcus aureus]
>gi|1340130|emb|CAA66626.1| ORF3 [Staphylococcus aureus]
>gi|1340129|emb|CAA66625.1| ORF2 [Staphylococcus aureus]
>gi|1340128|emb|CAA66624.1| ORF1 [Staphylococcus aureus]
>gi|758303|emb|CAA24957.1| staphylokinase [Staphylococcus aureus]
>gi|736295|emb|CAA27035.1| A protein [Staphylococcus aureus]
>gi|581591|emb|CAA34491.1| beta-lactamase (AA 1-281) [Staphylococcus aureus]
>gi|551590|emb|CAA36953.1| blaZ protein (AA 1-281) [Staphylococcus aureus]
>gi|581589|emb|CAA36950.1| binL protein (AA 1-197) [Staphylococcus aureus]
>gi|46765|emb|CAA25801.1| put. alpha-toxin precursor (aa −26 to 293) [Staphylococcus aureus]
>gi|46759|emb|CAA36952.1| blaR1 protein (AA 1-585) [Staphylococcus aureus]
>gi|46758|emb|CAA36951.1| blaI protein (AA 1-126) [Staphylococcus aureus]
>gi|46756|emb|CAA36949.1| ORF480 (pot. transposase) (AA 1-480) [Staphylococcus aureus]
>gi|46755|emb|CAA36948.1| ORF271 (pot. ATP-binding protein) (AA 1-271) [Staphylococcus aureus]
>gi|46753|emb|CAA31652.1| transposase (AA 1-224) [Staphylococcus aureus]
>gi|46752|emb|CAA31651.1| transposase (AA 1-224) [Staphylococcus aureus]
>gi|46751|emb|CAA31650.1| ORF140 (AA 1-140) [Staphylococcus aureus]
>gi|46750|emb|CAA31649.1| S1 DHFR (AA 1-161) [Staphylococcus aureus]
>gi|46749|emb|CAA31648.1| thymidylate synthetase (AA 1-318) [Staphylococcus aureus]
>gi|46748|emb|CAA31647.1| transposase (AA 1-224) [Staphylococcus aureus]
>gi|758275|emb|CAA29822.1| sak42D staphylokinase [Staphylococcus aureus]
>gi|581583|emb|CAA24596.1| protein A [Staphylococcus aureus]
>gi|459257|emb|CAA83066.1| Potential membrane spanning protein [Staphylococcus aureus]
>gi|459256|emb|CAA83065.1| Potential ABC transporter [Staphylococcus aureus]
>gi|46737|emb|CAA34476.1| precursor polypeptide (AA −26 to 632) [Staphylococcus aureus]
>gi|46691|emb|CAA43604.1| protein A [Staphylococcus aureus]
>gi|46687|emb|CAA68434.1| preproenzyme (AA −68 to 268) [Staphylococcus aureus]
>gi|1070386|emb|CAA63689.1| phosphoenolpyruvate-protein phosphatase [Staphylococcus aureus]
>gi|1070385|emb|CAA63688.1| histidin-containing protein [Staphylococcus aureus]
>gi|677852|emb|CAA45513.1| DNA-directed RNA polymerase beta' chain [Staphylococcus aureus]
>gi|677851|emb|CAA45512.1| DNA-directed RNA polymerase beta chain [Staphylococcus aureus]
>gi|677850|emb|CAA45511.1| hypothetical protein [Staphylococcus aureus]
>gi|677849|emb|CAA45510.1| ribosomal protein L7/L12 [Staphylococcus aureus]
>gi|581571|emb|CAA43217.1| chlorAMPhenicol acetyltransferase [Staphylococcus aureus]
>gi|551670|emb|CAA51251.1| lukS [Staphylococcus aureus]
>gi|551669|emb|CAA51250.1| ORF [Staphylococcus aureus]
>gi|288292|emb|CAA51252.1| leucocidin F [Staphylococcus aureus]
>gi|46652|emb|CAA43218.1| chlorAMPhenicol acetyltransferase [Staphylococcus aureus]
>gi|1333818|emb|CAA26369.1| pot. orfB (aa 1-92) (4557 is 2nd base in codon) [Staphylococcus aureus]
>gi|1333817|emb|CAA26368.1| pot. orfA [Staphylococcus aureus]
>gi|809754|emb|CAA26365.1| unidentified reading frame [Staphylococcus aureus]
>gi|581570|emb|CAA41339.1| dihydrolipoamide acetyltransferase: subunit E2 [Staphylococcus aureus]
>gi|581568|emb|CAA27733.1| beta-lactamase (aa 1-281) [Staphylococcus aureus]
>gi|488529|emb|CAA45728.1| S component of leucocodin R [Staphylococcus aureus]
>gi|483534|emb|CAA44177.1| penicillin-binding protein 2 [Staphylococcus aureus]
>gi|295834|emb|CAA39320.1| ORF 154 [Staphylococcus aureus]
>gi|48874|emb|CAA41340.1| dihydrolipoamide dehydrogenase: subunit E3 [Staphylococcus aureus]
>gi|48872|emb|CAA41338.1| pyruvate dehydrogenase (lipoamide): subunit E1beta [Staphylococcus
aureus]
>gi|46647|emb|CAA29842.1| ORF (repE) [Staphylococcus aureus]
>gi|46646|emb|CAA29841.1| orfD [Staphylococcus aureus]
>gi|46645|emb|CAA29840.1| ORF (rlx) [Staphylococcus aureus]
>gi|46644|emb|CAA29839.1| ORF (str) [Staphylococcus aureus]
>gi|46639|emb|CAA31314.1| ORF 1 (AA 1-330) [Staphylococcus aureus]
>gi|46638|emb|CAA31313.1| ORF 2 (AA 1-236) [Staphylococcus aureus]
>gi|46636|emb|CAA30291.1| RepM protein (AA 1-314) [Staphylococcus aureus]
>gi|46632|emb|CAA26367.1| CAT gene (aa 1-215) [Staphylococcus aureus]
>gi|46631|emb|CAA26366.1| repD (aa 1-311) [Staphylococcus aureus]
>gi|46629|emb|CAA68684.1| penicillin-binding protein (AA 1-670) [Staphylococcus aureus]
>gi|46622|emb|CAA42079.1| E.coli isoleucyl tRNA synthetase homologue [Staphylococcus aureus]
>gi|46620|emb|CAA42080.1| E.coli isoleucyl tRNA synthetase homologue [Staphylococcus aureus]
>gi|46611|emb|CAA36829.1| PBP2′ (AA 1-668) [Staphylococcus aureus]
>gi|46609|emb|CAA45729.1| F component of leucocodin R [Staphylococcus aureus]
>gi|1134886|emb|CAA54030.1| glutamine synthetase [Staphylococcus aureus]
>gi|581562|emb|CAA44726.1| fibronectin binding protein B [Staphylococcus aureus]
>gi|550424|emb|CAA57278.1| hlgB-like ORF [Staphylococcus aureus]
>gi|550423|emb|CAA57277.1| hlgC-like ORF [Staphylococcus aureus]
>gi|550422|emb|CAA57276.1| hlgA-like ORF [Staphylococcus aureus]
>gi|468509|emb|CAA54029.1| glutamine synthetase repressor [Staphylococcus aureus]
>gi|468508|emb|CAA54028.1| ORF1 [Staphylococcus aureus]
>gi|311976|emb|CAA50893.1| fibrinogen-binding protein [Staphylococcus aureus]
>gi|311974|emb|CAA50892.1| fibrinogen-binding protein [Staphylococcus aureus]
>gi|296396|emb|CAA50571.1| DNA gyrase [Staphylococcus aureus]
>gi|296395|emb|CAA50570.1| DNA gyrase [Staphylococcus aureus]
>gi|296394|emb|CAA50569.1| RecF [Staphylococcus aureus]
>gi|48713|emb|CAA36830.1| orf145 [Staphylococcus aureus]
>gi|46606|emb|CAA32936.1| lacD polypeptide (AA 1-326) [Staphylococcus aureus]
>gi|46605|emb|CAA32935.1| lacC polypeptide (AA 1-310) [Staphylococcus aureus]
>gi|46589|emb|CAA31770.1| ORF 2 (68 AA) (2187 is 2nd base in codon) [Staphylococcus aureus]
>gi|46588|emb|CAA31769.1| beta-hemolysin (AA 1-330) [Staphylococcus aureus]
>gi|46587|emb|CAA31768.1| ORF 1 (AA 1-121) (1 is 2nd base in codon) [Staphylococcus aureus]
>gi|46582|emb|CAA35680.1| ORF 419 protein [Staphylococcus aureus]
>gi|46581|emb|CAA35679.1| FemA protein [Staphylococcus aureus]
>gi|46580|emb|CAA35678.1| trpA protein (AA at 1) [Staphylococcus aureus]
>gi|46576|emb|CAA26883.1| Methylase (AA 1-11) (172 is 2nd base in codon) [Staphylococcus aureus]
>gi|809753|emb|CAA26103.1| pot. reading frame C (aa 51-230) (1 is 3rd base in codon) [Staphylococcus
aureus]
>gi|736294|emb|CAA68826.1| transposase [Staphylococcus aureus]
>gi|671632|emb|CAA78911.1| unknown [Staphylococcus aureus]
>gi|581558|emb|CAA53189.1| isoleucyl tRNA synthetase [Staphylococcus aureus]
>gi|438228|emb|CAA53191.1| ORF C [Staphylococcus aureus]
>gi|438227|emb|CAA53190.1| ORF B [Staphylococcus aureus]
>gi|49313|emb|CAA78910.1| dihydrofolate reductase [Staphylococcus aureus]
>gi|49018|emb|CAA44472.1| ethidium bromide resistance protein [Staphylococcus aureus]
>gi|49017|emb|CAA44471.1| ethidium bromide resistance protein [Staphylococcus aureus]
>gi|46573|emb|CAA38227.1| ermC [Staphylococcus aureus]
>gi|46571|emb|CAA35972.1| staphylococcal enterotoxin C3 [Staphylococcus aureus]
>gi|1015408|gb|AAA79054.1| chloramphenicol acetyltransferase
>gi|1015407|gb|AAA79053.1| cat leader peptide
>gi|1015406|gb|AAA79052.1| replication initiation protein
>gi|915308|gb|AAA74889.1| fibrinogen binding protein
>gi|409241|gb|AAA74375.1| penicillin-binding protein 2
>gi|153086|gb|AAA73952.1| DNA gyrase A subunit
>gi|153085|gb|AAA73951.1| DNA gyrase B subunit
>gi|153084|gb|AAA73950.1| homologue; putative
>gi|153062|gb|AAA72091.1| helicase
>gi|153061|gb|AAA72090.1| [pcrA] gene products
>gi|152957|gb|AAA71898.1| 5-enolpyruvylshikimate-3-phosphate synthase
>gi|152956|gb|AAA71897.1| 3-phosphoshikimate-1-carboxyvinyltransferase
>gi|152955|gb|AAA71896.1| 3-dehydroquinate synthase
>gi|845687|gb|AAA67855.1| lacA repressor, putative
>gi|845686|gb|AAA67853.1| ORF-27
>gi|153035|gb|AAA67854.1| lacR repressor
>gi|567036|gb|AAA64644.1| CapE
>gi|506709|gb|AAA64652.1| CapM
>gi|506708|gb|AAA64651.1| CapL
>gi|506707|gb|AAA64650.1| CapK
>gi|506706|gb|AAA64649.1| CapJ
>gi|506705|gb|AAA64648.1| CapI
>gi|506704|gb|AAA64647.1| CapH
>gi|506703|gb|AAA64646.1| CapG
>gi|506702|gb|AAA64645.1| CapF
>gi|506700|gb|AAA64643.1| CapD
>gi|506699|gb|AAA64642.1| CapC
>gi|506698|gb|AAA64641.1| CapB
>gi|506697|gb|AAA64640.1| CapA
>gi|684950|gb|AAA62477.1| staphylococcal accessory regulator A
>gi|567884|gb|AAA53114.1| lysyl-tRNA synthetase
>gi|561880|gb|AAA53116.1| gyrase-like protein alpha subunit
>gi|561879|gb|AAA53115.1| gyrase-like protein beta subunit
>gi|463285|gb|AAA50463.1| putative
>gi|551992|gb|AAA26680.1| 25 kD protein (putative); putative
>gi|551991|gb|AAA26674.1| enterotoxin B
>gi|495089|gb|AAA26675.1| recombinase
>gi|398085|gb|AAA26683.1| acetyltransferase
>gi|153125|gb|AAA26684.1| ATP-binding protein
>gi|153123|gb|AAA26682.1| toxic shock syndrome toxin-1 precursor
>gi|153121|gb|AAA26681.1| staphylococcal enterotoxin A precursor
>gi|153115|gb|AAA26678.1| tetM
>gi|153106|gb|AAA26677.1| protein A (ttg start codon)
>gi|153104|gb|AAA26676.1| protein A (ttg start codon)
>gi|153100|gb|AAA26673.1| Sau3AIM protein
>gi|153099|gb|AAA26672.1| Sau3AIR protein (ttg start codon)
>gi|153097|gb|AAA26671.1| bleomycin resistance protein
>gi|153096|gb|AAA26670.1| neomycin resistance protein
>gi|153090|gb|AAA26669.1| REP N protein (rep N)
>gi|153088|gb|AAA26668.1| ethidium resistance protein (ebr)
>gi|153082|gb|AAA26667.1| recombination and repair protein
>gi|153080|gb|AAA26666.1| resistance protein
>gi|153079|gb|AAA26665.1| transposase
>gi|153067|gb|AAA26662.1| peptidoglycan hydrolase
>gi|153057|gb|AAA26659.1| nuclease precursor
>gi|153055|gb|AAA26658.1| norA
>gi|537341|gb|AAA26647.1| beta-lactamase
>gi|537340|gb|AAA26646.1| beta-lactamase
>gi|537339|gb|AAA26645.1| beta-lactamase
>gi|537338|gb|AAA26644.1| beta-lactamase
>gi|537337|gb|AAA26643.1| beta-lactamase
>gi|537336|gb|AAA26642.1| beta-lactamase
>gi|475839|gb|AAA26654.1| leucocidin S component
>gi|393266|gb|AAA26634.1| glycerol ester hydrolase
>gi|295156|gb|AAA26639.1| gamma-hemolysin component B
>gi|295155|gb|AAA26638.1| gamma-hemolysin component C
>gi|295154|gb|AAA26637.1| gamma-hemolysin component A
>gi|295152|gb|AAA26632.1| fibronectin-binding protein precursor
>gi|153045|gb|AAA26653.1| prolipoprotein signal peptidase
>gi|153041|gb|AAA26651.1| lincosaminide nucleotidyltransferase
>gi|153039|gb|AAA26650.1| phospho-beta-galactosidase (lacG)
>gi|153038|gb|AAA26649.1| enzyme II-lac (lacE)
>gi|153037|gb|AAA26648.1| enzyme III-lac (lacF)
>gi|153033|gb|AAA26641.1| tagatose 6-phosphate isomerase
>gi|153027|gb|AAA26640.1| DNA invertase (ttg start codon)
>gi|153025|gb|AAA26636.1| gyrase A
>gi|153024|gb|AAA26635.1| gyrase B
>gi|153020|gb|AAA26633.1| lipase precursor (geh; EC 3.1.1.3)
>gi|295151|gb|AAA26623.1| enterotoxin
>gi|295149|gb|AAA26622.1| enterotoxin
>gi|295147|gb|AAA26621.1| enterotoxin
>gi|295145|gb|AAA26620.1| enterotoxin
>gi|295143|gb|AAA26619.1| enterotoxin
>gi|295141|gb|AAA26618.1| enterotoxin
>gi|153012|gb|AAA26628.1| ETB precursor
>gi|153008|gb|AAA26626.1| epidermolytic toxin A precursor
>gi|153006|gb|AAA26625.1| ETA precursor
>gi|153004|gb|AAA26624.1| enterotoxin C3
>gi|153002|gb|AAA26617.1| enterotoxin type E precursor
>gi|152998|gb|AAA26616.1| epidermal cell differentiation inhibitor
>gi|152982|gb|AAA26613.1| chloramphenicol acetyltransferase
>gi|152981|gb|AAA26612.1| precursor protein
>gi|152979|gb|AAA26611.1| putative
>gi|152978|gb|AAA26610.1| E1-E2 cadmium efflux adenosine triphosphatase
>gi|152977|gb|AAA26609.1| cadmium resistance protein
>gi|152976|gb|AAA26608.1| 125 codon reading frame that is similar to Tn554-encoded TnpC; RBS
retained, but no evidence as to whether transcribed or expressed; putative
>gi|152975|gb|AAA26607.1| 630 codon reading frame resembling tnpB of Tn554; transcription or
translation signals upstream of the reading frame removed by the deletion noted above; presumed to
be vestigial and not expressed; putative
>gi|152974|gb|AAA26606.1| 29 codon reading frame of which N-terminal 25 are similar to the
corresponding region of Tn554-encoded TnpA; TnpA is truncated by a 1006 bp deletion and is presumed
inactive; putative
>gi|152970|gb|AAA26605.1| beta-lactamase repressor
>gi|152953|gb|AAA26598.1| alpha-hemolysin
>gi|152951|gb|AAA26597.1| accessory gene regulator protein
>gi|152945|gb|AAA26596.1| aminocyclitol-3′-phosphotransferase
>gi|532967|gb|AAA21184.1| ORF
>gi|295159|gb|AAA21183.1| putative
>gi|295158|gb|AAA21182.1| mecR
>gi|387880|gb|AAA20874.1| collagen adhesin
>gi|310604|gb|AAA18516.1| ORF3
>gi|310603|gb|AAA18515.1| ORF2
>gi|310602|gb|AAA18514.1| ORF1
>gi|409063|gb|AAA17490.1| exfoliative toxin A
>gi|152985|gb|AAA16529.1| chloramphenicol acetyltransferase
>gi|152984|gb|AAA16528.1| precursor protein
>gi|458428|gb|AAA16512.1| FtsZ
>gi|425478|gb|AAA16442.1| phosphatidylinositol-specific phospholipase C
>gi|153053|gb|AAA16158.1| norA1199 protein

TABLE 7
MAP AND SEQUENCE POSITION OF THE 73 ORFs PREDICTED
TO BE ENCODED BY PHAGE 44AHJD THAT ARE GREATER
THAN 33 AMINO ACIDS
Phage 44AHJD ORFs list

| nb | Name | Frame | Position | Size (a.a.) | Key words |
|---|---|---|---|---|---|
| 1 | 44AHJDORF001 | −1 | 10342 . . . 12627 | 761 | DNA polymerase; |
| 2 | 44AHJDORF002 | 3 | 3789 . . . 5732 | 647 | Techoic acid; Staph; |
| 3 | 44AHJDORF003 | 2 | 6626 . . . 8389 | 587 | Tail; |
| 4 | 44AHJDORF004 | 1 | 8764 . . . 10227 | 487 | Serine protease motif; |
| 5 | 44AHJDORF005 | −1 | 12643 . . . 13890 | 415 | |
| 6 | 44AHJDORF006 | 2 | 803 . . . 2029 | 408 | |
| 7 | 44AHJDORF007 | 1 | 2044 . . . 3027 | 327 | Upper collar; |
| 8 | 44AHJDORF008 | 2 | 3020 . . . 3775 | 251 | Lower collar; |
| 9 | 44AHJDORF009 | 2 | 5744 . . . 6496 | 250 | Amidase; Staph; |
| 10 | 44AHJDORF010 | −2 | 13938 . . . 14420 | 160 | |
| 11 | 44AHJDORF012 | 3 | 8391 . . . 8813 | 140 | Holin; |
| 12 | 44AHJDORF013 | −2 | 14586 . . . 14996 | 136 | |
| 13 | 44AHJDORF113 | 1 | 199 . . . 600 | 133 | |
| 14 | 44AHJDORF011 | −2 | 15225 . . . 15593 | 122 | |
| 15 | 44AHJDORF114 | −2 | 15870 . . . 16172 | 100 | |
| 16 | 44AHJDORF014 | 3 | 6243 . . . 6521 | 92 | |
| 17 | 44AHJDORF015 | 1 | 15403 . . . 15645 | 80 | |
| 18 | 44AHJDORF016 | −1 | 15616 . . . 15852 | 78 | |
| 19 | 44AHJDORF017 | −2 | 10536 . . . 10757 | 73 | |
| 20 | 44AHJDORF018 | −1 | 886 . . . 1098 | 70 | |
| 21 | 44AHJDORF019 | −2 | 9630 . . . 9836 | 68 | |
| 22 | 44AHJDORF121 | −1 | 16165 . . . 16362 | 65 | |
| 23 | 44AHJDORF020 | 2 | 13865 . . . 14053 | 62 | |
| 24 | 44AHJDORF123 | 2 | 614 . . . 796 | 60 | |
| 25 | 44AHJDORF021 | −2 | 5634 . . . 5816 | 60 | |
| 26 | 44AHJDORF023 | −2 | 6315 . . . 6494 | 59 | |
| 27 | 44AHJDORF024 | 1 | 14275 . . . 14451 | 58 | |
| 28 | 44AHJDORF025 | −3 | 14999 . . . 15175 | 58 | |
| 29 | 44AHJDORF026 | −3 | 14426 . . . 14593 | 55 | |
| 30 | 44AHJDORF027 | 1 | 12916 . . . 13080 | 54 | |
| 31 | 44AHJDORF029 | −1 | 15019 . . . 15183 | 54 | |
| 32 | 44AHJDORF028 | −3 | 9071 . . . 9235 | 54 | |
| 33 | 44AHJDORF030 | 3 | 14487 . . . 14648 | 53 | |
| 34 | 44AHJDORF031 | 2 | 11039 . . . 11191 | 50 | |
| 35 | 44AHJDORF135 | 3 | 693 . . . 842 | 49 | |
| 36 | 44AHJDORF033 | −1 | 3646 . . . 3795 | 49 | |
| 37 | 44AHJDORF032 | −2 | 9306 . . . 9455 | 49 | |
| 38 | 44AHJDORF034 | −3 | 14000 . . . 14146 | 48 | |
| 39 | 44AHJDORF035 | −3 | 13811 . . . 13957 | 48 | |
| 40 | 44AHJDORF036 | −3 | 10019 . . . 10165 | 48 | |
| 41 | 44AHJDORF022 | −3 | 8468 . . . 8611 | 47 | |
| 42 | 44AHJDORF037 | 1 | 14788 . . . 14931 | 47 | |
| 43 | 44AHJDORF038 | −2 | 3528 . . . 3671 | 47 | |
| 44 | 44AHJDORF039 | 3 | 1743 . . . 1883 | 46 | |
| 45 | 44AHJDORF040 | 2 | 9740 . . . 9877 | 45 | |
| 46 | 44AHJDORF041 | 2 | 15836 . . . 15973 | 45 | |
| 47 | 44AHJDORF042 | −1 | 5014 . . . 5151 | 45 | |
| 48 | 44AHJDORF043 | −1 | 4402 . . . 4539 | 45 | |
| 49 | 44AHJDORF044 | −2 | 12783 . . . 12917 | 44 | |
| 50 | 44AHJDORF149 | −2 | 639 . . . 770 | 43 | |
| 51 | 44AHJDORF046 | 1 | 4891 . . . 5019 | 42 | |
| 52 | 44AHJDORF047 | 1 | 11911 . . . 12039 | 42 | |
| 53 | 44AHJDORF045 | 2 | 10655 . . . 10783 | 42 | |
| 54 | 44AHJDORF048 | −3 | 15212 . . . 15340 | 42 | |
| 55 | 44AHJDORF049 | 3 | 5784 . . . 5909 | 41 | |
| 56 | 44AHJDORF050 | 3 | 13158 . . . 13283 | 41 | |
| 57 | 44AHJDORF051 | −2 | 10944 . . . 11066 | 40 | |
| 58 | 44AHJDORF052 | −3 | 14216 . . . 14338 | 40 | |
| 59 | 44AHJDORF053 | 3 | 3348 . . . 3467 | 39 | |
| 60 | 44AHJDORF054 | 3 | 7551 . . . 7670 | 39 | |
| 61 | 44AHJDORF055 | 3 | 15705 . . . 15821 | 38 | |
| 62 | 44AHJDORF056 | 1 | 5512 . . . 5625 | 37 | |
| 63 | 44AHJDORF057 | 2 | 10121 . . . 10231 | 36 | |
| 64 | 44AHJDORF058 | 3 | 10767 . . . 10877 | 36 | |
| 65 | 44AHJDORF164 | −1 | 592 . . . 702 | 36 | |
| 66 | 44AHJDORF059 | −2 | 8250 . . . 8360 | 36 | |
| 67 | 44AHJDORF060 | −2 | 6147 . . . 6257 | 36 | |
| 68 | 44AHJDORF061 | 2 | 15551 . . . 15658 | 35 | |
| 69 | 44AHJDORF062 | 1 | 4285 . . . 4389 | 34 | |
| 70 | 44AHJDORF063 | −3 | 9383 . . . 9487 | 34 | |
| 71 | 44AHJDORF065 | 1 | 5029 . . . 5130 | 33 | |
| 72 | 44AHJDORF064 | 2 | 2609 . . . 2710 | 33 | |
| 73 | 44AHJDORF066 | −2 | 10380 . . . 10481 | 33 | |

TABLE 8

NUCLEOTIDE AND PREDICTED AMINO ACID SEQUENCE OF ALL 73 ORFs
IDENTIFIED IN PHAGE 44 AHJD

44AHJDORF001

| | |
|---|---|
| 12627 | atgggattactagaatgcatgcaatatcataaacatgaacgtcgaatgattttatactgggatatagaaacattagcgtacaat |
| 1 | M G L L E C M Q Y H K H E R R M I L Y W D I E T L A Y N |
| 12543 | aaagttaacggacgaaaaaaaccaaccaaatataaaaacgttacttattctgtagcaattggttggtttaatggttatgaaatt |
| 29 | K V N G R K K P T K Y K N V T Y S V A I G W F N G Y E I |
| 12459 | gatgttgaagtatttccgagtttcgaatctttttatgacgcattttatacgtatgtgaaaagacgtgatacaatcacaaaatca |
| 57 | D V E V F P S F E S F Y D A F Y T Y V K R R D T I T K S |
| 12375 | aaaacagatattatcatgattgcacataactgtaataaatacgataatcattttttacttaaagacaccatgcgttattttgat |
| 85 | K T D I I H I A H N C N K Y D N H F L L K D T M R Y F D |
| 12291 | aatattacacgcgaaaatatatatttaaaatctgcagaagaaaatgaacacacattaaaaatgaaagaggctactattttagcc |
| 113 | N I T R E N I Y L K S A E E N E H T L K M K E A T I L A |
| 12207 | aaaaatcaaaatgtaattttagaaaaacgtgttaaatcttcaatcaatttagatttaacaatgtttttaaatggttttaaattt |
| 141 | K N Q N V I L E K R V K S S I N L D L T M F L N G F K F |
| 12123 | aatattattgataactttatgaaaaccaatacatcaattgcaacattaggtaagaaattacttgatggtggttatttaacagaa |
| 169 | N I I D N F M K T N T S I A T L G K K L L D G G Y L T E |
| 12039 | tcacaacttaaaacagattttaattatacgatttttgataaagataatgatatgaatgatagtgaagcctatgactatgctgtg |
| 197 | S Q L K T D F N Y T I F D K D N D M N D S E A Y D Y A V |
| 11955 | aaatgttttgcaaaactcacacctgaacaacttacatacattcataatgacgtgattatattaggtatgtgccatattcattat |
| 225 | K C F A K L T P E Q L T Y I H N D V I I L G M C H I H Y |
| 11871 | agtgatatatttccaaattttsactataacaaattaacattttcattgaatattatggaatcttacttgaataatgaaatgaca |
| 253 | S D I F P N F D Y N K L T F S L N I M E S Y L N N E M T |
| 11787 | cgttttcagttactcaaccaatatcaagatattaaaatatcttatacacattatcatttccatgatatgaatttttatgactat |
| 281 | R F Q L L N Q Y Q D I K I S Y T H Y H F H D M N F Y D Y |
| 11703 | attaaatcattctatcgtggtggtttaaatatgtataacaccaaatacataaacaaactaattgatgagccttgtttttctatt |
| 309 | I K S F Y R G G L N M Y N T K Y I N K L I D E P C F S I |
| 11619 | gacatcaattcgagttatccttatgtgatgtatcatgaaaaaattccaacatggttatacttttacgaacactattcagaacca |
| 337 | D I N S S Y P Y V M Y H E K I P T W L Y F Y E H Y S E P |
| 11535 | acgttaatccctacttttttagatgatgacaattatttttcattatataagattgataaagatgtatttaacgatgatttatta |
| 365 | T L I P T F L D D D N Y F S L Y K I D K D V F N D D L L |
| 11451 | attaaaattaaatcacgtgtattacgtcaaatgattgtaaaatactataataatgataatgattacgttaatatcaatacaaat |
| 393 | I K I K S R V L R Q M I V K Y Y N N D N D Y V N I N T N |
| 11367 | acattaagaatgattcaagacattacgggtattgattgcatgcatatacgtgttaattcgtttgttatatatgaatgtgaatac |
| 421 | T L R M I Q D I T G I D C M H I R V N S F V I Y E C E Y |
| 11283 | tttcatgcacgtgatattatttttcaaaactattttattaaaacacaaggtaagttaaaaaacaaaatcaatatgacatcacct |
| 449 | F H A R D I I F Q N Y F I K T Q G K L K N K I N M T S P |
| 11199 | tacgactatcacattactgatgatatcaacgaacacccatactcaaatgaggaggttatgttatctaaagtcgttttaaatgga |
| 477 | Y D Y H I T D D I N E H P Y S N E E V M L S K V V L N G |
| 11115 | ttatatggcatacctgcattacgttcacattttaacttattccgtttagatgataacaatgaactatacaatatcattaacggt |
| 505 | L Y G I P A L R S H F N L F R L D D N N E L Y N I I N G |
| 11031 | tacaaaaacactgaacgtaatatattattctctacatttgtcacatcacgttcattgtataacttattggttcctttccaatac |
| 533 | Y K N T E R N I L F S T F V T S R S L Y N L L V P F Q Y |
| 10947 | ttaacggaaagtgaaattgacgacaattttatttattgcgatactgatagtttgtatatgaaatccgttgttaaacccttattg |
| 561 | L T E S E I D D N F I Y C D T D S L Y M K S V V K P L L |
| 10863 | aaccccagtttattcgacccgatagccttaggtaaatgggatattgaaaacgaacagatagataagatgtttgtactgaatcat |
| 589 | N P S L F D P I A L G K W D I E N E Q I D K M F V L N H |
| 10779 | aagaaatatgcatatgaagtgaatggaaagattaaaattgcttctgctggtataccgaaaaacgcctttgatacaagcgtcgat |
| 617 | K K Y A Y E V N G K I K I A S A G I P K N A F D T S V D |
| 10695 | tttgaaacctttgtacgtgaacaattctttgacggtgccattattgaaaacaataaaagtatctataatgagcaaggtacaata |
| 645 | F E T F V R E Q F F D G A I I E N N K S I Y N E Q G T I |
| 10611 | tcgatatatccgtctaaaactgaaattgtatgtggtaatgtatatgatgaatattttactgatgaacttaatatgaaacgtgaa |
| 673 | S I Y P S K T E I V C G N V Y D E Y F T D E L N M K R E |
| 10527 | tttatattaaaagacgctagagaaaatttcgaccatagtcaatttgatgatattctttatattgaaagtgacatcggttcattt |
| 701 | F I L K D A R E N F D H S Q F D D I L Y I E S D I G S F |
| 10443 | tcacttaacgacttatttccagttgaacgttcagtacataacaaatctgatttgcatatattaaaacgtgaacatgatgaaata |
| 729 | S L N D L F P V E R S V H N K S D L H I L K R E H D E I |
| 10359 | aaaaaaggcaactgttaa 10342 |
| 757 | K K G N C * |

44AHJDHKF002

| | |
|---|---|
| 3789 | atggcatataatgaaaacgattttaaatattttgatgacattcgtccatttttagacgaaatttataaaacgagagaacgttat |
| 1 | M A Y N E N D F K Y F D D I R P F L D E I Y K T R E R Y |
| 3873 | acaccgttttacgatgatagagcagattataatactaattcaaaatcatattatgattatatttcaagattatcaaaactaatt |
| 29 | T P F Y D D R A D Y N T N S K S Y Y D Y I S R L S K L I |
| 3957 | gaagtattagcacgtcgtatttgggactatgacaatgaattaaaaaaacgtttcaaaaattgggacgacttaatgaaagcattt |
| 57 | E V L A R R I W D Y D N E L K K R F K N W D D L M K A F |
| 4041 | ccagagcaagcgaaagacttatttagaggttggttaaacgacggtacgattgacagtattattcatgacgagtttaaaaaatat |
| 85 | P E Q A K D L F R G W L N D G T I D S I I H D E F K K Y |
| 4125 | agcgcaggattaacatcggcatttgctttatttaaagttactgaaatgaaacaaatgaatgactttaaatcagaagttaaagac |
| 113 | S A G L T S A F A L F K V T E M K Q H N D F K S H V K D |
| 4209 | ttaattaaagatattgaccgtttcgttaatgggtttgaattaaatgagcttqaaccaaagtttgtgatgggctttggtggtatt |
| 141 | L I K D I D R F V N G F H L N E L H P K F V M C F C C I |
| 4293 | cgcaacgcagttaaccaatctattaatattgataaagaaacaaatcacatgtactctacacaatccgattctcaaaaacctgaa |
| 169 | R N A V N Q S I N I D K E T N H H V S T Q S D S Q K P H |
| 4377 | ggtttttggataaataaattaacacctagtggtgacttaatttcaagcatgcgtattgtacagggtggtcatggtacaacaatc |
| 197 | G F W I N K L T P S G D L I S S M R I V Q G G H G T T I |
| 4461 | ggattagaacgtcaatccaatggtgaaatgaaaatctggttacatcacgatggtgttgcaaaactgttacaagtcgcatataaa |
| 225 | G L E R Q S N G E M K I W L H H D G V A K L L Q V A Y K |
| 4545 | gataattatgtattagatttagaagaggctaaaggtttaacagattatacaccacagtcacttttaaacaaacacacatttaca |
| 253 | D N Y V L D L E E A K G L T D Y T P Q S L L N K H T F T |
| 4629 | ccgttaattgatgaagcaaatgacaaactcattttaagattcggtgacggaacaatacaggttcgttcaagagcagacgtaaaa |
| 281 | P L I D E A N D K L I L R F G D G T I Q V R S R A D V K |
| 4713 | aatcacattgataatgtagaaaaagaaatgacaattgataattcagaaaacaatgataatcgttggatgcaaggcattgetgtt |
| 309 | N H I D N V E K E M T I D N S E N N D N R W M Q G I A V |
| 4797 | gatggtgatgatttatactggttaagtggtaacagttcagttaattcacatgttcaaatcggtaaatattcattaacaacaggt |
| 337 | D G D D L V H L S C N S S V N S H V Q I C K V S L T T G |
| 4881 | caaaagatttatgattatccatttaagttatcatatcaagacggtattaatttcccacgtgataactttaaagagcctgagggt |
| 365 | Q K I Y D Y P F K L S Y Q D G I N F P R D N F K H P E G |
| 4965 | atttgcatttatacaaatccaaaaacaaaacgtaaatcgttattacttgctatgacaaacggcggtggtggaaaacgtttccat |
| 393 | I C I Y T N P K T K R K S L L L A M T N G G G G K R F H |
| 5049 | aatttatatggtttcttccaacttggtgagtatgaacactttgaagcattacgcgcaagaggttcacaaaactataaattaaca |
| 421 | N L Y G F F Q L G E Y E H F E A L R A R G S Q N Y K L T |
| 5133 | aaagacgacggtcgtgcattatctattccagaccatatcgacgatttaaatgacttaacgcaagctggtttttattatattgac |
| 449 | K D D G R A L S I P D H I D D L N D L T Q A G F Y Y I D |
| 5217 | gggggtactgcagaaaaacttaagaatatgccaatgaatggtagcaagcgtataattgacgctggttgtttcattaatgtatac |
| 477 | G G T A E K L K N M P M N G S K R I I D A G C F I N V Y |
| 5301 | cctacaacacaaacattaggtacggttcaagaattaacacgtttctcaacaggtcgtaaaatggttaaaatggtgcgtggtatg |
| 505 | P T T Q T L G T V Q E L T R F S T G R K M V K M V R G M |
| 5385 | actttagacgtatttacgttaaaatgggattatggattatggacaacaatcaaaactgacgcaccatatcaagaatatttggaa |
| 533 | T L D V F T L K W D Y G L W T T I K T D A P Y Q E Y L E |
| 5469 | gcaagtcaatacaataactggattgcttatgtaacaacagctggtgagtattacattacaggtaaccaaatggaattatttaga |
| 561 | A S Q Y N N W I A Y V T T A G E Y Y I T G N Q M E L F R |
| 5553 | gacgcgccagaagaaattaaaaaagtgggtgcatggttacgtgtgtcaagtggtaacgcagtcggtgaagtaagacaaacatta |
| 589 | D A P E E I K K V G A W L R V S S G N A V G E V R Q T L |
| 5637 | gaggctaatatatcggaatataaagaattcttcagtaatgttaatgcggaaacaaaacatcgtgaatatggttgggtagcaaaa |
| 617 | E A N I S E Y K E F F S N V N A E T K H R E Y G W V A K |
| 5721 | catcaaaaatag 5732 |
| 645 | H Q K * |

44AHJDORF003

| | |
|---|---|
| 6626 | atgagaaagttaacgaattttaagtttttatataacacaccgtttacagactatcaaaacacgattcattttaatagtaataaa |
| 1 | M R K L T N F K F F Y N T P F T D Y Q N T I H F N S N K |
| 6710 | gaacgtgatgattattttttaaatggtcgtcattttaaatcgttagactattcaaaacaaccgtataattttatacgtgataga |
| 29 | E R D D Y F L N G R H F K S L D Y S K Q P Y N F I R D R |
| 6794 | atggaaatcaatgttgatatgcagtggcatgacgcacaaggtattaactacatgacgtttttatcagattttgaggatagaaga |
| 57 | M E I N V D M Q W H D A Q G I N Y M T F L S D F E D R R |
| 6878 | tattacgcttttgtaaaccaaatcgaatacgtgaatgacgttgtggttaaaatatattttgtcattgataccattatgacgtat |
| 85 | Y Y A F V N Q I E Y V N D V V V K I Y F V I D T I M T Y |
| 6962 | acacaagggaatgtattagagcaactctcaaacgtcaatattgaacgtcaacatttatcaaaacgcacgtataactatatgtta |
| 113 | T Q G N V L E Q L S N V N I E R Q H L S K R T Y N Y M L |
| 7046 | ccaatgttacgtaataatgat9atgtgttaaaagtatcaaataaaaactatgtttataaacaaatgcaacaatatttggaaaat |
| 141 | P M L R N N D D V L K V S N K N Y V Y N Q M Q Q Y L E N |
| 7130 | ttagtattattccagtcaapcgetgatttatcaaagaaatttggtactaaaaaagagccaaacttagatacgtcaaaaggtacg |
| 169 | L V L F Q S S A D L S K K F G T K K E P N L D T S K G T |
| 7214 | atttatgacaatatcacatcaccagtcaacttatacgttatggaatatggtgactttattaactttatggataaaatgagtgcc |
| 197 | I Y D N I T S P V N L Y V M E Y G D F I N F M D K M S A |
| 7298 | tatccatggattacycaaaactttcaaaaggttcaaatgttacctaaagactttattaatacaaaagacttagaggacgtsaaa |
| 225 | Y P W I T Q N F Q K V Q M L P K D F I N T K D L E D V K |
| 7382 | accagtgaaaaaattacaggattaaaaacattaaaacagggtggtaaatcaaaagaatggagtctaaaagatttatcattaagt |
| 253 | T S E K I T G L K T L K Q G G K S E K W S L K D L S L S |
| 7466 | ttctcaaatcttcaagagatgatgttatctaaaaaagatgaatttaaacatatgatacgtaatgagtatatgacaattgaattt |
| 281 | F S N L Q E M M L S K K D E F K H M I R N E Y M T I E F |
| 7550 | tatgactggaatggaaatacgatgttactcgacgctggtaagatttcacaaaaaactggtgttaagttacgtacaaaatcaatt |
| 309 | Y D W N G N T M L L D A G K I S Q K T G V K L R T K S I |
| 7634 | attggttatcataatgaagttcgagtatatccagtagattataacagtgctgaaaacgacagaccaatactcgctaaaaataaa |
| 337 | I G Y H N E V R V Y P V D Y N S A E N D R P I L A K N K |
| 7718 | gaaatattgattgatacqpgttcattcttaaatacaaatataacatttaatagttttgcacaagtaccaatattaatcaataat |
| 365 | E I L I D T G S F L N T N I T F N S F A Q V P I L I N N |
| 7802 | ggtatcttaggacaatcacaacaagccaacaqacaaaaaaatgcagaaagtcaattaattacaaatcgtattgataatgtatta |
| 393 | G I L G Q S Q Q A N R Q K N A E S Q L I T N R I D N V L |
| 7886 | aatggtagcgacccgaaatcacgcttttatgacgctgtgagtgtagcaagtaatttaagtccaactgctttatttggtaagttt |
| 421 | N G S D P K S R F Y D A V S V A S N L S P T A L F S K F |
| 7970 | aatgaagaatataatttctacaaacaacaacaagctgaatataaagatttagccttacaaccaccttctgtaactgaatcagaa |
| 449 | N E E Y N F Y K Q Q Q A K Y K D L A L Q P P S V T E S E |
| 8054 | atgggcaacgcattccaaattgcgaatagcattaacggtttaacgatgaaaattagtgtaccgtcacctaaagaaattacattt |
| 477 | M G N A F Q I A N S I N G L T M K I S V P S P K E I T F |
| 8138 | ttacaaaaatattatatgttgtttggttttgaagtgaatgactataattcatttattgaaccaattaacagtatgactgtttgc |
| 505 | L Q K Y Y M L F G F E V N D Y N S F I E P I N S M T V C |
| 8222 | aattatttaaaatgtacaggtacgtatactatacgtgacatcgaccccatgttaatggaacaattaaaagcaattttagaatct |
| 533 | N Y L K C T G T Y T I R D I D P M L M E Q L K A I L E S |
| 8306 | ggtgtaagattttggcataatgacggttcaggtaatccaatgttacaaaatccattaaataacaaatttagagagggggtataa 8389 |
| 561 | G V R F W H N D G S G N P M L Q N P L N N K F R E G V * |

44AHJDORF004

| | |
|---|---|
| 8764 | atgatactgaaaagagtgataacaatgaacgatcaagagaagatagasaaatttacgcastcctatattaatgatgattttggt |
| 1 | M I L K R V I T M N D Q E K I D K F T H S Y I N D D F G |
| 8848 | ttaacgatagaccagttagtccctaaagtaaaaggatatgggcgctttaatgtatggcttggtggtaatgaaagtaaaatcaga |
| 29 | L T I D Q L V P K V K G Y G R F N V W L G G N E S K I R |
| 8932 | caagtattaaaagcagtaaaagagataggtgtttcacctactctttttgccgtatatgaaaaaaatgagggttttagttctgga |
| 57 | Q V L K A V K E I G V S P T L F A V Y E K N E G F S S G |
| 9016 | cttggttggttaaaccatacgtctgcacgtggtgattatttaacagatgctaaattcatagcaagaaagttagtatcacaatca |
| 85 | L G W L N H T S A R G D Y L T D A K F I A R K L V S Q S |
| 9100 | aaacaagctggacaaccgtcttggtatgacgcaggtaacatcgtccactttgtaccacaagacgtacaaagaaaaggtaatgca |
| 113 | K Q A C Q P S W Y D A G N I V H F V P Q D V Q R K G N A |
| 9184 | gattttgcaaaaaatatgaaagcaggtacaattggacgtgcatatattccattaacagcagctgctacttgggcggcatattat |
| 141 | D F A K N M K A G T I G R A Y I P L T A A A T W A A Y Y |
| 9268 | cctttaggtttgaaagcatcatataacaaagtacaaaactatggtaatccatttttagacggtgcgaatactattctagcttgg |
| 169 | P L G L K A S Y N K V Q N Y G N P F L D G A N T I L A W |
| 9352 | ggtggtaaattagacggtaaaggtggatcacctagtgattcgtctgacagtggtagtagtggtgacagtggtagttcactactc |
| 197 | G G K L D G K G G S P S D S S D S G S S G D S G S S L L |
| 9436 | gctttagcaaaacaagccatgcaagaattattaaaaaaaatacaagacgcattacaatgggacgttcatagtattggtagtgat |
| 225 | A L A K Q A M Q E L L K K I Q D A L Q W D V H S I G S D |
| 9520 | aaattttttagtaatgattattttacattagaaaaaacatttaacaacacatatcatattaaaatgacgattggtttacttgat |
| 253 | K F F S N D Y F T L E K T F N N T Y H I K M T I G L L D |
| 9604 | tcattaaaaaaactgattgatagcgttcaagtagatagtgggagtagtagttctaatcctactgatgtgacggagaccataaa |
| 281 | S L K K L I D S V Q V D S G S S S S N P T D D D G D H K |
| 9688 | ccaattagtggtaaatcagtcaagccaaatggaaaaagtggtcgtgtgattggtggtaactggacatatgcacagttaccagaa |
| 309 | P I S G K S V K P N G K S G R V I G G N W T Y A Q L P E |
| 9772 | aaatataaaaaagcaattggtgtacctttattcaaaaaagaatacttatacaaaccaggtaacatatttcctcaaacgggtaat |
| 337 | K Y K K A I G V P L F K K E Y L Y K P G N I F P (3 T G N |
| 9856 | gcaggacaatgtacagaattaacatgggcgtatatgtcacaactacatggtaaaagacaacctaccgacgacggtcaaataaca |
| 365 | A G Q C T E L T W A Y M S Q L H G K R Q P T D D G F S S |
| 9940 | aacggtcagcgtgtatggtacgtctataaaaagttaggtgcaaaaacaacacataatccaacagtaggttatggtttctctagt |
| 393 | N G Q R V W Y V Y K K L G A K T T H N P T V G Y G F S S |
| 10024 | aaaccaccatacttacaagcaactgcatatggtattggtcacacaggtgttgttgtagcagtttttgaagatggttcgttttta |
| 421 | K P P Y L Q A T A Y G I G H T G V V V A V F E D G S F L |
| 10108 | gttgcaaactataatgtaccaccatatgttgcaccatcacgtgtggtattgtatacactcattaatggcgtaccaaataatgct |
| 449 | V A N Y N V P P Y V A P S R V V L Y T L I N G V P N N A |
| 10192 | ggtgataatatsgtattctttagtggtassgcttaa 10227 |
| 477 | G D N I V F F S G I A * |

44AHJDORF005

| | |
|---|---|
| 13890 | atggtaaaacaaaatcgtttagacatggtaagagattatcaaaatgctgtcaatcatgtcagaaaaaaaatcccagataagtat |
| 1 | M V K Q N R L D M V R D Y Q N A V N H V R K K I P D K Y |
| 13806 | aatcaaatagaattagttgatgaacttatgaatgatgatatagattattatatatctatttcaaaccgttctgatggaaaatcg |
| 29 | N Q I E L V D E L M N D D I D Y Y I S I S N R S D G K S |
| 13722 | ttcaactatgtttcattttttatttatttagctattaaacttgatataaaatttactttattatcacgtcattatacattacgt |
| 57 | F N Y V S F F I Y L A I K L D I K F T L L S R H Y T L R |
| 13638 | gacgcttaccgtgattttattgaagaaatcatagatgaaaatccactatttaaatcaaaacgtgtcacgttcagaagtgctagg |
| 85 | D A Y R D F I E E I I D E N P L F K S K R V T F R S A R |
| 13554 | gactatttagctattatctatcaagataaagaaattggtgtgattacagatttgaatagtgccactgatttaaaatatcattct |
| 113 | D Y L A I I Y Q D K E I G V I T D L N S A T D L K Y H S |
| 13470 | aactttttaaaacactatcctattattatatatgatgagtttttagcacttgaagatgattatttaattgatgagtgggataag |
| 141 | N F L K H Y P I I I Y D E F L A L E D D Y L I D E W D K |
| 13386 | ttaaaaacaatatatgaatcaatcgaccgtaaccatggtaacgttgattatattggattccctaaaatgtttttactaggtaat |
| 169 | L K T I Y E S I D R N H G N V D Y I G F P K M F L L G N |
| 13302 | gcagtcaacttttcaagtcctatattatccaatttaaatatatacaatttattacaaaagcataaaatgaatacatcaagactt |
| 197 | A V N F S S P I L S N L N I Y N L L Q K H K M N T S R L |
| 13218 | tacaaaaacatttttttagaaatgcgacgaaacgattacgtgaatgaaaaacgtaacacacgtgcgtttaattcaaatgacgac |
| 225 | Y K N I F L E M R R N D Y V N E K R N T R A F N S N D D |
| 13134 | gctatgacaactggagaatttgaatttaacgaatataatttggcggatgataatttaagaaatcacatcaatcaaaacggtgat |
| 253 | A M T T G E F E F N E Y N L A D D N L R N H I N Q N G D |
| 13050 | ttcttctatatcaaaactgatgataaatatattaaagtcatgtataatgtaactacttttatgacaaatattatcgttgtacca |
| 281 | F F Y I K T D D K Y I K V M Y N V T T F M T N I I V V P |
| 12966 | tatacaaaacaatatgaattttgtactaaaattagggatatagacaatcatgttacctatttacgtgatgatatgttttataaa |
| 309 | Y T K Q Y E F C T K I R D I D N H V T Y L R D D M F Y K |
| 12882 | gaaaacatggaacgttattactacaatccaagcaatttacattttgacaatgcttactctaaaaattacgtggttgataatgat |
| 337 | E N M E R Y Y Y N P S N L H F D N A Y S K N Y V V D N D |
| 12798 | agatatttatatttagatatgaataaaattataaaatttcatataaaaaatgaaatgaagaaaaatatgagtgagtttgaaaga |
| 365 | R Y L Y L D M N K I I K F H I K N E M K K N M S E F E R |
| 12714 | aaagaaaaaatatacgaagataactatatagagaatacgaaaaagtatctaatgaaacaatatggcttataa 12643 |
| 393 | K E K I Y E D N Y I E N T K K Y L M K Q Y G L * |

44AHJDORF006

| | |
|---|---|
| 803 | atggcacaacaatctacaaaaaatgaaactgcacttttagtagcaaagtcagctaaatcagcgttacaagattttaatcatgat |
| 1 | M A Q Q S T K N E T A L L V A K S A K S A L Q D F N H D |
| 887 | tattcaaaatcttggacatttggcgacaaatgggataattcaaatacaatgttcgaaacatttgtaaataaatatttattccct |
| 29 | Y S K S W T F G D K W D N S N T M F E T F V N K Y L F P |
| 971 | aagattaatgagactttattaatcgatattgcattaggtaatcgttttaattggttagctaaagagcaagattttattggacaa |
| 57 | K I N E T L L I D I A L G N R F N W L A K E Q D F I G Q |
| 1055 | tatagtgaagaatacgtgattatggacacagtaccaattaacatggacttatctaaaaatgaggaattaatgttgaaacgtaat |
| 85 | Y S E E Y V I M D T V P I N M D L S K N E E L M L K R N |
| 1139 | tatccacgtatggcaactaagttatatggtaacggaattgtgaagaaacaaaaattcacattaaacaacaatgatacacgtttc |
| 113 | Y P R M A T K L Y G N G I V K K Q K F T L N N N D T R F |
| 1223 | aatttccaaacattagcagacgcaactaattacgctttaggtgtatacaaaaagaaaatttctgatattaatgtattagaagaa |
| 141 | N F Q T L A D A T N Y A L G V Y K K K I S D I N V L E E |
| 1307 | aaagaaatgcgtgcaatgttagttgattactcattgaatcaattatccgaaacaaatgtacgtaaagcaacatcaaaagaagat |
| 169 | K E M R A M L V D Y S L N Q L S E T N V R K A T S K E D |
| 1391 | ttagcaagcaaagtttttgaagcaatcctaaacttacaaaacaacagtgctaaatataatgaagtacatcgtgcatcaggtggt |
| 197 | L A S K V F E A I L N L Q N N S A K Y N E V H R A S G G |
| 1475 | gcaattggacaatatacaactgtatcaaaattaaaagatattgtgattttaacaacagattcattaaaatcttatcttttagat |
| 225 | A I G Q Y T T V S K L K D I V I L T T D S L K S Y L L D |
| 1559 | actaagattgcaaacacattccagattgcaggcattgatttcacagatcacgttattagttttgacgacttaggtggcgtgttt |
| 253 | T K I A N T F Q I A G I D F T D H V I S F D D L G G V F |
| 1643 | aaagtaacaaaagaatttaagttacaaaaccaagattcaattgactttttacgtgcgtatggagattatcaatcacaattagga |
| 281 | K V T K E F K L Q N Q D S I D F L R A Y G D Y Q S Q L G |
| 1727 | gatacaattccagttggtgctgtatttacttatgatgtatctaaacttaaagagtttactggcaacgttgaagaaattaaacca |
| 309 | D T I P V G A V F T Y D V S K L K E F T G N V E E I K P |
| 1811 | aaatcagatttatatgcgtttattttggatattaattcaattaaatataaacgttacacaaaaggtatgttaaaaccaccattc |
| 337 | K S D L Y A F I L D I N S I K Y K R Y T K G M L K P P F |
| 1895 | cataaccctgaatttgatgaagttacacactggattcattactattcatttaaagccattagtccattctttaataaaatttta |
| 365 | H N P E F D E V T H W I H Y Y S F K A I S P F F N K I L |
| 1979 | attactgaccaagatgtaaatccaaaaccagaggaagaattacaagaataa 2029 |
| 393 | I T D Q D V N P K P E E E L Q E * |

44AHJDORF007

| | |
|---|---|
| 2044 | atgaacaacgataaaagaggtttaaacgttgagttatcaaaggaaatcagcaaaagagttgttgaacatcgcaacagatttaaa |
| 1 | M N N D K R G L N V E L S K E I S K R V V E H R N R F K |
| 2128 | cgtcttatgtttaatcgttatttggaatttttaccgctactaatcaactataccaatcgtgatacggttggtatagattttatt |
| 29 | R L M F N R Y L E F L P L L I N Y T N R D T V G I D F I |
| 2212 | cagttagaatcagctttaagacaaaacattaatgtagttgttggtgaagctagaaataagcaaattatgattcttggttatgta |
| 57 | Q L E S A L R Q N I N V V V G E A R N K Q I M I L G Y V |
| 2296 | aataacacttactttaatcaagcaccaaatttttcatcaaactttaatttccaatttcaaaaacgattaactaaagaagatata |
| 85 | N N T Y F N Q A P N F S S N F N F Q F Q K R L T K E D I |
| 2380 | tattttattgtacctgactatttaatacctgatgattgtctacaaattcataagctatatgataactgtatgagtggtaacttt |
| 113 | Y F I V P D Y L I P D D C L Q I H K L Y D N C M S G N F |
| 2464 | gttgtcatgcaaaataaaccaattcaatataatagtgatatagaaattatagaacattatactgatgaattagcagaagttgct |
| 141 | V V M Q N K P I Q Y N S D I E I I E H Y T D E L A E V A |
| 2548 | ttatctcgcttttctttaatcatgcaagcaaaatttagcaagatatttaaatcagaaattaatgacgagtcaatcaatcaactt |
| 169 | L S R F S L I M Q A K F S K I F K S E I N D E S I N Q L |
| 2632 | gtgtccgaaatatataacggtgcaccatttgttaaaatgtcacctatgtttaatgcagatgacgatatcattgatttaacaagt |
| 197 | V S E I Y N G A P F V K M S P M F N A D D D I I D L T S |
| 2716 | aatagcgtaatcccagcattaactgaaatgaaacgggaatatcaaaacaaaattagtgaattaagtaactatttaggcattaat |
| 225 | N S V I P A L T E M K R E Y Q N K I S E L S N Y L G I N |
| 2800 | tcattagccgttgataaagaaagcggtgtttcagacgaagaggcaaaaagtaatcgtggatttaccacatcaaacagtaatatc |
| 253 | S L A V D K E S G V S D E E A K S N R G F T T S N S N I |
| 2884 | tatttaaaaggtcgtgaaccaattacgtttttatcaaagcgttatggtttagatattaaaccgtattacgatgatgaaacaacg |
| 281 | Y L K G R E P I T F L S K R Y G L D I K P Y Y D D E T T |
| 2968 | tctaaaatatcaatggtagacacactttttaaagatgaaagcagtgatataaatggctag 3027 |
| 309 | S K I S M V D T L F K D E S S D I N G * |

44AHJDORF008

| | |
|---|---|
| 3020 | atggctagatacacaatgactttatacgatttcattaaatcagaattgattaaaaaaggtttcaatgaatttgtaaatgataat |
| 1 | M A R Y T M T L Y D F I K S E L I K K G F N E F V N D N |
| 3104 | aaattaacgttttatgatgatgaatttcaattcatgcaaaaaatgctgaagttcgacaaagacgttttagctatcgttaatgaa |
| 29 | K L T F Y D D E F Q F M Q K M L K F D K D V L A I V N E |
| 3188 | aaagtatttaaaggtttttcattgaaagatgaattatcagatttactttttaaaaaatcatttacgattcattttttagataga |
| 57 | K V F K G F S L K D E L S D L L F K K S F T I H F L D R |
| 3272 | gaaatcaacagacaaacagttgaagcatttggcatgcaagtgattactgtatgtattacacatgaggattatttaaatgtggtt |
| 85 | E I N R Q T V E A F G M Q V I T V C I T H E D Y L N V V |
| 3356 | tattcatcaagtgaagttgaaaaatacttacaatcacaaggcttcacagaacacaatgaagatacaacaagtaacactgatgaa |
| 113 | Y S S S E V E K Y L Q S Q G F T E H N E D T T S N T D E |
| 3440 | acatcgaatcaaaatgctacatctttagacaattcaactggcatgactgcaaacagaaacgcttatgtgtcattaccacaaagt |
| 141 | T S N Q N A T S L D N S T G M T A N R N A Y V S L P Q S |
| 3524 | gaggttaacattgatgttgataatacaacgttacgattcgctgataataatacgattgataacggtaaaactgtgaataaatcg |
| 169 | E V N I D V D N T T L R F A D N N T I D N G K T V N K S |
| 3608 | agtaacgaaagtaatcaaaacgcaaaacgtaatcaaaatcaaaaaggtaatgcaaaaggtacacaattcactaagcagtattta |
| 197 | S N E S N Q N A K R N Q N Q K G N A K G T Q F T K Q Y L |
| 3692 | attgataatattgataaagcgtacgatttaagaaagaaaattttaaatgaatttgataaaaaatgttttttacaaatttggtag 3775 |
| 225 | I D N I D K A Y D L R K K I L N E F D K K C F L Q I W * |

44AHJDORF009

| | |
|---|---|
| 5744 | atgaaatcacaacaacaagcaaaagaatggatatataagcatgagggggcaggtgttgactttgatggtgcatatggatttcaa |
| 1 | M K S Q Q Q A K E W I Y K H E G A G V D F D G A Y G F Q |
| 5828 | tgtatggacttatcagttgcttatgtgtattacattactgacggtaaagttcgcatgtggggtaatgctaaagacgcgataaat |
| 29 | C M D L S V A Y V Y Y I T D G K V R M W G N A K D A I N |
| 5912 | aatsactttaaasssttagcgacggsstataaaaatacaccgagctttaaacctcaattaggggacgttgctgtatatacaaat |
| 57 | N D F K G L A T V Y K N T P S F K P Q L G D V A V Y T N |
| 5996 | ggacaatatggacatattcaatgtgtgttaagtggaaatcttgattattatacatgcttagaacaaaactggttaggcggcggt |
| 85 | G Q Y G H I Q C V L S G N L D Y Y T C L E Q N W L G G G |
| 6080 | tttgacggttgggaaaaagcaaccattagaacacattattatgacggtgtaactcactttattagacctaaattttcaggtagt |
| 113 | F D G W E K A T I R T H Y Y D G V T H F I R P K F S G S |
| 6164 | aatagcaaagcattagaaacatcaaaagtaaatacatttggaaaatggaaacgaaaccaatacggcacatattatagaaatgaa |
| 141 | N S K A L E T S K V N T F G K W K R N Q Y G T Y Y R N E |
| 6248 | aatggtacatttacatgtggttttttaccaatatttgcacgtgtcggtagtccaaaattatcagaacctaatggctattggttc |
| 169 | N G T F T C G F L P I F A R V G S P K L S E P N G Y W F |
| 6332 | caaccaaacggttatacaccatataacgaagtttgtttatcagatggttacgtatggattggttataactggcaaggcacacgt |
| 197 | Q P N G Y T P Y N E V C L S D G Y V W I G Y N W Q G T R |
| 6416 | tattatttaccagtgcgccaatggaatggaaaaacaggtaatagttacagtgttggtattccttggggggtgttctcataa 6496 |
| 225 | Y Y L P V R Q W N G K T G N S Y S V G I P W G V F S * |

44AHJDORF010

| | |
|---|---|
| 14420 | ttggttagacatacgtctgaaatggatagatggaaaaaagaaagagaagctagaaaagagcaagaaaaagatttatttttaaat |
| 1 | L V R H V S E M D R W K K E R E A R K E Q E K D L F L N |
| 14336 | gattttagtaatgttaattttaaatttgatgataaagatttacaagaggcgtacattgacacatggaaacattttgcacatctg |
| 29 | D F S N V N F K F D D K D L Q E A Y I D T W K H F A H L |
| 14252 | ccctattttcctaaagaaagaaacgtatcatatgtaaatgctgtatcattggtaagaggttcaagacataaaaaattaaattat |
| 57 | P Y F P K E R N V S Y V N A V S L V R G S R H K K L N Y |
| 14168 | attcttgaaatatataaccgtaatgatgattctaataataaaaacgctaaaaagcataaatacgctttatataatttacaagct |
| 85 | I L E I Y N R N D D S N N K N A K K H K Y A L Y N L Q A |
| 14084 | aaaaataataattcttcaatgtataaatatattaaagaaatcgatactttatataaagaaattggtaaatcagatagaccagtg |
| 113 | K N N N S S M Y K Y I K E I D T L Y K E I G K S D R P V |
| 14000 | acaaatattgasgasgaasasgsgasgsasaacstsssasassasgcaacatttgacgaataa 13938 |
| 141 | T N I D D E D V R Y N F L Y Y A T F D E * |

44AHJDORF011

| | |
|---|---|
| 15593 | atgacaaacgtaaaagatattttatcaagacaccaaaacacattagcgagatttgaatttgaggaaaaagaaagagaatttatc |
| 1 | M T N V K D I L S R H Q N T L A R F E F E E K E R E F I |
| 15509 | aaactatcagaattagtagaaaaatacggtatgaaaaaagagtatatcgttagagcattattcacaaacaaagaatcaaaattc |
| 29 | K L S E L V E K Y G M K K E Y I V R A L F T N K E S K F |
| 15425 | ggtgaacaaggtgttatcgtcactgatgactataacgtaaacttaccgaaccacttaacagaattaattaaagaaatgagagca |
| 57 | G E Q G V I V T D D Y N V N L P N H L T E L I K E M R A |
| 15341 | gatgaggacgttgttgacattatcaatgctggagaagttcaattcacaatttatgaatatgaaaacaaaaaaggtcaaaaaggt |
| 85 | D E D V V D I I N A G E V Q F T I Y E Y E N K K G Q K G |
| 15257 | tactcaatcaattttggtcaagtatcattttaa 15225 |
| 113 | V S I N F G Q V S F * |

44AHJDORF012

| | |
|---|---|
| 8391 | atgaacgaagtaaaattcagatttacagactcagaagcgtttcacatgtttatatacgctggggatttaaaattactctacttt |
| 1 | M N E V K F R F T D S E A F H M F I Y A G D L K L L Y F |
| 8475 | ttatttstatsaasssscsssgasassassacaggsassscaaaagcaattaaaaataataacttatggtcaaaaaaatcaatg |
| 29 | L F V L M F V D I I T G I S K A I K N N N L W S K K S M |
| 8559 | agaggattttctaaaaaattattgatattctgtattatcattttagcaaacatcattgaccagattttacaattaaaaggtggt |
| 57 | R G F S K K L L I F C I I I L A N I I D Q I L Q L K G G |
| 8643 | ccagaacaaattaaagataaattaagagtcattaaaaatgatactgaaaagagtgataacaatgaacgatcaagagaagataga |
| 85 | L L M I T I F Y Y I A N E G L S I V E N C A E M D V L V |
| 8727 | ccagaacaaattaaagataaattaagagtcattaaaaatgatactgaaaagagtgataacaatgaacgatcaagagaagataga |
| 113 | P E Q I K D K L R V I K N D T E K S D N N E R S R E D R |
| 8811 | taa 8813 |
| 141 | * |

44AHJDORF013

| | |
|---|---|
| 14996 | atgaaaattaaaactacttttagattaaataatttaatttattaccttttaacaaatagagattattataatgataaatttgaa |
| 1 | M K I K T T F R L N N L I Y Y L L T N R D Y Y N D K F E |
| 14912 | AAATTTACTTCATCTAATAAAAAATGTATAGTAAAAATAAATATGGGTGATGTGTATATTGAGTTTGACAAACAATATGATGAT |
| 29 | K F T S S N K K C I V K I N M G D V Y I E F D K Q Y D D |
| 14828 | tttgaaattgaaaaagagttatttacgttagatatcgacattgatattaaaaaacatgtttttaatatacttgtattttattat |
| 57 | F E I E K E L F T L D I D I D I K K H V F N I L V F Y Y |
| 14744 | agaaattatttaagtaatgaattaataagagaaattttattaaacgttacaattgacgacgtattatcaaattttgataaacct |
| 85 | R N Y L S N E L I R E I L L N V T I D D V L S N F D K P |
| 14660 | cttgaaagcgaattaatgattatttatcaaaacaaagtcatatacgataatgggaaagtgattgaccatgaataa 14586 |
| 113 | L E S E L M I I Y Q N K V I Y D N G K V I D H E * |

44AHJDORF113

| | |
|---|---|
| 199 | atgacagaatttgatgaaatcgtaaaaccagacgacaaagaagaaacttcagaatcaactgaagaaaatttagaatcaactgaa |
| 1 | M T E F D E I V K P D D K E E T S E S T E E N L E S T E |
| 283 | gaaacttcagaatcaactgaagaatcaactgaagaatcaactgaagaatcaactgaagataaaacagtagaaacaatcgaagaa |
| 29 | E T S E S T E E S T E E S T E E S T E D K T V E T I E E |
| 367 | gaaaatgaaaacaaattagaacctactacaacagatgaagatagttcgaaatttgaccctgttgtattagaacaacgtattgct |
| 57 | E N E N K L E P T T T D E D S S K F D P V V L E Q R I A |
| 451 | tcattagaacaacaagtgactacttttttatcttcacaaatgcaacaaccacaacaagtacaacaaacacaatcagatgtaaca |
| 85 | S L E Q Q V T T F L S S Q M Q Q P Q Q V Q Q T Q S D V T |
| 535 | gaatcaaacaaagaagataacgactattcagatgaagaactagttgataagttagatttagattag 600 |
| 113 | E S N K E D N D Y S D E E L V D K L D L D * |

44AHJDORF114

| | |
|---|---|
| 16172 | atggttaatgttgataatgcaccagaagaaaaaggacaagcctatactgaaatgttgcaactattcaataaactgattcaatgg |
| 1 | M V N V D N A P E E K G Q A Y T E M L Q L F N K L I Q W |
| 16088 | aatccagcttatacatttgacaatgcaattaacttattatcggcttgccaacaactattattaaactataatagttctgttgtt |
| 29 | N P A Y T F D N A I N L L S A C Q Q L L L N Y N S S V V |
| 16004 | caattcttaaatgatgaactaaacaacgaaactaaaccagaatcaatattgtcttatattgctggtgatgacccaatagaacaa |
| 57 | Q F L N D E L N N E T K P E S I L S Y I A G D D P I E Q |
| 15920 | tggaatatgcataaaggattttatgaaacgtataacgtttacgttttttag 15870 |
| 85 | W N M H K G F Y E T Y N V Y V F * |

44AHJDORF014

| | |
|---|---|
| 6243 | atgaaaatggtacatttacatgtggttttttaccaatatttgcacgtgtcggtagtccaaaattatcagaacctaatggctatt |
| 1 | M K M V H L H V V F Y Q Y L H V S V V Q N Y Q N L M A I |
| 6327 | ggttccaaccaaacggttatacaccatataacgaagtttgtttatcagatggttacgtatggattggttataactggcaaggca |
| 29 | G S N Q T V I H H I T K F V Y Q M V T Y G L V I T G K A |
| 6411 | cacgttattatttaccagtgcgccaatggaatggaaaaacaggtaatagttacagtgttggtattccttggggggtgttctcat |
| 57 | H V I I Y Q C A N G M E K Q V I V T V L V F L G G C S H |
| 6495 | aatgggtattttagcctttttctttga 6521 |
| 85 | N G Y F S L F L * |

44AHJDORF015

| | |
|---|---|
| 15403 | gtgacgataacaccttgttcaccgaattttgattctttgtttgtgaataatgctctaacgatatactcttttttcataccgtat |
| 1 | V T I T P C S P N F D S L F V N N A L T I Y S F F I F Y |
| 15487 | ttttctactaattctgatagtttgataaattctctttctttttcctcaaattcaaatctcgctaatgtgttttggtgtcttgat |
| 29 | F S T N S D S L I N S L S F S S N S N L A N Y F W C L D |
| 15571 | aaaatatcttttacgtttgtcattttatttctcctcttatttaaattatttgctttctgcaattgcgatttgtag 15645 |
| 57 | K I S F T F V I L F L L L F K L F A F C N C D L * |

44AHJDORF016

| | |
|---|---|
| 15852 | atgaaagttgacgacattgttaccttacgtgtcaaaggttatatacttcattacttagatgatgataatgaatacattgaggaa |
| 1 | M K V D D I V T L R V K G Y I L H Y L D D D N E Y I E E |
| 15768 | tttttaccacttcacgagtatcatttaaccaaaacacaagcaaaagaattattaccagacacatgtaaactattgtccactaca |
| 29 | F L P L H E Y H L T K T Q A K E L L P D T C K L L S T T |
| 15684 | cgcacaacgaaaacaattcaagtttattacaatgatttactacaaatcgcaattgcagaaagcaaataa 15616 |
| 57 | R T T K T I Q V Y Y N D L L Q I A I A E S K * |

44AHJDORF017

| | |
|---|---|
| 10757 | atggaaagattaaaattgcttctgctggtataccgaaaaacgcctttgatacaagcgtcgattttgaaacctttgtacgtgaac |
| 1 | M E R L K L L L L V Y R K T P L I Q A S I L K P L Y V N |
| 10673 | aattctttgacggtgccattattgaaaacaataaaagtatctataatgagcaaggtacaatatcgatatatccgtctaaaactg |
| 29 | N S L T V P L L K T I K V S I H S K V Q Y R Y I R L K L |
| 10589 | aaattgtatgtggtaatgtatatgatgaatattttactgatgaacttaatatga 10536 |
| 57 | K L Y V V M Y H M N I L L H N L I * |

44AHJDORF018

| | |
|---|---|
| 1098 | atgttaattggtactgtgtccataatcacgtattcttcactatattgtccaataaaatcttgctctttagctaaccaattaaaa |
| 1 | M L I G T V S I I T Y S S L Y C P I K S C S L A N Q L K |
| 1014 | cgattacctaatgcaatatcgattaataaagtctcattaatcttagggaataaatatttatttacaaatgtttcgaacattgta |
| 29 | R L P N A I S I N K V S L I L G N K Y L F T N V S N I V |
| 930 | tttgaattataccatttgtcgccaaatgtccaagattttgaataa 886 |
| 57 | F E L S H L S P N V Q D F E * |

44AHJDORF019

| | |
|---|---|
| 9836 | atgttacctggtttgtataagtattcttttttgaataaaggtacaccaattgcttttttatatttttctggtaactgtgcatat |
| 1 | M L P G L Y K Y S F L N K G T P I A F L Y F S G N C A Y |
| 9752 | gtccagttacaaccaatcacacgaccactttttccatttggcttgactgatttaccactaattggtttatggtctccgtaatca |
| 29 | V Q L P P I T R P L F P F C L T D L P L I G L W S P S S |
| 9668 | tcagtaggattayaactactactcccactatctacttga 9630 |
| 57 | S V G L E L L L P L S T * |

44AHJDORF121

| | |
|---|---|
| 16362 | atggaaaatgaaacaaaaaacattgagttgaagcatgtttttcgttttaagaatggaagtttatgtatagcgttatttgataga |
| 1 | M E N S T K M X E L K H V F R F K N G S L C I A L F D R |
| 16278 | acagaaaatgaaatttcattttatgatgttgacattgatgaaattgaagatttaaatcataattctgttttacycgtaatttca |
| 29 | T E N E I S F Y D V D I D E I S S L N H N S V L R V I S |
| 16194 | actttattaggaagtgataataatggttaa 16165 |
| 57 | T L L G S D N N G * |

44AHJDORF020

| | |
|---|---|
| 13865 | atgtctaaacgattttgttttaccatgtttttgctccttgtaatagtttatgatgtcgtttacagtgttaaatttattcgtcaa |
| 1 | H S K R F C F T M F L L L V I V Y D V V Y S V K F I R Q |
| 13949 | atgttgcataatataaaaagttatacctcacatcttcatcatcaatatttgtcactggtctatctgatttaccaatttctttat |
| 29 | H L H N I K S Y T S H L H H Q V L S L V Y L I V Q F L V |
| 14033 | ataaagtatcgatttctttaa 14053 |
| 57 | I K Y R F L * |

44AHJDORF123

| | |
|---|---|
| 614 | atgtatgagggaaacaacatgcgttctatgatgggtacatcatatgaagattcaagattaaataaacgaacagaattaaatgaa |
| 1 | M V E G N M M R S M M C T S Y E D S R L N K R T E L M E |
| 698 | aacatgtcaattgatacaaataaaagtgaagatagttatggtgtacaaattcattcactttcaaaacaatcatttacaggtgac |
| 29 | N H S I D V N K S S D S Y G V Q I H S L S K Q S F T C S |
| 782 | gttgaggagpaataa 796 |
| 57 | V H S S * |

44AHJDORF021

| | |
|---|---|
| 5816 | atgcaccatcaaagtcaacacctgccccctcatgcttatatatccattcttttgcttgttgttgtgatttcatttatatcactc |
| 1 | M H H Q S Q H L P P H A Y I S I L L L V V V I S F I S L |
| 5732 | ctatttttgatgttttgctacccaaceatattcacgatgttttgtttccgcattaacattactgaagaattctttatattccga |
| 29 | L F L H F C V P T I F V H F C F R I N I V S E F F I F R |
| 5648 | tatattagcctctaa 5634 |
| 57 | Y I S L * |

44AHJDORF022

| | |
|---|---|
| 8611 | atgtttgctaaaatgataatacagaatatcaataattttttagaaaatcctctcattgatttttttgaccataagttattattt |
| 1 | M F A K M I I Q N I N N F L S N P L I D F F D H K L L F |
| 8527 | ttaattgcttttgaaatacctgtaataatatcaaogaacattaatacaaataaaaagtag 8468 |
| 29 | L I A F S I P V I I S T N I N T N K K * |

44AHJDORF023

| | |
|---|---|
| 8494 | atgagaacaccccccaaggaataccaacactgtaactattacctgtttttccattccattggogcacsggtaaataasaacgtg |
| 1 | M R T P P K E Y Q H C N Y Y L F F H S I G A L V N N N V |
| 6410 | tgccttgccapttataaccaatccatacgtaaccatctgataaacaaacttcgttatatggtgtataacogtttggstggaacc |
| 29 | C L A S Y N Q S I R N H L I N K L R Y H V Y N R L V C T |
| 6326 | aatagccattag 6315 |
| 57 | N S H * |

44AHJDORF024

| | |
|---|---|
| 14275 | gtgtcaatgtacgcctcttgtaaatctttatcatcaaatttaaaattaacattactaaaatcatttaaaaataaatctttttct |
| 1 | V S M Y A S C K S L S S N L K L T L L K S F K N K S F S |
| 14359 | tgctcttttctagcttctctttcttttttccatctatccatttcagacgtatgtctaaccaatgttatcaacctccatasaaag |
| 29 | C S F L A S L S F F H L S I S D V C L T N V I N L H I K |
| 14443 | cataaataa 14451 |
| 57 | H K * |

44AHJDORF025

| | |
|---|---|
| 15175 | atggaacgtaaatacaaaacgggtattattatattgcgatgagattaaaggacattttccacatcaatctcaatgtttgaagat |
| 1 | M E R K Y K T V L L Y C D E I K G H F P H Q I S M F E D |
| 15091 | ttatatgacgctaaagttgtatatscatattasgaatasaacctgttcactaaaaaasacgcgsatatcatagaatacattaag |
| 29 | L V D A K V V V S V V E Y N L F T K K V A V I I E V I K |
| 15007 | gagatataa 14999 |
| 57 | E I * |

44AHJDORF026

| | |
|---|---|
| 14593 | atgaataacctattaaacatagccattgttttccttttagcatttttaattacacttatcatacttatgacactgcatatacgc |
| 1 | M N N L L N I A I V F L L A F L I T L I I L M T L H I R |
| 14509 | gtgtcatttggtgttttattcactacattgattatattctatattatctttttaatggttatttatgctttatatggaggttga 14426 |
| 29 | V S F C V L F I T L I I F V I I F L M V I V A L V C C * |

44AHJDORF027

| | |
|---|---|
| 12916 | atgattgtcsatatcoctaassttagsacaaaattoasattgttttgsatasggsacaacgataatatttgtcataaaagtagt |
| 1 | M I V Y I P N F S T K F I L F C I W Y N D N I C H K S S |
| 13000 | tacattatacatgactttaatatatttatcatcagttttgatatagaagaaatcaccgttttgattgatgtgatttcttaa 13080 |
| 29 | Y I I H D F N I F I I S F D I E E I T V L I D V I S * |

44AHJDORF029

| | |
|---|---|
| 15183 | gtgtttaaatggaacgtaaatacaaaacggtattattatattgcgatgagattaaaggacattttccacatcaaatctcaatgt |
| 1 | V F K W N V N T K R T T V I A M R L K D I F N I K S Q C |
| 15099 | ttgaagatttatatgacgctaaagttgtatattcatattatgaatataacctgttcactaaaaaatacgcgtatatcatag 15019 |
| 29 | L K I V M T L K L V I N I M N I T C S L K N T R I S * |

44AHJDORF028

| | |
|---|---|
| 9235 | atggaatatatgcacgtccaattgtacctgctttcatattttttgcaaaatctgcattaccttttctttgtacgtcttgtggta |
| 1 | M E V M N V Q L T L L S T F L Q N L H T L F F V R L V V |
| 9151 | caaagtggacgatgttacctgcgtcataccaagacggttgtccagcttgttttgattgtgatactaactttcttgctatga 9071 |
| 29 | Q S C R C V L K N T K T V V Q L V L I V I L T F L L * |

44AHJDORF030

| | |
|---|---|
| 14487 | gtgaataaacaccaatgacacgcgtatatgcagtgtcataagtatgataagtgtaattaaaatgctaaaaggaaaacaatg |
| 1 | V N K T P N D T R I C S V I S M I S V I K N A K R K T M |
| 14571 | gctatgtttaataggttattcatggtcaatcactttcccattatcgtatatgactttgttttgataaataatcattaa 14648 |
| 29 | A H F N R L F H V N H F P I I V V D F V L I N N H * |

44AHJDORF031

| | |
|---|---|
| 11039 | atgatattgtatagttcattgttatcatctaaacggaataagttaaaatgtgaacgtaatgcaggtatgccatataatccattt |
| 1 | M I L Y S S L L S S K R N K L K C E R N A G M P Y N P F |
| 11123 | aaaacgactttagataacataacctcctcatttgagtatgggtgttcgttgatatcatcagtaatgtga 11191 |
| 29 | K T T L D N I T S S F E Y G C S L I S S V M * |

44AHJDORF135

| | |
|---|---|
| 693 | atgaaaacatgtcaattgatacaaataaaagtgaagatagttatggtgtacaaattcattcactttcaaaacaatcatttacag |
| 1 | M K T C Q L I Q I K V K I V M V Y K F I H F Q N N H L Q |
| 777 | gtgacgttgaggaggaataataaattatggcacaacaatctacaaaaaatgaaactgcacttttag 842 |
| 29 | V T L R R N N K L W H N N L Q K M K L H F * |

44AHJDDRF033

| | |
|---|---|
| 3795 | atgccattatttaaccactctaccaaatttgtaaaaaacattttttatcaaattcatttaaaattt5tctttcttaaatcgtac |
| 1 | M P L F N H L Y Q I C K K H F L S N S F K I F F L K S Y |
| 3711 | gctttatcaatattatcaattaaatactgcttagtgaattgtgtaccttttgcattacctttttga 3646 |
| 29 | A L S I L S I K Y C L V N C V P F A L P F * |

44AHJDORF032

| | |
|---|---|
| 9455 | atggcttgttttgctaaagcgagtagtgaactaccactgtcaccactactaccactgtcagacgaatcactaggtgatccacct |
| 1 | M A C F A K A S S E L P L S P L L P L S D E S L G D P P |
| 9371 | ttaccgtctaatttaccaccccaagctagaatagtattcgcaccgtctaaaaatggattaccatag 9306 |
| 29 | L P S N L P P Q A R I V F A P S K N G L P * |

44AHJDORF034

| | |
|---|---|
| 14146 | atgatgattctaataataaaaacgctaaaaagcataaatacgctttatataatttacaagctaaaaataataattcttcaatgt |
| 1 | M M I L I I K T L K S I N T L Y I I Y K L K I I I L Q C |
| 14062 | ataaatatattaaagaaatcgatactttatataaagaaattggtaaatcagatagaccagtga 14000 |
| 29 | I N I L K K S I L Y I K K L V N Q I D Q * |

44AHJDORF035

| | |
|---|---|
| 13957 | atgcaacatttgacgaataaatttaacactgtaaacgacatcataaactattacaaggagcaaaaacatggtaaaacaaaatcg |
| 1 | M Q H L T N K F N T V N D I I N Y Y K E Q K H G K T K S |
| 13873 | tttagacatggtaagagattatcaaaatgctgtcaatcatgtcagaaaaaaaatcccagataa 13811 |
| 29 | F R H G K R L S K C C Q S C Q K K N P R * |

44AHJDORF036

| | |
|---|---|
| 10165 | gtgtatacaataccacacgtgatggtgcaacatatggtggtacattatagtttgcaactaaaaacgaaccatcttcaaaaactg |
| 1 | V Y T I P H V M V Q H M V V H Y S L Q L K T N H L Q K L |
| 10081 | ctacaacaacacctgtgtgaccaataccatatgcagttgcttgtaagtatggtggtttactag 10019 |
| 29 | L Q Q H L C D Q Y H M Q L L V S M V V Y * |

44AHJDORF037

| | |
|---|---|
| 14788 | atgtcgatatctaacgtaaataactctttttcaatttcaaaatcatcatattgtttgtcaaactcaatatacacatcacccata |
| 1 | M S I S N V N N S F S I S K S S Y C L S N S I Y T S P I |
| 14872 | tttatttttactatacattttttattagatgaagtaaatttttcaaatttatcattataa 14931 |
| 29 | F I F T I H F L L D E V N F S N L S L * |

44AHJDORF038

| | |
|---|---|
| 3671 | gtgtaccttttgcattacctttttgattttgattacgttttgcgttttgattactttcgttactcgatttattcacagttttac |
| 1 | V Y L L H Y L F D F D Y V L R F D V F R Y S I Y S Q F Y |
| 3587 | cgttatcaatcgtattattatcagcgaatcgtaacgttgtattatcaacatcaatgttaa 3528 |
| 29 | R Y Q S Y Y Y Q R I V T L Y Y Q H Q C * |

44AHJDORF039

| | |
|---|---|
| 1743 | gtgctgtatttacttatgatgtatctaaacttaaagagtttactggcaacgttgaagaaattaaaccaaaatcagatttatatg |
| 1 | V L Y L L M M Y L N L K S L L A T L K K L N Q N Q I Y M |
| 1827 | cgtttattttggatattaattcaattaaatataaacgttacacaaaaggtatgttaa 1883 |
| 29 | R L F W I L I Q L N I N V T Q K V C * |

44AHJDORF040

| | |
|---|---|
| 9740 | gtggtaactggacatatgcacagttaccagaaaaatataaaaaagcaattggtgtacctttattaaaaaaagaatacttataca |
| 1 | V V T G H M H S Y Q K N I K K Q L V Y L Y S K K N T Y T |
| 9824 | aaccaggtaacatatttcctcaaacgggtaatgcaggacaatgtacagaattaa 9877 |
| 29 | N Q V T Y F L K R V M Q D N V Q N * |

44AHJDORF041

| | |
|---|---|
| 15836 | atgtcgtcaactttcattattatatcactcctttctaaaaaacgtaaacgttatacgtttcataaaatcctttatgcatattcc |
| 1 | M S S T F I I I S L L S K K R K R Y T F H K I L Y A Y S |
| 15920 | attgttctattgggtcatcaccagcaatataagacaatattgattctggtttag 15973 |
| 29 | I V L L G H H Q Q Y K T I L I L V * |

44AHJDORF042

| | |
|---|---|
| 5151 | atgcacgaccgtcgtcttttgttaatttatagttttgtgaacctcttgcgcgtaatgcttcaaagtgttcatactcaccaagtt |
| 1 | M H D R R L L L I Y S F V N L L R V M L Q S V H T H Q V |
| 5067 | ggaagaaaccatataaattatggaaacgttttccaccaccgccgtttgtcatag 5014 |
| 29 | G R N H I N Y G N V F H H R R L S * |

44AHJDORF043

| | |
|---|---|
| 4539 | atgcgacttgtaacagttttgcaacaccatcgtgatgtaaccapattttcatttcaccattggattgacgttctaatccgattg |
| 1 | M R L V T V L Q H H R D V T R F S F H H W I D V L I R L |
| 4455 | ttgtaccatgaccaccctgtacaatacgcatgcttgaaattaagtcaccactag 4402 |
| 29 | L Y H D H P V Q Y A C L K L S H H * |

44AHJDORF044

| | |
|---|---|
| 12917 | atgttacctatttacgtgatgatatgttttataaagaaaacatggaacgttattactacaatccaagcaatttacattttgaca |
| 1 | M L P I Y V M I C F I K K T W N V I T T I Q A I Y I L T |
| 12833 | atgcttactctaaaaattacgtggttgataatgatagatatttatatttag 12783 |
| 29 | M L T L K I T W L I M I D I Y I * |

44AHJDORF149

| | |
|---|---|
| 770 | atgattgttttgaaagtgaatgaatttgtacaccataactatcttcacttttatttgtatcaattgacatgttttcatttaatt |
| 1 | M I V L K V N E F V H H N Y L H F Y L Y Q L T C F H L I |
| 686 | ctgttcgtttatttaatcttgaatcttcatatgatgtacccatcatag 639 |
| 29 | L F V Y L I L N L H M M Y P S * |

44AHJDORF046

| | |
|---|---|
| 4891 | atgattatccatttaagttatcatatcaagacggtattaatttcccacgtgataactttaaagagcctgagggtatttgcattt |
| 1 | M I I H L S Y H I K T V L I S H V I T L K S L R V F A F |
| 4975 | atacaaatccaaaaacaaaacgtaaatcgttattacttgctatga 5019 |
| 29 | I Q I Q K Q N V N R Y Y L L * |

44AHJDORF047

| | |
|---|---|
| 11911 | atgaatgtatgtaagttgttcaggtgtgagttttgcaaaacatttcacagcatagtcataggcttcactatcattcatatcatt |
| 1 | M N V C K L F R C E F C K T F H S I V I G F T I I H I I |
| 11995 | atctttatcaaaaatcgtataattaaaatctgttttaagttgtga 12039 |
| 29 | I F I K N R I I K I C F K L * |

44AHJDORF045

| | |
|---|---|
| 10655 | atggcaccgtcaaagaattgttcacgtacaaaggtttcaaaatcgacgcttgtatcaaaggcgtttttcggtataccagcagaa |
| 1 | M A P S K N C S R T K V S K S T L V S K A F F G I P A E |
| 10739 | gcaattttaatctttccattcacttcatatgcatatttcttatga 10783 |
| 29 | A I L I F P F T S Y A Y F L * |

44AHJDORF048

| | |
|---|---|
| 15340 | atgaggacgttgttgacattatcaatgctggagaagttcaattcacaatttatgaatatgaaaacaaaaaaggtcaaaaaggtt |
| 1 | M R T L L T L S M L E K F N S Q F M N M K T K K V K K V |
| 15256 | actcaatcaattttggtcaagtatcattttaatacaatttcatag 15212 |
| 29 | T Q S I L V K Y H F N T I S * |

44AHJDORF049

| | |
|---|---|
| 5784 | atgagggggcaggtgttgactttgatggtgcatatggatttcaatgtatggacttatcagttgcttatgtgtattacattactg |
| 1 | M R G Q V L T L M V H M D F N V W T Y Q L L M C I T L L |
| 5868 | acggtaaagttcgcatgtggggtaatgctaaagacgcgataa 5909 |
| 29 | T V K F A C G V M L K T R * |

44AHJDORF050

| | |
|---|---|
| 13158 | gtgtgttacgtttttcattcacgtaatcgtttcgtcgcatttctaaaaaaatgtttttgtaaagtcttgatptattcattttat |
| 1 | V C Y V F H S R N R F V A F L K K C F C K V L M Y S F Y |
| 13242 | gcttttgtaataaattgtatatatttaaattggataatatag 13283 |
| 29 | A F V I N C I Y L N W I I * |

44AHJDORF051

| | |
|---|---|
| 11066 | atgataacaatgaactatacaatatcattaacggttacaaaaacactgaacgtaatatattattctctacatttgtcacatcac |
| 1 | M I T M N Y T I S L T V T K T L N V I Y Y S L H L S H H |
| 10982 | gttcattgtataacttattggttcctttccaatacttaa 10944 |
| 29 | V H C I T Y W F L S N T * |

44AHJDORF052

| | |
|---|---|
| 14338 | atgattttagtaatgttaattttaaatttgatgataaagatttacaagaggcgtacattgacacatggaaacattttgcacatc |
| 1 | M I L V M L I L N L M I K I Y K R R T L T H G N I L H I |
| 14254 | tgccctattttcctaaagaaagaaacgtatcatatgtaa 14216 |
| 29 | C P I F L K K E T Y H M * |

44AHJDORF053

| | |
|---|---|
| 3348 | atgtggtttattcatcaagtgaagttgaaaaatacttacaatcacaaggcttcacagaacacaatgaagatacaacaagtaaca |
| 1 | M W F I H Q V K L K N T Y N H K A S Q N T M K I Q Q V T |
| 3432 | ctgatgaaacatcgaatcaaaatgctacatctttag 3467 |
| 29 | L M K H R I K M L H L * |

44AHJDORF054

| | |
|---|---|
| 7551 | atgactggaatggaaatacgatgttactcgacgctggtaagacttcacaaaaaactggtgttaagttacgtacaaaatcaatta |
| 1 | M T G M E I R C Y S T L V R F H K K L V L S Y V Q N Q L |
| 7635 | ttggttatcataatgaagttcgagtatatccagtag 7670 |
| 29 | L V I I M K F E Y I Q * |

44AHJDORF055

| | |
|---|---|
| 15705 | atgtgtctggtaataattcttttgcttgtgttttggttaaatgatactcgtgaagtggtaaaaattcctcaatgtattcattat |
| 1 | M C L V I I L L L V F W L N D T R E V V K I P Q C I H Y |
| 15789 | catcatctaagtaatgaagtatataacctttga 15821 |
| 29 | H H L S N E V Y N L * |

44AHJDORF056

| | |
|---|---|
| 5512 | gtgagtattacattacaggtaaccaaatggaattatttagagacgcgccagaagaaattaaaaaagtgggtgcatggttacgtg |
| 1 | V S I T L Q V T K W N Y L E T R Q K K L K K W V H G Y V |
| 5596 | tgtcaagtggtaacgcagtcggtgaagtaa 5625 |
| 29 | C Q V V T Q S V K * |

44AHJDORF057

| | |
|---|---|
| 10121 | atgtaccaccatatgttgcaccatcacgtgtggtattgtatacactcattaatggcgtaccaaataatgctggtgataatattg |
| 1 | M Y H H M L H H H V W Y C I H S L M A Y Q I M L V I I L |
| 10205 | tattctttagtggtattgcttaattaa 10231 |
| 29 | Y S L V V L L N * |

44AHJDORF058

| | |
|---|---|
| 10707 | atgcatatttcttatgattcagtacaaacatcttatctatctgttcgttttcaatatcccatttacotaaggctatcgggtcga |
| 1 | M H I S Y D S V Q T S Y L S V R F Q Y P I Y L R L S G R |
| 10851 | ataaactggggttcaataagggtttaa 10877 |
| 29 | I N W G S I R V * |

44ABJDORF164

| | |
|---|---|
| 702 | atgttttcatttaattctgttcgtttatttaatcttgaatcttcatatgatgtacccatcatagaacgcatgttgtttccctca |
| 1 | M F S F N S V R L F N L E S S Y D V P I I E R M L F P S |
| 618 | tacatgtttaaattoctcctaatctaa 592 |
| 29 | Y M F K F L L I * |

44AHJDORF059

| | |
|---|---|
| 8360 | atggattttgtaacattggattacctgaaccgtcattatgccaaaatcttacaccagattctaaaattgcttttaattgttcca |
| 1 | M D F V T L D Y L N R H Y A K I L H Q I L K D L L I V P |
| 8276 | ttaacatggggtcgatgtcacgtatag 8250 |
| 29 | L T W G R C H V * |

44AHJDORF060

| | |
|---|---|
| 6257 | atgtaccattttcatttctataatatgtgccgtattggtttcgtttccattttccaaatgtatttacttttgatgtttctaatg |
| 1 | M Y H F H F Y N M C R I G F V S I F Q M Y L L L M F L M |
| 6173 | ctttgctattactacctgaaaatttag 6147 |
| 29 | L C Y Y Y D K I * |

44AHJDORF061

| | |
|---|---|
| 15551 | atgtgttttggtgtcttgataaaatatcttttacgtttgtcattttatttctcctcttatttaaattatttgctttctgcaatt |
| 1 | M C F G V L I K Y L L R L S F Y F S S Y L N Y L L S A I |
| 15635 | gcgatttgtagtaaatcattgtaa 15658 |
| 29 | A I C S K S L * |

44AHJDORF062

| | |
|---|---|
| 4285 | gtggtattcgcaacgcagttaaccaatotattaatattgataaagaaacaaatcacatgtactctacacaatccgattctcaaa |
| 1 | V V F A T Q L T N L L I L I K K Q I T C T L H N P I L K |
| 4369 | aacctgaaggtttttggataa 4389 |
| 29 | N L K V F G * |

44AHJDORF063

| | |
|---|---|
| 9487 | atgcgtcttgtattttttttaataattcttgcatggcttgttttgctaaagcgagtagtgaactaccactgtcaccactactac |
| 1 | M R L V F F L I I L A W L V L L K R V V N Y H C H H Y Y |
| 9403 | cactgtcapacgaatcactag 9383 |
| 29 | H C Q T N H * |

44AHJDORF065

| | |
|---|---|
| 5029 | gtggtggaaaacgtttccataatttatatggtttcttccaacttggtgagtatgaacactttgaagcattacgcgcaagaggtt |
| 1 | V V E N V S I I Y M V S S N L V S M N T L K H Y A Q E V |
| 5113 | cacaaaactataaattaa 5130 |
| 29 | H K T I N * |

44AHJDORF064

| | |
|---|---|
| 2609 | atgacgagtcaatcaatcaacttgtgtccgaaatatataacggtgcaccatttgttaaaatgtcacctatgtttaatgcagatg |
| 1 | MTSQSINLCPKYITVHHLLKCHLCLMQM |
| 2693 | acgatatcattgatttaa 2710 |
| 29 | TISLI* |

44AHJDORF066

| | |
|---|---|
| 10481 | atgatattctttatattgaaagtgacatcggttcattttcacttaacgacttatttccagttgaacgttcagtacataacaaat |
| 1 | M I F F I L K V T S V H F H L T T Y F Q L N V Q Y I T N |
| 10397 | ctgatttgcatatattaa 10380 |
| 29 | L I C I Y * |

We claim:

1. A method for identifying a target for antibacterial agents, comprising determining the bacterial target of a product of a bacteriophage 44AHJD open reading frame selected from the group consisting of open reading frames 12 and 25.

2. The method of claim 1, wherein said determining comprises identifying at least one bacterial protein which binds to said product or a fragment thereof.

3. The method of claim 2, wherein said binding is determined using affinity chromatography on a solid matrix.

4. The method of claim 1, wherein said determining comprises identifying at least one protein:protein interaction using a genetic screen.

5. The method of claim 4, wherein said genetic screen is a yeast two-hybrid screen.

6. The method of claim 1, wherein said determining comprises a co-immunoprecipitation assay or a protein-protein crosslinking assay.

7. The method of claim 1, wherein said determining comprises identifying a mutated bacterial coding sequence which protects a bacterium from said bacteriophage 44AHJD open reading frame product.

8. The method of claim 1, wherein said determining comprises identifying a bacterial coding sequence which protects a bacterium against said product when expressed at high levels in said bacterium.

9. The method of claim 1, wherein said determining further comprises identifying a bacterial nucleic acid sequence encoding a polypeptide target of said product of bacteriophage 44AHJD open reading frame.

10. The method of claim 9, wherein said nucleic acid sequence is identified by determining at least a fragment of the amino acid sequence of a bacterial protein target, and identifying a bacterial nucleic acid sequence which encodes said fragment.

11. The method of claim 1, wherein said bacterial target is from an animal pathogen.

12. The method of claim 11, wherein said bacterial target is a gene homologous to a gene from an animal pathogen.

13. The method of claim 11, wherein said pathogen is a human pathogen.

14. The method of claim 1, wherein said bacterial target is from a plant pathogen.

15. The method of claim 1, wherein said bacterial target is a gene homologous to a gene from a plant pathogen.

16. The method of claim 1, further comprising determining the cellular or biochemical function or both of said product of bacteriophage 44AHJD open reading frame.

17. The method of claim 1, wherein said identifying the bacterial target comprises identifying a phage-specific site of action.

18. An isolated, purified, or enriched nucleic acid sequence at least 15 nucleotides in length, wherein said sequence corresponds to at least a portion of a bacteriophage 44AHJD open reading frame 12 or 25 sequence.

19. The nucleic acid sequence of claim 18, wherein said sequence comprises at least 50 nucleotides.

20. The nucleic acid sequence of claim 18, wherein said nucleic acid sequence corresponds to a fragment of said bacteriophage 44AHJD open reading frame 12 or 25 sequence.

21. The nucleic acid sequence of claim 20, wherein said nucleic acid sequence encodes a polypeptide which provides a bacteria-inhibiting function.

22. The nucleic acid sequence of claim 21, wherein said nucleic acid sequence is transcriptionally linked with regulatory sequences enabling induction of expression of said sequence.

23. An isolated, purified, or enriched polypeptide comprising at least a fragment of a protein encoded by Staphylococcus aureus bacteriophage 44AHJD open reading frame 12 or 25, wherein said portion is at least 5 amino acid residues in length.

24. The polypeptide of claim 24, wherein said polypeptide comprises a fragment at least 10 amino acid residues in length of a said polypeptide normally encoded by said bacteriophage.

25. A recombinant vector comprising a nucleic acid sequence at least 24 nucleotides in length corresponding to a portion of bacteriophage 44AHJD open reading frame 12 or 25.

26. The vector of claim 25, wherein said vector is an expression vector.

27. The vector of claim 26, wherein expression of said ORF is inducible.

28. A recombinant cell comprising a vector, wherein said vector comprises a nucleic acid sequence at least 24 nucleotides in length corresponding to at least a fragment of bacteriophage 44AHJD open reading frame 12 or 25.

29. The cell of claim 28, wherein said vector is an expression vector and expression of said ORF is inducible.

30. A method for identifying an antibacterial agent, comprising identifying an active fragment of a product of a bacteria-inhibiting ORF of a bacteriophage.

31. The method of claim 30, further comprising constructing a synthetic peptidomimetic molecule, wherein the structure of said molecule corresponds to the structure of said active fragment.

32. A method for identifying a compound active on a bacterial target protein of a bacteriophage 44AHJD open reading frame 12 or 25 product, comprising the step of contacting said bacterial target protein with a test compound; and determining whether said compound binds to or reduces the level of activity of said target protein,
wherein binding of said compound with said target protein or a reduction of the level of activity of said protein is indicative that said compound is active on said target and wherein said target is uncharacterized.

33. The method of claim 32, wherein said contacting is carried out in vitro.

34. The method of claim 32, wherein said contacting is carried out in vivo in a cell.

35. The method of claim 32, wherein said compound is a small molecule.

36. The method of claim 32, wherein said compound is a peptidomimetic compound.

37. The method of claim 32, wherein said compound is a fragment of a bacteriophage inhibitor protein.

38. The method of claim 32, further comprising determining the site of action of said compound on said target protein.

39. A method of screening for potential antibacterial agents, comprising the step of determining whether any of a plurality of compounds is active on a target of a bacteriophage 44AHJD open reading frame 12 or 25 product,
wherein said target is naturally produced by a pathogenic bacterium. [one step method claim]

40. The method of claim 39, wherein said plurality of compounds are small molecules.

41. A method for inhibiting a bacterium, comprising the step of;
contacting said bacterium with a compound active on a target of a bacteriophage 44AHJD open reading frame 12 or 25 product, wherein said target or target site is uncharacterized.

42. The method of claim 41, wherein said compound is said protein or an active fragment thereof.

43. The method of claim 41, wherein said compound is a structural mimetic of said protein.

44. The method of claim 41, wherein said compound is a small molecule.

45. The method of claim 41, wherein said contacting is performed in vitro.

46. The method of claim 41, wherein said contacting is performed in vivo in an animal.

47. The method of claim 41, wherein said animal is a human.

48. The method of claim 41, wherein said contacting is carried out in vivo in a plant.

49. The method of claim 41, wherein said bacterium is pathogenic.

50. A method for treating a bacterial infection in an animal suffering from an infection, comprising administering to said animal a therapeutically effective amount of compound active on a target of a bacteriophage 44AHJD open reading frame 12 or 25 product in a bacterium involved in said infection,
wherein said target is an uncharacterized target or the compound is active at an uncharacterized target site.

51. The method of claim 50, wherein said compound is a small molecule.

52. The method of claim 50, wherein said compound is a peptidomimetic compound.

53. The method of claim 50, wherein said compound is a fragment of a bacteriophage inhibitor protein.

54. The method of claim 50, wherein said animal is a mammal.

55. The method of claim 54, wherein said mammal is a human.

56. A method for propylactically treating an animal at risk of an infection, comprising administering to said animal a prophylactically effective amount of a compound active on a target of a bacteriophage 44AHJD open reading frame 12 or 25 product,
wherein said target is an uncharacterized target or the site of action of said compound is an uncharacterized target site.

57. The method of claim 56, wherein said compound is a small molecule.

58. The method of claim 56, wherein said compound is a peptidomimetic compound.

59. The method of claim 56, wherein said compound is a fragment of a bacteriophage inhibitor protein.

60. The method of claim 56, wherein said animal is a mammal.

61. The method of claim 60, wherein said mammal is a human.

62. An antibacterial agent active on a target of a bacteriophage 44AHJD open reading frame 12 or 25 product, wherein said target is an uncharacterized target or said agent is active at a phage-specific site on said target.

63. The agent of claim 62, wherein said agent is a pepetidomimetic of a bacteriophage inhibitor polypeptide.

64. The agent of claim 62, wherein said agent is a small molecule.

65. The agent of claim 62, wherein said agent is a fragment of a bacteriophage inhibitor polypeptide.

66. The agent of claim 62, wherein said agent is active at a phage-specific site on said target.

67. A method of making an antibacterial agent, comprising the steps of:
identifying a target of a bacteriophage 44AHJD open reading frame 12 or 25 product;
screening a plurality of test compounds to identify a compound active on said target; and
synthesizing said compound in an amount sufficient to provide a therapeutic effect when administered to an organism infected by a bacterium naturally producing said target.

68. The method of claim 67, wherein said compound is a small molecule.

69. The method of claim 67, wherein said compound is a peptidomimetic compound.

70. The method of claim 67, wherein said compound is a fragment or derivative of a bacteriophage 44AHJD open reading frame product.

71. An antibody which binds a protein encoded by an open reading frame from Staphylococcus aureus bacteriophage 44AHJD.

72. The antibody of claim 71, wherein said antibody binds a protein which corresponds to a protein encoded by an open reading frame from Staphylococcus aureus bacteriophage 44AHJD.

73. A method for detecting a phage protein comprising the steps of, contacting said phage with an antibody,
wherein said antibody binds a protein encoded by an open reading frame from Staphylococcus aureus bacteriophage 44AHJD.

74. A method for detecting a virus comprising the steps of, contacting said virus with an antibody,
wherein said antibody binds a protein encoded by an open reading frame from Staphylococcus aureus bacteriophage 44AHJD.

75. The method of claim 74, wherein said virus is pathogenic to a mammal.

76. The method of claim 75, wherein said mammal is a human.

77. A method for determining the cellular and/or biochemical function of a bacterial target of a bacteriophage 44AHJD open reading frame product comprising;
contacting said bacterial target protein with a test compound;
determining whether said compound binds to or reduces the level of activity of said target protein, and
identifying homologous polypeptides and/or nucleic acid molecules to said target protein having known functions,
wherein binding of said compound with said target protein or a reduction of the level of activity of said protein is indicative that said compound is active on said target and wherein said target is uncharacterized.

78. The method of claim 77, wherein said contacting is carried out in vitro.

79. The method of claim 77, wherein said contacting is carried out in vivo in a cell.

80. The method of claim 77, wherein said compound is selected from the group consisting of a small molecule, a peptidomimetic compound, or a fragment or derivative of a bacteriophage inhibitor protein.

81. A method of screening for compounds that inhibit an S. aureus dnaN product, comprising
contacting said dnaN product with a bacteriophage 44AHJD ORF25 product or a fragment thereof and at least one test compound, and
determining whether any of said test compounds reduces the interaction between said dnaN product and said ORF25 product, wherein a reduction in said interaction is indicative that said test compound inhibits said dnaN product.

82. The method of claim 81, wherein said dnaN product has the amino acid sequence of SEQ ID NO:2.

83. The method of claim 81, wherein said determining comprises measuring the interaction between dnaN and ORF 25 product, wherein dnaN or ORF25 product is directly labeled.

84. The method of claim 83, wherein said dnaN product comprises an active portion, a mimetic, a corresponding isolated, enriched, or purified protein, or a homologous product.

85. The method of claim 83, wherein said dnaN or ORF25 product is indirectly labeled.

86. The method of claim 81, wherein said detecting comprises measurement by phage display.

87. The method of claim 81, wherein said detecting comprises measurement by surface plasmon resonance.

88. The method of claim 81, wherein said detecting comprises measurement by Fluorescence Resonance Energy Transfer.

89. The method of claim 81, wherein said detecting comprises measurement of fluorescence polarization changes.

90. The method of claim 81, wherein said detecting comprises a scintillation proximity assay.

91. The method of claim 81, wherein said detecting comprises a biosensor assay.

92. The method of claim 81, wherein said test compound is a small molecule, a peptidomimetic compound, or a fragment or derivative of a bacteriophage inhibitor protein.

93. The method of claim 91, wherein said bacteriophage inhibitor protein is from S. aureus bacteriophage AHJD 12 or 25.

94. The method of claim 81, wherein said test compound is a peptide.

95. The method of claim 94, wherein said peptide is an artificially synthesized peptide.

96. The method of claim 94, wherein said peptide is a peptide prepared in expression systems.

97. A method for inhibiting an S. aureus dnaN product, comprising
contacting said dnaN product with a bacteriophage 44AHJD 0RF25 product or fragment thereof at a concentration sufficient to inhibit said dnaN product.

98. The method of claim 97, wherein said contacting is in vitro.

99. The method of claim 97, wherein said contacting is in a cell.

100. The method of claim 97, wherein said contacting is in vivo.

101. The method of claim 97, wherein said contacting is in a mammal.

102. A method for inhibiting an S. aureus dnaN product, comprising
contacting said dnaN product with a structural mimetic of a bacteriophage 44AHJD ORF25 product or biologically active fragment at a concentration sufficient to inhibit said dnaN product.

103. The method of claim 102, wherein said contacting is in vitro.

104. The method of claim 102, wherein said contacting is in a cell.

105. The method of claim 102, wherein said contacting is in vivo.

106. The method of claim 102, wherein said contacting is in a mammal.

107. A pharmaceutical composition comprising
a pharmaceutically effective amount of a bacteriophage 44AHJD product or fragment thereof, and a pharmaceutically acceptable carrier.

108. A pharmaceutical composition comprising
a pharmaceutically effective amount of a structural mimetic of a bacteriophage 44AHJD product of fragment thereof, and a pharmaceutically acceptable carrier.

109. The pharmaceutical composition of claim 120, wherein said structural mimetic is a peptidomimetic.

110. The pharmaceutical composition of claim 120, wherein said structural mimetic is a synthetic mimetic.
